(19) United States
(12) Patent Application Publication
(10) Pub. No.: US 2025/0122223 A1
(43) Pub. Date: Apr. 17, 2025

(54) HETEROCYCLIC COMPOUNDS AS UBIQUITIN SPECIFIC PROTEASE 7 INHIBITORS

(71) Applicant: ALMAC DISCOVERY LIMITED, Craigavon (GB)

(72) Inventors: Frank BURKAMP, Craigavon (GB); Adam Piotr TREDER, Craigavon (GB); Colin O'DOWD, Craigavon (GB); Lauren Emma PROCTOR, Craigavon (GB); James Samuel Shane ROUNTREE, Craigavon (GB); Matthew HELM, Craigavon (GB); Aaron CRANSTON, Craigavon (GB)

(21) Appl. No.: 18/729,403

(22) PCT Filed: Jan. 20, 2023
(86) PCT No.: PCT/EP2023/051447

§ 371 Date: Jul. 16, 2024

(30) Foreign Application Priority Data
Jan. 21, 2022 (GB) 2200753.8

(51) Int. Cl. C07D 519/00; A61K 31/5377; A61K 31/538; A61K 31/5386; A61K 45/06; A61P 35/00; C07D 487/04

(52) U.S. Cl. CPC C07D 519/00; A61K 31/5377; A61K 31/538; A61K 31/5386; A61K 45/06; A61P 35/00; C07D 487/04

(57) ABSTRACT

The present invention concerns inhibitors of ubiquitin specific protease 7 (USP7), and methods of use thereof. Specifically, the invention provides compounds according to formula (I), including stereoisomers, pharmaceutically acceptable salt or solvate thereof:

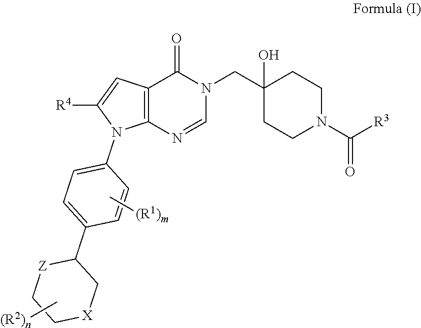

This application claims priority from GB application no. 2200753.8 filed 21 Jan. 2022, the contents and elements of which are herein incorporated by reference for all purposes.

### FIELD OF THE INVENTION

The present invention concerns inhibitors of ubiquitin specific protease 7 (USP7), and methods of use thereof.

### BACKGROUND OF THE INVENTION

Over the past three decades, protein ubiquitination has emerged as an important post-translational modification with roles in a plethora of cellular processes including: proteolysis, gene expression, DNA repair, immune response, metabolism and cell-cycle regulation. Dysregulation of the ubiquitin proteasome system (UPS) has also been implicated in the pathogenesis of multiple human diseases including (but not limited to): cancer (Hoeller et al., Nat. Rev. Cancer (2006), 6, 776-788), viral infection (Gao et al., Can. J. Physiol. Pharmacol. (2006), 84, 5-14), metabolic or neurodegenerative disorders (Loosdregt et al., Immunity (2013), 39, 259-271; Rubinsztein et al., Nature (2006), 443, 780-786) as well as immune and inflammatory-related medical conditions (Wang et al., Cell Mol. Immunol. (2006), 3, 255-261; Corn J. et al., Nat. Struct. Mol. Biol. (2014), 21, 297-300; Nicholson et al., Cell Biochem. Biophys. (2011), 60, 61-68).

The established and growing connections between USP7 and numerous proteins involved in human disease indicate that small molecule inhibitors of USP7 may have broad therapeutic applications beneficial to human health. Small molecule USP7 inhibitors have been reported in the following patent applications: US 2008/0103149 A1, WO 2010/114881 A1, WO 2010/081783 A1, WO 2011/086178 A1, WO 2013/030218 A1, EP 2565186 A1, EP 1749822 A1, WO 2016/109515 A1, WO 2016/109480 A1, WO 2016/126929 A1, WO 2016/126926 A1, WO 2016/126935 A1, WO 2016/150800 A1, WO2017/158381, WO2017/158388, WO2018/073602, and WO2021/161047.

There is nevertheless a need for improved USP7 inhibitors, for instance those having improved inhibitory activity, having improved solubility, improved in vivo stability, improved pharmacokinetic properties, or a combination of some or all of these properties. The present application addresses this need.

### SUMMARY OF THE INVENTION

The compounds described herein are able to selectively inhibit USP7 activity. The compounds provided herein may therefore be suitable for the treatment and prevention of, for example, cancer and neoplastic conditions. The compounds may be used as monotherapy or as combination therapy with radiation and/or additional therapeutic agents.

Therefore, in a first aspect is provided compounds according to formula (I) compound according to formula (I), including stereoisomers thereof:

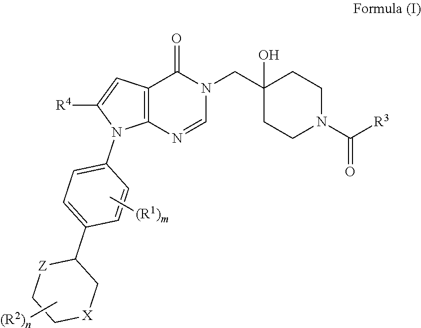

wherein:
“m” is 0, 1 or 2, and wherein each R¹ is independently selected from: halo; C₁-C₆ alkyl optionally substituted with halo; and C₃-C₆ cycloalkyl;
“n” is 0, 1 or 2, and wherein each R² is independently selected from optionally substituted C₁-C₆ alkyl, optionally substituted C₃-C₆ cycloalkyl, wherein each of the one or more optional substituents are independently selected from C₁-C₆ alkyl, and halo,
or wherein “n” is 2 and each R² together with the carbon ring atom or atoms to which they are attached combine to form a C₃-C₆ fused cycloalkyl ring, a C₃-C₆ spirocyclic cycloalkyl ring, or C₃-C₄ bridge;
R³ is selected from optionally substituted C₁-C₆ alkyl, optionally substituted C₃-C₆ cycloalkyl, optionally substituted phenyl, optionally substituted C₇-C₁₀ arylalkyl, optionally substituted C₈-C₁₀ arylalkenyl, and optionally substituted C₃-C₆ heteroaryl; wherein each of the one or more optional substituents is independently selected from: halo; C₁-C₆ alkyl optionally substituted with halo; and C₃-C₆ cycloalkyl optionally substituted with methyl or CF₃;
R⁴ is H, Cl, C₁-C₆ alkyl;
wherein one of X and Z is 0 and one of X and Z is NR′, such that the ring comprising X and Z is a morpholine ring, wherein R′ is H or C₁-C₆ alkyl, optionally H;
or a pharmaceutically acceptable salt or solvate thereof.

In a particular embodiment are provided compounds according to formula (Ia), including stereoisomers thereof:

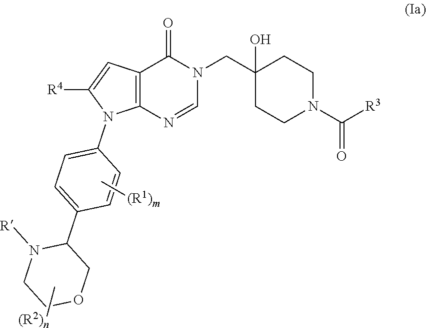

wherein:
“m”, “n”, R¹-R⁴ and R′ are as defined in relation to Formula (I),
or a pharmaceutically acceptable salt or solvate thereof.

Compounds according to the invention are particularly advantageous as they exhibit improved properties compared to USP7 inhibitors described in the art.

WO2018/073602 describes a USP7 inhibitor having the structure:

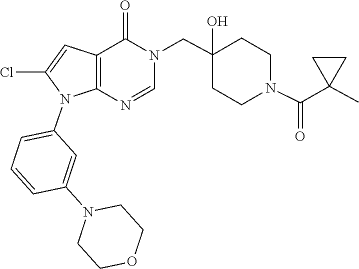 [SMILES: CC1(C(=O)N2CCC(O)(Cn3cnc4c(cc(Cl)n4-c4cccc(N5CCOCC5)c4)c3=O)CC2)CC1]

### (Comparative Example—Example 226 from WO2018/073602)

The compounds of the present invention differ from this comparative example at least in the fact that the pendant morpholinyl ring in Formula (I) is C-linked rather than N-inked, and is attached to the phenyl at the 4 (para) position rather than the 3 (meta) position.

Compared to the comparative Example 226, compounds according to the present invention exhibit improved inhibition of USP7. This is particularly the case when USP7 inhibition is determined using engagement of USP7 as a cellular target (Table 1).

TABLE 1

| | | Cellular target engagement: |
|---|---|---|
| | Biochemical: | HEK293 Stable clone M6C5 |
| | USP7 IC₅₀ (μM) | USP7 IC₅₀ (μM) |
| Example 8 | 0.077 | 0.066 |
| Example 14 | 0.018 | 0.040 |
| Example 21 | 0.11 | 0.18 |
| Example 32 | 0.048 | 0.13 |
| Example 62 | 0.038 | 0.10 |
| Example 66 | 0.063 | 0.074 |
| Example 67 | 0.053 | 0.072 |
| Example 82 | 0.0060 | 0.0094 |
| Example 83 | 0.028 | 0.076 |
| Comparative Example | 0.19 | >50 |
| (Ex 226 from | | |
| WO2018073602) | | |

Compounds of the invention also demonstrate improved solubility and microsomal stability compared to USP7 compounds in the prior art (Table 2).

TABLE 2

| | Kinetic | Liver Microsomes: | | | |
|---|---|---|---|---|---|
| | Solubility | CLₕₑₚ (mL/min/kg) | | | |
| | (μM) | Human | Rat | Mouse | Dog |
| Example 21 | 203 | 3.2 | 6.5 | 19.1 | 4.5 |
| Example 32 | 197 | 5.8 | 6.5 | 11.5 | 4.5 |
| Comparative Example | 151 | 14.5 | 27 | 63.9 | 22.3 |
| (Ex 226 from | | | | | |
| WO2018073602) | | | | | |

The present invention thus provides novel USP7 inhibitor compounds with improved properties compared to those in the prior art.

The invention includes the combination of all aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows electron density maps derived from the crystal structure determination of USP7 in complex with an inhibitor according to the present invention, as described in the Examples below. FIG. 1(a) is the initial Fo-Fc difference electron density map of the model (contoured at 3.0σ) resulting from refinement of the initial model prior to modelling of the compound with BUSTER. Shown is the region of the compound binding site in chain B. For clarity, the final refined coordinates are also shown; FIG. 1(b) is the final 2Fo-Fc electron density map (contoured at 1.5σ) resulting from refinement of the final model with BUSTER. Shown is the region of the compound binding site.

FIG. 2 shows the overall structure of USP7, in complex with an inhibitor according to the present invention.

### DETAILED DESCRIPTION

Aspects and embodiments of the present invention will now be discussed with reference to the accompanying figures. Further aspects and embodiments will be apparent to those skilled in the art. All documents mentioned in this text are incorporated herein by reference.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

As used in the specification and the appended claims, unless specified to the contrary, the following terms have the meaning indicated:

The term “alkyl group” (alone or in combination with another term(s)) means a straight- or branched-chain saturated hydrocarbon substituent typically containing 1 to 15 carbon atoms, such as 1 to 10, 1 to 8, 1 to 6, or 1 to 4 carbon atoms. A “Cₐ alkyl” group refers to an aliphatic group containing n carbon atoms. For example, a C₁-C₁₀ alkyl group contains 1, 2, 3, 4, 5, 6, 7, 8, 9 or 10 carbon atoms. Attachment to the alkyl group occurs through a carbon atom. Examples of such substituents include methyl, ethyl, n-propyl, isopropyl, n-butyl, isobutyl, sec-butyl, tert-butyl, pentyl (branched or unbranched), hexyl (branched or unbranched), heptyl (branched or unbranched), octyl (branched or unbranched), nonyl (branched or unbranched), and decyl (branched or unbranched).

The term “alkenyl group” (alone or in combination with another term(s)) means a straight- or branched-chain hydrocarbon substituent containing one or more double bonds and typically 2 to 15 carbon atoms; such as 2 to 10, 2 to 8, 2 to 6 or 2 to 4 carbon atoms. Examples of such substituents include ethenyl (vinyl), 1-propenyl, 3-propenyl, 1,4-pentadienyl, 1,4-butadienyl, 1-butenyl, 2-butenyl, 3-butenyl, pentenyl and hexenyl.

The term “alkynyl group” (alone or in combination with another term(s)) means a straight- or branched-chain hydrocarbon substituent containing one or more triple bonds and typically 2 to 15 carbon atoms; such as 2 to 10, 2 to 8, 2 to 6 or 2 to 4 carbon atoms. Examples of such substituents include ethynyl, 1-propynyl, 3-propynyl, 1-butynyl, 3-butynyl and 4-butynyl.

The term “cycloalkyl group” (alone or in combination with another term(s)) means a saturated cyclic hydrocarbon substituent containing 3 to 14 carbon ring atoms. A cycloalkyl may be a single carbon ring, which typically contains 3 to 8 carbon ring atoms and more typically 3 to 6 ring atoms. It is understood that attachment to a cycloalkyl group is via a ring atom of the cycloalkyl group. Examples of single-ring cycloalkyls include cyclopropyl, cyclobutyl, cyclopentyl, and cyclohexyl. A cycloalkyl may alternatively be polycyclic or contain more than one ring. Polycyclic cycloalkyls include bridged, fused, and spirocyclic cycloalkyls.

The term “aryl group” (alone or in combination with another term(s)) means an aromatic carbocyclyl containing from 6 to 14 carbon ring atoms, optionally 6 to 8, 6 to 7, optionally carbon ring atoms. A “Cₙ aryl” group refers to an aromatic group containing n carbon atoms. For example, a C₆-C₁₀ aryl group contains 6, 7, 8, 9 or 10 carbon atoms. Attachment to the aryl group occurs through a carbon atom. An aryl group may be monocyclic or polycyclic (i.e. may contain more than one ring). In the case of polycyclic aromatic rings, only one ring in the polycyclic system is required to be unsaturated while the remaining ring(s) may be saturated, partially saturated or unsaturated. Attachment to the aryl group occurs through a carbon atom contained in the ring. Examples of aryl groups include phenyl, naphthyl, acridinyl, indenyl, indanyl, and tetrahydronapthyl. Preferably an aryl group is phenyl.

The term “arylalkyl” refers to an aryl substituent attached via an alkyl chain. Examples of an arylalkyl substituent include phenylethyl/ethylbenzyl, where the ethyl chain links a phenyl group to the point of attachment. In a “Cₙ” arylalkyl, Cₙ includes the carbon atoms in the alkyl chain and in the aryl group. For example, ethylbenzene is a C₈ arylalkyl.

The term “arylalkenyl” refers to an aryl substituent attached via an alkenyl chain. Examples of an arylalkenyl substituent include phenylethenyl/ethenylbenzyl, where the ethenyl chain links to a phenyl group to the point of attachment. In a “Cₙ” arylalkyl, Cₙ includes the carbon atoms in the alkyl chain and in the aryl group. For example, phenylethenyl is a C₈ arylalkenyl.

The term “heteroaryl group” (alone or in combination with another term(s)) means an aromatic heterocyclyl containing from 5 to 14 ring atoms. A “Cₙ heteroaryl” group refers to an aromatic group containing n carbon atoms and at least one heteroatom. For example, a C₂-C₁₀ aryl group contains 2, 3, 4, 5, 6, 7, 8, 9 or 10 carbon atoms in addition to at least one heteroatom. Attachment to the heteroaryl group occurs through a carbon atom or through a heteroatom. A heteroaryl group may be monocyclic or polycyclic. A heteroaryl may be a single ring or 2 or 3 fused rings. Examples of monocyclic heteroaryl groups include 6-membered rings such as pyridyl, pyrazinyl, pyrimidinyl, pyridazinyl, and 1,3,5-, 1,2,4- or 1,2,3-triazinyl; 5-membered rings such as imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl. Polycyclic heteroaryl groups may be 2 or 3 fused rings. Examples of polycyclic heteroaryl groups include 6/5-membered fused ring groups such as benzothiofuranyl, benzisoxazolyl, benzoxazolyl, and purinyl; and 6/6-membered fused ring groups such as benzopyranyl, quinolinyl, isoquinolinyl, cinnolinyl, quinazolinyl, and benzoxazinyl. In the case of polycyclic heteroaryl groups, only one ring in the polycyclic system is required to be unsaturated while the remaining ring(s) may be saturated, partially saturated or unsaturated.

The term “amino group” refers to the —NR′R″ group. The amino group can be unsubstituted or substituted. In an unsubstituted amino group, R′ and R″ are hydrogen. In a substituted amino group R′ and R″ each independently may be, but are not limited to, hydrogen, an alkyl, heteroalkyl, cycloalkyl, heterocycloalkyl, alkylcycloalkyl, alkylheterocycloalkyl, alkoxy, sulfonyl, alkenyl, alkanoyl, aryl, arylalkyl, or a heteroaryl group, provided R′ and R″ are not both hydrogen. In a substituted amino group R′ and R″ may cyclise to form a heterocyclic group including the nitrogen to which they are attached (e.g. a pyrrolidine group). The heterocyclic group formed by R′ and R″ may optionally include additional heteroatoms, for example nitrogen or oxygen (e.g. the NR′R″ group may form morpholine or piperazine). The heterocyclic group formed by R′ and R″ may be monocyclic, polycyclic (e.g. bicyclic), spirocyclic or a bridged ring group (e.g. a diazabicyclo[3.2.1]octane group). Such a cyclic amino group may be optionally substituted, e.g. with an amino group, a methyl group, a hydroxyl group or an oxo group.

The term “hydroxyl” refers to an —OH group.

The term “oxo group” refers to the (═O) group, i.e. a substituent oxygen atom connected to another atom by a double bond. For example, a carbonyl group (—C(═O)—) is a carbon atom connected by a double bond to an oxygen atom, i.e. an oxo group attached to a carbon atom.

The term “halo group” refers to a group selected from chlorine, fluorine, bromine and iodine. Preferably, the halo group is selected from chlorine and fluorine.

The term “optionally substituted” means the group may be substituted with one or more substituents, which can be the same or different, or the group may have no substituents. The term “optionally monosubstituted” means the group may have a single substituent or may be unsubstituted.

A substituent can be attached through a carbon atom and/or a heteroatom in the alkyl, alkenyl, cycloalkyl, aryl, or group. The term “substituent” (or “radical”) includes but is not limited to alkyl, substituted alkyl, arylalkyl, substituted arylalkyl, alkenyl, substituted alkenyl, alkynyl, substituted alkynyl, halo, hydroxyl, cyano, amino, amido, cycloalkyl, substituted cycloalkyl, aryl, substituted aryl, heteroaryl, substituted heteroaryl, nitro, thio, alkanoyl, carboxyl, alkoxycarbonyl, and oxo.

In certain aspects, the substituent is alkyl, substituted alkyl, arylalkyl, substituted arylalkyl, alkenyl, substituted alkenyl, alkynyl, substituted alkynyl, halo, hydroxyl, cyano, amino, amido, cycloalkyl, substituted cycloalkyl, aryl, substituted aryl, heteroaryl, substituted heteroaryl, nitro, thio, alkanoyl, carboxyl, alkoxycarbonyl, and oxo.

In certain embodiments, each of the one or more substituents of any optionally substituted group is independently selected from OH, F, Cl, Br, I, CN, C₁-C₆ alkyl, CF₃, CHF₂, CH₂F, CH₂OH, C(O)CH₃, CH₂NHC(O)OCH₂CH₃, C₂-C₆ alkenyl, C₂-C₆ alkynyl, C₃-C₆ cycloalkyl, C₁-C₆ alkoxy, amino, C₁-C₆ alkylamine, C₅-C₆ aryl, C₃-C₆ heteroaryl, benzyl, oxo and amide or two adjacent substituents may together constitute a ring.

If a group is substituted with a further optionally substituted group, it is understood that the first substituent may itself be either unsubstituted or substituted.

For completeness, it is also noted that certain chemical formulae used herein define delocalized systems. This definition is known in the art as a definition of aromaticity and may indicate the presence of, for example, a planar mono-, di- or tri-cyclic system that contains (4n+2) electrons where n is an integer. In other words, these systems may display Hückel aromaticity.

In whatever aspect, the compounds of the present invention may possess some aspect of stereochemistry. For example, the compounds may possess chiral centres and/or planes and/or axes of symmetry. Unless otherwise specified, compounds according to the invention encompass all such stereoisomers. As such, the compounds may be provided as single stereoisomers, single diastereomers, mixtures of stereoisomers or as racemic mixtures, unless otherwise specified. Stereoisomers are known in the art to be molecules that have the same molecular formula and sequence of bonded atoms, but which differ in their spatial orientations of their atoms and/or groups.

In addition, the compounds of the present invention may exhibit tautomerism. Each tautomeric form is intended to fall within the scope of the invention.

In addition, the compounds of the present invention may be provided as a pro-drug. Pro-drugs are transformed, generally in vivo, from one form to the active forms of the drugs described herein.

In addition, it will be understood that the elements described herein may be the common isotope or an isotope other than the common isotope. For example, a hydrogen atom may be ¹H, ²H (deuterium) or ³H (tritium).

In addition, the compounds of the present invention may be provided in the form of their pharmaceutically acceptable salts or as co-crystals.

The term “pharmaceutically acceptable salt” refers to ionic compounds formed by the addition of an acid to a base. The term refers to such salts that are considered in the art as being suitable for use in contact with a patient, for example in vivo and pharmaceutically acceptable salts are generally chosen for their non-toxic, non-irritant characteristics.

The term “co-crystal” refers to a multi-component molecular crystal, which may comprise non-ionic interactions.

Pharmaceutically acceptable salts and co-crystals may be prepared by ion exchange chromatography or by reacting the free base or acidic form of a compound with stoichiometric amounts or with an excess of the desired salt-forming inorganic or organic acid or base in one or more suitable solvents, or by mixing the compound with another pharmaceutically acceptable compound capable of forming a co-crystal.

Salts known in the art to be generally suitable for use in contact with a patient include salts derived from inorganic and/or organic acids, including the hydrobromide, hydrochloride, sulfate, bisulfate, nitrate, acetate, oxalate, oleate, palmitate, stearate, laurate, benzoate, lactate, phosphate, tosylate, citrate, maleate, fumarate, succinate and tartrate. These may include cations based on the alkali and alkaline earth metals, such as sodium, potassium, calcium and magnesium, as well as ammonium, tetramethylammonium, tetraethylammonium. Further reference is made to the number of literature sources that survey suitable pharmaceutically acceptable salts, for example the handbook of pharmaceutical salts published by IUPAC.

In addition, the compounds of the present invention may sometimes exist as zwitterions, which are considered as part of the invention.

Accordingly, in a first aspect of the invention are provided compounds according to formula (I), including stereoisomers thereof:

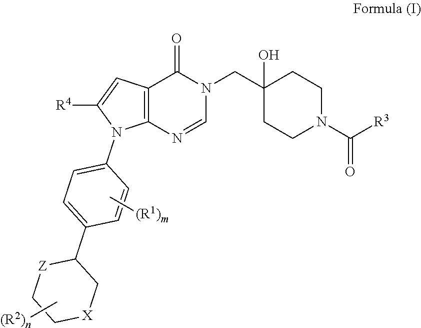

wherein:
“m” is 0, 1 or 2, and wherein each R¹ is independently selected from: halo; C₁-C₆ alkyl optionally substituted with halo; and C₃-C₆ cycloalkyl;
“n” is 0, 1 or 2, and wherein each R² is independently selected from optionally substituted C₁-C₆ alkyl, optionally substituted C₃-C₆ cycloalkyl, wherein each of the one or more optional substituents are independently selected from C₁-C₆ alkyl, and halo,
or wherein “n” is 2 and each R₂ together with the carbon ring atom or atoms to which they are attached combine to form a C₃-C₆ fused cycloalkyl ring, a C₃-C₆ spirocyclic cycloalkyl ring, or C₃-C₄ bridge;
R′ is H or C₁-C₆ alkyl, optionally H;
R³ is selected from optionally substituted C₁-C₆ alkyl, optionally substituted C₃-C₆ cycloalkyl, optionally substituted phenyl, optionally substituted C₇-C₁₀ arylalkyl, optionally substituted C₈-C₁₀ arylalkenyl, and optionally substituted C₃-C₆ heteroaryl; wherein each of the one or more optional substituents is independently selected from: halo; C₁-C₆ alkyl optionally substituted with halo; and C₃-C₆ cycloalkyl optionally substituted with methyl or CF₃;
R⁴ is H, Cl, C₁-C₆ alkyl;
wherein one of X and Z is O and one of X and Z is NR′, such that the ring comprising X and Z is a morpholine ring, wherein R′ is H or C₁-C₆ alkyl, optionally H;
or pharmaceutically acceptable salts or solvates thereof.

In accordance with Formula (I), each R¹ and R², when present, may be attached to any of the carbon atoms of their respective rings.

In accordance with Formula (I), where “m” is 2, each R¹ substituent is independently selected from the recited options and thus may be the same or different from each other.

Similarly, where “n” is 2, each R² substituent is independently selected from the recited options and thus may be the same as or different from each other.

In a preferred embodiment is provided compounds according to formula (Ia), including stereoisomers thereof:

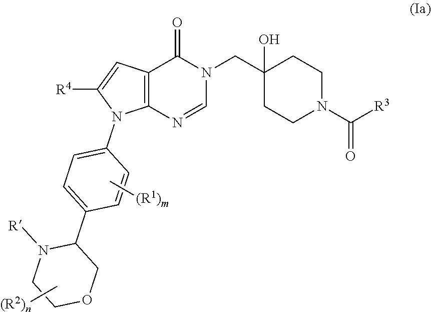

wherein:
“m”, “n”, R¹-R⁴ and R′ are as defined above in relation to Formula (I),
or pharmaceutically acceptable salts or solvates thereof.

In some embodiments of formula (I) or (Ia), R′ is preferably H.

In an alternative embodiment of formula (I), X is NR′ and Z is O.

In certain preferred embodiments of formula (I) or (Ia), “m” is zero or 1. In certain such embodiments “m” is zero—i.e. the phenyl ring is unsubstituted. In certain alternative embodiments, “m” is 1—i.e. the phenyl ring is monosubstituted.

In those embodiments where “m” is 1, R¹ is selected from: halo; C₁-C₆ alkyl optionally substituted with halo; and C₃-C₆ cycloalkyl. In certain such embodiments, R¹ is selected from fluoro, chloro, C₃-C₆ cycloalkyl, and C₁-C₆ alkyl optionally substituted with fluoro, for example methyl or CF₃. In certain preferred embodiments, R¹ is selected from: Cl; F; and methyl optionally substituted with F, optionally CF₃. In certain preferred embodiments, R¹ is Cl or F.

In certain preferred embodiments, R¹ is methyl optionally substituted with F. In certain preferred embodiments, R¹ is CF₃.

In certain preferred embodiments, R¹, when present as a monosubstituent, is at position 3 of the phenyl ring. That is, preferably the compounds of the invention are according to formula

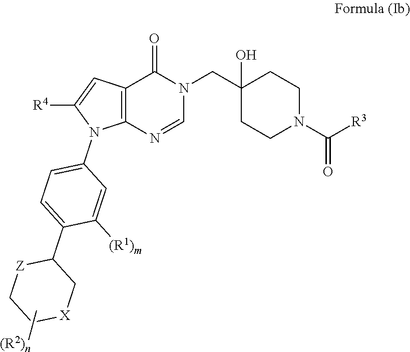

wherein “m” is 0 or 1, and the remaining integers are defined as in relation to formula (I).

In certain embodiments, preferred features of formulae (Ia) and (Ib) are combined such that Z is NH, X is O, and R¹, when present as a monosubstituent, is at position 3 of the phenyl ring. That is, certain preferred compounds of the invention are compounds according to formula (Ic):

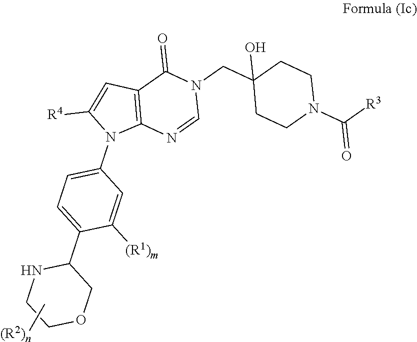

wherein “m” is 0 or 1, and the remaining integers are as defined in relation to formula (I).

In certain preferred embodiments of all aspects (including formulae (I), (Ia), (Ib), (Ic) and (Id)), “n” is 1 or 2. Alternatively, in certain embodiments “n” is zero and the morpholine ring has no R² substituent. In certain preferred embodiments, “n” is 1. In certain preferred embodiments, “n” is 2.

In certain embodiments, when “n” is 2, each R² substituent is at the same position of the morpholinyl ring. Alternatively, when “n” is 2, each R² substituent may be at a different position of the morpholinyl ring.

As will be appreciated by the person skilled in the art, and in accordance with IUPAC convention, the numbering of the ring atoms of a morpholine ring always starts at the oxygen atom. In the numbering used herein, the direction of numbering is such that the point of attachment of the morpholinyl ring to the phenyl ring has the lowest possible number.

Therefore, for compounds where Z is NR′ and X is O, the ring atoms are numbered as shown below:

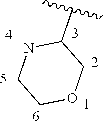

whereas, for compounds where Z is O and X is NR′, the ring atoms are numbered as shown below:

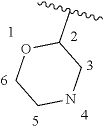 [SMILES: *C1CNCCO1]

References herein to substituents at certain positions of the morpholinyl ring may be construed accordingly.

In certain preferred embodiments, when “n” is 1 or 2, at least one R² substituent is at the 6 position of the morpholinyl ring. In preferred such embodiments, each R² substituent is at the 6 position of the morpholinyl ring.

In certain embodiments, when “n” is 1 or 2, at least one R² group is at the 2 position of the morpholinyl ring. In certain preferred such embodiments, each R² substituent is at the 2 position of the morpholinyl ring.

In certain embodiments, when “n” is 1 or 2, at least one R² group is at the 5 position of the morpholinyl ring. In certain preferred embodiments, each R² substituent is at the 5 position of the morpholinyl ring.

In certain embodiments “n” is two and each R² is attached to the same carbon ring atom. In certain alternative embodiments, “n” is two and each R² is attached to different carbon ring atoms, for example adjacent carbon ring atoms.

In certain preferred embodiments of all aspects, R² is independently selected from: methyl optionally substituted with F; ethyl; and cyclopropyl. In certain such embodiments, R² is selected from methyl, CHF₂, CF₃, ethyl, and cyclopropyl. In certain embodiments, R² is methyl. In certain embodiments, “n” is two and each R² is methyl. In certain such embodiments, each methyl group is attached to the same carbon ring atom. In certain such embodiments, each methyl group is attached at the 6-position of the morpholine ring. Alternatively, each methyl group can be attached to different carbon ring atoms.

Alternatively, in certain embodiments, “n” is one and R² is methyl.

In certain embodiments of all aspects (including formulae (I), (Ia), (Ib), (Ic) and (Id)) the presence of an R² substituent results in a chiral centre at the morpholinyl ring carbon to which it is attached. In certain preferred such embodiments, the R² substituent is in the R position. In certain alternative and also preferred embodiments, the R² substituent is in the S position.

For example, in certain embodiments of formula (Ic) wherein n is 1, and R² is at the 6-position of the morpholine ring, the invention encompasses compounds according to any of formulae (Ic)^{I}, (Ic)ⁱⁱ, (Ic)ⁱⁱⁱ, or (Ic)^{iv}:

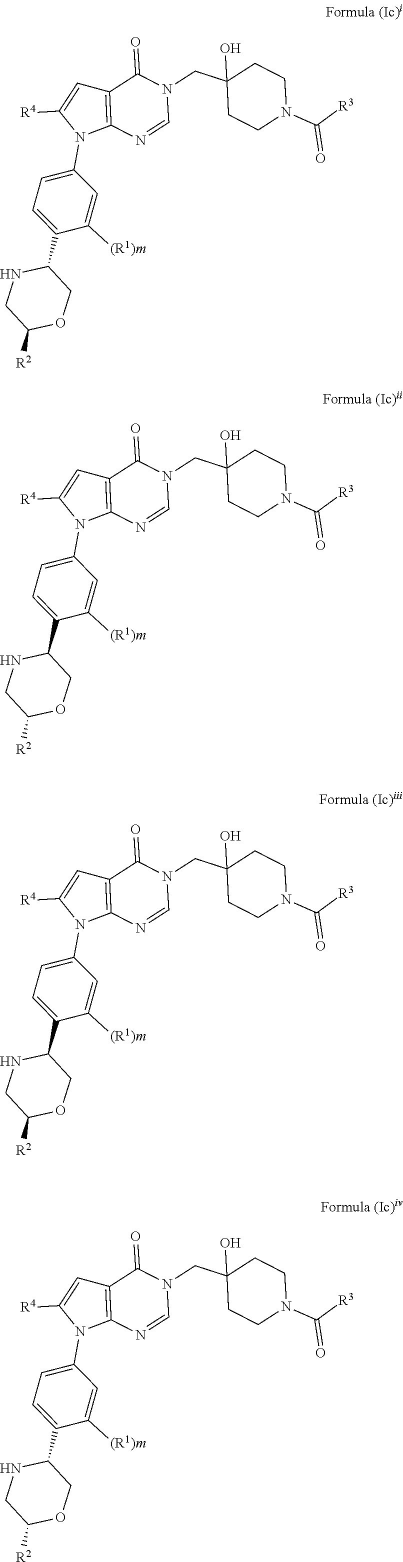

wherein m, R¹, R², R³ and R⁴ are as defined in relation to formula (Ic); with R² preferably being independently selected from: methyl, CHF₂, CF₃, ethyl, and cyclopropyl. In some such embodiments, R² is preferably methyl, ethyl or cyclopropyl. In some such embodiments, R² is methyl. In some such embodiments, R² is ethyl.

In certain embodiments, “n” is 2 and each R² together with the carbon ring atom or atoms to which they are attached combine to form a C₃-C₆ spirocyclic cycloalkyl ring. In certain preferred embodiments, “n” is 2 and each R² combine to form a C₃ or C₄ spirocyclic cycloalkyl ring together with the carbon ring atom to which they are attached. In certain preferred such embodiments, “n” is 2 and each R² combine to form:

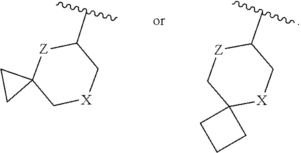

In certain such embodiments, Z is NR′ and X is O. Alternatively, X is NR′ and Z is O.

In certain preferred embodiments, “n” is 2 and each R² together with the carbon ring atoms to which they are attached combine to form a C₃-C₆ fused cycloalkyl ring.

In certain such embodiments, the two R² substituents are on adjacent carbon ring atoms.

In certain embodiments, “n” is 2 and each R² combine to form a C₃, C₅ or C₆ fused cycloalkyl ring, together with the carbon ring atoms to which they are attached. In certain such embodiments, each R² combine to form a C₃ fused cycloalkyl ring. In certain such embodiments, each R² combine to form a C₅ fused cycloalkyl ring. In certain such embodiments, each R² combine to form a C₆ fused cycloalkyl ring.

In certain embodiments, “n” is 2 and each R² combine to form:

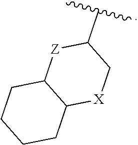

In certain such embodiments, Z is NR′ and X is O. Alternatively, X is NR′ and Z is O.

In certain embodiments, “n” is 2 and each R² combine to form:

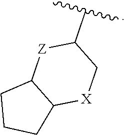

In certain such embodiments, Z is NR′ and X is O. Alternatively, X is NR′ and Z is O.

In certain embodiments, “n” is 2 and each R² combine to form:

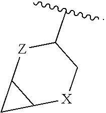

In certain such embodiments, Z is NR′ and X is O. Alternatively, X is NR′ and Z is O.

In certain embodiments, “n” is 2 and each R² combine together with the carbon ring atoms to which they are attached to form a C₃ or C₄ bridged ring. In certain embodiments, “n” is 2 and each R² combine together with the carbon ring atoms to which they are attached to form a C₄ bridged ring.

In certain embodiments, “n” is 2 and each R² combine together with the carbon ring atoms to which they are attached to form:

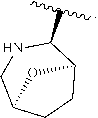 [SMILES: *[C@@H]1NC[C@@H]2CC[C@H]1O2]

In accordance with all aspects (including formulae (I), (Ia), (Ib), (Ic) and (Id), R³ is selected from optionally substituted C₁-C₆ alkyl, optionally substituted C₃-C₆ cycloalkyl, optionally substituted phenyl, optionally substituted C₇-C₁₀ arylalkyl, optionally substituted C₈-C₁₀ arylalkenyl, and optionally substituted C₃-C₆ heteroaryl; wherein each of the one or more optional substituents is independently selected from: halo; C₁-C₆ alkyl optionally substituted with halo; and C₃-C₆ cycloalkyl optionally substituted with methyl or CF₃.

In some embodiments (including of formulae (I), (Ia), (Ib), (Ic) and (Id)), R³ is selected from optionally substituted C₃-C₆ cycloalkyl, optionally substituted phenyl, optionally substituted C₇-C₁₀ arylalkyl, optionally substituted C₈-C₁₀ arylalkenyl, and optionally substituted C₃-C₆ heteroaryl; wherein each of the one or more optional substituents is independently selected from: halo; C₁-C₆ alkyl optionally substituted with halo; and C₃-C₆ cycloalkyl optionally substituted with methyl or CF₃.

In certain embodiments, R³ is optionally substituted C₃-C₆ cycloalkyl. In certain such embodiments, each of the one or more optional substituents is independently selected from: halo; C₁-C₂ alkyl optionally substituted with F; and C₃-C₆ cycloalkyl.

In certain embodiments, R³ is optionally substituted cyclopropyl. In certain such embodiments, each of the one or more optional substituents is independently selected from methyl, CHF₂, CF₃, and cyclopropyl. In certain embodiments, R³ is optionally substituted cyclopropyl, wherein one of the one or more optional substituents is methyl. In certain embodiments, R³ is optionally substituted cyclopropyl, wherein one of the one or more optional substituents is CHF₂. In certain embodiments, R³ is optionally substituted cyclopropyl, wherein one of the one or more optional substituents is CF₃. In certain embodiments, R³ is optionally substituted cyclopropyl, wherein one of the one or more optional substituents is cyclopropyl.

In certain embodiments, R³ is an optionally substituted cyclopropyl group of general formula:

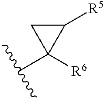

wherein R⁵ and R⁶ are each independently selected from hydrogen, halo; C₁-C₂ alkyl optionally substituted with F; and C₃-C₆ cycloalkyl.

That is, in some embodiments, the compounds of the invention may be according to formula (Id):

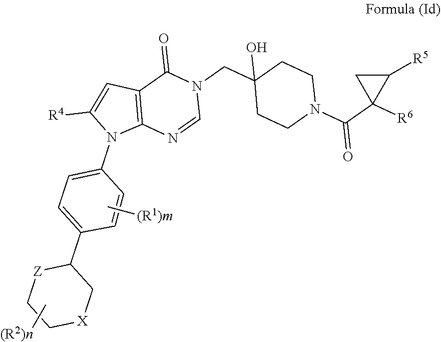

wherein R⁵ and R⁶ are as now defined and the remaining integers are as defined in relation to formulae (I), (Ia), (Ib) or (Ic).

R⁵ and R⁶ are substituents as defined above and are each independently selected from hydrogen, halo; C₁-C₂ alkyl optionally substituted with F; and C₃-C₆ cycloalkyl. In some embodiments, R⁵ and R⁶ are each independently selected from hydrogen, methyl, CHF₂, CF₃ and cyclopropyl.

In some embodiments, R⁵ is selected from hydrogen, methyl, CHF₂, and CF₃. In some embodiments, R⁵ is selected from hydrogen, methyl and CF₃. In some embodiments, R⁵ is selected from hydrogen and CF₃. In some embodiments, R⁵ is hydrogen. In some embodiments, R⁵ is CF₃.

In some embodiments, R⁶ is selected from hydrogen, methyl, CHF₂, CF₃ and cyclopropyl. In some embodiments, R⁶ is selected from methyl and cyclopropyl. In some embodiments, R⁶ is methyl.

In compounds of formula (Id) the presence of the R⁵ and/or R⁶ substituents may result in a chiral centre at the relevant cyclopropyl ring carbon. In certain such embodiments, the R⁵ substituent is in the R configuration. In certain alternative embodiments, the R⁵ substituent is in the S configuration. In certain such embodiments, the R⁶ substituent is in the R configuration. In certain alternative embodiments, the R⁶ substituent is in the S configuration.

For example, in certain embodiments of formula (Id) the compound may be a compound according to any one of formulae (Id)ⁱ, (Id)ⁱⁱ, (Id)ⁱⁱⁱ or (Id)^{iv}:

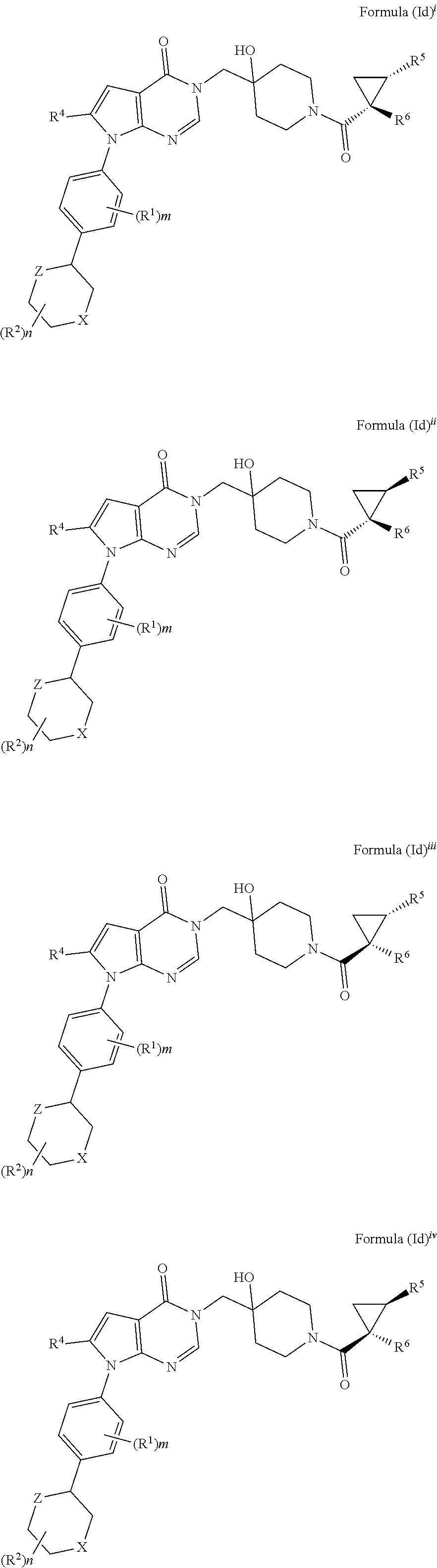

In certain other embodiments (i.e. of formula (I), (Ia), (Ib) or (Ic)) R³ is optionally substituted phenyl. In certain such embodiments, each of the one or more optional substituents is independently selected from: halo; C₁-C₂ alkyl optionally substituted with F; and C₃-C₆ cycloalkyl optionally substituted with methyl or CF₃.

In certain embodiments R³ is optionally substituted phenyl, wherein each of the one or more optional substituents is independently selected from Cl, F, methyl, CF₃, CHF₂, and cyclopropyl optionally substituted with methyl or CF₃. In certain embodiments R³ is optionally substituted phenyl, wherein one of the one or more optional substituents is Cl. In certain embodiments R³ is optionally substituted phenyl, wherein one of the one or more optional substituents is F. In certain embodiments R³ is optionally substituted phenyl, wherein one of the one or more optional substituents is methyl. In certain embodiments R³ is optionally substituted phenyl, wherein one of the one or more optional substituents is CF₃. In certain embodiments R³ is optionally substituted phenyl, wherein one of the one or more optional substituents is CHF₂. In certain embodiments R³ is optionally substituted phenyl, wherein one of the one or more optional substituents is cyclopropyl optionally substituted with methyl. In certain embodiments R³ is optionally substituted phenyl, wherein one of the one or more optional substituents is cyclopropyl optionally substituted with CF₃.

In certain embodiments R³ is optionally substituted C₇-C₁₀ arylalkyl. In certain such embodiments, each of the one or more optional substituents is independently selected from: halo; C₁-C₆ alkyl optionally substituted with halo; and C₃-C₆ cycloalkyl optionally substituted with methyl or CF₃.

In certain embodiments R³ is optionally substituted (CH₂)₂-phenyl. In certain such embodiments, each of the one or more optional substituents is independently selected from: halo; C₁-C₆ alkyl optionally substituted with halo; and C₃-C₆ cycloalkyl optionally substituted with methyl or CF₃. In certain embodiments R³ is optionally substituted (CH₂)₂-phenyl, wherein one of the one or more optional substituents is halo. In certain embodiments R3 is optionally substituted (CH₂)₂-phenyl, wherein one of the one or more optional substituents is C₁-C₆ alkyl optionally substituted with halo, for example CHF₂ of CF₃. In certain embodiments R³ is optionally substituted (CH₂)₂-phenyl, wherein one of the one or more optional substituents is C₃-C₆ cycloalkyl (e.g. cyclopropyl) optionally substituted with methyl. In certain embodiments R³ is optionally substituted (CH₂)₂-phenyl, wherein one of the one or more optional substituents is C₃-C₆ cycloalkyl (e.g. cyclopropyl) optionally substituted CF₃.

In certain embodiments R³ is optionally substituted (CH₂)₂-phenyl, wherein one of the one or more optional substituents is on the ethylene chain. In certain such embodiments, the optional substituent on the ethylene chain is selected from: methyl or ethyl, optionally substituted with F.

In certain embodiments R³ is optionally substituted (CH₂)₂-phenyl, wherein one of the one or more optional substituents is on the ethylene chain and is selected from methyl, CHF₂ and CF₃. In certain such embodiments the optional substituent is CHF₂. In certain such embodiments the optional substituent is CF₃.

In certain preferred embodiments when R³ is optionally substituted (CH₂)₂-phenyl, the optional substituent on the ethylene chain is on the second methylene group—i.e. R³ is: —CH₂—CHR-Ph, where “R” is the optional substituent.

In certain such embodiments, the substituent on the ethylene chain of R³ results in a chiral centre at the chain carbon. In certain preferred embodiments, the compound is the R stereoisomer at this position. In certain alternative preferred embodiments, the compound is the S stereoisomer at this position.

Preferably, when R³ is an arylalkyl group, each of the one or more optional substituents of the arylalkyl group is independently selected from F, Cl, methyl, ethyl, CHF₂, and CF₃.

In accordance with all aspects (including formulae (I), (Ia), (Ib), (Ic) and (Id)), R⁴ is selected from H, Cl, and C₁-C₆ alkyl.

In certain embodiments, R⁴ is chlorine.

In certain alternative embodiments, R⁴ is H or C₁-C₆ alkyl. For example, R⁴ can be H or methyl.

In certain embodiments of all aspects (including formulae (I), (Ia). (Ib), (Ic) and (Id)), the chiral centre at morpholinyl ring carbon by which it is attached to the phenyl ring is in the R position. embodiments of all aspects (including formulae (I), (Ia), (Ib), (Ic) and (Id)), the chiral centre at morpholinyl ring carbon by which it is attached to the phenyl ring is in the S position. That is, the invention encompasses compounds of general formula:

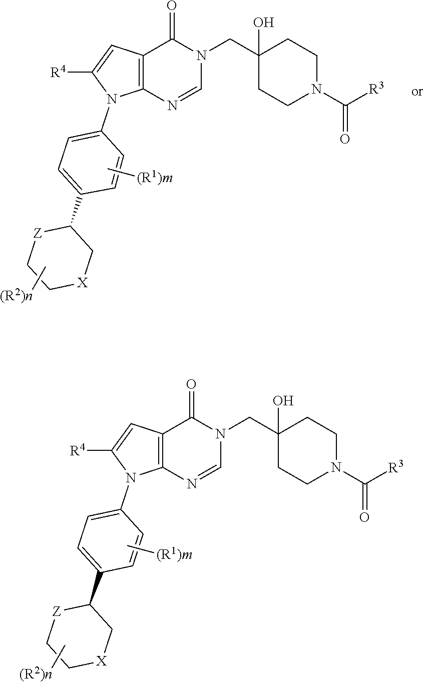

Also provided in accordance with the first aspect are the compounds (or their pharmaceutically acceptable salts or solvates):
rac-6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
rac-6-Chloro-3-((4-hydroxy-1-(1-methylcyclopropane-1-carbonyl)piperidin-4-yl)methyl)-7-(4-((3R,5S)-5-methylmorpholin-3-yl)phenyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-(3-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(3-methyl-4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((2S,3S)-2-methylmorpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((2R,3R)-2-methylmorpholin-3-yl)phenyl)-3,7-dihydro-4H-pyrrolo[2,3-d]pyrimidin-4-one
rac-6-Chloro-3-((1-(4-chlorobenzoyl)-4-hydroxypiperidin-4-yl)methyl)-7-(4-((3R,6R)-6-(trifluoromethyl)morpholin-3-yl)phenyl)-3,7-dihydro-4H-pyrrolo[2,3-d]pyrimidin-4-one
6-Chloro-7-(4-((S)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-7-(4-((R)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-7-(4-(5,5-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-(5,5-dimethylmorpholin-3-yl)phenyl)-3,7-dihydro-4H-pyrrolo[2,3-d]pyrimidin-4-one
rac-3-((1-(4-Chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
7-(4-(7-Oxa-4-azaspiro[2.5]octan-5-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
7-(4-(7-Oxa-4-azaspiro[2.5]octan-5-yl)phenyl)-6-chloro-3-((4-hydroxy-1-(1-methylcyclopropane-1-carbonyl)piperidin-4-yl)methyl)-3,7-dihydro-4H-pyrrolo[2,3-d]pyrimidin-4-one
3-((1-Benzoyl-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one
6-Chloro-3-((1-(4-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
(S)-6-Chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one
3-((1-Benzoyl-4-hydroxypiperidin-4-yl)methyl)-6-chloro-7-(4-((3S,6R)-6-methylmorpholin-3-yl)phenyl)-3,7-dihydro-4H-pyrrolo[2,3-d]pyrimidin-4-one
6-Chloro-3-((1-(4-fluorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,5S)-5-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,5R)-5-methylmorpholin-3-yl)phenyl)-3,7-dihydro-4H-pyrrolo[2,3-d]pyrimidin-4-one
(S)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one
(R)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one
6-Chloro-3-((1-(4-chloro-2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((4-hydroxy-1-((1S,2S)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
(R)-6-Chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one
6-Chloro-3-((1-(3-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(3,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(3-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2,3-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
(S)-3-((1-([1,1′-Bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one
(R)-3-((1-([1,1′-Bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one
3-((1-([1,1′-Bi(cyclopropane)]-1-carbonyl)-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one
7-(4-(5-Oxa-8-azaspiro[3.5]nonan-7-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one
7-(4-(5-Oxa-8-azaspiro[3.5]nonan-7-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
6-Chloro-7-(4-((3R,6S)-6-ethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one
6-Chloro-3-((1-(4-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
7-(4-((1R,2S,5S)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
7-(4-((1R,2S,5S)-8-Oxa-3-azabicyclo[3.2.1]octan-2-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one
6-Chloro-3-((1-(4-chloro-2-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(3-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-(octahydro-2H-benzo[b][1,4]oxazin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-(octahydrocyclopenta[b][1,4]oxazin-3-yl)phenyl)-3,7-dihydro-4H-pyrrolo[2,3-d]pyrimidin-4-one
6-Chloro-3-((4-hydroxy-1-(4-methylbenzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-(4-(trifluoromethyl)benzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2-chloro-4-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2,4-dimethylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2,4-dimethylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
7-(4-(2-Oxa-5-azabicyclo[4.1.0]heptan-4-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,6R)-6-cyclopropylmorpholin-3-yl)phenyl)-3,7-dihydro-4H-pyrrolo[2,3-d]pyrimidin-4-one
6-Chloro-3-((4-hydroxy-1-((1S,2S)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-cinnamoyl-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6S)-6-cyclopropylmorpholin-3-yl)phenyl)-3,7-dihydro-4H-pyrrolo[2,3-d]pyrimidin-4-one
6-Chloro-3-((4-hydroxy-1-(4-(trifluoromethyl)benzoyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-7-(4-((3R,6S)-6-ethylmorpholin-3-yl)phenyl)-3-((4-hydroxy-1-((1 S,2S)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((3R,6S)-6-ethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((R)-6,6-dimethylmorpholin-3-yl)phenyl)-3-((4-hydroxy-1-((1S,2S)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((S)-6,6-dimethylmorpholin-3-yl)phenyl)-3-((4-hydroxy-1-((1S,2S)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((R)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((S)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6R)-6-methylmorpholin-3-yl)phenyl)-3,7-dihydro-4H-pyrrolo[2,3-d]pyrimidin-4-one
(S)-6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one
(R)-6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one
6-Chloro-7-(3-fluoro-4-(morpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
(S)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one
(R)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-(4-methylmorpholin-3-yl)phenyl)-3,7-dihydro-4H-pyrrolo[2,3-d]pyrimidin-4-one
(S)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one
(R)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((S)-morpholin-3-yl)phenyl)-3,7-dihydro-4H-pyrrolo[2,3-d]pyrimidin-4-one
(S)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one
(R)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((R)-morpholin-3-yl)phenyl)-3,7-dihydro-4H-pyrrolo[2,3-d]pyrimidin-4-one
6-Chloro-3-((1-((R)-4,4-difluoro-3-phenylbutanoyl)-4-hydroxypiperidin-4-yl)methyl)-7-(4-((S)-morpholin-3-yl)phenyl)-3,7-dihydro-4H-pyrrolo[2,3-d]pyrimidin-4-one
6-Chloro-3-((4-hydroxy-1-(1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one
6-Chloro-3-((4-hydroxy-1-(1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one
6-Chloro-3-((1-((R)-4,4-difluoro-3-phenylbutanoyl)-4-hydroxypiperidin-4-yl)methyl)-7-(4-((R)-morpholin-3-yl)phenyl)-3,7-dihydro-4H-pyrrolo[2,3-d]pyrimidin-4-one
6-Chloro-7-(3-chloro-4-((S)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-7-(3-chloro-4-((R)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((S)-morpholin-3-yl)-3-(trifluoromethyl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((R)-morpholin-3-yl)-3-(trifluoromethyl)phenyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-(morpholin-2-yl)phenyl)-3,7-dihydro-4H-pyrrolo[2,3-d]pyrimidin-4-one
6-Chloro-3-((1-(4-chloro-2-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(4-chloro-2-methylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-(difluoromethyl)benzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-(difluoromethyl)benzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(4-cyclopropylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-(4-(1-methylcyclopropyl)benzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-((1 S,2S)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one
6-Chloro-3-((1-((1R,2R)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one
7-(4-((1 S,2R,5R)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
(R)-6-Chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
(S)-6-Chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((1-(2,2-dicyclopropylacetyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-7-(4-((3R,6S)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((4-hydroxy-1-(1-methylcyclobutane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(3-cyclopropylpropanoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-7-(4-((3S,6R)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-7-(4-((3S,6R)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-7-(4-((3R,6S)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((1S,2S)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one
6-Chloro-3-((1-((1 S,2S)-2-(difluoromethyl)-1-methylcyclopropane-1-carbonyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one,
and
6-Chloro-3-((1-((1R,2R)-2-(difluoromethyl)-1-methylcyclopropane-1-carbonyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one.

In certain embodiments, compounds of the present invention have an IC₅₀ value for USP7 of about 1 nM to about 10,000 nM, more preferably from about 1 nM to about 1000 nM, or from about 100 nM to about 1000 nM, or from about 100 nM to about 500 nM, or from about 100 nM to about 300 nM, or from about 100 nM to about 250 nM. In certain preferred embodiments, the compounds of the invention have an IC₅₀ value for USP7 of less than 500 nM, most preferably less than 250 nM. A method for determining the IC₅₀ value of a compound for USP7 is described below (see examples).

In certain embodiments, there is provided a compound of formula (I), (Ia), (Ib), (Ic), or (Id) according to the first aspect, or a pharmaceutically acceptable salt or solvate thereof, and at least one pharmaceutically acceptable excipient.

While it is possible for a compound of the invention to be administered alone, it is preferable to present it as a pharmaceutical formulation (e.g., composition, preparation, medicament) comprising at least one USP7 inhibitor as described herein, together with one or more other pharmaceutically acceptable ingredients well known to those skilled in the art, including pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

In a second aspect, therefore, the present invention provides a pharmaceutical composition comprising a compound according to any embodiment of the first aspect, for example a compound of formula (I), (Ia), (Ib), (Ic) or (Id), or a pharmaceutically acceptable salt or solvate thereof, and at least one pharmaceutically acceptable excipient.

Pharmaceutical compositions may be formulated according to their particular use and purpose by mixing, for example, excipient, binding agent, lubricant, disintegrating agent, coating material, emulsifier, suspending agent, solvent, stabilizer, absorption enhancer and/or ointment base. The composition may be suitable for oral, injectable, rectal, topical buccal, sublingual, transmucosal, transdermal, intranasal, pulmonary, ocular, vaginal or parenteral administration.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

Suitable pharmaceutically acceptable excipients would therefore be known by the person skilled in the art, for example: fats, water, physiological saline, alcohol (e.g. ethanol), glycerol, polyols, aqueous glucose solution, extending agent, disintegrating agent, binder, lubricant, wetting agent, stabilizer, emulsifier, dispersant, preservative, sweetener, colorant, seasoning agent or aromatizer, concentrating agent, diluent, buffer substance, solvent or solubilizing agent, chemical for achieving storage effect, salt for modifying osmotic pressure, coating agent or antioxidant, saccharides such as lactose or glucose; starch of corn, wheat or rice; fatty acids such as stearic acid; inorganic salts such as magnesium metasilicate aluminate or anhydrous calcium phosphate; synthetic polymers such as polyvinylpyrrolidone or polyalkylene glycol; alcohols such as stearyl alcohol or benzyl alcohol; synthetic cellulose derivatives such as methylcellulose, carboxymethylcellulose, ethylcellulose or hydroxypropylmethylcellulose; and other conventionally used additives such as gelatin, talc, plant oil and gum arabic.

For example, the pharmaceutical composition may be administered orally, such as in the form of tablets, coated tablets, hard or soft gelatine capsules, solutions, emulsions, or suspensions. Administration can also be carried out rectally, for example using suppositories, locally or percutaneously, for example using ointments, creams, gels or solution, or parenterally, for example using injectable solutions.

The formulations may be prepared by any methods well known in the art of pharmacy. Such methods include the step of bringing into association the compound with a carrier, which constitutes one or more accessory ingredients. In general, the formulations are prepared by uniformly and intimately bringing into association the compound with carriers (e.g., liquid carriers, finely divided solid carrier, etc.), and then shaping the product, if necessary.

For the preparation of tablets, coated tablets or hard gelatine capsules, the compounds of the present invention may be admixed with pharmaceutically inert, inorganic or organic excipients. Examples of suitable excipients include lactose, maize starch or derivatives thereof, talc or stearic acid or salts thereof. Suitable excipients for use with soft gelatine capsules include, for example, vegetable oils, waxes, fats and semi-solid or liquid polyols.

For the preparation of solutions and syrups, excipients include, for example, water, polyols, saccharose, invert sugar and glucose.

For injectable solutions, excipients include, for example, water, alcohols, polyols, glycerine and vegetable oil.

For suppositories and for local and percutaneous application, excipients include, for example, natural or hardened oils, waxes, fats and semi-solid or liquid polyols.

The pharmaceutical compositions may also contain preserving agents, solubilizing agents, stabilizing agents, wetting agents, emulsifiers, sweeteners, colorants, odorants, buffers, coating agents and/or antioxidants.

For combination therapies, the second drug may be provided in pharmaceutical composition with the present invention or may be provided separately.

Thus, a pharmaceutical formulation for oral administration may, for example, be granule, tablet, sugar-coated tablet, capsule, pill, suspension or emulsion. For parenteral injection for, for example, intravenous, intramuscular or subcutaneous use, a sterile aqueous solution may be provided that may contain other substances including, for example, salts and/or glucose to make the solution isotonic. The anti-cancer agent may also be administered in the form of a suppository or pessary, or may be applied topically in the form of a lotion, solution, cream, ointment or dusting powder.

In a third aspect the invention provides a compound or pharmaceutical composition according to any embodiment of the first aspect or second aspect for use in therapy, preferably in the treatment and/or prevention of cancer.

In a fourth aspect the invention provides a method of treating or preventing cancer comprising administering to a subject a compound according to any embodiment of the first aspect of the invention or a pharmaceutical composition according to any embodiment of the second aspect of the invention.

The term “treatment,” as used herein in the context of treating a disorder, pertains generally to treatment of a human or an animal (e.g., in veterinary applications), in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder. Treatment as a prophylactic measure (i.e., prophylaxis) is also included. For example, use with patients who have not yet developed the disorder, but who are at risk of developing the disorder, is encompassed by the term “treatment.” For example, treatment includes the prophylaxis of cancer, reducing the incidence of cancer, alleviating the symptoms of cancer, etc.

The subject/patient may be a mammal, a chordate, a vertebrate, a placental mammal, a marsupial (e.g., kangaroo, wombat), a monotreme (duck-billed platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human. Furthermore, the subject/patient may be any of its forms of development, for example, a foetus.

In preferred embodiments, the subject/patient is a human.

Cancers or neoplastic conditions suitable to be treated or prevented according to these methods include, for example, prostate cancer, colon cancer, breast cancer, lung cancer, kidney cancer, skin cancers (e.g. melanoma), liver cancers, pancreatic cancer, CNS cancers (e.g. neuroblastomas, glioblastomas), osteosarcoma, haematological malignancies (e.g. leukemia, multiple myeloma and mantle cell lymphoma). In certain preferred embodiments the cancer is associated with p53 dysregulation. In certain preferred embodiments, the cancer is selected from a haematological malignancy (e.g. mantle cell lymphoma, multiple myeloma), prostate cancer, a neuroblastoma, or a glioblastoma.

Cancers or neoplastic conditions suitable to be treated with the compounds or compositions according to the invention include, for example: prostate cancer, colon cancer, breast cancer, lung cancer, kidney cancer, skin cancers (e.g. melanoma), liver cancers, pancreatic cancer, CNS cancers (e.g. neuroblastomas, glioblastomas), osteosarcoma, haematological malignancies (e.g. leukemia, multiple myeloma and mantle cell lymphoma). In certain preferred embodiments the cancer is associated with p53 dysregulation. In certain preferred embodiments, the cancer is selected from a haematological malignancy (e.g. mantle cell lymphoma, multiple myeloma), prostate cancer, a neuroblastoma, or a glioblastoma.

It has been demonstrated in WO2021/161047 that USP7 inhibitors are capable of treating cancer by affecting the tumour microenvironment (TME), for example by inhibiting USP7 in fibroblasts. The contents and disclosure of WO2021/161047 are expressly incorporated herein by reference.

Accordingly, provided herein is a method of treating cancer by inhibiting USP7 activity in fibroblasts, the method comprising administering to a subject in need thereof a composition comprising a compound provided herein or a pharmaceutically acceptable salt or solvate thereof.

In certain embodiments, administration of a compound provided herein or a pharmaceutically acceptable salt or solvate thereof treats the cancer by inhibiting USP7 activity in cancer-associated fibroblasts (CAFs).

In certain embodiments, administration of a compound provided herein or a pharmaceutically acceptable salt or solvate thereof treats the cancer by reducing the level of VEGF in the serum of the subject. In certain embodiments administration of a compound provided herein or a pharmaceutically acceptable salt or solvate thereof treats the cancer by reducing the level of VEGF in the tumour microenvironment.

In certain embodiments, administration of a compound provided herein or a pharmaceutically acceptable salt or solvate thereof treats the cancer by inhibiting production of VEGF by cancer-associated fibroblasts (CAFs).

In a further aspect is provided a method of treating cancer by modulating the tumour immune environment, the method comprising administering to a subject in need thereof a compound provided herein or a pharmaceutically acceptable salt or solvate thereof, wherein administration of the compound modulates the tumour immune environment.

In a further aspect is provided a method of treating cancer by increasing tumour infiltrating lymphocytes (TILs), the method comprising administering to a subject in need thereof a compound provided herein or a pharmaceutically acceptable salt or solvate thereof, wherein administration of the compound increases the number of TILs, preferably CD8+ TILs.

In a further aspect is provided a method of treating cancer by decreasing the proportion of Treg cells relative to CD8+ T cells in the TME, the method comprising administering to a subject in need thereof a compound provided herein or a pharmaceutically acceptable salt or solvate thereof, wherein administration of the compound decreases the proportion of Treg cells relative to CD8+ T cells in the TME.

In a further aspect is provided a method of treating cancer by decreasing the number of macrophages in the TME, the method comprising administering to a subject in need thereof a compound provided herein or a pharmaceutically acceptable salt or solvate thereof, wherein administration of the compound decreases the number of macrophages in the TME.

It has also been demonstrated in WO2021/161047 that USP7 inhibitors used in combination with immune checkpoint inhibitors exhibit advantageous efficacy in the treatment of cancer (e.g. synergistic efficacy). The contents and disclosure of WO2021/161047 are expressly incorporated herein by reference.

In a further aspect is provided a method of treating cancer by administering to a subject in need thereof a combination therapy, the combination therapy comprising a composition comprising a compound provided herein or a pharmaceutically acceptable salt or solvate thereof, and a composition comprising an immune checkpoint inhibitor.

In a further aspect is provided a compound provided herein or a pharmaceutically acceptable salt or solvate thereof, for use in a method of treating cancer, the method comprising administering to a subject in need thereof a combination therapy, the combination therapy comprising a composition comprising a compound provided herein or a pharmaceutically acceptable salt or solvate thereof, and a composition comprising an immune checkpoint inhibitor.

In a further aspect is provided an immune checkpoint inhibitor for use in a method of treating cancer, the method comprising administering to a subject in need thereof a combination therapy, the combination therapy comprising a composition comprising a compound provided herein or a pharmaceutically acceptable salt or solvate thereof, and a composition comprising the immune checkpoint inhibitor.

In a further aspect is provided a combination therapy for use in a method of treating cancer, the method comprising administering to a subject in need thereof the combination therapy, the combination therapy comprising a composition comprising a compound provided herein or a pharmaceutically acceptable salt or solvate thereof, and a composition comprising an immune checkpoint inhibitor.

In certain embodiments, administration of a compound provided herein, or a pharmaceutically acceptable salt or solvate thereof, treats cancer by inhibiting extra-cellular matrix (ECM) remodelling by cancer-associated fibroblasts. In such embodiments the ECM is the ECM of the tumour microenvironment.

In certain embodiments, administration of a compound provided herein, or a pharmaceutically acceptable salt or solvate thereof, treats the cancer by inhibiting degradation of the basement membrane, optionally degradation of the tubular basement membrane.

In certain embodiments, administration of a compound provided herein, or a pharmaceutically acceptable salt or solvate thereof, treats the cancer by inhibiting angiogenesis, optionally neo-angiogenesis. In such embodiments angiogenesis is inhibited in the tumour microenvironment.

In certain embodiments, administration of a compound provided herein, or a pharmaceutically acceptable salt or solvate thereof, treats the cancer by modulation of the tumour immune environment, for example by promoting infiltration of CD8 T cells.

In certain embodiments, the cancer treated is formed of cancer cells, and the cancer cells are resistant to the administered compound (or pharmaceutically acceptable salt or solvate).

In certain embodiments, the cancer treated by the method is formed of cancer cells, and the cancer cells are resistant to the USP7 inhibitor in vitro.

The compound or composition of the invention may be used in monotherapy and/or a combination modality.

As already described, in certain aspects a compound provided herein (or pharmaceutically acceptable salt or solvate thereof) may be administered in combination with an immune checkpoint inhibitor. In certain embodiments the checkpoint inhibitor is selected from an inhibitor of PD1, PD-L1, CTLA4, TIGIT, 41 BB, OX40, GITR. In certain embodiments the checkpoint inhibitor is selected from an anti-PD1 antibody, an anti-PD-L1 antibody, an anti-CTLA4 antibody, an anti-41 BB antibody, an anti-OX40 antibody, an anti-GITR antibody, and an anti-ICOS antibody. In certain embodiments the checkpoint inhibitor is selected from an anti-PD1 antibody, an anti-PD-L1 antibody, and an anti-CTLA4 antibody. In certain embodiments the checkpoint inhibitor is selected from an anti-PD1 antibody and an anti-PD-L1 antibody. In certain embodiments the checkpoint inhibitor is an anti-CTLA4 antibody. In certain embodiments the checkpoint inhibitor is selected from: pembrolizumab (Keytruda™) nivolumab (Opdivo™), cemiplimab (Libtayo™), Atezolizumab (Tecentriq™), Avelumab (Bavencio™), Durvalumab (Imfinzi™), and Ipilimumab (Yervoy™) Other suitable agents that may be used in combination modalities with compounds or compositions according to the invention include one or more of anti-cancer agents, anti-inflammatory agents, immuno-modulatory agents, immuno-suppressive agents, neurological agents, anti-diabetic agents, anti-viral agents, anti-bacterial agents and/or radiation therapy.

Agents used in combination with the compounds of the present invention may target the same or a similar biological pathway to that targeted by the compounds of the present invention or may act on a different or unrelated pathway.

Depending on the disease to be treated, a variety of combination partners may be coadministered with the compounds of the present invention. The second active ingredient may include, but is not restricted to: alkylating agents, including cyclophosphamide, ifosfamide, thiotepa, melphalan, chloroethylnitrosourea and bendamustine; platinum derivatives, including cisplatin, oxaliplatin, carboplatin and satraplatin; antimitotic agents, including vinca alkaloids (vincristine, vinorelbine and vinblastine), taxanes (paclitaxel, docetaxel), epothilones and inhibitors of mitotic kinases including aurora and polo kinases; topoisomerase inhibitors, including anthracyclines, epipodophyllotoxins, camptothecin and analogues of camptothecin; antimetabolites, including 5-fluorouracil, capecitabine, cytarabine, gemcitabine, 6-mercaptopurine, 6-thioguanine, fludarabine, methotrexate and pemetrexed; protein kinase inhibitors, including imatinib, gefitinib, sorafenib, sunitinib, erlotinib, dasatinib, and lapatinib; proteosome inhibitors, including bortezomib; histone deacetylase inhibitors, including valproate and SAHA; antiangiogenic drugs, including bevacizumab; monoclonal antibodies, including trastuzumab, rituximab, alemtuzumab, tositumomab, cetuximab, panitumumab; conjugates of monoclonal antibodies, including Gemtuzumab ozogamicin, Ibritumomab tiuxetan; hormonal therapies, including antiestrogens (tamoxifen, raloxifen, anastrazole, letrozole, examestane) antiandrogens (Flutamide, Biclutamide) and Luteinising Hormone Analogues or antagonists.

It will be appreciated by one of skill in the art that appropriate dosages of the compounds of the invention, and compositions comprising the compounds, can vary from patient to patient. Determining the optimal dosage will generally involve the balancing of the level of therapeutic benefit against any risk or deleterious side effects. Dosage for therapeutic effects may differ from the dosage required for diagnostic effects. The selected dosage level will depend on a variety of factors including the activity of the particular compound, the route of administration, the time of administration, the rate of excretion of the compound, the duration of the treatment, other drugs, compounds, and/or materials used in combination, the severity of the disorder, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In regard to aspects of the invention relating to therapeutic use of compounds according to the invention, the compounds may be administered to the subject in need of treatment in an “effective amount”. The term “effective amount” refers to the amount or dose of a compound which, upon single or multiple dose administration to a subject, provides therapeutic efficacy in the treatment of disease. Therapeutically effective amounts of a compound according to the invention can comprise an amount in the range of from about 0.1 mg/kg to about 20 mg/kg per single dose. A therapeutic effective amount for any individual patient can be determined by the healthcare professional by methods understood by the skilled person. The amount of compound administered at any given time point may be varied so that optimal amounts of the compound, whether employed alone or in combination with any other therapeutic agent, are administered during the course of treatment. It is also contemplated to administer compounds according to the invention, or pharmaceutical compositions comprising such compounds, in combination with any other cancer treatment, as a combination therapy.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

The present invention will now be described in relation to several examples.

The examples indicated below were synthesised according to the methods described subsequently. Biochemical inhibition IC₅₀ values were determined as described below and are represented in the following tables.

TABLE 3a
USP7 inhibition by exemplified compounds.

| Example Number | USP7 IC₅₀ activity |
|---|---|
| 1 | *** |
| 2 | *** |
| 3 | ** |
| 4 | ** |
| 5 | ** |
| 6 | *** |
| 7 | *** |
| 8 | *** |
| 9 | * |
| 10 | *** |
| 11 | *** |
| 12 | ** |
| 13 | *** |
| 14 | *** |
| 15 | ** |
| 16 | ** |
| 17 | ** |
| 18 | * |
| 19 | * |
| 20 | * |
| 21 | ** |
| 22 | ** |
| 23 | ** |
| 24 | ** |
| 25 | ** |
| 26 | ** |
| 27 | ** |
| 28 | ** |
| 29 | ** |
| 30 | ** |
| 31 | ** |
| 32 | *** |
| 33 | ** |
| 34 | *** |
| 35 | ** |
| 36 | * |
| 37 | * |
| 38 | * |
| 39 | * |
| 40 | ** |
| 41 | *** |
| 42 | *** |
| 43 | *** |
| 44 | ** |
| 45 | ** |
| 46 | ** |
| 47 | *** |
| 48 | * |
| 49 | ** |
| 50 | ** |
| 51 | *** |
| 52 | ** |
| 53 | * |
| 54 | ** |
| 55 | ** |
| 56 | ** |
| 57 | *** |
| 58 | * |
| 59 | * |
| 60 | * |
| 61 | ** |
| 62 | *** |
| 63 | ** |
| 64 | ** |
| 65 | ** |
| 66 | *** |
| 67 | *** |
| 68 | * |
| 69 | * |
| 70 | *** |
| 71 | *** |
| 72 | ** |
| 73 | *** |
| 74 | ** |
| 75 | * |
| 76 | *** |
| 77 | *** |
| 78 | *** |
| 79 | *** |
| 80 | *** |
| 81 | *** |
| 82 | *** |
| 83 | *** |
| 84 | *** |
| 85 | *** |
| 86 | ** |
| 87 | * |
| 88 | ** |
| 89 | ** |
| 90 | * |
| 91 | ** |
| 92 | ** |
| 93 | ** |
| 94 | ** |
| 95 | ** |
| 96 | *** |
| 97 | ** |
| 98 | *** |
| 99 | * |
| 100 | *** |
| 101 | *** |
| 102 | *** |
| 103 | ** |
| 104 | *** |
| 105 | *** |
| 106 | ** |
| 107 | *** |
| 108 | *** |
| 109 | ** |
| 110 | *** |
| 111 | ** |
| 112 | *** |

The USP7 inhibitory activities in Table 3a are classified as the following:

| | *** | ** | * |
|---|---|---|---|
| USP7 IC₅₀ [μM] | IC₅₀ < 0.1 | 0.1 ≤ IC₅₀ < 0.25 | 0.25 ≤ IC₅₀ < 2 |

TABLE 3b
USP7 inhibition by exemplified compounds.

| Example Number | USP7 IC₅₀ activity |
|---|---|
| 1 | 0.030 |
| 2 | 0.046 |
| 3 | 0.132 |
| 4 | 0.222 |
| 5 | 0.225 |
| 6 | 0.050 |
| 7 | 0.016 |
| 8 | 0.077 |
| 9 | 0.420 |
| 10 | 0.017 |
| 11 | 0.080 |
| 12 | 0.130 |
| 13 | 0.028 |
| 14 | 0.018 |
| 15 | 0.169 |
| 16 | 0.249 |
| 17 | 0.175 |
| 18 | 0.378 |
| 19 | 0.372 |
| 20 | 0.379 |
| 21 | 0.108 |
| 22 | 0.222 |
| 23 | 0.143 |
| 24 | 0.250 |
| 25 | 0.147 |
| 26 | 0.161 |
| 27 | 0.194 |
| 28 | 0.218 |
| 29 | 0.118 |
| 30 | 0.164 |
| 31 | 0.144 |
| 32 | 0.048 |
| 33 | 0.226 |
| 34 | 0.088 |
| 35 | 0.224 |
| 36 | 0.511 |
| 37 | 0.254 |
| 38 | 0.302 |
| 39 | 1.148 |
| 40 | 0.156 |
| 41 | 0.079 |
| 42 | 0.091 |
| 43 | 0.050 |
| 44 | 0.113 |
| 45 | 0.176 |
| 46 | 0.162 |
| 47 | 0.091 |
| 48 | 0.320 |
| 49 | 0.223 |
| 50 | 0.164 |
| 51 | 0.258 |
| 52 | 0.179 |
| 53 | 0.264 |
| 54 | 0.151 |
| 55 | 0.159 |
| 56 | 0.113 |
| 57 | 0.084 |
| 58 | 0.414 |
| 59 | 0.545 |
| 60 | 0.313 |
| 61 | 0.128 |
| 62 | 0.038 |
| 63 | 0.221 |
| 64 | 0.110 |
| 65 | 0.156 |
| 66 | 0.063 |
| 67 | 0.053 |
| 68 | 0.702 |
| 69 | 0.586 |
| 70 | 0.022 |
| 71 | 0.094 |
| 72 | 0.211 |
| 73 | 0.018 |
| 74 | 0.112 |
| 75 | 2.094 |
| 76 | 0.038 |
| 77 | 0.052 |
| 78 | 0.016 |
| 79 | 0.039 |
| 80 | 0.041 |
| 81 | 0.015 |
| 82 | 0.006 |
| 83 | 0.028 |
| 84 | 0.045 |
| 85 | 0.006 |
| 86 | 0.134 |
| 87 | 0.292 |
| 88 | 0.158 |
| 89 | 0.204 |
| 90 | 0.293 |
| 91 | 0.167 |
| 92 | 0.111 |
| 93 | 0.160 |
| 94 | 0.193 |
| 95 | 0.132 |
| 96 | 0.037 |
| 97 | 0.174 |
| 98 | 0.045 |
| 99 | 0.263 |
| 100 | 0.079 |
| 101 | 0.057 |
| 102 | 0.050 |
| 103 | 0.211 |
| 104 | 0.094 |
| 105 | 0.073 |
| 106 | 0.121 |
| 107 | 0.093 |
| 108 | 0.042 |
| 109 | 0.182 |
| 110 | 0.079 |
| 111 | 0.145 |
| 112 | 0.018 |

### EXPERIMENTAL SECTION

### Abbreviations and Acronyms

B₂Pin₂: bis(pinacolato)diboron; Boc: tert-butyloxycarbonyl; Boc₂O: di-tert-butyl decarbonate; br: broad; c.a.: catalytic amount; Cu(OTf)₂: copper(II) trifluoromethanesulfonate; DCM: dichloromethane; d: doublet (spectral); DIPEA: diisopropylethylamine; DMAP: 4-dimethylaminopyridine; DMF: N,N-dimethylformamide; DMSO: dimethylsulfoxide; dp: datapoint; Pd(dppf)Cl₂·CH₂Cl₂: [1,1′-bis(diphenylphosphino)ferrocene]dichloropalladium(II), complex with dichloromethane; EDO: N-(3-dimethylaminopropyl)-N′-ethylcarbodiimide hydrochloride; ee: enantiomeric excess; equiv.: equivalents; EtOAc: ethyl acetate; Ex.: Example; h: hour(s); HATU: N-[(dimethylamino)-1H-1,2,3-triazolo-[4,5-b]pyridin-1-ylmethylene]-N-methylmethanaminium hexafluorophosphate N-oxide; hept: heptet (spectral); HFIP: hexafluoroisopropanol; HPLC: high pressure liquid chromatography; IPA: 2-propanol; KOAc: potassium acetate; LC: liquid chromatography; LCMS: liquid chromatography mass spectrometry; M: molar; m/z: mass-to-charge ratio; m-CPBA: 3-chloroperbenzoic acid; MeCN: acetonitrile; MeOH: methanol; min: minute(s); MS: mass spectrometry; m: multiplet (spectral); MTBE: methyl tert-butyl ether; NMR: nuclear magnetic resonance; p: pentet (spectral); Ph: phenyl; ppm: parts per million; prep: preparative; q: quartet (spectral); quint: quintet (spectral); RBF: round-bottom flask; R_{T}: retention time; rt: room temperature; s: singlet; TFA: trifluoroacetic acid; THF: tetrahydrofuran; t: triplet (spectral); UV: ultraviolet; v/v: volume per unit volume; w/w: weight per unit weight.

### General Experimental Conditions

### Solvents and Reagents

Common organic solvents that were used in reactions (e.g. THF, DMF, DCM, and MeOH) were purchased anhydrous from Sigma-Aldrich® in Sure/Seal™ bottles and were handled appropriately under nitrogen. Water was deionised using an Elga PURELAB Option-Q. All other solvents used (i.e. for work-up procedures and purification) were generally HPLC grade and were used as supplied from various commercial sources. Unless otherwise stated, all starting materials used were purchased from commercial suppliers and used as supplied.

### Microwave Synthesis

Microwave experiments were carried out using a Biotage Initiator™ Eight instrument. The system gives good reproducibility and control at temperature ranges from 60-250° C. and pressures of up to a maximum of 20 bar.

### Flash Chromatography

Unless otherwise stated, purification of compounds by flash chromatography was achieved using a Biotage Isolera Four system using Biotage® Sfär Silica D Duo 60 μm cartridge columns (10-340 g), Grace GraceResolv cartridge columns (4-330 g), SILICYCLE SiliaSep™ cartridge columns (4-12 g) or SILICYCLE SiliaSep PREMIUM cartridge columns (25-40 g). Appropriate gradients of cyclohexane, EtOAc, DCM and MeOH were used depending on compound polarity. In the case of more polar and basic compounds, Biotage® Sfär KP-Amino D cartridge columns (11-28 g) were used.

### NMR Spectroscopy

¹H NMR spectra were recorded at ambient temperature using a Bruker Avance (300 MHz), Bruker Avance III (400 MHz) or Bruker Ascend (500 MHz) spectrometer. All chemical shifts (δ) are expressed in ppm. Residual solvent signals were used as an internal standard and the characteristic solvent peaks were corrected to the reference data outlined in J. Org. Chem., 1997, 62, p 7512-7515; in other cases, NMR solvents contained tetramethylsilane, which was used as an internal standard.

### Liquid Chromatography Mass Spectrometry (LCMS)

Liquid Chromatography Mass Spectrometry (LCMS) experiments to determine retention times (R_{T}) and associated mass ions were performed using the following methods:

Method A: The system consisted of an Agilent Technologies 6130 quadrupole mass spectrometer linked to an Agilent Technologies 1290 Infinity LC system with UV diode array detector and autosampler. The spectrometer consisted of an electrospray ionization source operating in positive and negative ion mode. LCMS experiments were performed on each sample submitted using the following conditions: LC Column: Agilent Eclipse Plus C18 RRHD, 1.8 μm, 50×2.1 mm maintained at 40° C. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 0.5 | 80 | 20 |
| 1.80 | 0.5 | 0 | 100 |
| 2.20 | 0.5 | 0 | 100 |
| 2.50 | 0.5 | 80 | 20 |
| 3.00 | 0.5 | 80 | 20 |

Method B: The system consisted of an Agilent Technologies 6140 single quadrupole mass spectrometer linked to an Agilent Technologies 1290 Infinity LC system with UV diode array detector and autosampler. The spectrometer consisted of a multimode ionization source (electrospray and atmospheric pressure chemical ionizations) operating in positive and negative ion mode. LCMS experiments were performed on each sample submitted using the following conditions: LC Column: Zorbax Eclipse Plus C18 RRHD, 1.8 μm, 50×2.1 mm maintained at 40° C. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 1.0 | 95 | 5 |
| 1.80 | 1.0 | 0 | 100 |
| 2.20 | 1.0 | 0 | 100 |
| 2.21 | 1.0 | 95 | 5 |
| 2.50 | 1.0 | 95 | 5 |

Method C: The system consisted of either an Agilent Technologies 1100 Series LC/MSD system with UV diode array detector and evaporative light scattering detector (DAD/ELSD) and Agilent LC/MSD VL (G1956A), SL (G1956B) mass spectrometer or an Agilent 1200 Series LC/MSD system with DAD/ELSD and Agilent LC/MSD SL (G6130A), SL (G6140A) mass spectrometer. All of the LCMS data were obtained using the atmospheric pressure chemical ionization mode with positive and negative ion mode switching with a scan range of m/z 80-1000. LCMS experiments were performed on each sample submitted using the following conditions: LC Column: Zorbax SB-C18 RRHD, 1.8 μm, 4.6×15 mm. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 3.0 | 100 | 0 |
| 1.50 | 3.0 | 0 | 100 |
| 1.80 | 3.0 | 0 | 100 |
| 1.81 | 3.0 | 100 | 0 |

Method D: The system consisted of a Waters QDa mass spectrometer linked to a Waters iClass UPLC system with TUV detector. The spectrometer consisted of an electrospray ionization source operating in positive and negative ion mode. LCMS experiments were performed on each sample submitted using the following conditions: LC Column: Zorbax Eclipse Plus C18 RRHD, 1.8 μm, 50×2.1 mm maintained at 40° C. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 0.8 | 95 | 5 |
| 1.80 | 0.8 | 0 | 100 |
| 2.20 | 0.8 | 0 | 100 |
| 2.30 | 0.8 | 95 | 5 |
| 2.50 | 0.8 | 95 | 5 |

### Preparative High Pressure Liquid Chromatography

Basic conditions: LC Column: Waters XBridge™ Prep C18 5 μm OBD™ 19×50 mm column at rt using gradients of Mobile phases: A) 0.1% (v/v) ammonium hydroxide in water in B) 0.1% (v/v) ammonium hydroxide in 95:5, acetonitrile/water.

### Chiral Separation of Stereoisomers by Supercritical Fluid Chromatography (SFC)

The separation of mixtures of stereoisomers was performed using the following general procedure. The mixture of stereoisomers was dissolved to 50 mg/mL in methanol and purified by SFC under the stated conditions. Combined fractions of each of stereoisomer were evaporated to near dryness using a rotary evaporator, transferred into final vessels using DCM, which was removed under a stream of compressed air at 40° C., before being stored in a vacuum oven at 40° C. and 5 mbar for 16 h.

### Chiral Separation of Stereoisomers by HPLC

The separation of mixtures of stereoisomers was performed using the following general procedure. The mixture of stereoisomers was dissolved to 66 mg/mL in methanol and purified by HPLC under the stated conditions. Combined fractions of each of stereoisomer were evaporated to near dryness using a rotary evaporator, transferred into final vessels using MeOH, which was removed under a stream of compressed air at 35° C., before being stored in a vacuum oven at 35° C. and 5 mbar for 16 h.

### Chiral Purity Analysis

After chiral separation of mixtures of stereoisomers, each stereoisomer was analysed to determine chiral purity using the following analytical SFC or HPLC methods under the stated conditions.

### Method A (HPLC):

Column Details Chiralpak AS-H (4.6 mm×250 mm, 5 μm)
Column Temperature Ambient
Flow Rate 0.6 mL/min
Detector Wavelength 220 nm
Injection Volume 1 μL
Isocratic Conditions 95:5 hexane(0.1% TFA):IPA

### Method B (HPLC):

Column Details Chiralpak IG (4.6 mm×250 mm, 5 μm)
Column Temperature Ambient
Flow Rate 0.6 mL/min
Detector Wavelength 220 nm
Injection Volume 1 μL
Isocratic Conditions 90:10 hexane:IPA

### Method C (SFC):

Column Details Chiralpak IH (4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 40:60 MeOH:CO₂ (0.2% v/v NH₃)

### Method D (HPLC):

Column Details Chiralcel OD-H (4.6 mm×250 mm, 5 μm)
Column Temperature Ambient
Flow Rate 0.6 mL/min
Detector Wavelength 220 nm
Injection Volume 1 μL
Isocratic Conditions 95:5 hexane:IPA

### Method E (SFC):

Column Details Amy-C Neo (4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 20:80 IPA:CO₂ (0.2% v/v NH₃)

### Method F (SFC):

Column Details Chiralpak IH (4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 15:85 IPA:CO₂ (0.2% v/v NH₃)

### Method G (SFC):

Column Details Chiralpak IG (4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 60:40 MeOH:CO₂ (0.1% v/v NH₃)

### Method H (SFC):

Column Details Chiralpak IG (4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 30:70 MeOH:CO₂ (0.1% v/v NH₃)

### Method I (SFC):

Column Details Chiralpak IG (4.6 mm×250 mm, 5 μm)
Column Temperature 40° C.
Flow Rate 4 mL/min
BPR 125 BarG
Detector Wavelength 210-400 nm
Injection Volume 1 μL
Isocratic Conditions 50:50 EtOH:CO₂ (0.2% v/v NH₃)

### Method J (SFC):

Column Details Lux C₁ (4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 25:75 MeOH:CO₂ (0.1% v/v NH₃)

### Method K (SFC):

Column Details Amy-C(4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 40:60 MeOH:CO₂ (0.2% v/v NH₃)

### Method L (SFC):

Column Details Chiralpak IH (4.6 mm×250 mm, 5 μm)
Column Temperature 40° C.
Flow Rate 4 mL/min
Detector Wavelength 210-400 nm
Injection Volume 1 μL
BPR 125 BarG
Isocratic Conditions 10:90 MeOH:CO₂ (0.1% v/v NH₃)

### Nomenclature and Stereochemistry

Unless otherwise indicated, the nomenclature of structures was determined using the ‘Convert Structure to Name’ function of ChemDraw Professional 20.1 (CambridgeSoft/PerkinElmer). The relative stereochemistry of the morpholine substituents have been assigned based on literature precedence (Org. Lett. 2014, 16, 1236-1239; J. Am. Chem. Soc. 2019, 141, 9739-9745) and NMR studies. In the cases of Example 26, Example 27, Example 61, Example 64, Example 68 and Example 69 these are single non-racemic diastereoisomers of known relative stereochemistry however the absolute stereochemistry is unknown and has been assigned arbitrarily so may be opposite to that stated.

In the cases of Example 32, Example 33, Example 62 and Example 66 these are single non-racemic diastereoisomers where the absolute stereochemistry of the morpholine substituents are known however the absolute stereochemistry of the cyclopropyl substituents was unknown. In the case of Example 67 this is a single non-racemic diastereoisomer where the absolute stereochemistry of all three stereocentres was unknown. In the cases of Example 32, Example 33, Example 62, Example 66 and Example 67 the relative stereochemistry of the cyclopropyl substituents was assigned based on NOE experiments. The absolute stereochemistry of the substituents on the cyclopropane ring in Example 62 was subsequently determined, by X-ray analysis of a co-crystal of Example 62 in complex with USP7. The absolute stereochemistry of the substituents on the cyclopropane ring in related Examples 32, 33, 62, 66 and 67 was hence determined via the co-crystal structure of Example 62/USP7 (see Examples, below). The relative stereochemistry of the more active cyclopropane isomer is also consistent with Examples 95 and 96. The stereochemistry of Examples 108, 109, and 110, which are in the same series, was determined analogously. In the cases of Example 23, Example 28, Example 29, Example 34, Example 40, Example 41, Example 70, Example 71, Example 73, Example 74, Example 76, Example 77, Example 79 and Example 80 these are single enantiomers of unknown absolute configuration in which the stereochemistry has been assigned arbitrarily so may be opposite to that stated. In the cases of Example 1, Example 2, Example 9 and Example 17 these are racemic diastereoisomers of known relative stereochemistry. In the cases of Example 10, Example 86 and Example 87 these are each a 1:1 mixture of two diastereoisomers. In the cases of Example 53, Example 54 and Example 60 these are unknown mixtures of diastereoisomers. In the cases of Example 78, Example 81, Example 82 and Example 85 these are single non-racemic diastereoisomers in which the absolute stereochemistry of the stereocentre in the morpholine ring is unknown and has been assigned arbitrarily so may be opposite to that stated. In the cases of Example 83 and Example 84 these are 4:4:1:1 mixtures of diastereoisomers in which the absolute stereochemistry of the stereocentre in the morpholine ring is unknown and has been assigned arbitrarily so may be opposite to that stated. Examples 111 and 112 were named based on their respective activities.

### General Procedures

### General Procedure 1: HATU Coupling

The appropriate amine or amine hydrochloride salt (1 equiv.), carboxylic acid (1 equiv.) and HATU (1 equiv.) were dissolved in DCM before DIPEA (3 equiv.) was added. The reaction was stirred for the time stated before being quenched by the addition of saturated aqueous sodium hydrogen carbonate. The resulting mixture was extracted using DCM (×3) using an ISOLUTE® phase separator. The combined organic phases were concentrated under reduced pressure and the remaining residue was purified by flash chromatography to give the product.

General Procedure 2: m-CPBA Oxidation

A solution of m-CPBA (<77% w/w) (1.2 equiv.) in DCM (0.2 M) was added dropwise using a syringe or dropping funnel to a stirred solution of the alkene (1 equiv.) in DCM (0.2 M) at rt or 0° C. After the addition was complete the reaction mixture was stirred at rt for the indicated time. The excess m-CPBA was quenched by the addition of saturated aqueous sodium thiosulfate (˜0.4 mL/g m-CPBA) before the resulting mixture was transferred to a separating funnel containing saturated aqueous sodium hydrogen carbonate. The resulting phasic mixture was extracted with DCM (×3), the combined organic layers were passed through an ISOLUTE® phase separator and concentrated under reduced pressure. The remaining residue was purified by flash chromatography to give the product.

### General Procedure 3: Miyaura Borylation

A suspension of the appropriate aryl bromide (1 equiv.), B₂Pin₂ (1.5 equiv.), Pd(dppf)Cl₂ CH₂Cl₂ (0.1 equiv.) and KOAc (3 equiv.) in 1,4-dioxane (0.1 M) were stirred under heating by the stated method for the indicated time. Work-up A: Upon cooling to rt the reaction mixture was filtered through celite and the solids were washed with EtOAc. The resulting solution was concentrated under reduced pressure and the residue was purified by flash chromatography to give the product. Work-up B: Upon cooling to rt the reaction mixture was concentrated under reduced pressure and the residue was suspended in DCM. The resulting suspension was filtered through celite and the solids washed with DCM before the combined filtrates were concentrated under reduced pressure. The resulting residue was purified by flash chromatography to give the product.

### General Procedure 4: Chan-Lam Coupling

A suspension of the appropriate NH-heterocycle (1-1.5 equiv.), the appropriate aryl boronate (1-2 equiv.), the appropriate copper(II) salt (2 equiv.), 1,10-phenanthroline (2 equiv.) and boric acid (2-4 equiv.) in DMF were stirred at the indicated temperature under an atmosphere of air for the indicated time. Work-up A: Upon cooling to rt the reaction mixture was diluted with 1:1 brine/water and extracted with EtOAc. The organic layer was washed with 1:1 brine/water, dried (MgSO4), filtered and concentrated under reduced pressure. The residue was purified flash chromatography to give the product. Work-up B: Upon cooling to rt the reaction mixture was diluted with 10% aqueous ammonium hydroxide and the resulting suspension was extracted with DCM (×3) using an ISOLUTE® phase separator. The combined organic phases were concentrated under reduced pressure and the residue was purified by flash chromatography to give the product.

### General Procedure 5: Hydrolysis of Chloropyrimidine

A mixture of the chloropyrimidine (1 equiv.), aqueous sodium hydroxide (10-20 equiv.) and 1,4-dioxane was heated at 100° C. for the stated time. Upon cooling to rt the reaction mixture was acidified to ˜pH 4-5 by the addition of the stated acid and the product was extracted into DCM (×3). The resulting solution was passed though an ISOLUTE® phase separator and concentrated under reduced pressure to give the product which was used without further purification in the next step.

General Procedure 6: Epoxide Opening with a Pyrimidinone

The appropriate pyrimidinone (1 equiv.), the epoxide (1-1.5 equiv.) and Cs₂CO₃ (1-3 equiv.) were suspended in DMF and the reaction mixture was heated at 80° C. for the stated time. The reaction was allowed to cool to rt, saturated aqueous ammonium chloride or water were added and the resulting mixture was extracted using DCM or EtOAc (×3). The combined organic phases were dried (ISOLUTE® phase separator or MgSO₄), concentrated under reduced pressure and the remaining residue was purified by flash chromatography to give the product.

### General Procedure 7: Boc Deprotection to Free Base

The Boc protected amine (1 equiv.) was dissolved in DCM and TFA was added. The reaction was stirred at rt for the stated time before being concentrated under reduced pressure. The remaining residue was dissolved in a mixture of MeOH and DCM and loaded onto a pre-equilibrated SCX-2 cartridge. The cartridge was washed with a 4:1 mixture of DCM/MeOH and the basic compound was eluted using a 4:1 mixture of DCM/7 M NH₃ in MeOH. The ammoniacal fractions were concentrated to give the desired product.

### General Procedure 8: Synthesis of Saturated Heterocycles Using SnAP Reagents

To a solution of the SnAP reagent (1 equiv.) in DCM at rt was added the corresponding aldehyde (1 equiv.) and 4 Å molecular sieves (ca. 100 mg/mmol). The reaction mixture was stirred at rt for 2 h and filtered through a short layer of Celite. The filtrate was concentrated under reduced pressure to afford the imine.

Separately, 2,6-dimethylpyridine (1 equiv.) was added in one portion to a suspension of HFIP (0.25 M) and anhydrous Cu(OTf)₂ (1 equiv.). The resulting mixture was stirred at rt for 1 h, during which time a homogeneous suspension was formed. A solution of the imine (1 equiv.) in DCM (0.0625 M) was added in one portion and the resulting mixture was stirred at rt for 12 h. The reaction was quenched at rt with 10% aqueous ammonium hydroxide, and stirred vigorously for 15 min. The layers were separated and the aqueous layer was extracted with DCM (×3). The combined organic layers were washed with water (×3) and brine, dried over Na₂SO₄, filtered and concentrated under reduced pressure. The residue was purified by flash chromatography to give the product.

### General Procedure 9: Boc Protection

To a solution of the substituted morpholine (1 equiv.) in DCM was added Boc₂O (1.2 equiv.). After stirring at rt for 18 h, the reaction mixture was concentrated under reduced pressure and the residue was purified by flash chromatography to give the desired product.

### General Procedure 10: Boc Protection

To a solution of the substituted morpholine (1 equiv.) in MeCN was added Boc₂O (2 equiv.). After stirring at rt for 18 h, the reaction mixture was concentrated under reduced pressure and the residue was purified by flash chromatography to give the desired product.

### General Procedure 11: EDC Coupling

To a solution of the corresponding carboxylic acid (1 equiv.) in DCM (0.1 M) was added EDC (1.2 equiv) and the resulting mixture was stirred at rt for 15 min before 4,5,6,7-tetrachloroisoindoline-1,3-dione (1 equiv.) and DMAP (0.1 equiv.) were added. The resulting mixture was stirred at rt for a 18 h before the reaction mixture was purified directly by flash chromatography to give the desired product.

### Epoxide 1: (1-Methylcyclopropyl)(1-oxa-6-azaspiro[2.5]octan-6-yl)methanone

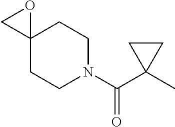

### Step 1: 4-Methylenepiperidine hydrochloride

A solution of 1-N-boc-4-methylene-piperidine (1.97 g, 10 mmol) in DCM (10 mL) and 4 M hydrogen chloride in 1,4-dioxane (25 mL, 100 mmol) was stirred at rt for 100 min before being concentrated under reduced pressure to give title compound (1.34 g, quant.) as a white solid. ¹H NMR (500 MHz, DMSO-d₆) δ 9.17 (s, 2H), 4.86 (s, 2H), 3.05 (t, J=6.1 Hz, 4H), 2.41 (t, J=6.1 Hz, 4H).

### Step 2: (1-Methylcyclopropyl)(4-methylenepiperidin-1-yl)methanone

General procedure 1 using 4-methylenepiperidine hydrochloride (1.34 g, 10 mmol), 1-methylcyclopropane-1-carboxylic acid (1.00 g, 10 mmol), HATU (3.80 g, 10 mmol), DIPEA (4.18 mL, 30 mmol) and DCM (100 mL) after 1 h gave the title compound (1.69 g, 94%) as a colourless oil. ¹H NMR (500 MHz, DMSO-d₆) δ 4.77 (s, 2H), 3.53 (t, J=6.0 Hz, 4H), 2.15 (t, J=5.9 Hz, 4H), 1.23 (s, 3H), 0.85-0.75 (m, 2H), 0.59-0.49 (m, 2H).

### Step 3: (1-Methylcyclopropyl)(1-oxa-6-azaspiro[2.5]octan-6-yl)methanone

General procedure 2 using (1-methylcyclopropyl)-(4-methylene-1-piperidyl)methanone (1.69 g, 9.43 mmol) in DCM (47 mL) at 0° C. and m-CPBA (<77% w/w) (2.54 g, 11.3 mmol) in DCM (47 mL) after 20 h at rt gave the title compound (1.51 g, 82%) as a pale yellow oil. ¹H NMR (500 MHz, DMSO-d₆) δ 3.78-3.68 (m, 2H), 3.62-3.47 (m, 2H), 2.68 (s, 2H), 1.69 (ddd, J=13.1, 8.6, 4.2 Hz, 2H), 1.43 (ddd, J=13.3, 6.5, 3.9 Hz, 2H), 1.23 (s, 3H), 0.83-0.77 (m, 2H), 0.58-0.50 (m, 2H).

### Epoxide 2: (4-Chlorophenyl)(1-oxa-6-azaspiro[2.5]octan-6-yl)methanone

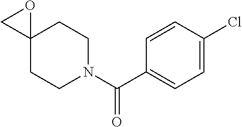 [SMILES: O=C(c1ccc(Cl)cc1)N1CCC2(CC1)CO2]

### Step 1: (4-Chlorophenyl)(4-methylenepiperidin-1-yl)methanone

General procedure 1 using 4-methylenepiperidine hydrochloride (267 mg, 2.00 mmol), 4-chlorobenzoic acid (313 mg, 2.00 mmol), HATU (760 mg, 2.00 mmol), DIPEA (1.05 mL, 6.00 mmol) and DCM (20 mL) after 1 h gave the title compound (405 mg, 85%) as colourless oil. LCMS (method A): R_{T}=1.41 min, m/z=236, 238 [M+H]⁺. ¹H NMR (500 MHz, DMSO-d₆) δ 7.54-7.48 (m, 2H), 7.46-7.41 (m, 2H), 4.79 (s, 2H), 3.60 (br. s, 2H), 3.34 (br. s, 2H (signal overlaps with HDO)), 2.20 (br. s, 4H).

### Step 2: (4-Chlorophenyl)(1-oxa-6-azaspiro[2.5]octan-6-yl)methanone

General procedure 2 using (4-chlorophenyl)-(4-methylene-1-piperidyl)methanone (405 mg, 1.72 mmol) in DCM (8.5 mL) at 0° C. and m-CPBA (<77% w/w) (462 mg, 2.06 mmol) in DCM (8.5 mL) after 20 h at rt gave the title compound (430 mg, 99%) as colourless oil that solidified upon standing. LCMS (method A): R_{T}=0.99 min, m/z=252, 254 [M+H]⁺. ¹H NMR (500 MHz, DMSO-d₆) δ 7.54-7.48 (m, 2H), 7.48-7.42 (m, 2H), 3.85 (br. s, 1H), 3.58 (br. s, 1H), 3.42 (br. s, 2H), 2.69 (s, 2H), 1.76 (br. s, 2H), 1.47 (br. s, 2H).

### Epoxide 3: (R)-4,4,4-Trifluoro-3-phenyl-1-(1-oxa-6-azaspiro[2.5]octan-6-yl)butan-1-one

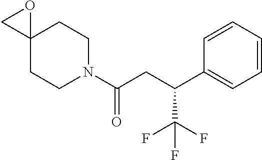 [SMILES: O=C(C[C@H](c1ccccc1)C(F)(F)F)N1CCC2(CC1)CO2]

### Step 1: (R)-4,4,4-Trifluoro-1-(4-methylenepiperidin-1-yl)-3-phenylbutan-1-one

General procedure 1 using 4-methylenepiperidine hydrochloride (59.5 mg, 0.445 mmol), (R)-4,4,4-trifluoro-3-phenylbutanoic acid (97.1 mg, 0.445 mmol), HATU (169 mg, 0.445 mmol), DIPEA (0.233 mL, 1.34 mmol) and DCM (4.5 mL) after 1 h at rt gave title compound (129.5 mg, 97%) as a colourless oil. LCMS (method A): R_{T}=1.54 min, m/z=298 [M+H]⁺. ¹H NMR (500 MHz, DMSO-d₆) δ 7.45-7.39 (m, 2H), 7.38-7.29 (m, 3H), 4.75 (s, 1H), 4.73 (s, 1H), 4.16-4.07 (m, 1H), 3.54-3.42 (m, 2H), 3.42-3.33 (m, 2H), 3.20 (dd, J=16.4, 9.4 Hz, 1H), 2.98 (dd, J=16.4, 4.2 Hz, 1H), 2.20-2.13 (m, 1H), 2.08-2.00 (m, 2H), 1.98-1.91 (m, 1H).

### Step 3: (R)-4,4,4-Trifluoro-3-phenyl-1-(1-oxa-6-azaspiro[2.5]octan-6-yl)butan-1-one

General procedure 2 using (R)-4,4,4-trifluoro-1-(4-methylenepiperidin-1-yl)-3-phenylbutan-1-one (130 mg, 0.436 mmol) in DCM (2.1 mL) at 0° C. and m-CPBA (<77% w/w) (117 mg, 0.523 mmol) in DCM (2.1 mL) after 20 h at rt gave title compound (143 mg, 94%) as a colourless oil. LCMS (method A): R_{T}=1.23 min, m/z=314 [M+H]⁺. ¹H NMR (500 MHz, DMSO-d₆) δ 7.46-7.40 (m, 2H), 7.40-7.29 (m, 3H), 4.18-4.07 (m, 1H), 3.70-3.49 (m, 3H), 3.44-3.34 (m, 1H), 3.21 (dd, J=16.4, 9.4 Hz, 1H), 3.00 (dd, J=16.4, 4.2 Hz, 1H), 2.70-2.59 (m, 2H), 1.76-1.68 (m, 0.5H), 1.62-1.52 (m, 1H), 1.50-1.41 (m, 1H), 1.36-1.29 (m, 1H), 1.27-1.21 (m, 0.5H).

### Epoxide 4: Phenyl(1-oxa-6-azaspiro[2.5]octan-6-yl)methanone

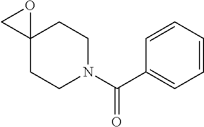 [SMILES: O=C(c1ccccc1)N1CCC2(CC1)CO2]

### Step 1: (4-Methylenepiperidin-1-yl)(phenyl)methanone

General procedure 1 using 4-methylenepiperidine hydrochloride (165 mg, 1.23 mmol), benzoic acid (151 mg, 1.23 mmol), HATU (470 mg, 1.23 mmol), DIPEA (645 μL, 3.70 mmol) and DCM (11 mL) after 30 min gave the title compound (196 mg, 78%) as a white solid. ¹H NMR (500 MHz, Chloroform-d) δ 7.56-7.29 (m, 5H), 4.80 (s, 2H), 3.76 (br. s, 2H), 3.40 (br. s, 2H), 2.33 (br. s, 2H), 2.18 (br. s, 2H).

### Step 2: Phenyl(1-oxa-6-azaspiro[2.5]octan-6-yl)methanone

General procedure 2 using (4-methylenepiperidin-1-yl)(phenyl)methanone (196 mg, 0.974 mmol) in DCM (4.5 mL) at 0° C. and m-CPBA (<77% w/w) (164 mg, 0.733 mmol) in DCM (4.5 mL) after 20 h at rt gave the title compound (163 mg, 77%) as a colourless oil. ¹H NMR (500 MHz, Chloroform-d) δ 7.54-7.32 (m, 5H), 4.25 (br. s, 1H), 3.76-3.42 (br. m, 3H), 2.73 (s, 2H), 2.08-1.79 (br. m, 2H), 1.68-1.30 (br. m, 2H (signals overlap with HDO)).

### Epoxide 5: (4-Fluorophenyl)(1-oxa-6-azaspiro[2.5]octan-6-yl)methanone

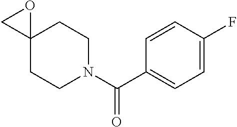 [SMILES: O=C(c1ccc(F)cc1)N1CCC2(CC1)CO2]

### Step 1: (4-Fluorophenyl)(4-methylenepiperidin-1-yl)methanone

General procedure 1 using 4-methylenepiperidine hydrochloride (2.67 g, 20.0 mmol), 4-fluorobenzoic acid (2.80 g, 20.0 mmol), HATU (7.60 g, 20.0 mmol), DIPEA (10.5 mL, 60.0 mmol) and DCM (200 mL) after 30 min gave the title compound (4.31 g, 98%) as a colourless oil. LCMS (method A): R_{T}=1.25 min, m/z=220 [M+H]⁺. ¹H NMR (500 MHz, DMSO-d₆) δ 7.51-7.45 (m, 2H), 7.31-7.24 (m, 2H), 4.79 (s, 2H), 3.59 (br. s, 2H), 3.33 (br. s, 2H (signal overlaps with HDO)), 2.20 (br. s, 4H).

### Step 2: (4-Fluorophenyl)(1-oxa-6-azaspiro[2.5]octan-6-yl)methanone

General procedure 2 using (4-fluorophenyl)(4-methylenepiperidin-1-yl)methanone (4.31 g, 19.7 mmol) in DCM (98 mL) at 0° C. and m-CPBA (<77% w/w) (5.29 g, 23.6 mmol) in DCM (98 mL) after 20 h at rt gave the title compound (3.74 g, 80%) as a colourless oil that solidified upon standing. LCMS (method A): R_{T}=0.82 min, m/z=236 [M+H]⁺. ¹H NMR (500 MHz, DMSO-d₆) δ 7.55-7.45 (m, 2H), 7.31-7.24 (m, 2H), 3.85 (br. s, 1H), 3.70-3.33 (br. m, 3H), 2.69 (s, 2H), 1.76 (br. s, 2H), 1.47 (br. s, 2H).

### Epoxide 6: (2,4-Difluorophenyl)(1-oxa-6-azaspiro[2.5]octan-6-yl)methanone

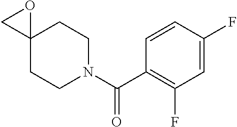 [SMILES: O=C(c1ccc(F)cc1F)N1CCC2(CC1)CO2]

### Step 1: (2,4-Difluorophenyl)(4-methylenepiperidin-1-yl)methanone

General procedure 1 using 4-methylenepiperidine hydrochloride (134 mg, 1.00 mmol), 2,4-difluorobenzoic acid (158 mg, 1.00 mmol), HATU (380 mg, 1.00 mmol), DIPEA (523 μL, 3.00 mmol) and DCM (10 mL) after 75 min gave the title compound (146 mg, 61%) as a viscous colourless oil. LCMS (method A): R_{T}=1.31 min, m/z=238 [M+H]⁺. ¹H NMR (500 MHz, DMSO-d₆) δ 7.54-7.46 (m, 1H), 7.40-7.31 (m, 1H), 7.22-7.14 (m, 1H), 4.81 (s, 1H), 4.78 (s, 1H), 3.64 (br. s, 2H), 3.26-3.15 (m, 2H), 2.30-2.19 (m, 2H), 2.18-2.06 (m, 2H).

### Step 2: (2,4-Difluorophenyl)(1-oxa-6-azaspiro[2.5]octan-6-yl)methanone

General procedure 2 using (2,4-difluorophenyl)(4-methylenepiperidin-1-yl)methanone (145 mg, 0.611 mmol) in DCM (2.7 mL) at rt and m-CPBA (<77% w/w) (164 mg, 0.733 mmol) in DCM (2.7 mL) after 22 h at rt gave the title compound (150 mg, 97%) as a viscous colourless oil. LCMS (method A): R_{T}=0.89 min, m/z=254 [M+H]⁺. ¹H NMR (500 MHz, DMSO-d6) δ 7.58-7.48 (m, 1H), 7.41-7.32 (m, 1H), 7.24-7.14 (m, 1H), 3.98-3.83 (m, 1H), 3.69-3.56 (m, 1H), 3.40-3.31 (m, 2H), 2.71 (d, J=4.8 Hz, 1H), 2.68 (d, J=4.8 Hz, 1H), 1.86-1.73 (m, 1H), 1.73-1.62 (m, 1H), 1.58-1.47 (m, 1H), 1.47-1.34 (m, 1H).

### Epoxide 7: (4-Chloro-2-fluorophenyl)(1-oxa-6-azaspiro[2.5]octan-6-yl)methanone

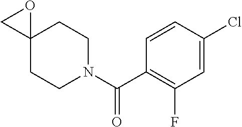 [SMILES: O=C(c1ccc(Cl)cc1F)N1CCC2(CC1)CO2]

### Step 1: (4-Chloro-2-fluorophenyl)(4-methylenepiperidin-1-yl)methanone

General procedure 1 using 4-methylenepiperidine hydrochloride (339 mg, 2.54 mmol), 4-chloro-2-fluorobenzoic acid (443 mg, 2.54 mmol), HATU (965 mg, 2.54 mmol), DIPEA (1.33 mL, 7.61 mmol) and DCM (20 mL) after 19 h gave the title compound (569 mg, 88%) as a colourless gum. LCMS (method A): R_{T}=1.46 min, m/z=254, 256 [M+H]⁺.

### Step 2: (4-Chloro-2-fluorophenyl)(1-oxa-6-azaspiro[2.5]octan-6-yl)methanone

General procedure 2 using (4-chloro-2-fluorophenyl)(4-methylenepiperidin-1-yl)methanone (569 mg, 2.24 mmol in DCM (22 mL) at 0° C. and m-CPBA (<77% w/w) (603 mg, 2.69 mmol) in DCM (22 mL) after 20 h at rt gave the title compound (574 mg, 94%) as a colourless oil. LCMS (method B): R_{T}=1.06 min, m/z=270, 272 [M+H]⁺.

### Epoxide 8: (2-Chlorophenyl)(1-oxa-6-azaspiro[2.5]octan-6-yl)methanone

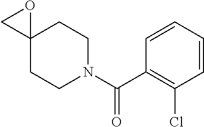 [SMILES: O=C(c1ccccc1Cl)N1CCC2(CC1)CO2]

### Step 1: (2-Chlorophenyl)(4-methylenepiperidin-1-yl)methanone

General procedure 1 using 4-methylenepiperidine hydrochloride (339 mg, 2.54 mmol), 2-chlorobenzoic acid (397 mg, 2.54 mmol), HATU (965 mg, 2.54 mmol), DIPEA (1.33 mL, 7.61 mmol) and DCM (20 mL) after 20 h gave the title compound (564 mg, 94%) as a colourless oil. LCMS (method A): R_{T}=1.34 min, m/z=236, 238 [M+H]⁺.

### Step 2: (2-Chlorophenyl)(1-oxa-6-azaspiro[2.5]octan-6-yl)methanone

General procedure 2 using (2-chlorophenyl)(4-methylenepiperidin-1-yl)methanone (564 mg, 2.39 mmol) in DCM (22 mL) at 0° C. and m-CPBA (<77% w/w) (644 mg, 2.87 mmol) in DCM (22 mL) after 20 h at rt gave the title compound (530 mg, 88%) as a colourless oil. LCMS (method B): R_{T}=0.85 min, m/z=252, 254 [M+H]⁺.

### Epoxide 9: ((1 S*,2S*)-1-Methyl-2-(trifluoromethyl)cyclopropyl)(1-oxa-6-azaspiro[2.5]octan-6-yl)methanone

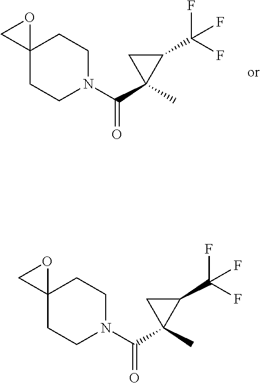 [SMILES: C[C@]1(C(=O)N2CCC3(CC2)CO3)C[C@@H]1C(F)(F)F ; C[C@@]1(C(=O)N2CCC3(CC2)CO3)C[C@H]1C(F)(F)F]

### Step 1: 4-nitrophenyl (1S,2S)-1-methyl-2-(trifluoromethyl)cyclopropane-1-carboxylate and 4-nitrophenyl (1R,2R)-1-methyl-2-(trifluoromethyl)cyclopropane-1-carboxylate

Commercially available 1-methyl-2-(trifluoromethyl)cyclopropane-1-carboxylic acid (dr 82:18) was purified by flash chromatography (5:95 THE in hexanes) to give the single diastereoisomers. The major was assigned as rac-(1R,2R)-1-methyl-2-(trifluoromethyl)cyclopropane-1-carboxylic acid based on NOE experiments. To a solution of rac-(1R,2R)-1-methyl-2-(trifluoromethyl)cyclopropane-1-carboxylic acid (10 g, 59.5 mmol) in DMF (178.5 mL) was added EDC (13.7 g, 71.4 mmol) and the resulting mixture was stirred at rt for 15 min before 4-nitrophenol (9.93 g, 71.4 mmol) and DMAP (73 mg, 0.595 mmol) were added. The resulting mixture was stirred at rt for 19 h before the reaction mixture was diluted with water and extracted with EtOAc (×3). The combined organic layers were washed with water, dried over Na₂SO₄, filtered, and concentrated under reduced pressure. The residue was purified by flash chromatography (0-20% EtOAc in hexanes) to give rac-4-nitrophenyl (1R,2R)-1-methyl-2-(trifluoromethyl)cyclopropane-1-carboxylate (12.7 g, 74%). rac-4-nitrophenyl (1R,2R)-1-methyl-2-(trifluoromethyl)cyclopropane-1-carboxylate (2 g) was resolved into the single stereoisomers by semi-preparative chiral HPLC using a Chiralpak AS-H (4.6 mm×250 mm, 5 μm) column with isocratic solvent conditions: 95:5 hexane (0.1% v/v TFA)/IPA. The first eluted material was arbitrarily assigned as 4-nitrophenyl (1S*,2S*)-1-methyl-2-(trifluoromethyl)cyclopropane-1-carboxylate (532 mg, 26% recovery). Chiral purity (method A): R_{T}=17.06 min, 100% ee. [α]_{D}²⁰=+44.72 (c 0.25, MeOH). The second eluted material was arbitrarily assigned as 4-nitrophenyl (1R*,2R*)-1-methyl-2-(trifluoromethyl)cyclopropane-1-carboxylate (449 mg, 22% recovery). Chiral purity (method A): R_{T}=18.41 min, 97.7% ee. [α]_{D}²¹=−43.92 (c 0.25, MeOH).

### Step 2: ((1S*,2S*)-1-methyl-2-(trifluoromethyl)cyclopropyl)(4-methylenepiperidin-1-yl)methanone

A mixture of 4-nitrophenyl (1S*,2S*)-1-methyl-2-(trifluoromethyl)cyclopropane-1-carboxylate (142 mg, 0.491 mmol), 4-methylenepiperidine hydrochloride (98.4 mg, 0.737 mmol), DIPEA (257 μL, 1.47 mmol) and DMF (4.9 mL) was stirred at rt for 70 min. The reaction mixture was diluted with saturated aqueous sodium hydrogen carbonate (30 mL) and extracted with DCM (3×20 mL) using an ISOLUTE® phase separator. The combined organic phases were concentrated under reduced pressure and the residue purified by flash chromatography to give the title compound (94.6 mg, 77%) as a viscous colourless oil. ¹H NMR (500 MHz, DMSO-d₆) δ 4.79 (s, 2H), 3.50 (br. s, 4H), 2.34-2.06 (m, 5H), 1.44-1.38 (m, 1H), 1.38-1.32 (m, 3H), 1.16-1.10 (m, 1H).

### Step 3: ((1S*,2S*)-1-Methyl-2-(trifluoromethyl)cyclopropyl)(1-oxa-6-azaspiro[2.5]octan-6-yl)methanone

General procedure 2 using ((1 S*,2S*)-1-methyl-2-(trifluoromethyl)cyclopropyl)(4-methylenepiperidin-1-yl)methanone (94.6 mg, 0.383 mmol) in DCM (1.9 mL) at rt and m-CPBA (<77% w/w) (103 mg, 0.459 mmol) in DCM (1.9 mL) after 22 h 30 min at rt gave the title compound (87 mg, 86%) as viscous colourless oil. LCMS (method A, 220 nm): R_{T}=0.99 min, m/z=264 [M+H]⁺. ¹H NMR (500 MHz, DMSO-d₆) δ 3.78-3.64 (m, 2H), 3.52 (br. s, 2H), 2.69 (s, 2H), 2.20-2.10 (m, 1H), 1.72 (br. s, 2H), 1.53-1.39 (m, 3H), 1.38-1.35 (m, 3H), 1.13 (t, J=5.8 Hz, 1H).

### Epoxide 10: ((1R*,2R*)-1-Methyl-2-(trifluoromethyl)cyclopropyl)(1-oxa-6-azaspiro[2.5]octan-6-yl)methanone

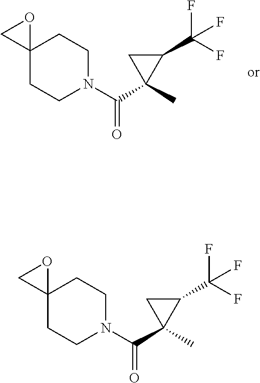 [SMILES: C[C@@]1(C(=O)N2CCC3(CC2)CO3)C[C@H]1C(F)(F)F ; C[C@]1(C(=O)N2CCC3(CC2)CO3)C[C@@H]1C(F)(F)F]

### Step 1: ((1R*,2R*)-1-methyl-2-(trifluoromethyl)cyclopropyl)(4-methylenepiperidin-1-yl)methanone

A mixture of 4-nitrophenyl (1R*,2R*)-1-methyl-2-(trifluoromethyl)cyclopropane-1-carboxylate (238 mg, 0.823 mmol), 4-methylenepiperidine hydrochloride (165 mg, 1.23 mmol), DIPEA (430 μL, 2.47 mmol) and DMF (8.2 mL) was stirred at rt for 80 min. The reaction mixture was diluted with saturated aqueous sodium hydrogen carbonate (30 mL) and extracted with DCM (3×20 mL) using an ISOLUTE® phase separator. The combined organic phases were concentrated under reduced pressure and the residue purified by flash chromatography to give the title compound (103 mg, 50%) as a viscous colourless oil. ¹H NMR (500 MHz, DMSO-d₆) δ 4.79 (s, 2H), 3.50 (s, 4H), 2.29-2.06 (m, 5H), 1.40 (dd, J=9.5, 5.5 Hz, 1H), 1.38-1.32 (m, 3H), 1.13 (t, J=5.9 Hz, 1H).

### Step 2: ((1R*,2R*)-1-Methyl-2-(trifluoromethyl)cyclopropyl)(1-oxa-6-azaspiro[2.5]octan-6-yl)methanone

General procedure 2 using ((1R*,2R*)-1-methyl-2-(trifluoromethyl)cyclopropyl)(4-methylenepiperidin-1-yl)methanone (102 mg, 0.413 mmol) in DCM (2 mL) at rt and m-CPBA (<77% w/w) (111 mg, 0.495 mmol) in DCM (2 mL) after 22 h at rt gave the title compound (107.9 mg, 99%) as a viscous colourless oil. LCMS (method A, 220 nm): R_{T}=1.00 min (220 nm), m/z=264 [M+H]⁺. ¹H NMR (500 MHz, DMSO-d₆) δ 3.78-3.64 (m, 2H), 3.53 (br. s, 2H), 2.69 (s, 2H), 2.20-2.10 (m, 1H), 1.72 (br. s, 2H), 1.56-1.38 (m, 3H), 1.38-1.31 (m, 3H), 1.13 (t, J=5.9 Hz, 1H).

### Epoxide 11: (3-Chlorophenyl)(1-oxa-6-azaspiro[2.5]octan-6-yl)methanone

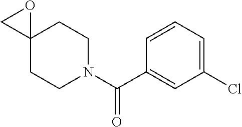 [SMILES: O=C(c1cccc(Cl)c1)N1CCC2(CC1)CO2]

### Step 1: (3-Chlorophenyl)(4-methylenepiperidin-1-yl)methanone

General procedure 1 using 4-methylenepiperidine hydrochloride (339 mg, 2.54 mmol), 3-chlorobenzoic acid (397 mg, 2.54 mmol), HATU (965 mg, 2.54 mmol), DIPEA (1.33 mL, 7.61 mmol) and DCM (20 mL) after 20 h gave the title compound (520 mg, 86%) as a colourless oil. LCMS (method B): R_{T}=1.41 min, m/z=236, 238 [M+H]⁺.

### Step 2: (3-Chlorophenyl)(1-oxa-6-azaspiro[2.5]octan-6-yl)methanone

General procedure 2 using (3-chlorophenyl)(4-methylenepiperidin-1-yl)methanone (520 mg, 2.21 mmol) in DCM (10 mL) at 0° C. and m-CPBA (<77% w/w) (593 mg, 2.65 mmol) in DCM (10 mL) after 20 h at rt gave the title compound (445 mg, 80%) as a colourless oil. LCMS (method B): R_{T}=0.98 min, m/z=252, 254 [M+H]⁺.

### Epoxide 12: (2-Fluorophenyl)(1-oxa-6-azaspiro[2.5]octan-6-yl)methanone

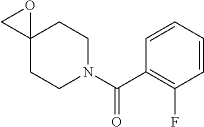 [SMILES: O=C(c1ccccc1F)N1CCC2(CC1)CO2]

### Step 1: (2-Fluorophenyl)(4-methylenepiperidin-1-yl)methanone

General procedure 1 using 4-methylenepiperidine hydrochloride (339 mg, 2.54 mmol), 2-fluorobenzoic acid (355 mg, 2.54 mmol), HATU (965 mg, 2.54 mmol), DIPEA (1.33 mL, 7.61 mmol) and DCM (5 mL) after 20 h gave the title compound (501 mg, 90%) as a viscous colourless oil. LCMS (method A): R_{T}=1.24 min, m/z=220 [M+H]⁺.

### Step 2: (2-Fluorophenyl)(1-oxa-6-azaspiro[2.5]octan-6-yl)methanone

General procedure 2 using (2-fluorophenyl)(4-methylenepiperidin-1-yl)methanone (501 mg, 2.29 mmol) in DCM (10 mL) at rt and m-CPBA (<77% w/w) (615 mg, 2.74 mmol) in DCM (10 mL) after 20 h at rt gave the title compound (501 mg, 93%) as a colourless oil. LCMS (method A): R_{T}=0.80 min, m/z=236 [M+H]⁺.

### Epoxide 13: (3,4-Difluorophenyl)(1-oxa-6-azaspiro[2.5]octan-6-yl)methanone

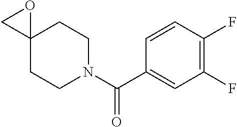 [SMILES: O=C(c1ccc(F)c(F)c1)N1CCC2(CC1)CO2]

### Step 1: (3,4-Difluorophenyl)(4-methylenepiperidin-1-yl)methanone

General procedure 1 using 4-methylenepiperidine hydrochloride (339 mg, 2.54 mmol), 3,4-difluorobenzoic acid (401 mg, 2.54 mmol), HATU (965 mg, 2.54 mmol), DIPEA (1.33 mL, 7.61 mmol) and DCM (15 mL) after 19 h gave the title compound (533, 88%) as a colourless gum. LCMS (method A): R_{T}=1.32 min, m/z=238 [M+H]⁺.

### Step 2: (3,4-Difluorophenyl)(1-oxa-6-azaspiro[2.5]octan-6-yl)methanone

General procedure 2 using (3,4-difluorophenyl)(4-methylenepiperidin-1-yl)methanone (533 mg, 2.25 mmol) in DCM (10 mL) at 0° C. and m-CPBA (<77% w/w) (604 mg, 2.70 mmol) in DCM (10 mL) after 20 h at rt gave the title compound (533 mg, 93%) as a colourless oil. LCMS (method B): R_{T}=0.91 min, m/z=254 [M+H]⁺.

### Epoxide 14: (3-Fluorophenyl)(1-oxa-6-azaspiro[2.5]octan-6-yl)methanone

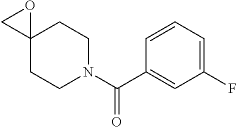 [SMILES: O=C(c1cccc(F)c1)N1CCC2(CC1)CO2]

### Step 1: (3-Fluorophenyl)(4-methylenepiperidin-1-yl)methanone

General procedure 1 using 4-methylenepiperidine hydrochloride (134 mg, 1.00 mmol), 3-fluorobenzoic acid (140 mg, 1.00 mmol), HATU (380 mg, 1.00 mmol), DIPEA (523 μL, 3.00 mmol) and DCM (10 mL) after 19 h gave the title compound (178 mg, 81%) as a viscous colourless oil. LCMS (method A): R_{T}=1.27 min, m/z=220 [M+H]⁺.

### Step 2: (3-Fluorophenyl)(1-oxa-6-azaspiro[2.5]octan-6-yl)methanone

General procedure 2 using (3-fluorophenyl)(4-methylenepiperidin-1-yl)methanone (178 mg, 0.812 mmol) in DCM (4 mL) at rt and m-CPBA (<77% w/w) (218 mg, 0.974 mmol) in DCM (4 mL) after 18 h at rt gave the title compound (183 mg, 95%) as colourless solid. LCMS (method A): R_{T}=0.85 min, m/z=236 [M+H]⁺. ¹H NMR (500 MHz, DMSO-d₆) δ 7.54-7.45 (m, 1H), 7.41-7.09 (m, 3H), 3.87 (br. s, 1H), 3.59 (br. s, 1H), 3.40 (br. s, 2H), 2.69 (s, 2H), 1.77 (br. s, 2H), 1.50 (br. s, 1H), 1.43 (br. s, 1H).

### Epoxide 15: (2,3-Difluorophenyl)(1-oxa-6-azaspiro[2.5]octan-6-yl)methanone

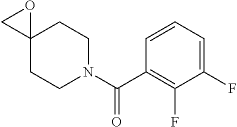 [SMILES: O=C(c1cccc(F)c1F)N1CCC2(CC1)CO2]

### Step 1: (2,3-Difluorophenyl)(4-methylenepiperidin-1-yl)methanone

General procedure 1 using 4-methylenepiperidine hydrochloride (339 mg, 2.54 mmol), 2,3-difluorobenzoic acid (401 mg, 2.54 mmol), HATU (965 mg, 2.54 mmol), DIPEA (1.33 mL, 7.61 mmol) and DCM (15 mL) after 19 h gave the title compound (504 mg, 83%) as a white solid. LCMS (method A): R_{T}=1.31 min, m/z=238 [M+H]⁺.

### Step 2: (2,3-Difluorophenyl)(1-oxa-6-azaspiro[2.5]octan-6-yl)methanone

General procedure 2 using (2,3-difluorophenyl)(4-methylenepiperidin-1-yl)methanone (504 mg, 2.12 mmol) in DCM (10 mL) at 0° C. and m-CPBA (<77% w/w) (571 mg, 2.55 mmol) in DCM (10 mL) after 20 h at rt gave the title compound (501 mg, 93%) as a colourless oil. LCMS (method B): R_{T}=0.89 min, m/z=254 [M+H]⁺.

### Epoxide 16: [1,1′-Bi(cyclopropan)]-1-yl(1-oxa-6-azaspiro[2.5]octan-6-yl)methanone

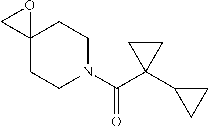 [SMILES: O=C(N1CCC2(CC1)CO2)C1(C2CC2)CC1]

### Step 1: [1,1′-Bi(cyclopropan)]-1-yl(4-methylenepiperidin-1-yl)methanone

General procedure 1 using 4-methylenepiperidine hydrochloride (267 mg, 2.00 mmol), [1,1′-bi(cyclopropane)]-1-carboxylic acid (252 mg, 2.00 mmol), DIPEA (1.05 mL, 6.00 mmol), HATU (760 mg, 2.00 mmol) and DCM (20 mL) after 19 h at rt gave the title compound (392 mg, 95%) as colourless oil. ¹H NMR (500 MHz, DMSO-d₆) δ 4.78 (s, 2H), 3.57 (br. s, 4H), 1.16 (tt, J=8.3, 5.1 Hz, 1H), 2.25-2.07 (m, 4H), 0.73-0.65 (m, 2H), 0.52-0.42 (m, 2H), 0.42-0.34 (m, 2H), 0.15-0.05 (m, 2H).

### Step 2: [1,1′-Bi(cyclopropan)]-1-yl(1-oxa-6-azaspiro[2.5]octan-6-yl)methanone

General procedure 2 using [1,1′-Bi(cyclopropan)]-1-yl(4-methylenepiperidin-1-yl)methanone (392 mg, 1.91 mmol) in DCM (9.5 mL) at 0° C. and m-CPBA (<77% w/w) (514 mg, 2.29 mmol) in DCM (9.5 mL) after 20 h at rt gave title compound (421 mg, 90%) as a colourless oil. LCMS (method A): R_{T}=0.79 min, m/z=222 [M+H]⁺. ¹H NMR (500 MHz, DMSO-d₆) δ 3.78 (br. s, 2H), 3.59 (br. s, 2H), 2.68 (s, 2H), 1.80-1.60 (m, 2H), 1.51-1.38 (m, 2H), 1.20-1.13 (m, 1H), 0.74-0.69 (m, 2H), 0.50-0.44 (m, 2H), 0.43-0.35 (m, 2H), 0.17-0.08 (m, 2H).

### Epoxide 17: (1-Oxa-6-azaspiro[2.5]octan-6-yl)(p-tolyl)methanone

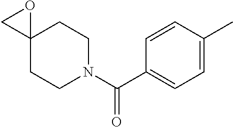 [SMILES: Cc1ccc(C(=O)N2CCC3(CC2)CO3)cc1]

### Step 1: (4-methylenepiperidin-1-yl)(p-tolyl)methanone

General procedure 1 using 4-methylenepiperidine hydrochloride (668 mg, 5.00 mmol), 4-methylbenzoic acid (681 mg, 5.00 mmol), HATU (1.90 g, 5.00 mmol), DIPEA (2.61 mL, 15.0 mmol) and DCM (50 mL) after 1 h gave the title compound (940 mg, 87%) as colourless oil. LCMS (method A): R_{T}=1.36 min, m/z=216 [M+H]⁺. ¹H NMR (500 MHz, DMSO-d₆) δ 7.30 (d, J=8.0 Hz, 2H), 7.25 (d, J=7.8 Hz, 2H), 4.78 (s, 2H), 3.58 (br. s, 2H), 3.34 (br. s, 2H (signal overlaps HDO)), 2.34 (s, 3H), 2.19 (br. s, 4H).

### Step 2: (1-Oxa-6-azaspiro[2.5]octan-6-yl)(p-tolyl)methanone

General procedure 2 using (4-methylenepiperidin-1-yl)(p-tolyl)methanone (940 mg, 4.37 mmol) in DCM (22 mL) at 0° C. and m-CPBA (<77% w/w) (1.17 g, 5.24 mmol) in DCM (22 mL) after 19 h at rt gave the title compound (984 mg, 97%) as a colourless oil that solidified upon standing. LCMS (method A): R_{T}=0.93 min, m/z=232 [M+H]⁺. ¹H NMR (500 MHz, DMSO-d₆) δ 7.33-7.30 (m, 2H), 7.25 (d, J=7.8 Hz, 2H), 3.98-3.33 (br. m, 4H), 2.68 (s, 2H), 2.34 (s, 3H), 1.73 (br. s, 2H), 1.46 (br. s, 2H).

### Epoxide 18: (1-Oxa-6-azaspiro[2.5]octan-6-yl)(4-(trifluoromethyl)phenyl)methanone

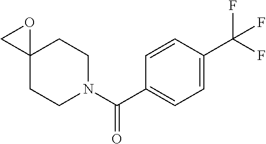 [SMILES: O=C(c1ccc(C(F)(F)F)cc1)N1CCC2(CC1)CO2]

### Step 1: (4-Methylenepiperidin-1-yl)(4-(trifluoromethyl)phenyl)methanone

General procedure 1 using 4-methylenepiperidine hydrochloride (668 mg, 5.00 mmol), 4-(trifluoromethyl)benzoic acid (951 mg, 5.00 mmol), HATU (1.90 g, 5.00 mmol), DIPEA (2.61 mL, 15.0 mmol) and DCM (50 mL) after 1 h gave the title compound (1.31 g, 97%) as a crystalline white solid. LCMS (method A): R_{T}=1.48 min, m/z=270 [M+H]⁺. ¹H NMR (500 MHz, DMSO-d₆) δ 7.82 (d, J=8.0 Hz, 2H), 7.64 (d, J=8.0 Hz, 2H), 4.86-4.73 (m, 2H), 3.64 (s, 2H), 3.26 (s, 2H), 2.27 (s, 2H), 2.16 (s, 2H).

### Step 2: (1-Oxa-6-azaspiro[2.5]octan-6-yl)(4-(trifluoromethyl)phenyl)methanone

General procedure 2 using (4-methylenepiperidin-1-yl)(4-(trifluoromethyl)phenyl)methanone (1.31 g, 4.87 mmol) in DCM (24 mL) at 0° C. and m-CPBA (<77% w/w) (1.31 g, 5.84 mmol) in DCM (24 mL) after 19 h at rt gave the title compound (1.29 g, 92%) as a white solid. LCMS (method A): R_{T}=1.12 min, m/z=286 [M+H]⁺. ¹H NMR (500 MHz, DMSO-d₆) δ 7.82 (d, J=8.0 Hz, 2H), 7.65 (d, J=7.9 Hz, 2H), 3.99-3.82 (m, 1H), 3.69-3.53 (m, 1H), 3.37 (s, 2H), 2.76-2.65 (m, 2H), 1.90-1.78 (m, 1H), 1.78-1.67 (m, 1H), 1.60-1.48 (m, 1H), 1.48-1.35 (m, 1H).

### Epoxide 19: (2-Chloro-4-methylphenyl)(1-oxa-6-azaspiro[2.5]octan-6-yl)methanone

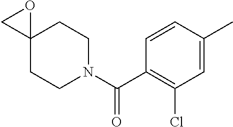 [SMILES: Cc1ccc(C(=O)N2CCC3(CC2)CO3)c(Cl)c1]

### Step 1: (2-Chloro-4-methylphenyl)(4-methylenepiperidin-1-yl)methanone

General procedure 1 using 4-methylenepiperidine hydrochloride (668 mg, 5.00 mmol), 2-chloro-4-methylbenzoic acid (853 mg, 5.00 mmol), HATU (1.90 g, 5.00 mmol), DIPEA (2.61 mL, 15.0 mmol) and DCM (50 mL) after 1 h gave the title compound (1.21 g, 96%) as a colourless oil. LCMS (method A): R_{T}=1.48 min, m/z=250, 252 [M+H]⁺. ¹H NMR (500 MHz, DMSO-d₆) δ 7.37-7.35 (m, 1H), 7.27 (d, J=7.7 Hz, 1H), 7.22 (ddd, J=7.8, 1.5, 0.7 Hz, 1H), 4.83-4.79 (m, 1H), 4.78-4.74 (m, 1H), 3.73-3.66 (m, 1H), 3.65-3.54 (m, 1H), 3.18-3.08 (m, 2H), 2.33 (s, 3H), 2.28-2.19 (m, 2H), 2.19-2.04 (m, 2H).

### Step 2: (2-Chloro-4-methylphenyl)(1-oxa-6-azaspiro[2.5]octan-6-yl)methanone

General procedure 2 using (2-chloro-4-methylphenyl)(4-methylenepiperidin-1-yl)methanone (1.21 g, 4.85 mmol) in DCM (24 mL) at 0° C. and m-CPBA (<77% w/w) (1.30 g, 5.81 mmol) in DCM (24 mL) after 19 h at rt gave the title compound (1.29 g, 92%) as a white solid. LCMS (method A): R_{T}=1.16 min, m/z=266, 268 [M+H]⁺. ¹H NMR (500 MHz, DMSO-d₆) δ 7.36 (s, 1H), 7.29 (t, J=7.1 Hz, 1H), 7.22 (d, J=7.8 Hz, 1H), 3.97-3.89 (m, 0.5H), 3.89-3.81 (m, 0.5H), 3.67 (ddd, J=13.0, 8.5, 4.0 Hz, 0.5H), 3.58 (ddd, J=12.8, 8.9, 3.8 Hz, 0.5H), 3.30-3.16 (m, 2H), 2.74-2.68 (m, 1H), 2.68-2.62 (m, 1H), 2.33 (s, 3H), 1.83 (ddd, J=13.4, 8.8, 4.1 Hz, 0.5H), 1.78-1.63 (m, 1.5H), 1.58-1.51 (m, 0.5H), 1.51-1.34 (m, 1.5H).

### Epoxide 20: (2,4-Dimethylphenyl)(1-oxa-6-azaspiro[2.5]octan-6-yl)methanone

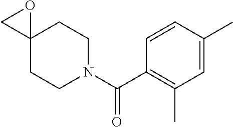 [SMILES: Cc1ccc(C(=O)N2CCC3(CC2)CO3)c(C)c1]

### Step 1: (2,4-Dimethylphenyl)(4-methylenepiperidin-1-yl)methanone

General procedure 1 using 4-methylenepiperidine hydrochloride (339 mg, 2.54 mmol), 2,4-dimethylbenzoic acid (401 mg, 2.54 mmol), HATU (965 mg, 2.54 mmol), DIPEA (1.33 mL, 7.61 mmol) and DCM (15 mL) after 19 h gave the title compound (572 mg, 98%) as a white solid. LCMS (method B): R_{T}=1.31 min, m/z=230 [M+H]⁺.

### Step 2: (2,4-Dimethylphenyl)(1-oxa-6-azaspiro[2.5]octan-6-yl)methanone

General procedure 2 using (2,4-dimethylphenyl)(4-methylenepiperidin-1-yl)methanone (572 mg, 2.49 mmol) in DCM (10 mL) at 0° C. and m-CPBA (<77% w/w) (671 mg, 2.99 mmol) in DCM (10 mL) after 20 h at rt gave the title compound (519 mg, 84%) as a colourless oil. LCMS (method A): R_{T}=1.00 min, m/z=246 [M+H]⁺.

### Epoxide 21: 6-((4-Nitrophenyl)sulfonyl)-1-oxa-6-azaspiro[2.5]octane

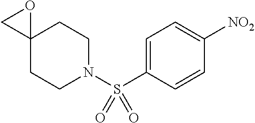 [SMILES: O=[N+]([O-])c1ccc(S(=O)(=O)N2CCC3(CC2)CO3)cc1]

### Step 1: 4-Methylene-1-((4-nitrophenyl)sulfonyl)piperidine

A mixture of 4-nitrobenzenesulfonyl chloride (1.12 g, 5.07 mmol), 4-methylenepiperidine hydrochloride (678 mg, 5.074 mmol), DIPEA (2.65 mL, 15.2 mmol) and THE (50 mL) was stirred at rt overnight. The reaction mixture was diluted with saturated aqueous sodium hydrogen carbonate (100 mL) and extracted with DCM (3×100 mL). The combined organic phases were concentrated under reduced pressure and the residue purified by flash chromatography to give the title compound (1.43 mg, 99%) as white solid. LCMS (method B): R_{T}=1.34 min, mass ion not observed. ¹H NMR (500 MHz, Chloroform-d) δ 8.43-8.32 (m, 2H), 8.00-7.88 (m, 2H), 4.80-4.69 (m, 2H), 3.20-3.06 (m, 4H), 2.39-2.26 (m, 4H).

### Step 2: 6-((4-Nitrophenyl)sulfonyl)-1-oxa-6-azaspiro[2.5]octane

General procedure 2 using 4-methylene-1-((4-nitrophenyl)sulfonyl)piperidine (1.43 g, 5.07 mmol) in DCM (50 mL) at 0° C. and m-CPBA (<77% w/w) (1.36 g, 6.08 mmol) in DCM (50 mL) after 20 h at rt gave the title compound. LCMS (method B): R_{T}=1.07 min, mass ion not observed. ¹H NMR (500 MHz, Chloroform-d) δ 8.48-8.34 (m, 2H), 8.04-7.91 (m, 2H), 3.72-3.57 (m, 2H), 2.92 (td, J=11.3, 3.2 Hz, 2H), 2.68 (s, 2H), 2.17-2.01 (m, 2H), 1.51-1.41 (m, 2H).

### Epoxide 22: (R)-4,4-Difluoro-3-phenyl-1-(1-oxa-6-azaspiro[2.5]octan-6-yl)butan-1-one

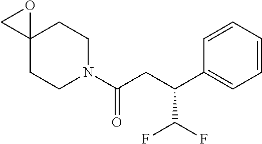 [SMILES: O=C(C[C@H](c1ccccc1)C(F)F)N1CCC2(CC1)CO2]

### Step 1: (R)-4,4-difluoro-1-(4-methylenepiperidin-1-yl)-3-phenylbutan-1-one

General procedure 1 using 4-methylenepiperidine hydrochloride (138 mg, 1.03 mmol), (R)-4,4-difluoro-3-phenylbutanoic acid (207 mg, 1.03 mmol), HATU (393 mg, 1.03 mmol), DIPEA (0.540 mL, 3.10 mmol) and DCM (10.3 mL) after 3 days gave the title compound (255 mg, 88%) as a colourless oil. LCMS (method A): R_{T}=1.46 min, m/z=280 [M+H]⁺. ¹H NMR (500 MHz, DMSO-d₆) δ 7.36-7.24 (m, 5H), 6.24 (td, J=56.5, 3.9H, 1H), 4.74 (d, J=6.2 Hz, 2H), 3.71-3.59 (m, 1H), 3.47-3.33 (m, 4H), 2.95 (dd, J=16.2, 8.2 Hz, 1H), 2.87 (dd, J=16.2, 5.8 Hz, 1H), 2.18-2.10 (m, 1H), 2.09-1.92 (m, 3H).

### Step 2: (R)-4,4-Difluoro-3-phenyl-1-(1-oxa-6-azaspiro[2.5]octan-6-yl)butan-1-one

General procedure 2 using (R)-4,4-difluoro-1-(4-methylenepiperidin-1-yl)-3-phenylbutan-1-one (250 mg, 0.895 mmol) in DCM (4.5 mL) at 0° C. and m-CPBA (<77% w/w) (241 mg, 1.074 mmol) in DCM (4.5 mL) after 20 h at rt gave the title compound (245 mg, 92%) as a colourless oil. LCMS (method A): R_{T}=1.08 min, m/z=296 [M+H]⁺.

### Epoxide 23: (1-Methyl-2-(trifluoromethyl)cyclopropyl)(1-oxa-6-azaspiro[2.5]octan-6-yl)methanone

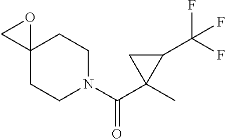 [SMILES: CC1(C(=O)N2CCC3(CC2)CO3)CC1C(F)(F)F]

### Step 1: (1-Methyl-2-(trifluoromethyl)cyclopropyl)(4-methylenepiperidin-1-yl)methanone

To a stirred solution of 1-methyl-2-(trifluoromethyl)cyclopropane-1-carboxylic acid (4:1 mixture diastereoisomers, 5.00 g, 29.8 mmol) in DMF (25 mL) at rt were sequentially added HATU (11.3 g, 29.8 mmol) and DIPEA (18.1 mL, 104 mmol). After stirring at rt for 3 min 4-methylenepiperidine hydrochloride (3.97 g, 29.8 mmol) was added in one portion and the resulting mixture was stirred at rt overnight. The reaction mixture was poured on to 150 mL of water and the resulting mixture was extracted with EtOAc (3×100 mL). The combined organic layers were washed with water and brine. The organic layer was dried over Na₂SO₄, filtered, concentrated under reduced pressure, and the product dried (0.1 mbar/50° C. for 5 h) to give the title compound (6.69 g, 91%). This material was used for the next step without purification.

### Step 2: (1-Methyl-2-(trifluoromethyl)cyclopropyl)(1-oxa-6-azaspiro[2.5]octan-6-yl)methanone

General procedure 2 using (1-Methyl-2-(trifluoromethyl)cyclopropyl)(4-methylenepiperidin-1-yl)methanone (4:1 mixture diastereoisomers, 6.69 g, 27.1 mmol) in DCM (200 mL) at 0° C. and m-CPBA (<70% w/w) (7.84 g, 3.5 mmol) added as solid after 1 h at rt gave the title compound (4.42 g, 62%) as a yellow oil. LCMS (method C): R_{T}=0.95 min, m/z 10=264 [M+H]⁺. ¹H NMR (500 MHz, Chloroform-d) δ 1.08 (t, J=5.9 Hz, 1H), 1.40-1.54 (m, 6H), 1.79-1.93 (m, 3H), 2.72 (s, 2H), 3.27-3.74 (m, 2H), 3.89-4.09 (m, 2H).

### Epoxide 24: (4-Chloro-2-methylphenyl)(1-oxa-6-azaspiro[2.5]octan-6-yl)methanone

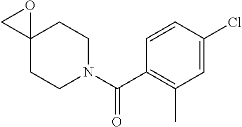 [SMILES: Cc1cc(Cl)ccc1C(=O)N1CCC2(CC1)CO2]

### Step 1: (4-Chloro-2-methylphenyl)(4-methylenepiperidin-1-yl)methanone

General procedure 1 using 4-methylenepiperidine hydrochloride (356 mg, 2.66 mmol), 4-chloro-2-methylbenzoic acid (455 mg, 2.66 mmol), HATU (1.01 g, 2.66 mmol), DIPEA (1.39 mL, 7.99 mmol) and DCM (20 mL) after 20 h gave the title compound (548 mg, 82%) as a white solid. LCMS (method A): R_{T}=1.34 min, m/z=250, 252 [M+H]⁺.

### Step 2: (4-Chloro-2-methylphenyl)(1-oxa-6-azaspiro[2.5]octan-6-yl)methanone

General procedure 2 using (4-chloro-2-methylphenyl)(4-methylenepiperidin-1-yl)methanone (545 mg, 2.18 mmol) in DCM (10 mL) at 0° C. and m-CPBA (<77% w/w) (587 mg, 2.62 mmol) in DCM (10 mL) after 20 h at rt gave the title compound (554 mg, 95%) as a colourless oil. LCMS (method B): R_{T}=1.04 min, m/z=266, 268 [M+H]⁺.

### Epoxide 25: (4-(Difluoromethyl)phenyl)(1-oxa-6-azaspiro[2.5]octan-6-yl)methanone

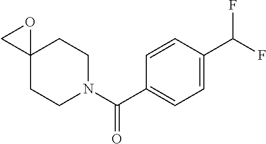 [SMILES: O=C(c1ccc(C(F)F)cc1)N1CCC2(CC1)CO2]

### Step 1: (4-(Difluoromethyl)phenyl)(4-methylenepiperidin-1-yl)methanone

General procedure 1 using 4-methylenepiperidine hydrochloride (372 mg, 2.78 mmol), 4-(difluoromethyl)benzoic acid (479 mg, 2.78 mmol), HATU (1.06 g, 2.78 mmol), DIPEA (1.45 mL, 8.35 mmol) and DCM (20 mL) after 20 h gave the title compound (595 mg, 85%) as a white solid. LCMS (method A): R_{T}=1.20 min, m/z=252 [M+H]⁺.

### Step 2: (4-(Difluoromethyl)phenyl)(1-oxa-6-azaspiro[2.5]octan-6-yl)methanone

General procedure 2 using (4-(difluoromethyl)phenyl)(4-methylenepiperidin-1-yl)methanone (595 mg, 2.37 mmol) in DCM (10 mL) at 0° C. and m-CPBA (<77% w/w) (637 mg, 2.84 mmol) in DCM (10 mL) after 20 h at rt gave the title compound (581 mg, 91%) as a colourless oil. LCMS (method B): R_{T}=0.91 min, m/z=268 [M+H]⁺.

### Epoxide 26: (4-Cyclopropylphenyl)(1-oxa-6-azaspiro[2.5]octan-6-yl)methanone

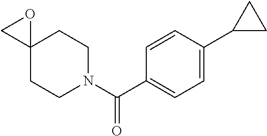 [SMILES: O=C(c1ccc(C2CC2)cc1)N1CCC2(CC1)CO2]

### Step 1: (4-Cyclopropylphenyl)(4-methylenepiperidin-1-yl)methanone

General procedure 1 using 4-methylenepiperidine hydrochloride (1.00 g, 7.49 mmol), 4-cyclopropylbenzoic acid (1.26 g, 7.77 mmol), HATU (2.85 g, 7.49 mmol), DIPEA (3.91 mL, 22.5 mmol) and DMF (12 mL) after 20 h gave the title compound (1.33 g, 71%) as a pale yellow oil. LCMS (method C): R_{T}=1.26 min, m/z=242 [M+H]⁺. ¹H NMR (400 MHz, Chloroform-d) δ 7.31 (d, J=7.4 Hz, 2H), 7.09 (d, J=7.2 Hz, 2H), 4.79 (s, 2H), 3.72 (br. s, 2H), 3.45 (br. s, 2H), 2.25 (br. s, 4H), 1.91 (tt, J=8.6, 4.9 Hz, 1H), 1.09-0.93 (m, 2H), 0.81-0.61 (m, 2H).

### Step 2: (4-Cyclopropylphenyl)(1-oxa-6-azaspiro[2.5]octan-6-yl)methanone

General procedure 2 using (4-cyclopropylphenyl)(4-methylenepiperidin-1-yl)methanone (1.33 g, 5.52 mmol) in DCM (50 mL) at 0° C. and m-CPBA (<70% w/w) (1.50 g, 6.08 mmol) in DCM (50 mL) after 20 h at rt gave the title compound (653 mg, 46%) as a pale yellow oil. LCMS (method C): R_{T}=1.21 min, m/z=258 [M+H]⁺. ¹H NMR (500 MHz, Chloroform-d) δ 7.32 (d, J=7.8 Hz, 2H), 7.09 (d, J=7.8 Hz, 2H), 4.35-4.07 (m, 1H), 3.81-3.45 (m, 3H), 2.73 (s, 2H), 2.07-1.76 (m, 3H), 1.53-1.32 (m, 2H), 1.08-0.91 (m, 2H), 0.79-0.62 (m, 2H).

### Epoxide 27: (4-(1-Methylcyclopropyl)phenyl)(1-oxa-6-azaspiro[2.5]octan-6-yl)methanone

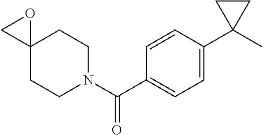 [SMILES: CC1(c2ccc(C(=O)N3CCC4(CC3)CO4)cc2)CC1]

### Step 1: (4-(1-Methylcyclopropyl)phenyl)(4-methylenepiperidin-1-yl)methanone

General procedure 1 using 4-methylenepiperidine hydrochloride (1.63 g, 12.2 mmol), 4-(1-methylcyclopropyl)benzoic acid (2.16 g, 12.2 mmol), HATU (4.64 g, 12.2 mmol), DIPEA (5.10 mL, 36.6 mmol) and DMF (18 mL) after 20 h gave the title compound (2.31 g, 74%) as a pale yellow oil. LCMS (method C): R_{T}=1.34 min, m/z=256 [M+H]⁺. ¹H NMR (400 MHz, Chloroform-d) δ 7.33 (d, J=7.9 Hz, 2H), 7.26 (d, J=7.7 Hz, 2H), 4.79 (s, 2H), 3.68 (br. s, 2H), 3.49 (br. s, 2H), 2.25 (br. s, 4H), 1.41 (s, 3H), 0.99-0.82 (m, 2H), 0.82-0.63 (m, 2H).

### Step 2: (4-(1-Methylcyclopropyl)phenyl)(1-oxa-6-azaspiro[2.5]octan-6-yl)methanone

General procedure 2 using (4-(1-methylcyclopropyl)phenyl)(4-methylenepiperidin-1-yl)methanone (2.31 g, 9.06 mmol) in DCM (50 mL) at 0° C. and m-CPBA (<70% w/w) (1.50 g, 6.08 mmol) in DCM (50 mL) after 20 h at rt gave the title compound (1.19 g, 49%) as a pale yellow oil. LCMS (method C): R_{T}=1.16 min, m/z=272 [M+H]⁺. ¹H NMR (400 MHz, Chloroform-d) δ 7.31 (d, J=8.5 Hz, 2H), 7.24 (d, J=8.5 Hz, 2H), 4.35-4.04 (m, 1H), 3.79-3.46 (m, 3H), 2.71 (s, 2H), 2.03-1.72 (m, 2H), 1.62-1.32 (m, 5H), 0.90-0.83 (m, 2H), 0.78-0.72 (m, 2H). ¹H NMR (400 MHz, Chloroform-d) δ 7.34 (d, J=8.3 Hz, 2H), 7.26 (d, J=8.1 Hz, 2H), 4.22 (br. s, 1H), 3.65 (br. s, 1H), 3.60-3.36 (m, 2H), 2.73 (s, 2H), 1.89 (br. s, 2H), 1.49 (br. s, 2H), 1.41 (s, 3H), 0.99-0.83 (m, 2H), 0.83-0.65 (m, 2H).

### Epoxide 28: ((1 S,2S)-1,2-Dimethylcyclopropyl)(1-oxa-6-azaspiro[2.5]octan-6-yl)methanone

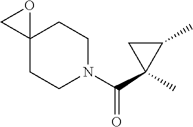

### Step 1: 4-Nitrophenyl (1S,2S)-1,2-dimethylcyclopropane-1-carboxylate

To a solution of (1S,2S)-1,2-dimethylcyclopropane-1-carboxylic acid (404 mg, 3.54 mmol) in DMF (10.6 mL) was added EDC (814 mg, 4.25 mmol) and the resulting mixture was stirred at rt for 15 min before 4-nitrophenol (542 mg, 3.89 mmol) and DMAP (43 mg, 0.354 mmol) were added. The resulting mixture was stirred at rt for 19 h before the reaction mixture was diluted with water and extracted with EtOAc (×3). The combined organic layers were washed with water, dried over Na₂SO₄, filtered, and concentrated under reduced pressure. The residue was purified by flash chromatography (0-20% EtOAc in hexanes) to give the title compound (633 mg, 76%) as a pale-yellow oil. ¹H NMR (500 MHz, Chloroform-d) δ 8.26 (d, J=9.1 Hz, 2H), 7.25 (d, J=9.1 Hz, 2H), 1.76-1.66 (m, 1H), 1.61 (dd, J=9.3, 4.1 Hz, 1H), 1.40 (s, 3H), 1.22 (d, J=6.3 Hz, 3H), 0.58 (dd, J=6.9, 4.1 Hz, 1H).

### Step 2: ((1S,2S)-1,2-Dimethylcyclopropyl)(4-methylenepiperidin-1-yl)methanone

A solution of 1-N-boc-4-methylene-piperidine (796 mg, 4.04 mmol) in 4 M HCl in 1,4-dioxane (3.36 mL, 13.5 mmol) and DCM (2.3 mL) was stirred at rt for 1 h before being concentrated under reduced pressure. To the resulting solid was added a solution of 4-nitrophenyl (1 S,2S)-1,2-dimethylcyclopropane-1-carboxylate (633 mg, 2.69 mmol) in DMF (15.6 mL) and DIPEA (1.41 mL, 8.07 mmol). The resulting mixture was stirred at rt for 90 min before being diluted with saturated aqueous sodium hydrogen carbonate (30 mL). The resulting mixture was extracted with EtOAc (×3), the combined organic layers were washed with 1:1 water/brine (×4), passed through a phase separator and concentrated under reduced pressure. The residue purified by flash chromatography to give the title compound (456 mg, 87%) as a yellow oil. LCMS (method B): R_{T}=1.12 min (220 nm), m/z=194 [M+H]⁺. ¹H NMR (500 MHz, Chloroform-d) δ 4.78 (s, 2H), 3.61-3.51 (m, 4H), 2.21 (t, J=5.8 Hz, 4H), 1.25 (s, 3H), 1.17-1.05 (m, 5H), 0.19 (dd, J=5.4, 4.4 Hz, 1H).

### Step 3: ((1S,2S)-1,2-Dimethylcyclopropyl)(1-oxa-6-azaspiro[2.5]octan-6-yl)methanone

General procedure 2 using ((1S,2S)-1,2-dimethylcyclopropyl)(4-methylenepiperidin-1-yl)methanone (455 mg, 2.35 mmol) in DCM (11.7 mL) at 0° C. and m-CPBA (<77% w/w) (633 mg, 2.82 mmol) in DCM (11.7 mL) after 20 h at rt gave the title compound (413 mg, 83%) as a viscous colourless oil. LCMS (method B): R_{T}=0.79 min (220 nm), m/z=210 [M+H]⁺. ¹H NMR (500 MHz, Chloroform-d) 6 ¹H NMR (500 MHz, Chloroform-d) δ 4.01-3.86 (m, 2H), 3.61-3.42 (m, 2H), 2.72 (s, 2H), 1.84 (ddd, J=14.0, 9.8, 4.5 Hz, 2H), 1.49 (ddd, J=5.2, 3.6, 1.3 Hz, 1H), 1.46 (ddd, J=5.1, 3.8, 1.4 Hz, 1H), 1.26 (s, 3H), 1.19-1.05 (m, 5H), 0.21 (t, J=4.8 Hz, 1H).

### Epoxide 29: ((1R,2R)-1,2-Dimethylcyclopropyl)(1-oxa-6-azaspiro[2.5]octan-6-yl)methanone

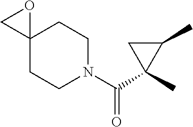

### Step 1: 4-Nitrophenyl (1R,2R)-1,2-dimethylcyclopropane-1-carboxylate

To a solution of (1R,2R)-1,2-dimethylcyclopropane-1-carboxylic acid (598 mg, 5.24 mmol) in DMF (15.7 mL) was added EDC (1.21 g, 6.29 mmol) and the resulting mixture was stirred at rt for 15 min before 4-nitrophenol (802 mg, 5.76 mmol) and DMAP (64 mg, 0.524 mmol) were added. The resulting mixture was stirred at rt for 19 h before the reaction mixture was diluted with water and extracted with EtOAc (×3). The combined organic layers were washed with water, dried over Na₂SO₄, filtered, and concentrated under reduced pressure. The residue was purified by flash chromatography (0-20% EtOAc in hexanes) to give the title compound (912 mg, 74%) as a pale-yellow oil. ¹H NMR (500 MHz, Chloroform-d) δ 8.26 (d, J=9.1 Hz, 2H), 7.25 (d, J=9.1 Hz, 2H), 1.76-1.66 (m, 1H), 1.61 (dd, J=9.3, 4.1 Hz, 1H), 1.40 (s, 3H), 1.22 (d, J=6.3 Hz, 3H), 0.58 (dd, J=6.9, 4.1 Hz, 1H).

### Step 2: ((1R,2R)-1,2-Dimethylcyclopropyl)(4-methylenepiperidin-1-yl)methanone

A solution of 1-N-boc-4-methylene-piperidine (1.15 g, 5.82 mmol) in 4 M HCl in 1,4-dioxane (4.85 mL, 19.4 mmol) and DCM (3.7 mL) was stirred at rt for 1 h before being concentrated under reduced pressure. To the resulting solid was added a solution of 4-nitrophenyl (1R,2R)-1,2-dimethylcyclopropane-1-carboxylate (912 mg, 3.88 mmol) in DMF (22.5 mL) and DIPEA (2.03 mL, 11.6 mmol). The resulting mixture was stirred at rt for 90 min before being diluted with saturated aqueous sodium hydrogen carbonate (30 mL). The resulting mixture was extracted with EtOAc (×3), the combined organic layers were washed with 1:1 water/brine (×4), passed through a phase separator and concentrated under reduced pressure. The residue purified by flash chromatography to give the title compound (722 mg, 96%) as a yellow oil. LCMS (method B): R_{T}=1.12 min (220 nm), m/z=194 [M+H]⁺. ¹H NMR (500 MHz, Chloroform-d) δ 4.78 (s, 2H), 3.61-3.51 (m, 4H), 2.21 (t, J=5.8 Hz, 4H), 1.25 (s, 3H), 1.17-1.05 (m, 5H), 0.19 (dd, J=5.4, 4.4 Hz, 1H).

### Step 3: ((1R,2R)-1,2-Dimethylcyclopropyl)(1-oxa-6-azaspiro[2.5]octan-6-yl)methanone

General procedure 2 using ((1R,2R)-1,2-dimethylcyclopropyl)(4-methylenepiperidin-1-yl)methanone (722 mg, 3.74 mmol) in DCM (18.7 mL) at 0° C. and m-CPBA (<77% w/w) (1.00 g, 4.48 mmol) in DCM (18.7 mL) after 20 h at rt gave the title compound (618 mg, 79%) as a viscous colourless oil. LCMS (method B): R_{T}=0.79 min (220 nm), m/z=210 [M+H]⁺. ¹H NMR (500 MHz, Chloroform-d) 6 ¹H NMR (500 MHz, Chloroform-d) δ 4.01-3.86 (m, 2H), 3.61-3.42 (m, 2H), 2.72 (s, 2H), 1.84 (ddd, J=14.0, 9.8, 4.5 Hz, 2H), 1.49 (ddd, J=5.2, 3.6, 1.3 Hz, 1H), 1.46 (ddd, J=5.1, 3.8, 1.4 Hz, 1H), 1.26 (s, 3H), 1.19-1.05 (m, 5H), 0.21 (t, J=4.8 Hz, 1H).

### Epoxide 30: 2,2-Dicyclopropyl-1-(1-oxa-6-azaspiro[2.5]octan-6-yl)ethan-1-one

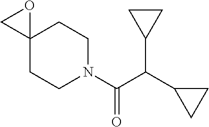 [SMILES: O=C(C(C1CC1)C1CC1)N1CCC2(CC1)CO2]

### Step 1: 2,2-Dicyclopropyl-1-(4-methylenepiperidin-1-yl)ethan-1-one

General procedure 1 using 4-methylenepiperidine hydrochloride (159 mg, 1.19 mmol), 2,2-dicyclopropylacetic acid (167 mg, 1.19 mmol), HATU (452 mg, 1.19 mmol), DIPEA (0.622 mL, 3.57 mmol) and DCM (21 mL) after 19 h gave the title compound (180 mg, 69%) as a colourless oil. LCMS (method B): R_{T}=1.22 min, m/z=220 [M+H]⁺. ¹H NMR (500 MHz, DMSO-d₆) δ 4.77 (s, 2H), 3.49 (br s, 2H), 3.42 (br s, 2H), 2.11 (br s, 4H), 1.70 (t, J=8.8 Hz, 1H), 1.07-0.98 (m, 2H), 0.49-0.42 (m, 2H), 0.35-0.23 (m, 4H), 0.11-0.04 (m, 2H).

### Step 2: 2,2-Dicyclopropyl-1-(1-oxa-6-azaspiro[2.5]octan-6-yl)ethan-1-one

General procedure 2 using 2,2-dicyclopropyl-1-(4-methylenepiperidin-1-yl)ethan-1-one (180 mg, 0.821 mmol) in DCM (4.1 mL) at 0° C. and m-CPBA (<77% w/w) (221 mg, 0.985 mmol) in DCM (4.1 mL) after 20 h at rt gave the title compound (187 mg, 97%) as a colourless oil. LCMS (method B): R_{T}=0.90 min, m/z=236 [M+H]⁺. ¹H NMR (500 MHz, DMSO-d₆) δ 3.77-3.64 (m, 1H), 3.64-3.54 (m, 1H), 3.54-3.45 (m, 2H), 2.67 (s, 2H), 1.71 (t, J=8.8 Hz, 1H), 1.68-1.58 (m, 2H), 1.44-1.33 (m, 2H), 1.08-0.98 (m, 2H), 0.50-0.41 (m, 2H), 0.37-0.23 (m, 4H), 0.13-0.03 (m, 2H).

### Epoxide 31: (1-Methylcyclobutyl)(1-oxa-6-azaspiro[2.5]octan-6-yl)methanone

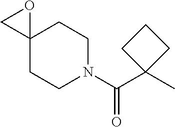

### Step 1: (1-Methylcyclobutyl)(4-methylenepiperidin-1-yl)methanone

General procedure 1 using 4-methylenepiperidine hydrochloride (97 mg, 0.726 mmol), 1-methylcyclobutane-1-carboxylic acid (83 mg, 0.726 mmol), HATU (276 mg, 0.726 mmol), DIPEA (0.379 mL, 2.18 mmol) and DCM (7.2 mL) after 1 h gave the title compound (131 mg, 93%) as a colourless oil. ¹H NMR (500 MHz, DMSO-d₆) δ 4.76 (s, 2H), 3.54-3.33 (m, 2H), 3.28-3.12 (m, 2H), 2.46-2.37 (m, 2H), 2.21-2.04 (m, 4H), 1.98-1.85 (m, 1H), 1.83-1.73 (m, 2H), 1.69-1.55 (m, 1H), 1.35 (s, 3H).

### Step 2: (1-Methylcyclobutyl)(1-oxa-6-azaspiro[2.5]octan-6-yl)methanone

General procedure 2 using (1-methylcyclobutyl)(4-methylenepiperidin-1-yl)methanone (129 mg, 0.667 mmol) in DCM (3.5 mL) at 0° C. and m-CPBA (<77% w/w) (179 mg, 0.801 mmol) in DCM (3.5 mL) after 22.5 h at rt gave the title compound (133 mg, 95%) as a pale-yellow oil. ¹H NMR (500 MHz, DMSO-d₆) δ 3.74-3.59 (m, 1H), 3.48-3.32 (m, 3H), 2.67 (s, 2H), 2.46-2.38 (m, 2H), 1.97-1.86 (m, 1H), 1.83-1.76 (m, 2H), 1.74-1.57 (m, 3H), 1.46-1.37 (m, 2H), 1.36 (s, 3H).

### Epoxide 32: 3-Cyclopropyl-1-(1-oxa-6-azaspiro[2.5]octan-6-yl)propan-1-one

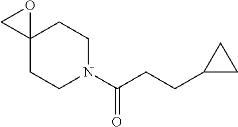 [SMILES: O=C(CCC1CC1)N1CCC2(CC1)CO2]

### Step 1: 3-Cyclopropyl-1-(4-methylenepiperidin-1-yl)propan-1-one

General procedure 1 using 4-methylenepiperidine hydrochloride (356 mg, 2.66 mmol), 3-cyclopropylpropanoic acid (304 mg, 2.66 mmol), HATU (1.01 g, 2.66 mmol), DIPEA (1.39 mL, 7.99 mmol) and DCM (20 mL) after 19 h gave the title compound (269 mg, 52%) as a colourless gum. ¹H NMR (500 MHz, Chloroform-d) δ 4.79-4.76 (m, 2H), 3.60 (t, J=5.9 Hz, 2H), 3.50-3.44 (m, 2H), 2.50-2.42 (m, 2H), 2.22 (dt, J=16.3, 5.9 Hz, 4H), 1.58-1.50 (m, 2H), 0.78-0.67 (m, 1H), 0.49-0.38 (m, 2H), 0.11-0.02 (m, 2H).

### Step 2: 3-Cyclopropyl-1-(1-oxa-6-azaspiro[2.5]octan-6-yl)propan-1-one

General procedure 2 using 3-cyclopropyl-1-(4-methylenepiperidin-1-yl)propan-1-one (269 mg, 1.39 mmol) in DCM (6 mL) at 0° C. and m-CPBA (<77% w/w) (374 mg, 1.67 mmol) in DCM (6 mL) after 20 h at rt gave the title compound (200 mg, 68%) as a colourless oil. ¹H NMR (500 MHz, Chloroform-d) δ 4.15-4.07 (m, 1H), 3.75-3.67 (m, 1H), 3.56 (ddd, J=13.5, 9.9, 3.5 Hz, 1H), 3.42 (ddd, J=13.4, 9.9, 3.6 Hz, 1H), 2.72 (s, 2H), 2.53-2.41 (m, 2H), 1.90-1.79 (m, 2H), 1.58-1.52 (m, 2H), 1.52-1.41 (m, 2H), 0.78-0.68 (m, 1H), 0.49-0.39 (m, 2H), 0.12-0.03 (m, 2H).

### Epoxide 33: rac-((1R,2R)-2-(Difluoromethyl)-1-methylcyclopropyl)(1-oxa-6-azaspiro[2.5]octan-6-yl)methanone

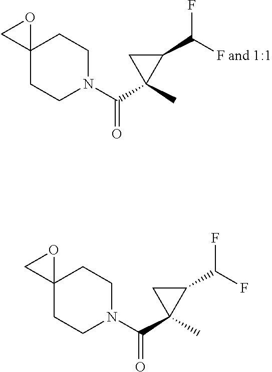

### Step 1: rac-Ethyl (1R,2R)-2-(dimethoxymethyl)-1-methylcyclopropane-1-carboxylate

To a solution of ethyl 2-(diethoxyphosphoryl)propanoate (6.67 mL, 31.1 mmol) and 2-(dimethoxymethyl)oxirane (5.00 g, 42.3 mmol) in 1,2-dimethoxyethane (60 mL) at −78° C. was added dropwise n-butyllithium (2.4 M in hexane, 13 mL, 0.0311 mol). The reaction mixture was allowed to warm up to the rt and stirred for a further 15 min. The reaction mixture was transferred to an autoclave pressure-reactor and heated at 145° C. for 18 h. Upon cooling to rt the reaction mixture was quenched with saturated aqueous ammonium chloride (200 mL), and the product was extracted with MTBE (3×200 mL). The combined organic extracts were dried over sodium sulfate, filtered, and concentrated under reduced pressure to give the title compound (6.80 g, 108%) as a pale-yellow oil. This material was used for next step without purification. ¹H NMR (400 MHz, Chloroform-d) δ 4.28 (d, J=7.1 Hz, 1H), 4.09 (q, J=7.1 Hz, 2H), 3.39 (s, 6H), 1.84 (dt, J=9.5, 6.8 Hz, 1H), 1.41 (dd, J=9.5, 4.2 Hz, 1H), 1.33 (s, 3H), 1.28-1.11 (m, 3H), 0.74 (dd, J=6.6, 4.3 Hz, 1H).

### Step 2: rac-Ethyl (1R,2R)-2-formyl-1-methylcyclopropane-1-carboxylate

To a solution of crude rac-ethyl (1R,2R)-2-(dimethoxymethyl)-1-methylcyclopropane-1-carboxylate (6.80 g, 31.1 mmol) in THE (100 mL) and water (32 mL) was added concentrated aqueous hydrochloric acid (8 mL). The resulting mixture was stirred at rt for 2 h before the volatiles were removed under reduced pressure. The residue was diluted with brine (200 mL) and the product was extracted into DCM (3×100 mL). The combined organic layers were washed with saturated aqueous sodium hydrogen carbonate and brine. The organic layer was dried over sodium sulfate, filtered, and concentrated under reduced pressure to give the title compound (4.60 g, 94% (2 steps)) as a yellow oil. This material was used without further purification. ¹H NMR (500 MHz, Chloroform-d) δ 9.51 (d, J=4.3 Hz, 1H), 4.18-4.11 (m, 2H), 2.55-2.48 (m, 1H), 1.72 (dd, J=8.7, 4.4 Hz, 1H), 1.53-1.48 (m, 1H), 1.44 (s, 3H), 1.26 (t, J=7.2 Hz, 3H).

### Step 3: rac-Ethyl (1R,2R)-2-(difluoromethyl)-1-methylcyclopropane-1-carboxylate

To a stirred solution of crude rac-ethyl (1R,2R)-2-formyl-1-methylcyclopropane-1-carboxylate (4.60 g, 29.5 mmol) in DCM (80 mL) at 0° C. was added morpholinosulfur trifluoride (7.65 g, 43.7 mmol). The reaction was stirred at rt for 19 h before being slowly poured into a vigorously stirred mixture of saturated aqueous sodium hydrogen carbonate and crushed ice. Upon the ice melting the resulting mixture was transferred to a separating funnel, the layers were separated, and the aqueous layer was further extracted with DCM (2×50 mL). The combined organic layers were dried over sodium sulfate, filtered, and concentrated under reduced pressure to give the title compound (5.14 g, 97%) as a yellow oil. This material was used without further purification. ¹H NMR (500 MHz, Chloroform-d) δ 5.60 (td, J=55.9, 6.1 Hz, 1H), 4.13 (q, J=7.1 Hz, 2H), 2.07-1.97 (m, 1H), 1.54-1.48 (m, 1H), 1.39 (s, 3H), 1.26 (t, J=7.1 Hz, 3H), 0.94-0.88 (m, 1H). ¹⁹F NMR (Protons are decoupled, 376 MHz, Chloroform-d) 6-109.20 (d, ²J_{F-F}=289.2 Hz), −113.89 (d, ²J_{F-F}=289.2 Hz).

### Step 4: rac-(1R,2R)-2-(Difluoromethyl)-1-methylcyclopropane-1-carboxylic acid

To a stirred solution rac-ethyl (1R,2R)-2-(difluoromethyl)-1-methylcyclopropane-1-carboxylate (5.14 g, 28.8 mmol) in MeOH (40 mL) was added a solution of sodium hydroxide (2.62 g, 0.0655 mmol) in water (10 mL). The resulting mixture was stirred at rt for 19 h before the volatiles were removed under reduced pressure. The residue was diluted with water (100 mL) and the mixture was acidified to pH 3-4 by the addition of concentrated aqueous hydrochloric acid before the product was extracted into DCM (3×50 mL). The combined organic layers were dried over sodium sulfate, filtered, concentrated under reduced pressure and the residue purified by flash chromatography (SiO₂, 2-5% THE in hexane) to give the title compound (1.77 g, 40%) as a pale-grey crystalline solid. ¹H NMR (600 MHz, Chloroform-d) δ 11.38 (br. s, 1H), 5.63 (td, J=55.8, 5.8 Hz, 1H), 2.15-2.05 (m, 1H), 1.62-1.55 (m, 1H), 1.41 (s, 3H), 1.06-0.99 (m, 1H). ¹⁹F NMR (Protons are decoupled, 376 MHz, Chloroform-d) 6-109.84 (d, J=289.7 Hz), −114.24 (d, J=289.7 Hz). The product was assigned as rac-(1R,2R)-2-(difluoromethyl)-1-methylcyclopropane-1-carboxylic acid based on NOE experiments.

### Step 5: rac-((1R,2R)-2-(Difluoromethyl)-1-methylcyclopropyl)(4-methylenepiperidin-1-yl)methanone

To a stirred solution of rac-(1R,2R)-2-(difluoromethyl)-1-methylcyclopropane-1-carboxylic acid (751 mg, 5.00 mmol) and DMF (19.36 μL, 0.250 mmol) in DCM (5 mL) at 0° C. under an atmosphere of nitrogen was dropwise added oxalyl chloride (2 M in DCM, 3.00 mL, 6.00 mmol). The reaction mixture was allowed to warm to rt and stirred for a further 2 h before being added via syringe to a stirred solution of 4-methylenepiperidine hydrochloride (802 mg, 6.00 mmol) in DCM (5 mL) under an atmosphere of nitrogen. The resulting solution was cooled to 0° C. and triethylamine (2.09 mL, 15.0 mmol) was added dropwise. Upon complete addition the ice-bath was removed, and the reaction mixture was stirred at rt for 1 h before saturated aqueous sodium hydrogen carbonate (60 mL) was added. The resulting mixture was extracted with DCM (3×20 mL) using a phase separator and the combined organic layers were concentrated under reduced pressure to give the title compound (1.25 g, 109%) as a yellow oil. This material was used without further purification. ¹H NMR (500 MHz, DMSO-d₆) δ 5.94 (ddd, J=56.2, 54.5, 7.2 Hz, 1H), 4.81-4.73 (m, 2H), 3.70-3.36 (m, 4H), 2.26-2.07 (m, 4H), 1.72-1.61 (m, 1H), 1.33 (s, 3H), 1.25-1.18 (m, 1H), 0.95-0.91 (m, 1H).

### Step 6: rac-((1R,2R)-2-(Difluoromethyl)-1-methylcyclopropyl)(1-oxa-6-azaspiro[2.5]octan-6-yl)methanone

General procedure 2 using crude rac-((1R,2R)-2-(difluoromethyl)-1-methylcyclopropyl)(4-methylenepiperidin-1-yl)methanone (1.25 g, 5.00 mmol) in DCM (25 mL) at rt and m-CPBA (<77% w/w) (1.46 g, 6.50 mmol) in DCM (25 mL) after 19 h at rt gave the title compound (1.16 g, 94% (2 steps)) as a yellow oil. ¹H NMR (500 MHz, DMSO-d₆) δ 5.94 (ddd, J=56.2, 54.6, 7.2 Hz, 1H), 3.94-3.63 (m, 2H), 3.53 (br. s, 2H), 2.71-2.65 (m, 2H), 1.83-1.61 (m, 3H), 1.54-1.38 (m, 2H), 1.34 (s, 3H), 1.25-1.20 (m, 1H), 0.96-0.90 (m, 1H).

### Example 1: rac-6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

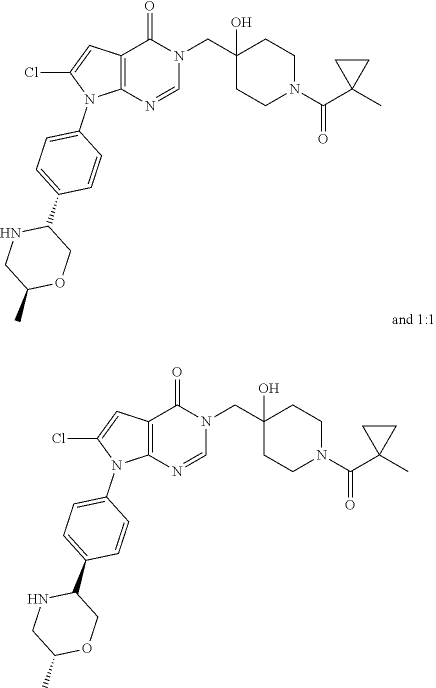

### Step 1: rac-(2R,5S)-5-(4-Bromophenyl)-2-methylmorpholine

General procedure 8 using 4-bromobenzaldehyde (1.85 g, 10.0 mmol), 2-((tributylstannyl)methoxy)propan-1-amine (3.78 g, 10.0 mmol), Cu(OTf)₂ (3.62 g, 10 mmol), 2,6-dimethylpyridine (1.07 g, 10.0 mmol), HFIP (40 mL), 4 Å molecular sieves (ca. 1 g) and DCM (160 mL) gave the title compound (1 g, 39%). ¹H NMR (400 MHz, Chloroform-d) δ 7.43 (d, J=8.4 Hz, 2H), 7.34-7.18 (m, 2H), 3.92-3.73 (m, 2H), 3.65 (ddd, J=10.2, 6.2, 2.4 Hz, 1H), 3.37 (t, J=9.8 Hz, 1H), 3.01 (dd, J=11.7, 2.4 Hz, 1H), 2.70 (dd, J=11.6, 10.1 Hz, 1H), 1.17 (d, J=6.2 Hz, 3H).

### Step 2: rac-tert-Butyl (2R,5S)-5-(4-bromophenyl)-2-methylmorpholine-4-carboxylate

General procedure 9 using rac-(2R,5S)-5-(4-Bromophenyl)-2-methylmorpholine (1 g, 3.90 mmol) and Boc₂O (1.02 g, 4.68 mmol) gave the title compound (1.39 g, quant.). LCMS (method C), R_{T}=1.47 min, m/z=256, 258 [M−Boc+H]⁺. ¹H NMR (500 MHz, Chloroform-d) δ 7.46 (d, J=8.5 Hz, 2H), 7.26 (d, J=8.5 Hz, 2H), 4.77 (t, J=5.4 Hz, 1H), 4.02 (dd, J=12.3, 4.6 Hz, 1H), 3.99-3.92 (m, 1H), 3.72 (dd, J=12.3, 5.8 Hz, 1H), 3.51 (dd, J=13.5, 4.0 Hz, 1H), 3.37 (dd, J=13.5, 4.8 Hz, 1H), 1.35 (s, 9H), 1.29 (d, J=6.5 Hz, 3H).

### Step 3: rac-tert-Butyl (2R,5S)-2-methyl-5-(4-(4,4,5,5-tetramethyl-1,3,2-dioxaborolan-2-yl)phenyl)morpholine-4-carboxylate

General procedure 3 (Work-up A) using rac-tert-butyl (2R,5S)-5-(4-bromophenyl)-2-methylmorpholine-4-carboxylate (600 mg, 1.68 mmol), B₂Pin₂ (642 mg, 2.53 mmol), Pd(dppf)Cl₂·CH₂Cl₂ (143 mg, 0.168 mmol), KOAc (496 mg, 5.05 mmol) and 1,4-dioxane (17 mL) after stirring at 120° C. (microwave irradiation) for 30 min gave the title compound (648 mg, 95%) as a yellow oil. LCMS (method A): R_{T}=1.97 min, m/z=404 [M+H]⁺.

### Step 4: rac-tert-Butyl (2R,5S)-5-(4-(4,6-dichloro-7H-pyrrolo[2,3-d]pyrimidin-7-yl)phenyl)-2-methylmorpholine-4-carboxylate

General procedure 4 (Work-up A) using 4,6-dichloro-7H-pyrrolo[2,3-d]pyrimidine (80 mg, 0.426 mmol), rac-tert-butyl (2R,5S)-2-methyl-5-(4-(4,4,5,5-tetramethyl-1,3,2-dioxaborolan-2-yl)phenyl)morpholine-4-carboxylate (343 mg, 0.851 mmol), copper(II) acetate (155 mg, 0.851 mmol), 1,10-phenanthroline (153 mg, 0.851 mmol), boric acid (105 mg, 1.70 mmol) and DMF (8 mL) after 3 days gave the title compound (98 mg, 49%) as a colourless glass. LCMS (method A): R_{T}=1.90 min, m/z=463, 465 [M+H]⁺.

### Step 5: rac-tert-Butyl (2R,5S)-5-(4-(6-chloro-4-oxo-3,4-dihydro-7H-pyrrolo[2,3-d]pyrimidin-7-yl)phenyl)-2-methylmorpholine-4-carboxylate

General procedure 5 using rac-tert-butyl (2R,5S)-5-(4-(4,6-dichloro-7H-pyrrolo[2,3-d]pyrimidin-7-yl)phenyl)-2-methylmorpholine-4-carboxylate (98 mg, 0.212 mmol), aqueous sodium hydroxide (4 M, 1.06 mL, 4.23 mmol) and 1,4-dioxane (1.1 mL) after 18 h at 100° C. using AcOH for acidification gave the title compound (95 mg, quant.) as a yellow oil. LCMS (method B): R_{T}=1.22 min, m/z=445 [M+H]⁺.

### Step 6: rac-tert-Butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using rac-tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (94 mg, 0.211 mmol), Epoxide 1 (42 mg, 0.211 mmol), Cs₂CO₃ (207 mg, 0.634 mmol) and DMF (2 mL) after 18 h at 80° C. gave the title compound (31 mg, 22%) as colourless glass. LCMS (method A): R_{T}=1.42 min, m/z=640, 642 [M+H]⁺.

### Step 7: rac-6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using rac-tert-butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (31 mg, 0.0484 mmol), TFA (1 mL) and DCM (2 mL) gave, after freeze-drying, the title compound (14 mg, 53%) as white solid. LCMS (method B): R_{T}=0.71 min, m/z=540, 542 [M+H]⁺. ¹H NMR (500 MHz, DMSO-d₆) δ 8.07 (s, 1H), 7.61 (d, J=8.4 Hz, 2H), 7.40 (d, J=8.4 Hz, 2H), 6.81 (s, 1H), 4.91 (s, 1H), 4.01 (s, 2H), 3.99-3.90 (m, 2H), 3.85 (dd, J=10.0, 3.2 Hz, 1H), 3.81 (dd, J=10.6, 3.2 Hz, 1H), 3.61-3.51 (m, 1H), 3.26 (t, J=10.3 Hz, 1H), 3.16 (s, 2H), 2.96 (dd, J=11.7, 2.3 Hz, 1H), 2.94-2.85 (m, 1H), 2.58-2.54 (m, 1H), 1.59-1.36 (m, 4H), 1.21 (s, 3H), 1.10 (d, J=6.2 Hz, 3H), 0.80-0.73 (m, 2H), 0.55-0.49 (m, 2H).

### Example 2: rac-6-Chloro-3-((4-hydroxy-1-(1-methylcyclopropane-1-carbonyl)piperidin-4-yl)methyl)-7-(4-((3R,5S)-5-methylmorpholin-3-yl)phenyl)-3,7-dihydro-4H-pyrrolo[2,3-d]pyrimidin-4-one

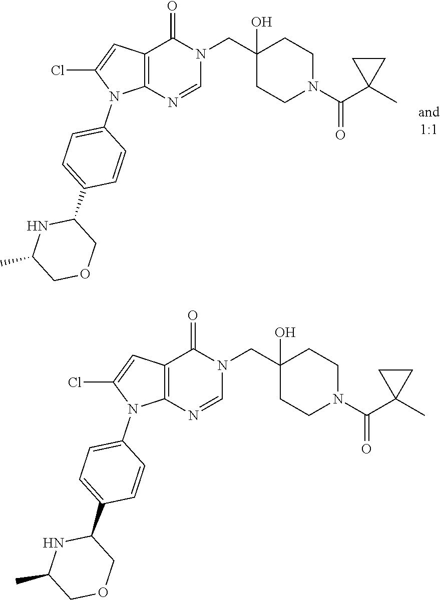

### Step 1: rac-(3R,5S)-3-(4-Bromophenyl)-5-methylmorpholine

General procedure 8 using 4-bromobenzaldehyde (2.83 g, 15.3 mmol), 1-((tributylstannyl)methoxy)propan-2-amine (5.78 g, 15.3 mmol), Cu(OTf)₂ (5.51 g, 15.3 mmol), 2,6-dimethylpyridine (1.63 g, 15.3 mmol), HFIP (61 mL), 4 Å molecular sieves (ca. 1.5 g) and DCM (243 mL) gave the title compound (737 mg, 19%). LCMS (method C): R_{T}=0.68 min, m/z=256, 258 [M+H]⁺.

### Step 2: rac-tert-Butyl (3R,5S)-3-(4-bromophenyl)-5-methylmorpholine-4-carboxylate

General procedure 9 using rac-(3R,5S)-3-(4-Bromophenyl)-5-methylmorpholine (737 mg, 2.88 mmol) and Boc₂O (754 mg, 3.45 mmol) gave the title compound (820 mg, 80%). LCMS (method C), R_{T}=1.50 min, 256, 258 [M−Boc+H]⁺. ¹H NMR (500 MHz, Chloroform-d) δ 7.48 (d, J=8.6 Hz, 2H), 7.43 (d, J=8.7 Hz, 2H), 5.07 (d, J=3.9 Hz, 1H), 4.54-4.46 (m, 1H), 4.11 (dt, J=7.1, 2.5 Hz, 1H), 3.77 (dd, J=12.2, 4.0 Hz, 1H), 3.71 (d, J=2.5 Hz, 2H), 1.50 (s, 9H), 0.91 (d, J=7.1 Hz, 3H).

### Step 3: rac-tert-Butyl (3R,5S)-3-methyl-5-(4-(4,4,5,5-tetramethyl-1,3,2-dioxaborolan-2-yl)phenyl)morpholine-4-carboxylate

General procedure 3 (Work-up A) using rac-tert-butyl (3R,5S)-3-(4-bromophenyl)-5-methylmorpholine-4-carboxylate (600 mg, 1.68 mmol), B₂Pin₂ (642 mg, 2.53 mmol), Pd(dppf)Cl₂·CH₂Cl₂ (143 mg, 0.168 mmol), KOAc (496 mg, 5.05 mmol) and 1,4-dioxane (17 mL) after stirring at 120° C. (microwave irradiation) for 60 min gave the title compound (588 mg, 86%) as a colourless oil. LCMS (method B): R_{T}=1.74 min, m/z=304 [M−Boc+H]⁺.

### Step 4: rac-tert-Butyl (3R,5S)-3-(4-(4,6-dichloro-7H-pyrrolo[2,3-d]pyrimidin-7-yl)phenyl)-5-methylmorpholine-4-carboxylate

General procedure 4 (Work-up A) using 4,6-dichloro-7H-pyrrolo[2,3-d]pyrimidine (548 mg, 2.91 mmol), rac-tert-butyl (3R,5S)-3-methyl-5-(4-(4,4,5,5-tetramethyl-1,3,2-dioxaborolan-2-yl)phenyl)morpholine-4-carboxylate (1.76 mg, 4.37 mmol), copper(II) trifluoroacetate hydrate (1.69 g, 5.83 mmol), 1,10-phenanthroline (1.05 g, 5.83 mmol), boric acid (721 mg, 11.7 mmol) and DMF (55 mL) after 3 days gave the title compound (882 mg, 65%) as a colourless glass. LCMS (method B): R_{T}=1.69 min, m/z=463, 465 [M+H]⁺. ¹H NMR (500 MHz, Chloroform-d) δ 8.62 (s, 1H), 7.89-7.77 (m, 2H), 7.41-7.33 (m, 2H), 6.74 (s, 1H), 5.22 (d, J=3.6 Hz, 1H), 4.59 (dd, J=12.3, 1.2 Hz, 1H), 4.23-4.14 (m, 1H), 3.85 (dd, J=12.3, 4.0 Hz, 1H), 3.80-3.74 (m, 2H), 1.53 (s, 9H), 1.01 (d, J=7.1 Hz, 3H).

### Step 5: rac-tert-Butyl (3R,5S)-3-(4-(6-chloro-4-oxo-3,4-dihydro-7H-pyrrolo[2,3-d]pyrimidin-7-yl)phenyl)-5-methylmorpholine-4-carboxylate

General procedure 5 using rac-tert-butyl (3R,5S)-3-(4-(4,6-dichloro-7H-pyrrolo[2,3-d]pyrimidin-7-yl)phenyl)-5-methylmorpholine-4-carboxylate (145 mg, 0.313 mmol), aqueous sodium hydroxide (2 M, 2.35 mL, 4.69 mmol) and 1,4-dioxane (2.5 mL) after 18 h at 100° C. gave the title compound (140 mg, quant.) as a white solid. LCMS (method B): R_{T}=1.22 min, m/z=445, 446 [M+H]⁺.

### Step 6: rac-tert-Butyl (3R,5S)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-5-methylmorpholine-4-carboxylate

General procedure 6 using rac-tert-butyl (3R,5S)-3-(4-(6-chloro-4-oxo-3,4-dihydro-7H-pyrrolo[2,3-d]pyrimidin-7-yl)phenyl)-5-methylmorpholine-4-carboxylate (140 mg, 0.315 mmol), Epoxide 1 (62 mg, 0.315 mmol), Cs₂CO₃ (205 mg, 0.629 mmol) and DMF (2.5 mL) after 18 h at 80° C. gave the title compound (120 mg, 59%) as a white solid. LCMS (method B): R_{T}=1.33 min, m/z=584, 586 [M−butene+H]⁺, 540, 542 [M−Boc+H]⁺.

### Step 7: rac-6-Chloro-3-((4-hydroxy-1-(1-methylcyclopropane-1-carbonyl)piperidin-4-yl)methyl)-7-(4-((3R,5S)-5-methylmorpholin-3-yl)phenyl)-3,7-dihydro-4H-pyrrolo[2,3-d]pyrimidin-4-one

General procedure 7 using rac-tert-butyl (3R,5S)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-5-methylmorpholine-4-carboxylate (10 mg, 0.0156 mmol), TFA (0.25 mL) and DCM (0.5 mL) gave, after freeze-drying, the title compound (7.1 mg, 78%) as a white solid. LCMS (method B): R_{T}=0.68 min, m/z=540, 542 [M+H]⁺. ¹H NMR (500 MHz, DMSO-d₆) δ 8.07 (s, 1H), 7.62 (d, J=8.1 Hz, 2H), 7.40 (d, J=7.9 Hz, 2H), 6.81 (s, 1H), 4.91 (s, 1H), 4.01 (s, 2H), 3.95 (dt, J=13.0, 4.6 Hz, 3H), 3.75 (dd, J=36.0, 10.4 Hz, 2H), 3.25-3.07 (m, 3H), 2.97 (s, 3H), 1.50 (d, J=12.0 Hz, 2H), 1.39 (d, J=13.3 Hz, 2H), 1.21 (s, 3H), 0.96 (d, J=5.9 Hz, 3H), 0.77 (q, J=4.1 Hz, 2H), 0.53-0.50 (m, 2H).

### Example 3: 6-Chloro-3-((4-hydroxy-1-(1-methylcyclopropane-1-carbonyl)piperidin-4-yl)methyl)-7-(4-(3-methylmorpholin-3-yl)phenyl)-3,7-dihydro-4H-pyrrolo[2,3-d]pyrimidin-4-one

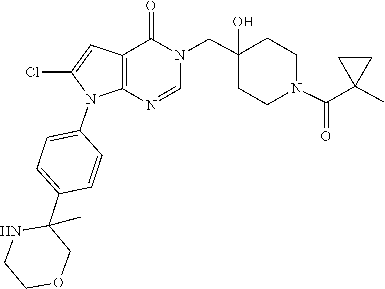 [SMILES: CC1(C(=O)N2CCC(O)(Cn3cnc4c(cc(Cl)n4-c4ccc(C5(C)COCCN5)cc4)c3=O)CC2)CC1]

### Step 1: N-(2-(4-Bromophenyl)-1-hydroxypropan-2-yl)-2-chloroacetamide

To a suspension of 2-amino-2-(4-bromophenyl)propan-1-ol hydrochloride (7.25 g, 27.2 mmol) in DCM was added triethylamine (7.96 ml, 57.1 mmol). The resulted solution was cooled to −30° C. and chloroacetyl chloride (2.16 ml, 27.2 mmol) was added dropwise. The resulting mixture was stirred at rt overnight before being diluted with water. The resulting phases were separated and the organic layer was concentrated under reduced pressure. The residue was purified by flash chromatography to give the title compound (5.00 g, 60%). LCMS (method C): R_{T}=1.12 min, m/z=306, 308 [M+H]⁺. ¹H NMR (500 MHz, Chloroform-d) δ 7.47 (d, J=8.5 Hz, 2H), 7.20 (d, J=8.5 Hz, 2H), 7.13 (br. s, 1H), 4.06 (d, J=2.3 Hz, 2H), 3.87 (d, J=11.8 Hz, 1H), 3.68 (d, J=11.8 Hz, 1H), 3.45 (br. s, 1H), 1.68 (s, 3H).

### Step 2: 5-(4-Bromophenyl)-5-methylmorpholin-3-one

To a solution of N-(2-(4-bromophenyl)-1-hydroxypropan-2-yl)-2-chloroacetamide (5.00 g, 16.3 mmol) in THE (100 mL) at 0° C. was added sodium hydride (55% dispersion in mineral oil, 1.53 g, 35.1 mmol). The resulting mixture was stirred at rt for 18 h before saturated aqueous sodium hydrogen carbonate (70 mL) was added and the mixture was extracted with EtOAc (3×100 mL). The combined organic layers were washed with brine, dried over Na₂SO₄, filtered, and concentrated under reduced pressure. The crude product was purified by flash chromatography to give the title compound (3.35 g, 76%). LCMS (method C): R_{T}=1.11 min, m/z=270, 272 [M+H]⁺. ¹H NMR (400 MHz, Chloroform-d) δ 7.49 (d, J=7.2 Hz, 2H), 7.25 (d, J=7.2 Hz, 2H), 4.17 (s, 2H), 3.78 (d, J=11.9 Hz, 1H), 3.69 (d, J=11.9 Hz, 1H), 1.62 (s, 3H), 0.84 (br. s, 1H).

### Step 3: 3-(4-Bromophenyl)-3-methylmorpholine

To a solution of 5-(4-bromophenyl)-5-methylmorpholin-3-one (3.35 g, 12.4 mmol) in THE (50 mL) at 0° C. under a nitrogen atmosphere was added borane dimethylsulfide complex (5.88 mL, 62.0 mmol). The resulting mixture was allowed to warm to rt and stirred for a further 16 h. To quench the excess borane MeOH (120 mL) was dropwise added and the resulting mixture was stirred at 70° C. for 1 h. Upon cooling to rt the reaction mixture was concentrated under reduced pressure and 5% aqueous hydrochloric acid was added to the residue. The resulting mixture, pH 1, was extracted with EtOAc (2×30 mL) to remove the by-products. The pH of the aqueous layer was adjusted to pH 10 by the addition of Na₂CO₃ before the product was extracted into EtOAc (3×40 mL). The combined organic layers were washed with brine, dried over Na₂SO₄, filtered, and concentrated under reduced pressure to give the title compound (700 mg, 22%). LCMS (method C): R_{T}=1.36 min, m/z=256, 258 [M+H]⁺. ¹H NMR (400 MHz, Chloroform-d) δ 7.49 (d, J=8.6 Hz, 2H), 7.42 (d, J=8.6 Hz, 2H), 4.06 (d, J=11.9 Hz, 1H), 3.69 (m, 2H), 3.55 (d, J=11.9 Hz, 1H), 2.88 (m, 1H), 2.80 (m, 1H), 1.67 (br. s, 1H), 1.34 (s, 3H).

### Step 4: tert-Butyl 3-(4-bromophenyl)-3-methylmorpholine-4-carboxylate

To a solution of 3-(4-bromophenyl)-3-methylmorpholine (700 mg, 2.73 mmol) in THE (10 mL) was added triethylamine (0.457 mL, 3.28 mmol), Boc₂O (0.691 mL, 3.01 mmol) and DMAP (c.a.). The resulted solution was stirred at rt for 16 h before Boc₂O (0.457 ml, 3.28 mmol) was added. After the starting material was consumed, trifluoroethanol (0.43 mL) was added to decompose an excess of Boc₂O. After 5 h the reaction mixture was concentrated under reduced pressure and the residue was purified by flash chromatography to give the title compound (800 mg, 82%). LCMS (method C): R_{T}=1.61 min, m/z=300, 302 [M−butene+H]⁺. ¹H NMR (500 MHz, Chloroform-d) δ 7.43 (d, J=7.2 Hz, 2H), 7.26 (d, J=7.2 Hz, 2H), 4.02 (m, 1H), 3.74 (m, 2H), 3.43 (m, 3H), 1.70 (s, 3H), 1.14 (s, 9H).

### Step 5: tert-Butyl 3-methyl-3-(4-(4,4,5,5-tetramethyl-1,3,2-dioxaborolan-2-yl)phenyl)morpholine-4-carboxylate

General procedure 3 (Work-up A) using tert-butyl 3-(4-bromophenyl)-3-methylmorpholine-4-carboxylate (180 mg, 0.505 mmol), B₂Pin₂ (192 mg, 0.758 mmol), Pd(dppf)Cl₂·CH₂Cl₂ (43 mg, 0.0505 mmol), KOAc (149 mg, 1.52 mmol) and 1,4-dioxane (4 mL) after stirring at 120° C. (microwave irradiation) for 30 min gave the title compound (171 mg, 84%) as a yellow oil. LCMS (method A): R_{T}=1.97 min, m/z=404, 406 [M+H]⁺.

### Step 6: tert-Butyl 3-(4-(4,6-dichloro-7H-pyrrolo[2,3-d]pyrimidin-7-yl)phenyl)-3-methylmorpholine-4-carboxylate

General procedure 4 (Work-up A) using 4,6-dichloro-7H-pyrrolo[2,3-d]pyrimidine (40 mg, 0.213 mmol), tert-butyl 3-methyl-3-(4-(4,4,5,5-tetramethyl-1,3,2-dioxaborolan-2-yl)phenyl)morpholine-4-carboxylate (171 mg, 0.426 mmol), copper(II) acetate (77.3 mg, 0.426 mmol), 1,10-phenanthroline (76.7 mg, 0.426 mmol), boric acid (78.9 mg, 1.28 mmol) and DMF (2 mL) after 3 days gave the title compound (32 mg, 32%) as a colourless film. LCMS (method B): R_{T}=1.65 min, m/z=463, 465 [M+H]⁺.

### Step 4: tert-Butyl 3-(4-(6-chloro-4-oxo-3,4-dihydro-7H-pyrrolo[2,3-d]pyrimidin-7-yl)phenyl)-3-methylmorpholine-4-carboxylate

General procedure 5 using tert-butyl 3-(4-(4,6-dichloro-7H-pyrrolo[2,3-d]pyrimidin-7-yl)phenyl)-3-methylmorpholine-4-carboxylate (32.0 mg, 0.0691 mmol), aqueous sodium hydroxide (2 M, 0.52 mL, 1.04 mmol) and 1,4-dioxane (1 mL) after 18 h at 100° C. gave the title compound (31 mg, quant.) as a white solid. LCMS (method B): R_{T}=1.19 min, m/z=445, 447 [M+H]⁺.

### Step 7: tert-Butyl 3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-3-methylmorpholine-4-carboxylate

General procedure 6 using tert-butyl 3-(4-(6-chloro-4-oxo-3,4-dihydro-7H-pyrrolo[2,3-d]pyrimidin-7-yl)phenyl)-3-methylmorpholine-4-carboxylate (31 mg, 0.0697 mmol), Epoxide 1 (13.6 mg, 0.0697 mmol), Cs₂CO₃ (45 mg, 0.139 mmol) and DMF (0.7 mL) after 3 days at 80° C. gave the title compound (25.4 mg, 56%) as a white solid. LCMS (method B): R_{T}=1.30 min, m/z=584, 586 [M−butene+H]⁺; 540, 542 [M−Boc+H]⁺.

### Step 8: 6-Chloro-3-((4-hydroxy-1-(1-methylcyclopropane-1-carbonyl)piperidin-4-yl)methyl)-7-(4-(3-methylmorpholin-3-yl)phenyl)-3,7-dihydro-4H-pyrrolo[2,3-d]pyrimidin-4-one

General procedure 7 using tert-butyl 3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-3-methylmorpholine-4-carboxylate (25 mg, 0.0391 mmol), TFA (0.5 mL) and DCM (1 mL) gave, after freeze-drying, the title compound (11.3 mg, 51%) as an off-white solid. LCMS (method B): R_{T}=0.674 min, m/z=540, 542 [M+H]⁺. ¹H NMR (500 MHz, DMSO-d₆) δ 8.08 (s, 1H), 7.76-7.72 (m, 2H), 7.41 (d, J=8.5 Hz, 2H), 6.81 (s, 1H), 4.91 (s, 1H), 4.08 (d, J=11.3 Hz, 1H), 4.01 (s, 2H), 3.95 (dt, J=12.9, 4.2 Hz, 2H), 3.58-3.49 (m, 3H), 3.16 (br. s, 2H), 2.71 (dd, J=53.7, 7.1 Hz, 3H), 1.50 (d, J=12.7 Hz, 2H), 1.40 (d, J=13.4 Hz, 2H), 1.27 (s, 3H), 1.21 (s, 3H), 0.77 (q, J=4.1 Hz, 2H), 0.53-0.50 (m, 2H).

### Example 4: 6-Chloro-3-((1-(4-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

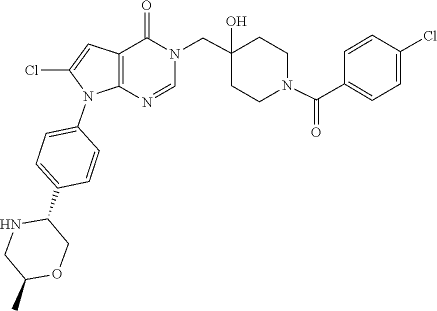 [SMILES: C[C@H]1CN[C@H](c2ccc(-n3c(Cl)cc4c(=O)n(CC5(O)CCN(C(=O)c6ccc(Cl)cc6)CC5)cnc43)cc2)CO1]

### Step 1: (2S,5R)-5-(4-Bromophenyl)-2-methylmorpholine

General procedure 8 using 4-bromobenzaldehyde (1.85 g, 10.0 mmol), (S)-2-((tributylstannyl)methoxy)propan-1-amine (made according to the procedure outlined in Org. Lett. 2014, 16 (4), 1236-1239 starting from (S)-1-aminopropan-2-ol) (3.78 g, 10.0 mmol), Cu(OTf)₂ (3.62 g, 10.0 mmol), 2,6-dimethylpyridine (1.07 g, 10.0 mmol), HFIP (40 mL), 4 Å molecular sieves (ca. 1 g) and DCM (160 mL) gave the title compound (1.37 g, 53%). LCMS (method C): R_{T}=0.63 min, m/z=256, 258 [M+H]⁺. ¹H NMR (500 MHz, Chloroform-d) δ 7.58-7.39 (m, 2H), 7.32-7.19 (m, 2H), 3.94-3.76 (m, 2H), 3.66 (td, J=7.4, 6.8, 3.3 Hz, 1H), 3.38 (t, J=10.1 Hz, 1H), 3.02 (dd, J=11.7, 2.6 Hz, 1H), 2.71 (t, J=10.8 Hz, 1H), 1.92 (s, 1H), 1.18 (d, J=6.2 Hz, 3H).

### Step 2: tert-Butyl (2S,5R)-5-(4-bromophenyl)-2-methylmorpholine-4-carboxylate

General procedure 9 using (2S,5R)-5-(4-bromophenyl)-2-methylmorpholine (1.37 g, 5.37 mmol) and Boc₂O (1.4 g, 6.42 mmol) gave the title compound (1.7 g, 89%). LCMS (method C): R_{T}=1.32 min, m/z=256, 258 [M−Boc+H]⁺. ¹H NMR (500 MHz, Chloroform-d) δ 7.45 (d, J=8.4 Hz, 2H), 7.24 (d, J=8.8 Hz, 2H), 4.76 (t, J=5.2 Hz, 1H), 4.01 (dd, J=12.3, 4.6 Hz, 1H), 3.98-3.92 (m, 1H), 3.71 (dd, J=12.3, 5.8 Hz, 1H), 3.51 (dd, J=13.5, 3.8 Hz, 1H), 3.36 (dd, J=13.5, 4.8 Hz, 1H), 1.34 (s, 9H), 1.28 (d, J=6.4 Hz, 3H). Chiral purity (method B): R_{T}=11.32 min, >98% ee.

### Step 3: tert-Butyl (2S,5R)-2-methyl-5-(4-(4,4,5,5-tetramethyl-1,3,2-dioxaborolan-2-yl)phenyl)morpholine-4-carboxylate

General procedure 3 (Work-up A) using tert-butyl (2S,5R)-5-(4-bromophenyl)-2-methylmorpholine-4-carboxylate (2 g, 5.61 mmol), B₂Pin₂ (2.14 g, 8.42 mmol), Pd(dppf)Cl₂·CH₂Cl₂ (475 mg, 0.561 mmol), KOAc (1.65 g, 16.8 mmol) and 1,4-dioxane (55 mL) after stirring at 100° C. (thermal) for 2 h gave the title compound (2.10 g, 92%) as pale yellow oil. LCMS (method B): R_{T}=1.71 min, m/z=304 [M−Boc+H]⁺.

### Step 4: tert-Butyl (2S,5R)-5-(4-(4,6-dichloro-7H-pyrrolo[2,3-d]pyrimidin-7-yl)phenyl)-2-methylmorpholine-4-carboxylate

General procedure 4 (Work-up A) using 4,6-dichloro-7H-pyrrolo[2,3-d]pyrimidine (652 mg, 3.48 mmol), tert-butyl (2S,5R)-2-methyl-5-(4-(4,4,5,5-tetramethyl-1,3,2-dioxaborolan-2-yl)phenyl)morpholine-4-carboxylate (2.10 g, 5.20 mmol), copper(II) trifluoroacetate hydrate (2.01 g, 6.94 mmol), 1,10-phenanthroline (1.25 g, 6.94 mmol), boric acid (858 mg, 13.9 mmol) and DMF (65 mL) after 24 hours at 75-80° C. gave the title compound (1.07 g, 66%) as a colourless glass. LCMS (method B): R_{T}=1.68 min, m/z=463, 465 [M+H]⁺.

### Step 5: tert-Butyl (2S,5R)-5-(4-(6-chloro-4-oxo-3,4-dihydro-7H-pyrrolo[2,3-d]pyrimidin-7-yl)phenyl)-2-methylmorpholine-4-carboxylate

General procedure 5 using tert-butyl (2S,5R)-5-(4-(4,6-dichloro-7H-pyrrolo[2,3-d]pyrimidin-7-yl)phenyl)-2-methylmorpholine-4-carboxylate (1.07 g, 2.30 mmol), aqueous sodium hydroxide (4 M, 17.2 mL, 69.0 mmol) and 1,4-dioxane (17.3 mL) after 18 h at 100° C. using 1 M aqueous potassium bisulfate for acidification gave the title compound (1.08 g, >100%) as a yellow glass. LCMS (method B): R_{T}=1.30 min, m/z=445, 447 [M+H]⁺.

### Step 6: tert-Butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (442 mg, 0.994 mmol), Epoxide 2 (375 mg, 1.49 mmol), Cs₂CO₃ (971 mg, 2.98 mmol) and DMF (10 mL) after 18 h at 80° C. gave the title compound (498 mg, 72%) as a colourless glass. LCMS (method B): R_{T}=1.50 min, m/z=640, 642 [M−butene+H]⁺.

### Step 7: 6-Chloro-3-((1-(4-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (498 mg, 0.715 mmol), TFA (5 mL) and DCM (10 mL) gave, after freeze-drying, the title compound (385 mg, 90%) as a white solid. LCMS (method B): R_{T}=0.83 min, m/z=596, 598 [M+H]⁺. ¹H NMR (500 MHz, DMSO-d₆) δ 8.06 (s, 1H), 7.65-7.55 (m, 2H), 7.55-7.48 (m, 2H), 7.47-7.34 (m, 4H), 6.79 (s, 1H), 4.95 (s, 1H), 4.31-4.08 (m, 1H), 4.02 (s, 2H), 3.85 (dd, J=10.1, 3.3 Hz, 1H), 3.80 (dd, J=10.6, 3.2 Hz, 1H), 3.62-3.49 (m, 1H), 3.48-3.33 (m, 1H), 3.29-3.20 (m, 2H), 3.20-3.05 (m, 1H), 3.04-2.77 (m, 2H), 2.58-2.52 (m, 1H), 1.69-1.53 (m, 2H), 1.53-1.25 (m, 2H), 1.09 (d, J=6.2 Hz, 3H). Chiral purity (method C): R_{T}=2.98 min, 99.8% ee.

### Example 5: 6-Chloro-3-((1-(4-chlorobenzoyl)-4-hydroxypiperidin-4-yl)methyl)-7-(3-methyl-4-((3R,6S)-6-methylmorpholin-3-yl)phenyl)-3,7-dihydro-4H-pyrrolo[2,3-d]pyrimidin-4-one

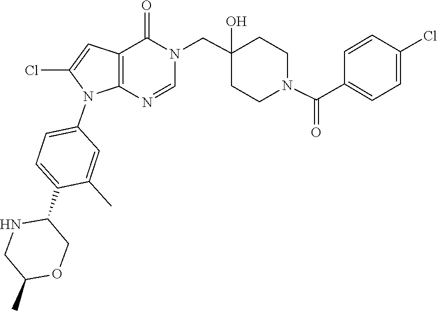 [SMILES: Cc1cc(-n2c(Cl)cc3c(=O)n(CC4(O)CCN(C(=O)c5ccc(Cl)cc5)CC4)cnc32)ccc1[C@@H]1CO[C@@H](C)CN1]

### Step 1: (2S,5R)-5-(4-Bromo-2-methylphenyl)-2-methylmorpholine

General procedure 8 using 4-bromo-2-methylbenzaldehyde (1.99 g, 10.0 mmol), (S)-2-((tributylstannyl)methoxy)propan-1-amine (made according to the procedure outlined in Org. Lett. 2014, 16 (4), 1236-1239 starting from (S)-1-aminopropan-2-ol) (3.78 g, 10.0 mmol), Cu(OTf)₂ (3.62 g, 10.0 mmol), 2,6-dimethylpyridine (1.07 g, 10.0 mmol), HFIP (40 mL), 4 Å molecular sieves (ca. 1 g) and DCM (160 mL) gave the title compound (1.45 g, 53%). LCMS (method C): R_{T}=0.63 min, m/z=270, 272 [M+H]⁺.

### Step 2: tert-Butyl (2S,5R)-5-(4-bromo-2-methylphenyl)-2-methylmorpholine-4-carboxylate

General procedure 9 using (2S,5R)-5-(4-Bromo-2-methylphenyl)-2-methylmorpholine (1.45 g, 5.37 mmol) and Boc₂O (1.4 g, 6.42 mmol) gave the title compound (1.77 g, 89%). LCMS (method C with 5 min gradient): R_{T}=3.54 min, m/z=270, 272 [M−Boc+H]⁺. ¹H NMR (500 MHz, Chloroform-d) δ 7.36-7.12 (m, 3H), 4.72 (dd, J=9.1, 4.9 Hz, 1H), 3.92 (td, J=7.2, 6.8, 4.1 Hz, 1H), 3.84 (dd, J=12.3, 4.7 Hz, 1H), 3.76 (dd, J=13.5, 3.8 Hz, 1H), 3.45 (dd, J=12.3, 8.8 Hz, 1H), 3.23 (dd, J=13.5, 6.5 Hz, 1H), 2.31 (s, 3H), 1.25 (d, J=5.9 Hz, 3H), 1.19 (s, 9H). Chiral purity (method D): R_{T}=5.371 min, >98% ee.

### Step 3: tert-butyl (2S,5R)-2-methyl-5-(2-methyl-4-(4,4,5,5-tetramethyl-1,3,2-dioxaborolan-2-yl)phenyl)morpholine-4-carboxylate

General procedure 3 (Work-up A) using tert-butyl (2S,5R)-5-(4-bromo-2-methylphenyl)-2-methylmorpholine-4-carboxylate (600 mg, 1.62 mmol), B₂Pin₂ (617 mg, 2.43 mmol), Pd(dppf)Cl₂·CH₂Cl₂ (137 mg, 0.162 mmol), KOAc (477 mg, 4.86 mmol) and 1,4-dioxane (17 mL) after stirring at 120° C. (microwave irradiation) for min gave the title compound (432 mg, 63%) as pale yellow oil. LCMS (method B): R_{T}=1.83 min, m/z=318 [M−Boc+H]⁺.

### Step 4: tert-Butyl (2S,5R)-5-(4-(4,6-dichloro-7H-pyrrolo[2,3-d]pyrimidin-7-yl)-2-methylphenyl)-2-methylmorpholine-4-carboxylate

General procedure 4 (Work-up A) using 4,6-dichloro-7H-pyrrolo[2,3-d]pyrimidine (97 mg, 0.516 mmol), tert-butyl (2S,5R)-2-methyl-5-(2-methyl-4-(4,4,5,5-tetramethyl-1,3,2-dioxaborolan-2-yl)phenyl)morpholine-4-carboxylate (431 mg, 1.03 mmol), copper(II) acetate (187 mg, 1.03 mmol), 1,10-phenanthroline (186 mg, 1.03 mmol), boric acid (128 mg, 2.06 mmol) and DMF (10 mL) after 6 days at 50° C. gave the title compound (107 mg, 43%) as a colourless glass. LCMS (method B): R_{T}=1.76 min, m/z=477, 479 [M+H]⁺.

### Step 5: tert-Butyl (2S,5R)-5-(4-(6-chloro-4-oxo-3,4-dihydro-7H-pyrrolo[2,3-d]pyrimidin-7-yl)-2-methylphenyl)-2-methylmorpholine-4-carboxylate

General procedure 5 using tert-butyl (2S,5R)-5-(4-(4,6-dichloro-7H-pyrrolo[2,3-d]pyrimidin-7-yl)-2-methylphenyl)-2-methylmorpholine-4-carboxylate (107 mg, 0.224 mmol), aqueous sodium hydroxide (4 M, 1.12 mL, 4.48 mmol) and 1,4-dioxane (1.12 mL) after 18 h at 100° C. using AcOH for acidification gave the title compound (104 mg, >100%) as a yellow glass. LCMS (method B): R_{T}=1.28 min, m/z=459, 461 [M+H]⁺.

### Step 6: tert-Butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)-2-methylphenyl)-2-methylmorpholine-4-carboxylate (52 mg, 0.113 mmol), Epoxide 2 (42.8 mg, 0.170 mmol), Cs₂CO₃ (111 mg, 0.340 mmol) and DMF (2 mL) after 18 h at 80° C. gave the title compound (33 mg, 41%) as a colourless glass. LCMS (method B): R_{T}=1.55 min, m/z=654, 656 [M−butene+H]⁺.

### Step 7: 6-Chloro-3-((1-(4-chlorobenzoyl)-4-hydroxypiperidin-4-yl)methyl)-7-(3-methyl-4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (33 mg, 0.0464 mmol), TFA (1 mL) and DCM (2 mL) gave, after freeze-drying, the title compound (26 mg, 91%) as a white solid. LCMS (method B): R_{T}=0.87 min, m/z=610, 612 [M+H]⁺. ¹H NMR (500 MHz, DMSO-d₆) δ 8.05 (s, 1H), 7.73 (d, J=7.9 Hz, 1H), 7.54-7.47 (m, 2H), 7.42 (d, J=8.4 Hz, 2H), 7.27-7.19 (m, 2H), 6.77 (s, 1H), 4.95 (s, 1H), 4.22-4.09 (m, 1H), 4.01 (s, 2H), 3.98 (dd, J=10.0, 3.0 Hz, 1H), 3.79 (dd, J=10.9, 3.0 Hz, 1H), 3.61-3.53 (m, 1H), 3.43-3.33 (m, 1H), 3.28-3.07 (m, 3H), 2.97 (dd, J=11.6, 2.3 Hz, 1H), 2.84-2.71 (m, 1H), 2.61-2.52 (m, 1H), 2.41 (s, 3H), 1.68-1.52 (m, 2H), 1.52-1.29 (m, 2H), 1.10 (d, J=6.2 Hz, 3H).

### Example 6: 6-Chloro-3-((1-(4-chlorobenzoyl)-4-hydroxypiperidin-4-yl)methyl)-7-(4-((2S,3S)-2-methylmorpholin-3-yl)phenyl)-3,7-dihydro-4H-pyrrolo[2,3-d]pyrimidin-4-one

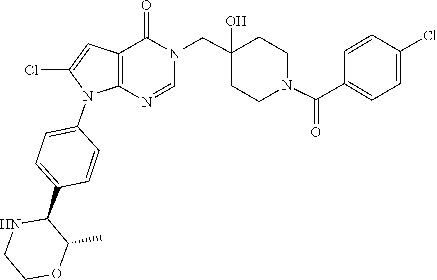 [SMILES: C[C@@H]1OCCN[C@H]1c1ccc(-n2c(Cl)cc3c(=O)n(CC4(O)CCN(C(=O)c5ccc(Cl)cc5)CC4)cnc32)cc1]

### Step 1: 4-(tert-Butyl) 3-(4,5,6,7-tetrachloro-1,3-dioxoisoindolin-2-yl) (2S,3R)-2-methylmorpholine-3,4-dicarboxylate

General procedure 11 using (2S,3R)-4-(tert-butoxycarbonyl)-2-methylmorpholine-3-carboxylic acid (1.11 g, 4.54 mmol), 4,5,6,7-tetrachloro-2-hydroxyisoindoline-1,3-dione (1.37 g, 4.54 mmol), EDC (1.04 g, 5.45 mmol) and DMAP (55 mg, 0.454 mmol) gave the title compound (1.10 g, 46%) as a yellow solid. ¹H NMR (400 MHz, Chloroform-d) δ 4.60-4.15 (m, 2H), 3.95-3.86 (m, 1H), 3.73-3.61 (m, 1H), 3.60-3.35 (m, 2H), 1.61-1.43 (m, 12H).

### Step 2: tert-Butyl (2S,3S)-3-(4-bromophenyl)-2-methylmorpholine-4-carboxylate

To 4-(tert-butyl) 3-(4,5,6,7-tetrachloro-1,3-dioxoisoindolin-2-yl) (2S,3R)-2-methylmorpholine-3,4-dicarboxylate (363 mg, 0.687 mmol), (4-bromophenyl)boronic acid (414 mg, 2.06 mmol) in a 100 mL RBF under nitrogen was added 1,4-dioxane (26 mL). The reaction mixture was heated to 75° C. (internal temperature) before triethylamine (958 μL, 6.87 mmol) and NiCl₂ 6H₂O: bathophenanthroline complex in DMF (0.05 M, 2.6 mL, 0.130 mmol (Angew. Chem. Int. Ed. 2016, 55, 9676-9679)) were sequentially added. The resulting mixture was stirred at 75° C. overnight. Upon cooling to rt the reaction mixture was diluted with EtOAc (40 mL) and washed with brine. The organic layer was dried over MgSO₄, filtered and concentrated under reduced pressure. The residue was purified by flash chromatography to give the title compound (103 mg, 42%) as a colourless glass. LCMS (method B): R_{T}=1.61 min, m/z=256, 258 [M−Boc+H]⁺.

### Step 3: tert-Butyl (2S,3S)-2-methyl-3-(4-(4,4,5,5-tetramethyl-1,3,2-dioxaborolan-2-yl)phenyl)morpholine-4-carboxylate

General procedure 3 (Work-up A) using tert-butyl (2S,3S)-3-(4-bromophenyl)-2-methylmorpholine-4-carboxylate (243 mg, 0.682 mmol), B₂Pin₂ (346 mg, 1.36 mmol), Pd(dppf)Cl₂·CH₂Cl₂ (58 mg, 0.0682 mmol), KOAc (201 mg, 2.046 mmol) and 1,4-dioxane (7 mL) after stirring at 120° C. (microwave irradiation) for 30 min gave the title compound (133 mg, 48%) as a colourless glass. LCMS (method B): R_{T}=1.73 min, m/z=304 [M−Boc+H]⁺. ¹H NMR (500 MHz, Chloroform-d) δ 7.81-7.74 (m, 2H), 7.46-7.38 (m, 2H), 4.68 (d, J=4.1 Hz, 1H), 4.29-4.16 (m, 1H), 3.97 (td, J=10.9, 4.1 Hz, 1H), 3.84-3.75 (m, 1H), 3.74-3.62 (m, 1H), 3.34-3.19 (m, 1H), 1.38 (s, 9H), 1.34 (s, 12H), 1.31 (d, J=6.4 Hz, 3H).

### Step 4: tert-Butyl (2S,3S)-3-(4-(4,6-dichloro-7H-pyrrolo[2,3-d]pyrimidin-7-yl)phenyl)-2-methylmorpholine-4-carboxylate

General procedure 4 (Work-up A) using 4,6-dichloro-7H-pyrrolo[2,3-d]pyrimidine (62 mg, 0.330 mmol), tert-butyl (2S,3S)-2-methyl-3-(4-(4,4,5,5-tetramethyl-1,3,2-dioxaborolan-2-yl)phenyl)morpholine-4-carboxylate (133 mg, 0.330 mmol), copper(II) acetate (120 mg, 0.660 mmol), 1,10-phenanthroline (119 mg, 0.660 mmol), boric acid (82 mg, 1.32 mmol) and DMF (6.2 mL) after 5 days at 50° C. gave the title compound (40 mg, 26%) as a colourless glass. LCMS (method B): R_{T}=1.68 min, m/z=463, 465 [M+H]⁺.

### Step 5: tert-Butyl (2S,3S)-3-(4-(6-chloro-4-oxo-3,4-dihydro-7H-pyrrolo[2,3-d]pyrimidin-7-yl)phenyl)-2-methylmorpholine-4-carboxylate

General procedure 5 using tert-butyl (2S,3S)-3-(4-(4,6-dichloro-7H-pyrrolo[2,3-d]pyrimidin-7-yl)phenyl)-2-methylmorpholine-4-carboxylate (40 mg, 0.0863 mmol), aqueous sodium hydroxide (4 M, 0.43 mL, 1.73 mmol) and 1,4-dioxane (0.43 mL) after 18 h at 100° C. using AcOH for acidification gave the title compound (38 mg, 98%) as a yellow glass. LCMS (method B): R_{T}=1.22 min, m/z=445, 447 [M+H]⁺.

### Step 6: tert-Butyl (2S,3S)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,3S)-3-(4-(6-chloro-4-oxo-3,4-dihydro-7H-pyrrolo[2,3-d]pyrimidin-7-yl)phenyl)-2-methylmorpholine-4-carboxylate (39 mg, 0.0877 mmol), Epoxide 2 (33.1 mg, 0.132 mmol), Cs₂CO₃ (85.7 mg, 0.263 mmol) and DMF (2 mL) after 18 h at 80° C. gave the title compound (14 mg, 22%) as a colourless glass. LCMS (method B): R_{T}=1.48 min, m/z=640, 642 [M−butene+H]⁺.

### Step 7: 6-Chloro-3-((1-(4-chlorobenzoyl)-4-hydroxypiperidin-4-yl)methyl)-7-(4-((2S,3S)-2-methylmorpholin-3-yl)phenyl)-3,7-dihydro-4H-pyrrolo[2,3-d]pyrimidin-4-one

General procedure 7 using tert-butyl (2S,3S)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (14 mg, 0.0201 mmol), TFA (1 mL) and DCM (2 mL) gave, after freeze-drying, the title compound (11 mg, 91%) as a white solid. LCMS (method B): R_{T}=0.85 min, m/z=596, 598 [M+H]⁺. ¹H NMR (500 MHz, DMSO-d₆) δ 8.07 (s, 1H), 7.61-7.54 (m, 2H), 7.54-7.48 (m, 2H), 7.45-7.41 (m, 2H), 7.41-7.36 (m, 2H), 6.79 (s, 1H), 4.95 (s, 1H), 4.22-4.11 (m, 1H), 4.02 (s, 2H), 3.80 (dd, J=10.7, 1.9 Hz, 1H), 3.62 (td, J=11.0, 3.0 Hz, 1H), 3.44 (d, J=8.7 Hz, 1H), 3.39 (dq, J=8.6, 6.0 Hz, 2H), 3.28-3.21 (m, 1H), 3.19-3.07 (m, 1H), 2.93-2.75 (m, 3H), 1.67-1.52 (m, 2H), 1.52-1.27 (m, 2H), 0.86 (d, J=6.0 Hz, 3H).

### Example 7: 6-Chloro-3-((4-hydroxy-1-((R)-4,4,4-trifluoro-3-phenylbutanoyl)piperidin-4-yl)methyl)-7-(4-((3R,6S)-6-methylmorpholin-3-yl)phenyl)-3,7-dihydro-4H-pyrrolo[2,3-d]pyrimidin-4-one

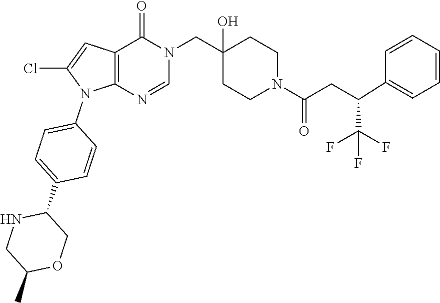 [SMILES: C[C@H]1CN[C@H](c2ccc(-n3c(Cl)cc4c(=O)n(CC5(O)CCN(C(=O)C[C@H](c6ccccc6)C(F)(F)F)CC5)cnc43)cc2)CO1]

### Step 1: tert-Butyl (2S,5R)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (41 mg, 0.0922 mmol), Epoxide 3 (43.3 mg, 0.138 mmol), Cs₂CO₃ (90.1 mg, 0.277 mmol) and DMF (2 mL) after 18 h at 80° C. gave the title compound (15 mg, 21%) as a colourless glass. LCMS (method B): R_{T}=1.58 min, m/z=702, 704 [M−butene+H]⁺.

### Step 2: 6-Chloro-3-((4-hydroxy-1-((R)-4,4,4-trifluoro-3-phenylbutanoyl)piperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2-methylmorpholine-4-carboxylate (15 mg, 0.0198 mmol), TFA (1 mL) and DCM (2 mL) gave, after freeze-drying, the title compound (12 mg, 92%) as a white solid. LCMS (method B): R_{T}=0.89 min, m/z=658, 660 [M+H]⁺. ¹H NMR (500 MHz, DMSO-d₆) δ 8.05 (s, 0.5H), 8.03 (s, 0.5H), 7.64-7.56 (m, 2H), 7.46-7.26 (m, 7H), 6.79 (s, 0.5H), 6.79 (s, 0.5H), 4.88 (s, 1H), 4.18-4.04 (m, 1H), 4.03-3.68 (m, 6H), 3.62-3.50 (m, 1H), 3.30-3.09 (m, 3H), 3.01-2.78 (m, 3H), 2.57-2.52 (m, 2H), 1.68-1.16 (m, 4H), 1.09 (d, J=6.2 Hz, 3H).

### Example 8: 6-Chloro-3-((1-(4-chlorobenzoyl)-4-hydroxypiperidin-4-yl)methyl)-7-(4-((2R,3R)-2-methylmorpholin-3-yl)phenyl)-3,7-dihydro-4H-pyrrolo[2,3-d]pyrimidin-4-one

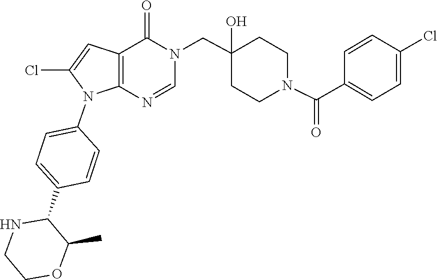 [SMILES: C[C@H]1OCCN[C@@H]1c1ccc(-n2c(Cl)cc3c(=O)n(CC4(O)CCN(C(=O)c5ccc(Cl)cc5)CC4)cnc32)cc1]

### Step 1: 4-(tert-Butyl) 3-(4,5,6,7-tetrachloro-1,3-dioxoisoindolin-2-yl) (2R,3S)-2-methylmorpholine-3,4-dicarboxylate

General procedure 11 using (2R,3S)-4-(tert-butoxycarbonyl)-2-methylmorpholine-3-carboxylic acid (581 mg, 2.37 mmol), 4,5,6,7-tetrachloro-2-hydroxyisoindoline-1,3-dione (713 mg, 2.37 mmol), EDC (546 mg, 2.84 mmol) and DMAP (29 mg, 0.237 mmol) gave the title compound (513 mg, 41%) as a yellow solid. ¹H NMR (400 MHz, Chloroform-d) δ 4.60-4.15 (m, 2H), 3.95-3.86 (m, 1H), 3.73-3.61 (m, 1H), 3.60-3.35 (m, 2H), 1.61-1.43 (m, 12H).

### Step 2: tert-Butyl (2R,3R)-3-(4-bromophenyl)-2-methylmorpholine-4-carboxylate

To 4-(tert-butyl) 3-(4,5,6,7-tetrachloro-1,3-dioxoisoindolin-2-yl) (2R,3S)-2-methylmorpholine-3,4-dicarboxylate (513 mg, 0.971 mmol), (4-bromophenyl)boronic acid (585 mg, 2.91 mmol) in a 100 mL RBF under nitrogen was added 1,4-dioxane (37 mL). The reaction mixture was heated to 75° C. (internal temperature) before triethylamine (1.35 mL, 9.71 mmol) and NiCl₂-6H₂O: bathophenanthroline complex in DMF (0.05 M, 3.7 mL, 0.185 mmol, (Angew. Chem. Int. Ed. 2016, 55, 9676-9679)) were sequentially added. The resulting mixture was stirred at 75° C. overnight. Upon cooling to rt the reaction mixture was diluted with EtOAc (40 mL) and washed with brine. The organic layer was dried over MgSO₄, filtered and concentrated under reduced pressure. The residue was purified by flash chromatography to give the title compound (118 mg, 34%) as a colourless glass. LCMS (method B): R_{T}=1.61 min, m/z=256, 258 [M−Boc+H]⁺.

### Step 3: tert-Butyl (2R,3R)-2-methyl-3-(4-(4,4,5,5-tetramethyl-1,3,2-dioxaborolan-2-yl)phenyl)morpholine-4-carboxylate

General procedure 3 (Work-up A) using tert-butyl (2R,3R)-3-(4-bromophenyl)-2-methylmorpholine-4-carboxylate (118 mg, 0.331 mmol), B₂Pin₂ (168 mg, 0.663 mmol), Pd(dppf)Cl₂·CH₂Cl₂ (28.1 mg, 0.0331 mmol), KOAc (97.5 mg, 0.994 mmol) and 1,4-dioxane (4 mL) after stirring at 120° C. (microwave irradiation) for 1 h gave the title compound (65 mg, 48%) as a white solid. LCMS (method B): R_{T}=1.76 min, m/z=304 [M−Boc+H]⁺.

### Step 4: tert-Butyl (2R,3R)-3-(4-(4,6-dichloro-7H-pyrrolo[2,3-d]pyrimidin-7-yl)phenyl)-2-methylmorpholine-4-carboxylate

General procedure 4 (Work-up A) using 4,6-dichloro-7H-pyrrolo[2,3-d]pyrimidine (30 mg, 0.160 mmol), tert-butyl (2R,3R)-2-methyl-3-(4-(4,4,5,5-tetramethyl-1,3,2-dioxaborolan-2-yl)phenyl)morpholine-4-carboxylate (64.4 mg, 0.160 mmol), copper(II) acetate (58 mg, 0.319 mmol), 1,10-phenanthroline (57.5 mg, 0.319 mmol), boric acid (39.5 mg, 0.638 mmol) and DMF (3 mL) after 6 days at 50° C. gave the title compound (24 mg, 32%) as a colourless glass. LCMS (method A): R_{T}=1.89 min, m/z=463, 465 [M+H]⁺.

### Step 5: tert-Butyl (2R,3R)-3-(4-(6-chloro-4-oxo-3,4-dihydro-7H-pyrrolo[2,3-d]pyrimidin-7-yl)phenyl)-2-methylmorpholine-4-carboxylate

General procedure 5 using tert-butyl (2R,3R)-3-(4-(4,6-dichloro-7H-pyrrolo[2,3-d]pyrimidin-7-yl)phenyl)-2-methylmorpholine-4-carboxylate (24 mg, 0.0518 mmol), aqueous sodium hydroxide (4 M, 0.39 mL, 1.55 mmol) and 1,4-dioxane (0.4 mL) after 18 h at 100° C. using AcOH for acidification gave the title compound (24 mg, >100%) as a yellow glass. LCMS (method B): R_{T}=1.22 min, m/z=445, 447 [M+H]⁺.

### Step 6: tert-Butyl (2R,3R)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,3R)-3-(4-(6-chloro-4-oxo-3,4-dihydro-7H-pyrrolo[2,3-d]pyrimidin-7-yl)phenyl)-2-methylmorpholine-4-carboxylate (24 mg, 0.0539 mmol), Epoxide 2 (20.4 mg, 0.0809 mmol), Cs₂CO₃ (52.7 mg, 0.162 mmol) and DMF (2 mL) after 18 h at 80° C. gave the title compound (12 mg, 31%) as a colourless glass. LCMS (method B): R_{T}=1.48 min, m/z=640, 642 [M−butene+H]⁺.

### Step 7: 6-Chloro-3-((1-(4-chlorobenzoyl)-4-hydroxypiperidin-4-yl)methyl)-7-(4-((2R,3R)-2-methylmorpholin-3-yl)phenyl)-3,7-dihydro-4H-pyrrolo[2,3-d]pyrimidin-4-one

General procedure 7 using tert-butyl (2R,3R)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (12 mg, 0.0172 mmol), TFA (1 mL) and DCM (2 mL) gave, after freeze-drying, the title compound (10 mg, 97%) as an off-white solid. LCMS (method B): R_{T}=0.94 min, m/z=596, 598 [M+H]⁺. ¹H NMR (500 MHz, DMSO-d₆) δ 8.08 (s, 1H), 7.63-7.55 (m, 2H), 7.56-7.48 (m, 2H), 7.46-7.35 (m, 4H), 6.80 (s, 1H), 4.96 (s, 1H), 4.28-4.09 (m, 1H), 4.03 (s, 2H), 3.88-3.72 (m, 1H), 3.63 (td, J=11.0, 3.0 Hz, 1H), 3.45 (d, J=8.7 Hz, 1H), 3.40 (dq, J=8.7, 6.0 Hz, 2H), 3.30-3.20 (m, 1H), 3.20-3.05 (m, 1H), 2.93-2.75 (m, 3H), 1.75-1.54 (m, 2H), 1.53-1.25 (m, 2H), 0.86 (d, J=6.0 Hz, 3H).

### Example 9: rac-6-Chloro-3-((1-(4-chlorobenzoyl)-4-hydroxypiperidin-4-yl)methyl)-7-(4-((3R,6R)-6-(trifluoromethyl)morpholin-3-yl)phenyl)-3,7-dihydro-4H-pyrrolo[2,3-d]pyrimidin-4-one

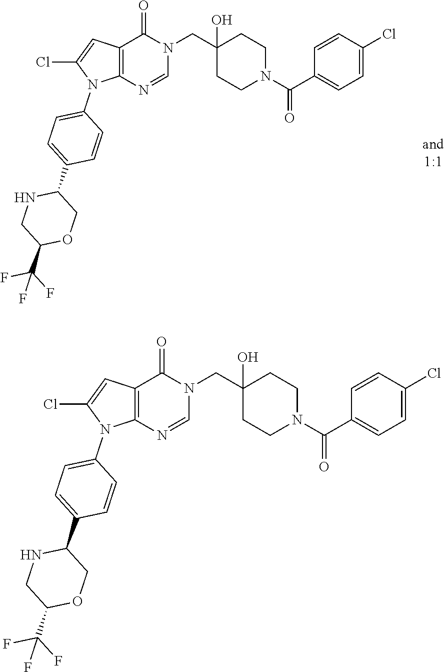 [SMILES: O=C(c1ccc(Cl)cc1)N1CCC(O)(Cn2cnc3c(cc(Cl)n3-c3ccc([C@@H]4CO[C@@H](C(F)(F)F)CN4)cc3)c2=O)CC1 ; O=C(c1ccc(Cl)cc1)N1CCC(O)(Cn2cnc3c(cc(Cl)n3-c3ccc([C@H]4CO[C@H](C(F)(F)F)CN4)cc3)c2=O)CC1]

### Step 1: rac-(2R,5R)-5-(4-Bromophenyl)-2-(trifluoromethyl)morpholine

General procedure 8 using 4-bromobenzaldehyde (5.55 g, 30.0 mmol), 3,3,3-trifluoro-2-((tributylstannyl)methoxy)propan-1-amine (made according to the procedure outlined in Org. Lett. 2014, 16 (4), 1236-1239 starting from 3-amino-1,1,1-trifluoropropan-2-ol) (13.0 g, 30.0 mmol), Cu(OTf)₂ (10.9 g, 30.0 mmol), 2,6-dimethylpyridine (3.21 g, 30.0 mmol), HFIP (120 mL), 4 Å molecular sieves (ca. 3 g) and DCM (480 mL) gave the title compound which was used in the next step without further purification.

### Step 2: rac-tert-Butyl (2R,5R)-5-(4-bromophenyl)-2-(trifluoromethyl)morpholine-4-carboxylate

General procedure 10 using rac-(2R,5R)-5-(4-bromophenyl)-2-(trifluoromethyl)morpholine (1 g, 3.9 mmol) and Boc₂O (10.9 g, 50 mmol) gave the title compound (150 mg, 1% (2 steps)). LCMS (method C): R_{T}=1.63 min, mass ion not observed. This material was used without further purification.

### Step 3: rac-tert-Butyl (2R,5R)-5-(4-(4,4,5,5-tetramethyl-1,3,2-dioxaborolan-2-yl)phenyl)-2-(trifluoromethyl)morpholine-4-carboxylate

General procedure 3 (Work-up A) using rac-tert-butyl (2R,5R)-5-(4-bromophenyl)-2-(trifluoromethyl)morpholine-4-carboxylate (150 mg, 0.366 mmol), B₂Pin₂ (139 mg, 0.549 mmol), Pd(dppf)Cl₂·CH₂Cl₂ (31 mg, 0.0366 mmol), KOAc (108 mg, 1.10 mmol) and 1,4-dioxane (4 mL) after stirring at 120° C. (microwave irradiation) for 1 h gave the title compound (135 mg, 80%) as a colourless glass. LCMS (method B): R_{T}=1.75 min, m/z=402 [M−butene+H]⁺.

### Step 4: rac-tert-Butyl (2R,5R)-5-(4-(4,6-dichloro-7H-pyrrolo[2,3-d]pyrimidin-7-yl)phenyl)-2-(trifluoromethyl)morpholine-4-carboxylate

General procedure 4 (Work-up A) using 4,6-dichloro-7H-pyrrolo[2,3-d]pyrimidine (55 mg, 0.293 mmol), rac-tert-butyl (2R,5R)-5-(4-(4,4,5,5-tetramethyl-1,3,2-dioxaborolan-2-yl)phenyl)-2-(trifluoromethyl)morpholine-4-carboxylate (134 mg, 0.293 mmol), copper(II) acetate (106 mg, 0.585 mmol), 1,10-phenanthroline (105 mg, 0.585 mmol), boric acid (72 mg, 1.17 mmol) and DMF (5.5 mL) after 6 days at 50° C. gave the title compound (17 mg, 11%) as a colourless glass. LCMS (method A): R_{T}=1.71 min, m/z=517, 519 [M+H]⁺.

### Step 5: rac-tert-Butyl (2R,5R)-5-(4-(6-chloro-4-oxo-3,4-dihydro-7H-pyrrolo[2,3-d]pyrimidin-7-yl)phenyl)-2-(trifluoromethyl)morpholine-4-carboxylate

General procedure 5 using rac-tert-butyl (2R,5R)-5-(4-(4,6-dichloro-7H-pyrrolo[2,3-d]pyrimidin-7-yl)phenyl)-2-(trifluoromethyl)morpholine-4-carboxylate (17 mg, 0.0329 mmol), aqueous sodium hydroxide (4 M, 0.25 mL, 1.00 mmol) and 1,4-dioxane (0.25 mL) after 18 h at 100° C. using AcOH for acidification gave the title compound (17 mg, >100%) as a yellow glass. LCMS (method B): R_{T}=1.33 min, m/z=499, 501 [M+H]⁺.

### Step 6: rac-tert-Butyl (2R,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-(trifluoromethyl)morpholine-4-carboxylate

General procedure 6 using rac-tert-Butyl (2R,5R)-5-(4-(6-chloro-4-oxo-3,4-dihydro-7H-pyrrolo[2,3-d]pyrimidin-7-yl)phenyl)-2-(trifluoromethyl)morpholine-4-carboxylate (17 mg, 0.0341 mmol), Epoxide 2 (12.9 mg, 0.0511 mmol), Cs₂CO₃ (33.3 mg, 0.102 mmol) and DMF (1 mL) after 18 h at 80° C. gave the title compound (12 mg, 46%) as a colourless glass. LCMS (method B): R_{T}=1.55 min, m/z=694, 696 [M−butene+H]⁺.

### Step 7: rac-6-Chloro-3-((1-(4-chlorobenzoyl)-4-hydroxypiperidin-4-yl)methyl)-7-(4-((3R,6R)-6-(trifluoromethyl)morpholin-3-yl)phenyl)-3,7-dihydro-4H-pyrrolo[2,3-d]pyrimidin-4-one

General procedure 7 using rac-tert-butyl (2R,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-(trifluoromethyl)morpholine-4-carboxylate (12 mg, 0.0160 mmol), TFA (1 mL) and DCM (2 mL) gave, after freeze-drying, the title compound (7 mg, 67%) as an off-white solid. LCMS (method B): R_{T}=1.00 min, m/z=650, 652 [M+H]⁺. ¹H NMR (500 MHz, DMSO-d₆) δ 8.07 (s, 1H), 7.64 (d, J=8.1 Hz, 2H), 7.54-7.49 (m, 2H), 7.46 (d, J=8.0 Hz, 2H), 7.45-7.39 (m, 2H), 6.81 (s, 1H), 4.96 (s, 1H), 4.31-4.12 (m, 2H), 4.14-3.92 (m, 4H), 3.59-3.45 (m, 1H), 3.45-3.34 (m, 2H), 3.29-3.04 (m, 3H), 3.03-2.83 (m, 1H), 1.69-1.27 (m, 4H).

### Example 10: 6-Chloro-7-(4-((S)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one and 6-chloro-7-(4-((R)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one

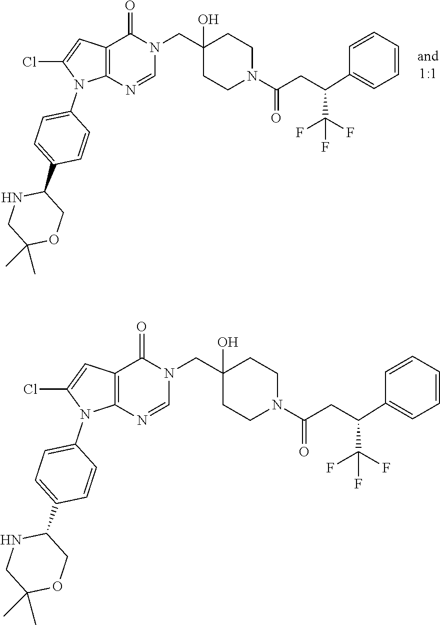 [SMILES: CC1(C)CN[C@@H](c2ccc(-n3c(Cl)cc4c(=O)n(CC5(O)CCN(C(=O)C[C@H](c6ccccc6)C(F)(F)F)CC5)cnc43)cc2)CO1 ; CC1(C)CN[C@H](c2ccc(-n3c(Cl)cc4c(=O)n(CC5(O)CCN(C(=O)C[C@H](c6ccccc6)C(F)(F)F)CC5)cnc43)cc2)CO1]

### Step 1: 5-(4-Bromophenyl)-2,2-dimethylmorpholine

General procedure 8 using 4-bromobenzaldehyde (1.85 g, 10.0 mmol), 2-methyl-2-((tributylstannyl)methoxy)propan-1-amine (made according to the procedure outlined in Org. Lett. 2014, 16 (4), 1236-1239 starting from 1-amino-2-methylpropan-2-ol) (3.92 g, 10.0 mmol), Cu(OTf)₂ (3.62 g, 10.0 mmol), 2,6-dimethylpyridine (1.07 g, 10.0 mmol), HFIP (40 mL), 4 Å molecular sieves (ca. 1 g) and DCM (160 mL) gave the title compound (1.74 g, 64%). LCMS (method C): R_{T}=0.75 min, m/z=270, 272 [M+H]⁺. ¹H NMR (500 MHz, Chloroform-d) δ 7.45 (d, J=7.6 Hz, 2H), 7.30 (d, J=7.6 Hz, 2H), 3.86-3.76 (m, 1H), 3.64-3.51 (m, 2H), 2.90-2.79 (m, 2H), 1.43 (s, 3H), 1.22 (s, 3H).

### Step 2: tert-Butyl 5-(4-bromophenyl)-2,2-dimethylmorpholine-4-carboxylate

General procedure 9 using 5-(4-bromophenyl)-2,2-dimethylmorpholine (1.74 g, 6.44 mmol) and Boc₂O (1.69 g, 7.73 mmol) gave the title compound (2.10 g, 88%). LCMS (method C): R_{T}=1.64 min, m/z=270, 272 [M−Boc+H]⁺. ¹H NMR (500 MHz, Chloroform-d) δ 7.46 (d, J=8.9 Hz, 2H), 7.33 (d, J=8.1 Hz, 2H), 5.04 (s, 1H), 4.14-3.96 (m, 2H), 3.65 (d, J=13.6 Hz, 1H), 2.79 (d, J=13.6 Hz, 1H), 1.46 (s, 9H), 1.27 (s, 3H), 1.17 (s, 3H).

### Step 3: tert-Butyl 2,2-dimethyl-5-(4-(4,4,5,5-tetramethyl-1,3,2-dioxaborolan-2-yl)phenyl)morpholine-4-carboxylate

General procedure 3 (Work-up A) using 5-(4-bromophenyl)-2,2-dimethylmorpholine (1.40 g, 3.78 mmol), B₂Pin₂ (1.44 mg, 5.67 mmol), Pd(dppf)Cl₂·CH₂Cl₂ (320 mg, 0.378 mmol), KOAc (1.11 g, 11.3 mmol) and 1,4-dioxane (36 mL) after stirring at 100° C. (thermal) for 2 h gave the title compound (1.52 g, 96%) as a colourless glass. LCMS (method B): R_{T}=1.78 min, m/z=318 [M−Boc+H]⁺.

### Step 4: tert-Butyl 5-(4-(4,6-dichloro-7H-pyrrolo[2,3-d]pyrimidin-7-yl)phenyl)-2,2-dimethylmorpholine-4-carboxylate

General procedure 4 (Work-up A) using 4,6-dichloro-7H-pyrrolo[2,3-d]pyrimidine (457 mg, 2.43 mmol), tert-butyl 2,2-dimethyl-5-(4-(4,4,5,5-tetramethyl-1,3,2-dioxaborolan-2-yl)phenyl)morpholine-4-carboxylate (1.52 g, 3.65 mmol), copper(II) trifluoroacetate hydrate (1.41 mg, 4.86 mmol), 1,10-phenanthroline (876 mg, 4.86 mmol), boric acid (601 mg, 9.72 mmol) and DMF (46 mL) after 4 days at 50° C. gave the title compound (772 mg, 66%) as a colourless glass. LCMS (method B): R_{T}=1.74 min, m/z=477, 479 [M+H]⁺.

### Step 5: tert-Butyl 5-(4-(6-chloro-4-oxo-3,4-dihydro-7H-pyrrolo[2,3-d]pyrimidin-7-yl)phenyl)-2,2-dimethylmorpholine-4-carboxylate

General procedure 5 using tert-butyl 5-(4-(4,6-dichloro-7H-pyrrolo[2,3-d]pyrimidin-7-yl)phenyl)-2,2-dimethylmorpholine-4-carboxylate (97 mg, 0.203 mmol), aqueous sodium hydroxide (4 M, 1.52 mL, 6.09 mmol) and 1,4-dioxane (1.6 mL) after 18 h at 100° C. using AcOH for acidification gave the title compound (94 mg, quant.) as a yellow glass. LCMS (method B): R_{T}=1.30 min, m/z=459,461 [M+H]⁺.

### Step 6: tert-Butyl (S)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2,2-dimethylmorpholine-4-carboxylate and tert-butyl (R)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2,2-dimethylmorpholine-4-carboxylate (1:1)

General procedure 6 using tert-butyl 5-(4-(6-chloro-4-oxo-3,4-dihydro-7H-pyrrolo[2,3-d]pyrimidin-7-yl)phenyl)-2,2-dimethylmorpholine-4-carboxylate (47 mg, 0.102 mmol), Epoxide 3 (48.1 mg, 0.154 mmol), Cs₂CO₃ (100 mg, 0.307 mmol) and DMF (3 mL) after 18 h at 80° C. gave the title compound (27 mg, 34%) as a colourless glass. LCMS (method B): R_{T}=1.62 min, m/z=716, 718 [M−butene+H]⁺.

### Step 7: 6-Chloro-7-(4-((S)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one and 6-chloro-7-(4-((R)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one (1:1)

General procedure 7 using tert-butyl (S)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2,2-dimethylmorpholine-4-carboxylate and tert-butyl (R)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2,2-dimethylmorpholine-4-carboxylate (1:1) (27 mg, 0.0350 mmol), TFA (1 mL) and DCM (2 mL) gave, after freeze-drying, the title compound (23 mg, 97%) as an off-white solid. LCMS (method B): R_{T}=0.94 min, m/z=672, 674 [M+H]⁺. ¹H NMR (500 MHz, DMSO-d₆) δ 8.05 (s, 0.5H), 8.03 (s, 0.5H), 7.68-7.61 (m, 2H), 7.47-7.27 (m, 7H), 6.80 (s, 0.5H), 6.80 (s, 0.5H), 4.88 (s, 1H), 4.17-4.05 (m, 1H), 4.03-3.80 (m, 4H), 3.80-3.71 (m, 1H), 3.62-3.55 (m, 1H), 3.55-3.44 (m, 1H), 3.28-3.11 (m, 2H), 3.00-2.66 (m, 5H), 1.63-1.56 (m, 0.5H), 1.46-1.26 (m, 3H), 1.36 (s, 3H), 1.25-1.16 (m, 0.5H), 1.14 (s, 3H).

### Example 11: 6-Chloro-3-((1-(4-chlorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one

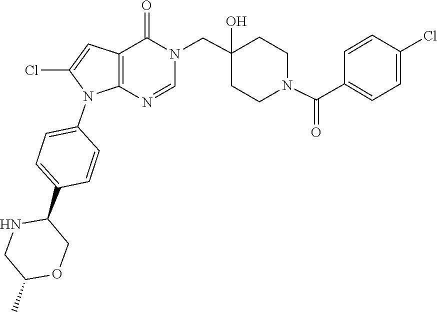 [SMILES: C[C@@H]1CN[C@@H](c2ccc(-n3c(Cl)cc4c(=O)n(CC5(O)CCN(C(=O)c6ccc(Cl)cc6)CC5)cnc43)cc2)CO1]

### Step 1: (2R,5S)-5-(4-Bromophenyl)-2-methylmorpholine

General procedure 8 using 4-bromobenzaldehyde (1.85 g, 10.0 mmol), (R)-2-((tributylstannyl)methoxy)propan-1-amine (made according to the procedure outlined in Org. Lett. 2014, 16 (4), 1236-1239 starting from (R)-1-aminopropan-2-ol) (3.78 g, 10.0 mmol), Cu(OTf)₂ (3.62 g, 10.0 mmol), 2,6-dimethylpyridine (1.07 g, 10.0 mmol), HFIP (40 mL), 4 Å molecular sieves (ca. 1 g) and DCM (160 mL) gave the title compound (1.10 g, 43%). LCMS (method C): R_{T}=0.81 min, m/z=256, 258 [M+H]⁺.

### Step 2: tert-Butyl (2R,5S)-5-(4-bromophenyl)-2-methylmorpholine-4-carboxylate

General procedure 9 using (2R,5S)-5-(4-bromophenyl)-2-methylmorpholine (1.10 g, 4.30 mmol) and Boc₂O (1.12 g, 5.15 mmol) gave the title compound (1.4 g, 91%). LCMS (method C): R_{T}=1.60 min, m/z=256, 258 [M−Boc+H]⁺. ¹H NMR (500 MHz, Chloroform-d) δ 7.45 (d, J=8.4 Hz, 2H), 7.24 (d, J=8.8 Hz, 2H), 4.76 (t, J=5.2 Hz, 1H), 4.01 (dd, J=12.3, 4.6 Hz, 1H), 3.98-3.92 (m, 1H), 3.71 (dd, J=12.3, 5.8 Hz, 1H), 3.51 (dd, J=13.5, 3.8 Hz, 1H), 3.36 (dd, J=13.5, 4.8 Hz, 1H), 1.34 (s, 9H), 1.28 (d, J=6.4 Hz, 3H). Chiral purity (method B): R_{T}=10.61 min, >99% ee.

### Step 3: tert-Butyl (2R,5S)-2-methyl-5-(4-(4,4,5,5-tetramethyl-1,3,2-dioxaborolan-2-yl)phenyl)morpholine-4-carboxylate

General procedure 3 (Work-up A) using tert-butyl (2R,5S)-5-(4-bromophenyl)-2-methylmorpholine-4-carboxylate (600 mg, 1.68 mmol), B₂Pin₂ (642 mg, 2.53 mmol), Pd(dppf)Cl₂·CH₂Cl₂ (143 mg, 0.168 mmol), KOAc (496 mg, 5.05 mmol) and 1,4-dioxane (16.8 mL) after stirring at 120° C. (thermal) for 1 h gave the title compound (541 mg, 79%) as a colourless glass. LCMS (method B): R_{T}=1.77 min, m/z=348 [M−butene+H]⁺. ¹H NMR (500 MHz, DMSO-d₆) δ 7.64 (d, J=8.0 Hz, 2H), 7.32 (d, J=8.0 Hz, 2H), 4.76 (t, J=5.4 Hz, 1H), 3.97-3.88 (m, 2H), 3.66 (q, J=6.1 Hz, 1H), 3.46 (dd, J=13.6, 4.1 Hz, 1H), 3.34 (dd, J=13.5, 4.5 Hz, 1H), 1.28 (s, 12H), 1.27 (s, 9H), 1.17 (s, 3H).

### Step 4: tert-Butyl (2R,5S)-5-(4-(4,6-dichloro-7H-pyrrolo[2,3-d]pyrimidin-7-yl)phenyl)-2-methylmorpholine-4-carboxylate

General procedure 4 (Work-up A) using 4,6-dichloro-7H-pyrrolo[2,3-d]pyrimidine (127 mg, 0.676 mmol), tert-butyl (2R,5S)-2-methyl-5-(4-(4,4,5,5-tetramethyl-1,3,2-dioxaborolan-2-yl)phenyl)morpholine-4-carboxylate (545 mg, 1.35 mmol), copper(II) acetate (245 mg, 1.35 mmol), 1,10-phenanthroline (243 mg, 1.35 mmol), boric acid (167 mg, 2.70 mmol) and DMF (6.8 mL) after 4 days at 50° C. gave the title compound (122 mg, 38%) as a colourless glass. LCMS (method A): R_{T}=1.69 min, m/z=463, 465 [M+H]⁺.

### Step 5: tert-Butyl (2R,5S)-5-(4-(6-chloro-4-oxo-3,4-dihydro-7H-pyrrolo[2,3-d]pyrimidin-7-yl)phenyl)-2-methylmorpholine-4-carboxylate

A solution of tert-butyl (2R,5S)-5-(4-(4,6-dichloro-7H-pyrrolo[2,3-d]pyrimidin-7-yl)phenyl)-2-methylmorpholine-4-carboxylate (100 mg, 0.216 mmol) and aqueous sodium hydroxide (2 M, 1.62 mL, 3.24 mmol) in 1,4-dioxane (2 mL) was heated at 100° C. for 18 h. Upon cooling at rt water was added and the aqueous phase was extracted with DCM (3×15 mL). The combined organic phases were concentrated under reduced to give the title compound (96 mg, quant.) as a white solid. LCMS (method B): R_{T}=1.24 min, m/z=445, 447 [M+H]⁺.

### Step 6: tert-Butyl (2R,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 2 (27.1 mg, 0.108 mmol), Cs₂CO₃ (64.4 mg, 0.198 mmol) and DMF (1 mL) after 18 h at 80° C. gave the title compound (20 mg, 31%) as a white solid. LCMS (method B): R_{T}=1.49 min, m/z=640, 642 [M−butene+H]⁺.

### Step 7: 6-Chloro-3-((1-(4-chlorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (20 mg, 0.0287 mmol), TFA (0.35 mL) and DCM (0.7 mL) gave, after freeze-drying, the title compound (8.5 mg, 47%) as a white solid. LCMS (method B): R_{T}=0.97 min, m/z=596, 598 [M+H]⁺. ¹H NMR (500 MHz, DMSO-d₆) δ 8.06 (s, 1H), 7.62-7.58 (m, 2H), 7.52-7.48 (m, 2H), 7.44-7.37 (m, 4H), 6.79 (s, 1H), 4.95 (s, 1H), 4.22-4.12 (m, 1H), 4.02 (s, 2H), 3.87-3.78 (m, 2H), 3.60-3.50 (m, 1H), 3.42-3.33 (m, 1H), 3.29-3.21 (m, 2H), 3.17-3.08 (m, 1H), 2.95 (dd, J=11.7, 2.3 Hz, 1H), 2.93-2.84 (m, 1H), 2.57-2.53 (m, 1H), 1.68-1.53 (m, 2H), 1.51-1.41 (m, 1H), 1.38-1.27 (m, 1H), 1.09 (d, J=6.2 Hz, 3H). Chiral purity (method C): R_{T}=3.96 min, 100% ee.

### Example 12: 6-Chloro-3-((1-(4-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

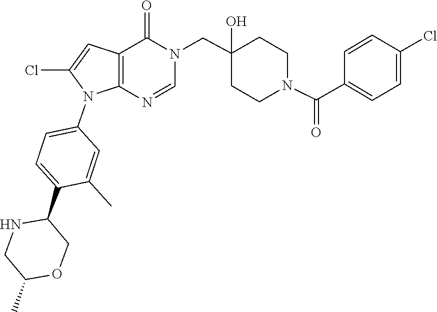 [SMILES: Cc1cc(-n2c(Cl)cc3c(=O)n(CC4(O)CCN(C(=O)c5ccc(Cl)cc5)CC4)cnc32)ccc1[C@H]1CO[C@H](C)CN1]

### Step 1: (2R,5S)-5-(4-Bromo-2-methylphenyl)-2-methylmorpholine

General procedure 8 using 4-bromo-2-methylbenzaldehyde (1.99 g, 10.0 mmol), (R)-2-((tributylstannyl)methoxy)propan-1-amine (made according to the procedure outlined in Org. Lett. 2014, 16 (4), 1236-1239 starting from (R)-1-aminopropan-2-ol) (3.78 g, 10.0 mmol), Cu(OTf)₂ (3.62 g, 10.0 mmol), 2,6-dimethylpyridine (1.07 g, 10.0 mmol), HFIP (40 mL), 4 Å molecular sieves (ca. 1 g) and DCM (160 mL) gave the title compound (1.73 g, 66%). LCMS (method C): R_{T}=0.88 min, m/z=270, 272 [M+H]⁺.

### Step 2: tert-Butyl (2R,5S)-5-(4-bromo-2-methylphenyl)-2-methylmorpholine-4-carboxylate

General procedure 9 using (2R,5S)-5-(4-Bromo-2-methylphenyl)-2-methylmorpholine (1.73 g, 6.60 mmol) and Boc₂O (1.4 g, 7.92 mmol) gave the title compound (2.25 g, 92%). LCMS (method C): R_{T}=1.69 min, m/z=270, 272 [M−Boc+H]⁺. ¹H NMR (500 MHz, Chloroform-d) δ 7.36-7.12 (m, 3H), 4.72 (dd, J=9.1, 4.9 Hz, 1H), 3.92 (td, J=7.2, 6.8, 4.1 Hz, 1H), 3.84 (dd, J=12.3, 4.7 Hz, 1H), 3.76 (dd, J=13.5, 3.8 Hz, 1H), 3.45 (dd, J=12.3, 8.8 Hz, 1H), 3.23 (dd, J=13.5, 6.5 Hz, 1H), 2.31 (s, 3H), 1.25 (d, J=5.9 Hz, 3H), 1.19 (s, 9H). Chiral purity (method D): R_{T}=4.742 min, >99% ee.

### Step 3: tert-butyl (2R,5S)-2-methyl-5-(2-methyl-4-(4,4,5,5-tetramethyl-1,3,2-dioxaborolan-2-yl)phenyl)morpholine-4-carboxylate

General procedure 3 (Work-up B) using tert-butyl (2R,5S)-5-(4-bromo-2-methylphenyl)-2-methylmorpholine-4-carboxylate (1.57 g, 4.24 mmol), B₂Pin₂ (1.62 g, 6.36 mmol), Pd(dppf)Cl₂·CH₂Cl₂ (359 mg, 0.424 mmol), KOAc (1.25 g, 12.7 mmol) and 1,4-dioxane (42.4 mL) after stirring at 100° C. (thermal) for 65 min gave the title compound (1.92 g, >100%) as a yellow oil. LCMS (method A): R_{T}=2.03 min, m/z=362 [M−butene+H]⁺.

### Step 4: tert-Butyl (2R,5S)-5-(4-(4,6-dichloro-7H-pyrrolo[2,3-d]pyrimidin-7-yl)-2-methylphenyl)-2-methylmorpholine-4-carboxylate

General procedure 4 (Work-up B) using 4,6-dichloro-7H-pyrrolo[2,3-d]pyrimidine (620 mg, 3.30 mmol), tert-butyl (2R,5S)-2-methyl-5-(2-methyl-4-(4,4,5,5-tetramethyl-1,3,2-dioxaborolan-2-yl)phenyl)morpholine-4-carboxylate (1.79 g, 4.29 mmol), copper(II) trifluoroacetate hydrate (1.91 g, 6.60 mmol), 1,10-phenanthroline (1.19 g, 6.60 mmol), boric acid (530 mg, 8.57 mmol) and DMF (33 mL) after 5 days at 50° C. gave the title compound (970 mg, 61%) as a white foam. LCMS (method A): R_{T}=1.99 min, m/z=477, 479 [M+H]⁺. ¹H NMR (500 MHz, DMSO-d₆) δ 8.61 (s, 1H), 7.54 (d, J=8.1 Hz, 1H), 7.36-7.28 (m, 2H), 7.08 (s, 1H), 4.81 (dd, J=8.9, 5.0 Hz, 1H), 4.03-3.97 (m, 1H), 3.95 (dd, J=12.1, 5.0 Hz, 1H), 3.81 (dd, J=13.4, 4.0 Hz, 1H), 3.52 (dd, J=12.1, 9.0 Hz, 1H), 3.23 (dd, J=13.5, 6.5 Hz, 1H), 2.40 (s, 3H), 1.22-1.16 (m, 12H).

### Step 5: tert-Butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate

General procedure 5 using tert-butyl (2R,5S)-5-(4-(4,6-dichloro-7H-pyrrolo[2,3-d]pyrimidin-7-yl)-2-methylphenyl)-2-methylmorpholine-4-carboxylate (968 mg, 2.03 mmol), aqueous sodium hydroxide (4 M, 5.07 mL, 20.3 mmol) and 1,4-dioxane (10.1 mL) after 47 h at 100° C. using 2 M aqueous hydrochloric acid for acidification gave the title compound (1.07 g, >100%) as a brown foam. LCMS (method A): R_{T}=1.36 min, m/z=459, 461 [M+H]⁺. 1H NMR (500 MHz, DMSO-d6) δ 12.16 (br. s, 1H), 7.84 (s, 1H), 7.48 (d, J=8.1 Hz, 1H), 7.25-7.19 (m, 2H), 6.69 (s, 1H), 4.80 (dd, J=8.9, 5.1 Hz, 1H), 4.04-3.95 (m, 1H), 3.93 (dd, J=12.1, 5.1 Hz, 1H), 3.79 (dd, J=13.5, 4.0 Hz, 1H), 3.50 (dd, J=12.1, 9.0 Hz, 1H), 3.23 (dd, J=13.5, 6.3 Hz, 1H), 2.38 (s, 3H), 1.20-1.16 (m, 12H).

### Step 6: tert-Butyl (2R,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (150 mg, 0.327 mmol), Epoxide 2 (123 mg, 0.490 mmol), Cs₂CO₃ (213 mg, 0.654 mmol) and DMF (3.3 mL) after 18 h at 80° C. gave the title compound (119 mg, 52%) as an orange glass. LCMS (method B): R_{T}=1.54 min, m/z=654, 656 [M−butene+H]⁺.

### Step 7: 6-Chloro-3-((1-(4-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (119 mg, 0.168 mmol), TFA (2 mL) and DCM (4 mL) gave, after purification by flash chromatography and freeze-drying, the title compound (66 mg, 64%) as a white solid. LCMS (method B): R_{T}=0.85 min, m/z=610, 612 [M+H]⁺. ¹H NMR (500 MHz, DMSO-d₆) δ 8.06 (s, 1H), 7.73 (d, J=7.9 Hz, 1H), 7.56-7.48 (m, 2H), 7.49-7.38 (m, 2H), 7.23 (s, 2H), 6.78 (s, 1H), 4.96 (s, 1H), 4.17 (s, 1H), 4.00 (d, J=21.0 Hz, 3H), 3.80 (dd, J=10.9, 3.0 Hz, 1H), 3.46-3.33 (m, 1H), 3.30-3.08 (m, 3H), 3.02-2.92 (m, 1H), 2.83-2.71 (m, 1H), 2.58 (t, J=10.7 Hz, 1H), 2.42 (s, 3H), 1.71-1.26 (m, 4H), 1.10 (d, J=6.2 Hz, 3H).

### Example 13: 6-Chloro-3-((4-hydroxy-1-((R)-4,4,4-trifluoro-3-phenylbutanoyl)piperidin-4-yl)methyl)-7-(4-((3S,6R)-6-methylmorpholin-3-yl)phenyl)-3,7-dihydro-4H-pyrrolo[2,3-d]pyrimidin-4-one

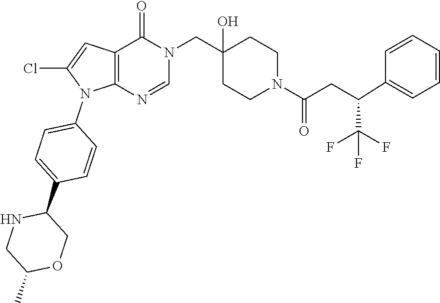 [SMILES: C[C@@H]1CN[C@@H](c2ccc(-n3c(Cl)cc4c(=O)n(CC5(O)CCN(C(=O)C[C@H](c6ccccc6)C(F)(F)F)CC5)cnc43)cc2)CO1]

### Step 1: tert-Butyl (2R,5S)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 3 (33.8 mg, 0.108 mmol), Cs₂CO₃ (64.4 mg, 0.198 mmol) and DMF (1 mL) after 18 h at 80° C. gave the title compound (18.5 mg, 27%) as a colourless glass. LCMS (method B): R_{T}=1.57 min, m/z=702, 704 [M−butene+H]⁺.

### Step 2: 6-Chloro-3-((4-hydroxy-1-((R)-4,4,4-trifluoro-3-phenylbutanoyl)piperidin-4-yl)methyl)-7-(4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)-2-methylmorpholine-4-carboxylate (18.5 mg, 0.0244 mmol), TFA (0.35 mL) and DCM (0.7 mL) gave, after freeze-drying, the title compound (7.4 mg, 43%) as a white solid. LCMS (method B): R_{T}=0.89 min, m/z=658, 660 [M+H]⁺. ¹H NMR (500 MHz, DMSO-d₆) δ 8.05 (s, 0.5H), 8.03 (s, 0.5H), 7.76-7.13 (m, 9H), 6.79 (s, 0.5H), 6.79 (s, 0.5H), 4.88 (s, 1H), 4.17-3.69 (m, 7H), 3.60-3.51 (m, 1H), 3.29-3.10 (m, 3H), 3.06-2.72 (m, 4H), 2.57-2.52 (m, 1H), 1.64-1.55 (m, 0.5H), 1.49-1.27 (m, 3H), 1.22-1.17 (m, 0.5H), 1.09 (d, J=6.2 Hz, 3H).

### Example 14: 6-Chloro-3-((4-hydroxy-1-((R)-4,4,4-trifluoro-3-phenylbutanoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

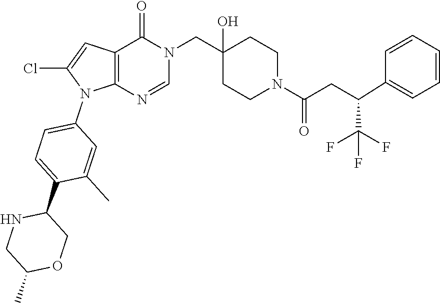 [SMILES: Cc1cc(-n2c(Cl)cc3c(=O)n(CC4(O)CCN(C(=O)C[C@H](c5ccccc5)C(F)(F)F)CC4)cnc32)ccc1[C@H]1CO[C@H](C)CN1]

### Step 1: tert-Butyl (2R,5S)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate

A suspension of tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (40 mg, 0.0872 mmol), Epoxide 3 (34 mg, 0.109 mmol) and DIPEA (76 μL, 0.436 mmol) in DMF (0.87 mL) were stirred at 100° C. for 20 h. Upon cooling to rt the reaction mixture was diluted with saturated aqueous sodium hydrogen carbonate (15 mL) and the resulting suspension was extracted with DCM (3×10 mL) using an ISOLUTE® phase separator. The combined organic phases were concentrated under reduced pressure and the residue was purified by flash chromatography to give the title compound (38.6 mg, 57%) as white solid. LCMS (method A): R_{T}=1.61 min, m/z=716, 718 [M−butene+H]⁺.

### Step 2: 6-Chloro-3-((4-hydroxy-1-((R)-4,4,4-trifluoro-3-phenylbutanoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate (38.6 mg, 0.0500 mmol) TFA (0.25 mL) and DCM (0.5 mL) gave, after freeze-drying, the title compound (31.3 mg, 91%) as an off-white solid. LCMS (method B): R_{T}=0.96 min, m/z=672, 674 [M+H]⁺. ¹H NMR (500 MHz, DMSO-d₆) δ 8.04 (s, 0.5H), 8.02 (s, 0.5H), 7.73 (d, J=8.0 Hz, 1H), 7.51-7.28 (m, 5H), 7.28-7.11 (m, 2H), 6.78 (s, 1H), 4.88 (s, 1H), 4.17-4.04 (m, 1H), 4.05-3.85 (m, 4H), 3.84-3.71 (m, 2H), 3.58 (ddd, J=8.9, 6.3, 2.3 Hz, 1H), 3.29-3.10 (m, 3H), 3.04-2.67 (m, 4H), 2.58 (t, J=10.9 Hz, 1H), 2.41 (s, 3H), 1.63-1.56 (m, 0.5H), 1.45-1.27 (m, 3H), 1.23-1.17 (m, 0.5H), 1.10 (d, J=6.2 Hz, 3H).

### Example 15: 6-Chloro-7-(4-(5,5-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

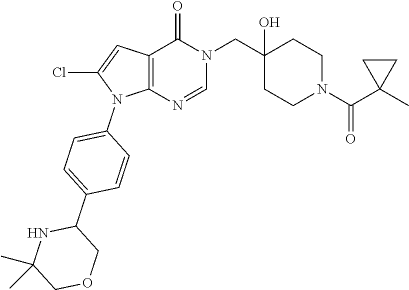

### Step 1: 5-(4-Bromophenyl)-3,3-dimethylmorpholine

General procedure 8 using 4-bromobenzaldehyde (1.85 g, 10.0 mmol), 2-methyl-1-((tributylstannyl)methoxy)propan-2-amine (Helv. Chim. Acta 2020, 103, e2000179) (3.92 g, 10.0 mmol), Cu(OTf)₂ (3.62 g, 10.0 mmol), 2,6-dimethylpyridine (1.07 g, 10.0 mmol), HFIP (40 mL), 4 Å molecular sieves (ca. 1 g) and DCM (160 mL) gave the title compound (360 mg, 13%). LCMS (method C): R_{T}=0.78 min, m/z=270, 272 [M+H]⁺. ¹H NMR (500 MHz, Chloroform-d) δ 7.44 (d, J=8.9 Hz, 2H), 7.28 (d, J=8.4 Hz, 2H), 4.19 (dd, J=10.5, 3.6 Hz, 1H), 3.82 (dd, J=10.9, 3.6 Hz, 1H), 3.52 (d, J=10.8 Hz, 1H), 3.28 (d, J=10.9 Hz, 2H), 3.16 (d, J=10.6 Hz, 1H), 1.35 (s, 3H), 1.06 (s, 3H).

### Step 2: tert-Butyl 5-(4-bromophenyl)-3,3-dimethylmorpholine-4-carboxylate

General procedure 10 using 5-(4-bromophenyl)-3,3-dimethylmorpholine (360 mg, 1.33 mmol) and Boc₂O (580 mg, 2.66 mmol) gave the title compound (310 mg, 63%). LCMS (method C): R_{T}=1.60 min, m/z=270, 272 [M−Boc+H]⁺. ¹H NMR (500 MHz, Chloroform-d) δ 7.44 (d, J=8.4 Hz, 2H), 7.26 (d, J=7.4 Hz, 2H), 4.92 (t, J=4.7 Hz, 1H), 4.12-4.03 (m, 2H), 3.48 (d, J=11.6 Hz, 1H), 3.31 (d, J=11.7 Hz, 1H), 1.47 (s, 3H), 1.37 (d, J=3.2 Hz, 12H).

### Step 3: tert-Butyl 3,3-dimethyl-5-(4-(4,4,5,5-tetramethyl-1,3,2-dioxaborolan-2-yl)phenyl)morpholine-4-carboxylate

General procedure 3 (Work-up B) using tert-butyl 5-(4-bromophenyl)-3,3-dimethylmorpholine-4-carboxylate (310 mg, 0.8372 mmol), B₂Pin₂ (319 mg, 1.26 mmol), Pd(dppf)Cl₂·CH₂Cl₂ (70.9 mg, 0.0837 mmol), KOAc (247 mg, 2.51 mmol) and 1,4-dioxane (8.4 mL) after stirring at 100° C. (thermal) for 2 h 10 min gave the title compound (390 mg, >100%) as a slightly yellow oil. LCMS (method A): R_{T}=2.09 min, m/z=418 [M+H]⁺. ¹H NMR (500 MHz, DMSO-d₆) δ 7.64 (d, J=8.1 Hz, 2H), 7.35 (d, J=7.8 Hz, 2H), 4.89 (t, J=4.6 Hz, 1H), 4.02 (d, J=4.6 Hz, 2H), 3.36 (d, J=11.7 Hz, 1H), 3.30 (d, J=11.8 Hz, 1H (signal overlaps with HDO)), 1.13-1.49 (m, 27H).

### Step 4: tert-Butyl 5-(4-(4,6-dichloro-7H-pyrrolo[2,3-d]pyrimidin-7-yl)phenyl)-3,3-dimethylmorpholine-4-carboxylate

General procedure 4 (Work-up B) using 4,6-dichloro-7H-pyrrolo[2,3-d]pyrimidine (130 mg, 0.692 mmol), tert-butyl 3,3-dimethyl-5-(4-(4,4,5,5-tetramethyl-1,3,2-dioxaborolan-2-yl)phenyl)morpholine-4-carboxylate (346 mg, 0.830 mmol), copper(II) trifluoroacetate hydrate (290 mg, 1.38 mmol), 1,10-phenanthroline (249 mg, 1.38 mmol), boric acid (103 mg, 1.66 mmol) and DMF (7 mL) after 4 days at 50° C. gave the title compound (181 mg, 54%) as an off-white foam. LCMS (method B): R_{T}=1.77 min, m/z=477, 479 [M+H]⁺. ¹H NMR (500 MHz, DMSO-d₆) δ 8.62 (s, 1H), 7.58-7.53 (m, 2H), 7.53-7.48 (m, 2H), 7.10 (s, 1H), 4.96 (t, J=4.9 Hz, 1H), 4.11 (dd, J=11.8, 4.8 Hz, 1H), 4.03 (dd, J=11.7, 5.1 Hz, 1H), 3.46 (d, J=11.7 Hz, 1H), 3.40 (d, J=11.6 Hz, 1H), 1.44 (s, 3H), 1.42 (s, 3H), 1.30 (s, 9H).

### Step 5: tert-Butyl 5-(4-(6-chloro-4-oxo-3,4-dihydro-7H-pyrrolo[2,3-d]pyrimidin-7-yl)phenyl)-3,3-dimethylmorpholine-4-carboxylate

General procedure 5 using tert-butyl 5-(4-(4,6-dichloro-7H-pyrrolo[2,3-d]pyrimidin-7-yl)phenyl)-3,3-dimethylmorpholine-4-carboxylate (179 mg, 0.375 mmol), aqueous sodium hydroxide (4 M, 937 μL, 3.75 mmol) and 1,4-dioxane (1.9 mL) after 18 h 30 min at 100° C. using 2 M aqueous hydrochloric acid for acidification gave the title compound (171.3 mg, 99.5%) as yellow solid. LCMS (method A): R_{T}=1.43 min, m/z=459, 461 [M+H]⁺. ¹H NMR (500 MHz, DMSO-d₆) δ 12.16 (br. s, 1H), 7.84 (s, 1H), 7.52-7.47 (m, 2H), 7.42-7.37 (m, 2H), 6.68 (s, 1H), 4.94 (t, J=4.8 Hz, 1H), 4.10 (dd, J=11.7, 4.8 Hz, 1H), 4.04 (dd, J=11.7, 5.0 Hz, 1H), 3.45 (d, J=11.7 Hz, 1H), 3.38 (d, J=11.6 Hz, 1H), 1.43 (s, 3H), 1.41 (s, 3H), 1.30 (s, 9H).

### Step 6: tert-Butyl 5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-3,3-dimethylmorpholine-4-carboxylate

General procedure 6 using tert-butyl 5-(4-(6-chloro-4-oxo-3,4-dihydro-7H-pyrrolo[2,3-d]pyrimidin-7-yl)phenyl)-3,3-dimethylmorpholine-4-carboxylate (10 mg, 0.0218 mmol), Epoxide 1 (6.4 mg, 0.0327 mmol), Cs₂CO₃ (14.2 mg, 0.0436 mmol) and DMF (0.44 mL) after 19 h at 80° C. gave the title compound (8 mg, 56%) as a yellow glass. LCMS (method A): R_{T}=1.55 min, m/z=654, 656 [M+H]⁺.

### Step 7: 6-chloro-7-(4-(5,5-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

A solution of tert-butyl 5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-3,3-dimethylmorpholine-4-carboxylate (8 mg, 0.0122 mmol) in TFA (0.2 mL) and DCM (0.4 mL) was stirred for 30 min before the reaction mixture was concentrated under reduced pressure. The residue was dissolved 1:1 DCM/triethylamine (1 mL) and the resulting solution was purified by flash chromatography (Biotage® Sfär KP-Amino D 11 g cartridge, 0-100% DCM in cyclohexane then 0-10% MeOH in DCM) to give the title compound (6.6 mg, 94%) as a white solid after lyophilisation. LCMS (method B): R_{T}=0.71 min, m/z=554, 556 [M+H]⁺. ¹H NMR (500 MHz, DMSO-d₆) δ 8.07 (s, 1H), 7.66-7.58 (m, 2H), 7.42-7.34 (m, 2H), 6.80 (s, 1H), 4.91 (s, 1H), 4.22 (dd, J=10.5, 3.4 Hz, 1H), 4.00 (s, 2H), 3.95 (dt, J=13.2, 4.1 Hz, 2H), 3.84 (dd, J=10.5, 3.5 Hz, 1H), 3.46 (d, J=10.6 Hz, 1H), 3.22-2.98 (m, 4H), 2.43 (s, 1H), 1.57-1.45 (m, 2H), 1.43-1.35 (m, 2H), 1.28 (s, 3H), 1.21 (s, 3H), 1.02 (s, 3H), 0.81-0.72 (m, 2H), 0.57-0.45 (m, 2H).

### Example 16: 6-Chloro-3-((1-(4-chlorobenzoyl)-4-hydroxypiperidin-4-yl)methyl)-7-(4-(5,5-dimethylmorpholin-3-yl)phenyl)-3,7-dihydro-4H-pyrrolo[2,3-d]pyrimidin-4-one

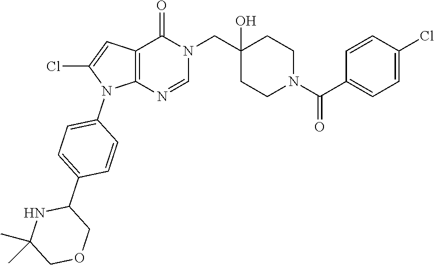 [SMILES: CC1(C)COCC(c2ccc(-n3c(Cl)cc4c(=O)n(CC5(O)CCN(C(=O)c6ccc(Cl)cc6)CC5)cnc43)cc2)N1]

### Step 1: tert-Butyl 5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-3,3-dimethylmorpholine-4-carboxylate

General procedure 6 using tert-butyl 5-(4-(6-chloro-4-oxo-3,4-dihydro-7H-pyrrolo[2,3-d]pyrimidin-7-yl)phenyl)-3,3-dimethylmorpholine-4-carboxylate (10 mg, 0.0218 mmol), Epoxide 2 (8.2 mg, 0.0327 mmol), Cs₂CO₃ (14.2 mg, 0.0436 mmol) and DMF (0.44 mL) after 19 h at 80° C. gave the title compound (8.1 mg, 52%) as yellow glass. LCMS (method A): R_{T}=1.74 min, m/z=710, 712 [M+H]⁺.

### Step 2: 6-Chloro-3-((1-(4-chlorobenzoyl)-4-hydroxypiperidin-4-yl)methyl)-7-(4-(5,5-dimethylmorpholin-3-yl)phenyl)-3,7-dihydro-4H-pyrrolo[2,3-d]pyrimidin-4-one

A solution of tert-butyl 5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-3,3-dimethylmorpholine-4-carboxylate (8.1 mg, 0.0114 mmol) in TFA (0.2 mL) and DCM (0.4 mL) was stirred for 30 min before the reaction mixture was concentrated under reduced pressure. The residue was dissolved 1:1 DCM/triethylamine (1 mL) and the resulting solution was purified by flash chromatography (Biotage® Sfär KP-Amino D 11 g cartridge, 0-100% DCM in cyclohexane then 0-10% MeOH in DCM) to give the title compound (7.1 mg, 94%) as a white solid after lyophilisation. LCMS (method B): R_{T}=0.84 min, m/z=610, 612 [M+H]⁺. ¹H NMR (500 MHz, DMSO-d₆) δ 8.06 (s, 1H), 7.66-7.59 (m, 2H), 7.53-7.47 (m, 2H), 7.44-7.40 (m, 2H), 7.40-7.35 (m, 2H), 6.79 (s, 1H), 4.95 (s, 1H), 4.25-4.19 (m, 1H), 4.16 (br. s, 1H), 4.02 (s, 2H), 3.84 (dd, J=10.6, 3.4 Hz, 1H), 3.46 (d, J=10.6 Hz, 1H), 3.41-3.03 (m, 5H (signals overlap with HDO)), 2.42 (br. s, 1H), 1.60 (br. s, 2H), 1.46 (br. s, 1H), 1.33 (br. s, 1H), 1.27 (s, 3H), 1.02 (s, 3H).

### Example 17: rac-3-((1-(4-Chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

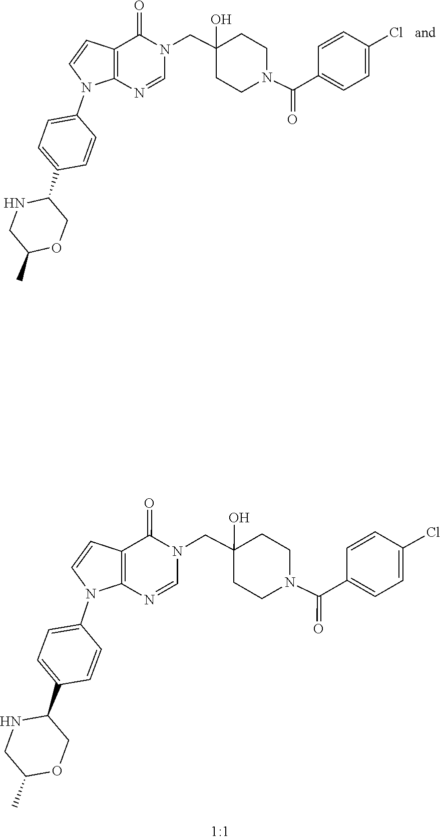 [SMILES: C[C@H]1CN[C@H](c2ccc(-n3ccc4c(=O)n(CC5(O)CCN(C(=O)c6ccc(Cl)cc6)CC5)cnc43)cc2)CO1 ; C[C@@H]1CN[C@@H](c2ccc(-n3ccc4c(=O)n(CC5(O)CCN(C(=O)c6ccc(Cl)cc6)CC5)cnc43)cc2)CO1]

### Step 1: rac-tert-Butyl (2R,5S)-5-(4-(4-chloro-7H-pyrrolo[2,3-d]pyrimidin-7-yl)phenyl)-2-methylmorpholine-4-carboxylate

General procedure 4 (Work-up A) using 4-chloro-7H-pyrrolo[2,3-d]pyrimidine (41 mg, 0.267 mmol), rac-tert-butyl (2R,5S)-2-methyl-5-(4-(4,4,5,5-tetramethyl-1,3,2-dioxaborolan-2-yl)phenyl)morpholine-4-carboxylate (215 mg, 0.534 mmol), copper(II) acetate (97 mg, 0.534 mmol), 1,10-phenanthroline (96 mg, 0.534 mmol), boric acid (66 mg, 1.07 mmol) and DMF (23 mL) after 5 days at 50° C. gave the title compound (95 mg, 83%) as a colourless glass. LCMS (method B): R_{T}=1.57 min, m/z=429, 431 [M+H]⁺.

### Step 2: rac-tert-Butyl (2R,5S)-2-methyl-5-(4-(4-oxo-3,4-dihydro-7H-pyrrolo[2,3-d]pyrimidin-7-yl)phenyl)morpholine-4-carboxylate

A solution of rac-tert-butyl (2R,5S)-5-(4-(4-chloro-7H-pyrrolo[2,3-d]pyrimidin-7-yl)phenyl)-2-methylmorpholine-4-carboxylate (95 mg, 0.222 mmol) and aqueous sodium hydroxide (2 M, 1.66 mL, 3.32 mmol) in 1,4-dioxane (2.5 mL) was heated at 100° C. for 18 h. Upon cooling to rt water was added and the resulting mixture was extracted with DCM (3×15 mL). The combined organic phases were concentrated under reduced pressure to give the title compound (90.3 mg, 99%) as a white solid. LCMS (method B): R_{T}=1.14 min, m/z=411 [M+H]⁺.

### Step 3: rac-tert-Butyl (2R,5S)-5-(4-(3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using rac-tert-butyl (2R,5S)-2-methyl-5-(4-(4-oxo-3,4-dihydro-7H-pyrrolo[2,3-d]pyrimidin-7-yl)phenyl)morpholine-4-carboxylate (90 mg, 0.219 mmol), Epoxide 2 (66 mg, 0.263 mmol), Cs₂CO₃ (157 mg, 0.482 mmol) and DMF (1.5 mL) after 18 h at 80° C., after purification by prep-HPLC, gave the title compound (73.1 mg, 50%) as a white solid. LCMS (method B): R_{T}=1.40 min, m/z=606, 608 [M−butene+H]⁺.

### Step 4: rac-3-((1-(4-Chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using rac-tert-butyl (2R,5S)-5-(4-(3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (73 mg, 0.110 mmol), TFA (0.25 mL) and DCM (0.25 mL) gave, after freeze-drying, the title compound (61 mg, 97%) as a white solid. LCMS (method B): R_{T}=0.77 min, m/z=562, 564 [M+H]⁺. ¹H NMR (500 MHz, DMSO-d₆) δ 8.13 (s, 1H), 7.80-7.64 (m, 2H), 7.64-7.54 (m, 2H), 7.54-7.45 (m, 3H), 7.45-7.35 (m, 2H), 6.74-6.65 (m, 1H), 4.96 (s, 1H), 4.17 (br. s, 1H), 4.05 (br. s, 3H), 3.87 (br. s, 1H), 3.69 (br. s, 1H), 3.62-3.20 (m, 3H (signals overlap with HDO)), 3.13 (br. s, 3H), 2.69 (br. s, 1H), 1.61 (br. s, 2H), 1.48 (br. s, 1H), 1.34 (br. s, 1H), 1.13 (br. s, 3H).

### Example 18: 7-(4-(7-Oxa-4-azaspiro[2.5]octan-5-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

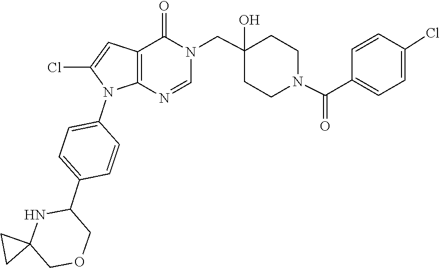 [SMILES: O=C(c1ccc(Cl)cc1)N1CCC(O)(Cn2cnc3c(cc(Cl)n3-c3ccc(C4COCC5(CC5)N4)cc3)c2=O)CC1]

### Step 1: 5-(4-Bromophenyl)-4-(4-methoxybenzyl)morpholin-3-one

A solution of 5-(4-bromophenyl)morpholin-3-one (3.30 g, 12.9 mmol) in DMF (129 mL) was cooled down to 0° C. before sodium hydride (60% in mineral oil) (670 mg, 27.9 mmol) was added. The reaction mixture was allowed to warm to rt and stirred for 1 h before 1-(chloromethyl)-4-methoxybenzene (2.22 g, 14.2 mmol) was added slowly. After stirring at rt for 12 h the reaction mixture was diluted with EtOAc before being washed with water and brine. The organic phase was dried over Na₂SO₄, filtered and concentrated under reduced pressure. The residue was purified by flash chromatography to give the title compound (3.9 g, 80%). LCMS (method C): R_{T}=1.36, m/z=376, 378 [M+H]⁺. ¹H NMR (500 MHz, Chloroform-d) δ 7.52 (d, J=8.4 Hz, 2H), 7.10 (d, J=8.1 Hz, 2H), 7.06 (d, J=8.1 Hz, 2H), 6.84 (d, J=8.0 Hz, 2H), 5.48 (d, J=14.6 Hz, 1H), 4.41 (d, J=16.4 Hz, 1H), 4.31 (dd, J=16.8, 1.6 Hz, 1H), 4.28-4.23 (m, 1H), 3.91 (ddd, J=12.0, 3.9, 1.6 Hz, 1H), 3.80 (s, 3H), 3.78-3.73 (m, 1H), 3.35 (d, J=14.3 Hz, 1H).

### Step 2: 5-(4-Bromophenyl)-4-(4-methoxybenzyl)-7-oxa-4-azaspiro[2.5]octane

A solution of ethylmagnesium bromide (3.4 M in THF) (9.15 mL, 31.1 mmol) was added to a solution of titanium(IV) isopropoxide (3.24 g, 11.4 mmol) in anhydrous THE (55 mL) at −60° C. A solution of 5-(4-bromophenyl)-4-(4-methoxybenzyl)morpholin-3-one (3.90 g, 10.4 mmol) in THE (10 mL) was added dropwise and the mixture stirred at −60° C. for 2 h. The mixture was quenched with saturated aqueous ammonium chloride/water and the volatiles were removed under reduced pressure. The residue was diluted with water and washed with EtOAc. The organic layer was dried with sodium sulfate, filtered, and concentrated under reduced pressure to give the title compound (0.9 g, 22%). LCMS (method C): R_{T}=1.58, m/z=388, 390 [M+H]⁺. ¹H NMR (400 MHz, Chloroform-d) δ 7.33 (d, J=8.5 Hz, 2H), 7.24 (d, J=8.5 Hz, 2H), 7.17 (d, J=8.6 Hz, 2H), 6.81 (d, J=8.6 Hz, 2H), 4.04 (dd, J=11.9, 3.5 Hz, 1H), 3.95 (dd, J=11.9, 5.6 Hz, 1H), 3.81-3.76 (m, 4H), 3.74-3.70 (m, 1H), 3.66 (d, J=11.2 Hz, 1H), 3.54-3.45 (m, 2H), 0.36-0.28 (m, 2H), 0.68-0.60 (m, 1H), 0.43-0.36 (m, 1H).

### Step 3: 5-(4-Bromophenyl)-7-oxa-4-azaspiro[2.5]octane

A solution 5-(4-bromophenyl)-4-(4-methoxybenzyl)-7-oxa-4-azaspiro[2.5]octane (0.9 g, 2.32 mmol) in TFA (10 mL) was heated at 70° C. overnight. The reaction mixture was concentrated under reduced pressure and the residue was purified by flash chromatography to give the title compound (0.3 g, 47%). LCMS (method C): R_{T}=0.72, m/z=268, 270 [M+H]⁺. ¹H NMR (500 MHz, Chloroform-d) δ 7.44 (d, J=8.1 Hz, 2H), 7.21 (d, J=8.4 Hz, 2H), 4.08 (dd, J=10.3, 3.2 Hz, 1H), 3.97-3.90 (m, 2H), 3.35 (d, J=11.3 Hz, 1H), 3.16 (d, J=11.2 Hz, 1H), 0.79-0.69 (m, 1H), 0.67-0.53 (m, 3H).

### Step 4: tert-Butyl 5-(4-bromophenyl)-7-oxa-4-azaspiro[2.5]octane-4-carboxylate

A solution of 5-(4-bromophenyl)-7-oxa-4-azaspiro[2.5]octane (0.3 g, 1.10 mmol) and Boc₂O (0.48 g, 2.2 mmol) in MeCN (2 mL) was stirred at 70° C. overnight. Upon cooling to rt the reaction mixture was concentrated under reduced pressure and the residue was purified by flash chromatography to give the title compound (368 mg, 91%). LCMS (method C): R_{T} 1.50 min, m/z=268, 270 [M−Boc+H]⁺. ¹H NMR (500 MHz, Chloroform-d) δ 7.59-7.35 (m, 4H), 5.29-5.20 (m, 1H), 4.37 (dd, J=12.3, 2.2 Hz, 1H), 4.05 (dd, J=11.0, 2.2 Hz, 1H), 3.91 (dd, J=12.2, 4.2 Hz, 1H), 3.00 (d, J=11.0 Hz, 1H), 1.50 (s, 9H), 1.23 (dt, J=10.0, 7.1 Hz, 1H), 0.65 (dt, J=9.9, 6.8 Hz, 1H), 0.38 (ddd, J=10.0, 7.2, 5.2 Hz, 1H), −0.03 (ddd, J=10.9, 7.7, 5.4 Hz, 1H).

### Step 5: tert-Butyl 5-(4-(4,4,5,5-tetramethyl-1,3,2-dioxaborolan-2-yl)phenyl)-7-oxa-4-azaspiro[2.5]octane-4-carboxylate

General procedure 3 (Work-up A) using tert-butyl 5-(4-bromophenyl)-7-oxa-4-azaspiro[2.5]octane-4-carboxylate (385 mg, 1.05 mmol), B₂Pin₂ (398 mg, 1.57 mmol), Pd(dppf)Cl₂·CH₂Cl₂ (88.5 mg, 0.105 mmol), KOAc (308 mg, 3.14 mmol) and 1,4-dioxane (11 mL) after stirring at 100° C. (microwave) for 1 h gave the title compound (374 mg, 85%) as a slightly yellow oil. LCMS (method B): R_{T}=1.74 min, m/z=316 [M−Boc+H]⁺.

### Step 6: tert-Butyl 5-(4-(4,6-dichloro-7H-pyrrolo[2,3-d]pyrimidin-7-yl)phenyl)-7-oxa-4-azaspiro[2.5]octane-4-carboxylate

General procedure 4 (Work-up A) using 4,6-dichloro-7H-pyrrolo[2,3-d]pyrimidine (113 mg, 0.601 mmol), tert-butyl 5-(4-(4,4,5,5-tetramethyl-1,3,2-dioxaborolan-2-yl)phenyl)-7-oxa-4-azaspiro[2.5]octane-4-carboxylate (374 mg, 0.902 mmol), copper(II) trifluoroacetate hydrate (348 mg, 1.20 mmol), 1,10-phenanthroline (217 mg, 1.20 mmol), boric acid (149 mg, 2.40 mmol) and DMF (11.3 mL) after 1 day at 65° C. and 12 h at 80° C. gave the title compound (62 mg, 21%) as a colourless glass. LCMS (method B): R_{T}=1.74 min, m/z=475, 477 [M+H]⁺.

### Step 7: tert-Butyl 5-(4-(6-chloro-4-oxo-3,4-dihydro-7H-pyrrolo[2,3-d]pyrimidin-7-yl)phenyl)-7-oxa-4-azaspiro[2.5]octane-4-carboxylate

General procedure 5 using tert-butyl 5-(4-(4,6-dichloro-7H-pyrrolo[2,3-d]pyrimidin-7-yl)phenyl)-7-oxa-4-azaspiro[2.5]octane-4-carboxylate (62 mg, 0.130 mmol), aqueous sodium hydroxide (4 M, 0.98 mL, 3.91 mmol) and 1,4-dioxane (1 mL) after 18 h at 100° C. using 1 M aqueous potassium bisulfate for acidification gave the title compound (60 mg, quant.) as a yellow glass. LCMS (method B): R_{T}=1.29 min, m/z=457, 459 [M+H]⁺.

### Step 8: tert-Butyl 5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-7-oxa-4-azaspiro[2.5]octane-4-carboxylate

General procedure 6 using tert-butyl 5-(4-(6-chloro-4-oxo-3,4-dihydro-7H-pyrrolo[2,3-d]pyrimidin-7-yl)phenyl)-7-oxa-4-azaspiro[2.5]octane-4-carboxylate (30 mg, 0.0657 mmol), Epoxide 2 (24.8 mg, 0.0985 mmol), Cs₂CO₃ (64.2 mg, 0.197 mmol) and DMF (1.3 mL) after 18 h at 80° C. gave the title compound (24 mg, 51%) as a colourless glass. LCMS (method B): R_{T}=1.54 min, m/z=652, 654 [M−butene+H]⁺.

### Step 9: 7-(4-(7-Oxa-4-azaspiro[2.5]octan-5-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

General procedure 7 using tert-butyl 5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-7-oxa-4-azaspiro[2.5]octane-4-carboxylate (24 mg, 0.0339 mmol), TFA (1 mL) and DCM (2 mL) gave, after freeze-drying, the title compound (20 mg, 97%) as an off-white solid. LCMS (method B): R_{T}=0.83 min, m/z=608, 610 [M+H]⁺. ¹H NMR (500 MHz, DMSO-d₆) δ 8.06 (s, 1H), 7.64-7.56 (m, 2H), 7.54-7.46 (m, 2H), 7.46-7.40 (m, 2H), 7.40-7.35 (m, 2H), 6.79 (s, 1H), 4.95 (s, 1H), 4.28-4.10 (m, 1H), 4.09-4.03 (m, 1H), 4.02 (s, 2H), 3.91 (dd, J=10.7, 3.2 Hz, 1H), 3.88-3.82 (m, 1H), 3.44-3.33 (m, 1H), 3.30-3.20 (m, 2H), 3.20-3.03 (m, 2H), 2.83-2.68 (m, 1H), 1.70-1.25 (m, 4H), 0.64-0.45 (m, 4H).

### Example 19: 7-(4-(7-Oxa-4-azaspiro[2.5]octan-5-yl)phenyl)-6-chloro-3-((4-hydroxy-1-(1-methylcyclopropane-1-carbonyl)piperidin-4-yl)methyl)-3,7-dihydro-4H-pyrrolo[2,3-d]pyrimidin-4-one

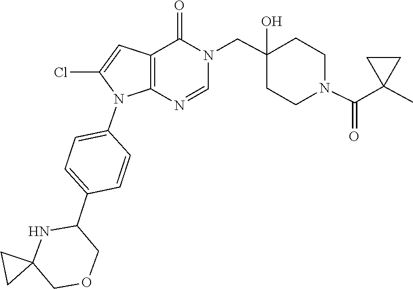 [SMILES: CC1(C(=O)N2CCC(O)(Cn3cnc4c(cc(Cl)n4-c4ccc(C5COCC6(CC6)N5)cc4)c3=O)CC2)CC1]

### Step 1: tert-Butyl 5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-7-oxa-4-azaspiro[2.5]octane-4-carboxylate

General procedure 6 using tert-butyl 5-(4-(6-chloro-4-oxo-3,4-dihydro-7H-pyrrolo[2,3-d]pyrimidin-7-yl)phenyl)-7-oxa-4-azaspiro[2.5]octane-4-carboxylate (30 mg, 0.0657 mmol), Epoxide 1 (19.23 mg, 0.0985 mmol), Cs₂CO₃ (64.2 mg, 0.197 mmol) and DMF (1.3 mL) after 18 h at 80° C. gave the title compound (25 mg, 58%) as a colourless glass. LCMS (method B): R_{T}=1.39 min, m/z=596, 598 [M−butene+H]⁺.

### Step 2: 7-(4-(7-Oxa-4-azaspiro[2.5]octan-5-yl)phenyl)-6-chloro-3-((4-hydroxy-1-(1-methylcyclopropane-1-carbonyl)piperidin-4-yl)methyl)-3,7-dihydro-4H-pyrrolo[2,3-d]pyrimidin-4-one

General procedure 7 using tert-butyl 5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-7-oxa-4-azaspiro[2.5]octane-4-carboxylate (25 mg, 0.0383 mmol), TFA (1 mL) and DCM (2 mL) gave, after freeze-drying, the title compound (21 mg, 99%) as an off-white solid. LCMS (method B): R_{T}=0.70 min, m/z=552, 554 [M+H]⁺. ¹H NMR (500 MHz, DMSO-d₆) δ 8.07 (s, 1H), 7.67-7.54 (m, 2H), 7.45-7.30 (m, 2H), 6.80 (s, 1H), 4.91 (s, 1H), 4.10-4.03 (m, 1H), 4.00 (s, 2H), 3.98-3.88 (m, 3H), 3.88-3.82 (m, 1H), 3.30-3.02 (m, 4H), 2.76 (d, J=6.2 Hz, 1H), 1.62-1.32 (m, 4H), 1.21 (s, 3H), 0.81-0.72 (m, 2H), 0.61-0.44 (m, 6H).

### Example 20: 3-((1-Benzoyl-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one

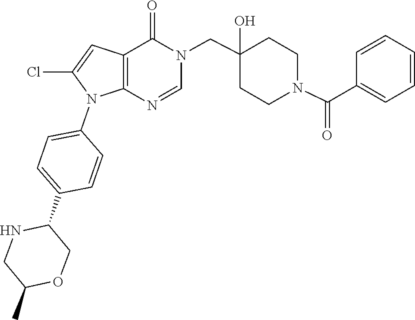 [SMILES: C[C@H]1CN[C@H](c2ccc(-n3c(Cl)cc4c(=O)n(CC5(O)CCN(C(=O)c6ccccc6)CC5)cnc43)cc2)CO1]

### Step 1: tert-Butyl (2S,5R)-5-(4-(3-((1-benzoyl-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 4 (29.3 mg, 0.135 mmol), Cs₂CO₃ (88 mg, 0.270 mmol) and DMF (2 mL) after 18 h at 80° C. gave the title compound (40 mg, 67%) as a colourless glass. LCMS (method A): R_{T}=1.51 min, m/z=606, 608 [M−butene+H]⁺.

### Step 2: 3-((1-Benzoyl-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(3-((1-benzoyl-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate (40 mg, 0.0604 mmol), TFA (1 mL) and DCM (2 mL) gave, after freeze-drying, the title compound (32 mg, 94%) as an off-white solid. LCMS (method B): R_{T}=0.75 min, m/z=562, 564 [M+H]⁺. ¹H NMR (500 MHz, DMSO-d₆) δ 8.07 (s, 1H), 7.65-7.56 (m, 2H), 7.49-7.42 (m, 3H), 7.42-7.33 (m, 4H), 6.80 (s, 1H), 4.95 (s, 1H), 4.32-4.11 (m, 1H), 4.03 (s, 2H), 3.91-3.75 (m, 2H), 3.63-3.49 (m, 1H), 3.47-3.34 (m, 1H), 3.30-3.21 (m, 2H), 3.21-3.07 (m, 1H), 3.04-2.81 (m, 2H), 2.60-2.53 (m, 1H), 1.74-1.22 (m, 4H), 1.10 (d, J=6.2 Hz, 3H).

### Example 21: 6-Chloro-3-((1-(4-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

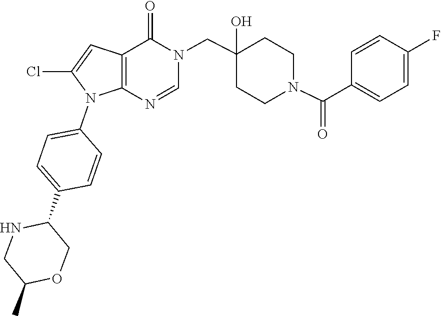 [SMILES: C[C@H]1CN[C@H](c2ccc(-n3c(Cl)cc4c(=O)n(CC5(O)CCN(C(=O)c6ccc(F)cc6)CC5)cnc43)cc2)CO1]

### Step 1: tert-Butyl (2S,5R)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 5 (31.7 mg, 0.135 mmol), Cs₂CO₃ (88 mg, 0.270 mmol) and DMF (2 mL) after 18 h at 80° C. gave the title compound (36 mg, 58%) as a colourless glass. LCMS (method B): R_{T}=1.42 min, m/z=624, 626 [M−butene+H]⁺.

### Step 2: 6-Chloro-3-((1-(4-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (36 mg, 0.0529 mmol), TFA (1 mL) and DCM (2 mL) gave, after freeze-drying, the title compound (29 mg, 94%) as an off-white solid. LCMS (method B): R_{T}=0.77 min, m/z=580, 582 [M+H]⁺. ¹H NMR (500 MHz, DMSO-d₆) δ 8.07 (s, 1H), 7.63-7.58 (m, 2H), 7.51-7.43 (m, 2H), 7.43-7.36 (m, 2H), 7.32-7.23 (m, 2H), 6.80 (s, 1H), 4.96 (s, 1H), 4.32-4.07 (m, 1H), 4.03 (s, 2H), 3.88-3.78 (m, 2H), 3.61-3.50 (m, 1H), 3.49-3.35 (m, 1H), 3.30-3.22 (m, 2H), 3.22-3.03 (m, 1H), 2.99-2.93 (m, 1H), 2.93-2.84 (m, 1H), 2.60-2.53 (m, 1H), 1.76-1.25 (m, 4H), 1.10 (d, J=6.2 Hz, 3H).

### Example 22: 6-Chloro-3-((1-(2,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

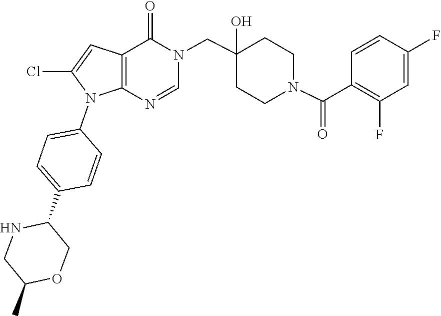 [SMILES: C[C@H]1CN[C@H](c2ccc(-n3c(Cl)cc4c(=O)n(CC5(O)CCN(C(=O)c6ccc(F)cc6F)CC5)cnc43)cc2)CO1]

### Step 1: tert-Butyl (2S,5R)-5-(4-(6-chloro-3-((1-(2,4-difluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 6 (34.2 mg, 0.135 mmol), Cs₂CO₃ (88 mg, 0.270 mmol) and DMF (2 mL) after 18 h at 80° C. gave the title compound (38 mg, 60%) as a colourless glass. LCMS (method B): R_{T}=1.44 min, m/z=642, 644 [M−butene+H]⁺.

### Step 2: 6-Chloro-3-((1-(2,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(2,4-difluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (38 mg, 0.0544 mmol), TFA (1 mL) and DCM (2 mL) gave, after freeze-drying, the title compound (31 mg, 95%) as a white solid. LCMS (method B): R_{T}=0.79 min, m/z=598, 600 [M+H]⁺. ¹H NMR (500 MHz, DMSO-d₆) δ 8.07 (s, 1H), 7.64-7.58 (m, 2H), 7.55-7.44 (m, 1H), 7.43-7.31 (m, 3H), 7.18 (td, J=8.4, 2.4 Hz, 1H), 6.80 (s, 1H), 4.99 (s, 1H), 4.28-4.16 (m, 1H), 4.13-3.91 (m, 2H), 3.91-3.76 (m, 2H), 3.61-3.50 (m, 1H), 3.30-3.20 (m, 3H), 3.19-3.11 (m, 1H), 3.04-2.81 (m, 2H), 2.59-2.53 (m, 1H), 1.68-1.44 (m, 3H), 1.42-1.28 (m, 1H), 1.10 (d, J=6.2 Hz, 3H).

### Example 23: (S)-6-chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one

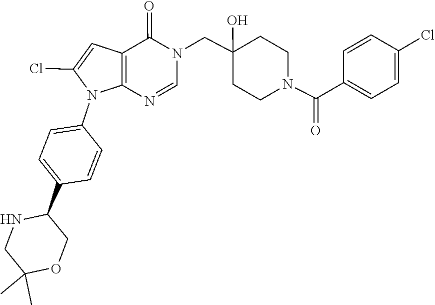 [SMILES: CC1(C)CN[C@@H](c2ccc(-n3c(Cl)cc4c(=O)n(CC5(O)CCN(C(=O)c6ccc(Cl)cc6)CC5)cnc43)cc2)CO1]

### Step 1: tert-Butyl (S)-5-(4-(4,6-dichloro-7H-pyrrolo[2,3-d]pyrimidin-7-yl)phenyl)-2,2-dimethylmorpholine-4-carboxylate and tert-butyl (R)-5-(4-(4,6-dichloro-7H-pyrrolo[2,3-d]pyrimidin-7-yl)phenyl)-2,2-dimethylmorpholine-4-carboxylate

tert-Butyl 5-(4-(4,6-dichloro-7H-pyrrolo[2,3-d]pyrimidin-7-yl)phenyl)-2,2-dimethylmorpholine-4-carboxylate (776 mg) was resolved into the single stereoisomers by chiral SFC using a Lux A1 (21.2 mm×250 mm, 5 μm) column with isocratic solvent conditions: 25:75 MeOH:CO₂. The first eluted material was arbitrarily assigned as tert-butyl (S)-5-(4-(4,6-dichloro-7H-pyrrolo[2,3-d]pyrimidin-7-yl)phenyl)-2,2-dimethylmorpholine-4-carboxylate (288 mg, 37% recovery). Chiral purity (method E): R_{T}=1.45 min, 100% ee. The first eluted material was arbitrarily assigned as tert-butyl (R)-5-(4-(4,6-dichloro-7H-pyrrolo[2,3-d]pyrimidin-7-yl)phenyl)-2,2-dimethylmorpholine-4-carboxylate (320.9 mg, 41% recovery) as an off-white solid. Chiral purity (method E): R_{T}=2.25 min, 100% ee.

### Step 2: tert-Butyl (S)-5-(4-(6-chloro-4-oxo-3,4-dihydro-7H-pyrrolo[2,3-d]pyrimidin-7-yl)phenyl)-2,2-dimethylmorpholine-4-carboxylate

General procedure 5 using tert-butyl (S)-5-(4-(4,6-dichloro-7H-pyrrolo[2,3-d]pyrimidin-7-yl)phenyl)-2,2-dimethylmorpholine-4-carboxylate (288 mg, 0.603 mmol), aqueous sodium hydroxide (4 M, 4.52 mL, 18.1 mmol) and 1,4-dioxane (4.5 mL) after 18 h at 100° C. using 1 M aqueous potassium bisulfate for acidification gave the title compound (286 mg, >100%) as a yellow glass. LCMS (method B): R_{T}=1.40 min, m/z=459, 461 [M+H]⁺.

### Step 3: tert-Butyl (S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate

General procedure 6 using tert-butyl (S)-5-(4-(6-chloro-4-oxo-3,4-dihydro-7H-pyrrolo[2,3-d]pyrimidin-7-yl)phenyl)-2,2-dimethylmorpholine-4-carboxylate (80 mg, 0.174 mmol), Epoxide 2 (65.8 mg, 0.262 mmol), Cs₂CO₃ (170 mg, 0.523 mmol) and DMF (3 mL) after 18 h at 80° C. gave the title compound (85 mg, 68%) as a colourless glass. LCMS (method B): R_{T}=1.77 min, m/z=654, 656 [M−butene+H]⁺.

### Step 4: (S)-6-Chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one

General procedure 7 using tert-butyl (S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate (85 mg, 0.120 mmol), TFA (2 mL) and DCM (4 mL) gave, after freeze-drying, the title compound (69 mg, 94%) as an off-white solid. LCMS (method B): R_{T}=0.86 min, m/z=610, 612 [M+H]⁺. ¹H NMR (500 MHz, DMSO-d₆) δ 8.07 (s, 1H), 7.69-7.60 (m, 2H), 7.54-7.47 (m, 2H), 7.47-7.34 (m, 4H), 6.80 (s, 1H), 4.96 (s, 1H), 4.32-4.09 (m, 1H), 4.03 (s, 2H), 3.81 (dd, J=10.3, 3.5 Hz, 1H), 3.58 (dd, J=11.1, 3.6 Hz, 1H), 3.47 (t, J=10.7 Hz, 1H), 3.43-3.33 (m, 1H), 3.30-2.98 (m, 2H), 2.90-2.72 (m, 2H), 2.68 (d, J=11.5 Hz, 1H), 1.75-1.23 (m, 7H), 1.14 (s, 3H).

### Example 24: 3-((1-Benzoyl-4-hydroxypiperidin-4-yl)methyl)-6-chloro-7-(4-((3S,6R)-6-methylmorpholin-3-yl)phenyl)-3,7-dihydro-4H-pyrrolo[2,3-d]pyrimidin-4-one

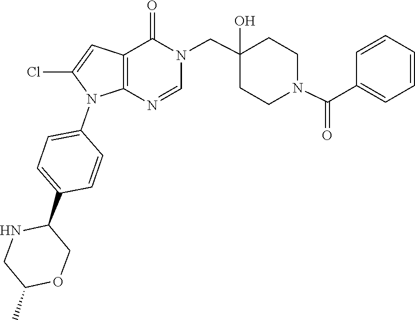 [SMILES: C[C@@H]1CN[C@@H](c2ccc(-n3c(Cl)cc4c(=O)n(CC5(O)CCN(C(=O)c6ccccc6)CC5)cnc43)cc2)CO1]

### Step 1: tert-Butyl (2R,5S)-5-(4-(3-((1-benzoyl-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (70 mg, 0.157 mmol), Epoxide 4 (41 mg, 0.189 mmol), Cs₂CO₃ (113 mg, 0.346 mmol) and DMF (1.5 mL) after 18 h at 80° C. gave, after purification by prep-HPLC, the title compound (64 mg, 61%) as a white solid. LCMS (method B): R_{T}=1.39 min, m/z=606, 608 [M−butene+H]⁺.

### Step 2: 3-((1-Benzoyl-4-hydroxypiperidin-4-yl)methyl)-6-chloro-7-(4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(3-((1-benzoyl-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate (64 mg, 0.0967 mmol), TFA (1 mL) and DCM (2 mL) gave, after purification by flash chromatography and freeze-drying, the title compound (29.1 mg, 53%) as a white solid.

LCMS (method B): R_{T}=0.74 min, m/z=562, 564 [M+H]⁺. ¹H NMR (500 MHz, DMSO-d₆) δ 8.06 (s, 1H), 7.62-7.57 (m, 2H), 7.46-7.41 (m, 3H), 7.38 (td, J=7.6, 7.0, 2.8 Hz, 4H), 6.79 (s, 1H), 4.95 (s, 1H), 4.17 (s, 1H), 4.02 (s, 2H), 3.82 (ddd, J=19.5, 10.3, 3.2 Hz, 2H), 3.58-3.51 (m, 1H), 3.39 (s, 1H), 3.29-3.05 (m, 3H), 2.99-2.82 (m, 2H), 2.57-2.52 (m, 1H), 1.69-1.25 (m, 4H), 1.09 (d, J=6.2 Hz, 3H).

### Example 25: 6-Chloro-3-((1-(4-fluorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one

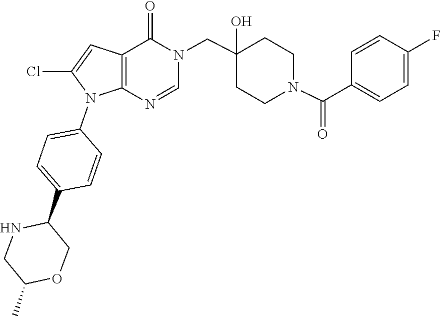 [SMILES: C[C@@H]1CN[C@@H](c2ccc(-n3c(Cl)cc4c(=O)n(CC5(O)CCN(C(=O)c6ccc(F)cc6)CC5)cnc43)cc2)CO1]

### Step 1: tert-Butyl (2R,5S)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (70 mg, 0.157 mmol), Epoxide 5 (44.4 mg, 0.189 mmol), Cs₂CO₃ (113 mg, 0.346 mmol) and DMF (1.5 mL) after 18 h at 80° C. gave, after purification by prep-HPLC, the title compound (91.4 mg, 85%) as a white solid. LCMS (method B): R_{T}=1.42 min, m/z=624, 626 [M−butene+H]⁺.

### Step 2: 6-Chloro-3-((1-(4-fluorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (95.4 mg, 0.140 mmol), TFA (1.5 mL) and DCM (3 mL) gave, after purification by flash chromatography and freeze-drying, the title compound (42.8 mg, 52%) as a white solid. LCMS (method B): R_{T}=0.73 min, m/z=580, 582 [M+H]⁺. ¹H NMR (500 MHz, DMSO-d₆) δ 8.06 (s, 1H), 7.62-7.57 (m, 2H), 7.48-7.43 (m, 2H), 7.41-7.36 (m, 2H), 7.29-7.23 (m, 2H), 6.79 (s, 1H), 4.95 (s, 1H), 4.16 (s, 1H), 4.02 (s, 2H), 3.82 (ddd, J=19.6, 10.3, 3.3 Hz, 2H), 3.55 (dqd, J=12.4, 6.1, 2.1 Hz, 1H), 3.39 (s, 1H), 3.28-3.04 (m, 3H), 2.99-2.82 (m, 2H), 2.57-2.52 (m, 1H), 1.53 (d, J=72.6 Hz, 4H), 1.09 (d, J=6.2 Hz, 3H).

### Example 26: 6-Chloro-3-((1-(4-chlorobenzoyl)-4-hydroxypiperidin-4-yl)methyl)-7-(4-((3R,5S)-5-methylmorpholin-3-yl)phenyl)-3,7-dihydro-4H-pyrrolo[2,3-d]pyrimidin-4-one

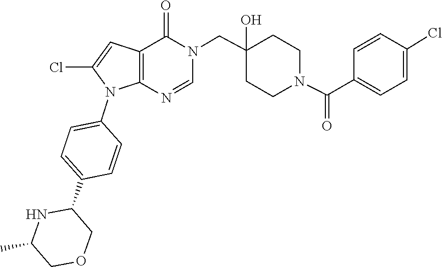 [SMILES: C[C@H]1COC[C@@H](c2ccc(-n3c(Cl)cc4c(=O)n(CC5(O)CCN(C(=O)c6ccc(Cl)cc6)CC5)cnc43)cc2)N1]

### Step 1: tert-Butyl (3R,5S)-3-(4-(4,6-dichloro-7H-pyrrolo[2,3-d]pyrimidin-7-yl)phenyl)-5-methylmorpholine-4-carboxylate and tert-butyl (3S,5R)-3-(4-(4,6-dichloro-7H-pyrrolo[2,3-d]pyrimidin-7-yl)phenyl)-5-methylmorpholine-4-carboxylate

rac-tert-Butyl (3R,5S)-3-(4-(4,6-dichloro-7H-pyrrolo[2,3-d]pyrimidin-7-yl)phenyl)-5-methylmorpholine-4-carboxylate (664.7 mg) was resolved into the single stereoisomers by chiral SFC using a Chiralpak IH (20 mm×250 mm, 5 μm) column with isocratic solvent conditions: 15:85 MeOH:CO₂. The first eluted material was arbitrarily assigned as tert-butyl (3R,5S)-3-(4-(4,6-dichloro-7H-pyrrolo[2,3-d]pyrimidin-7-yl)phenyl)-5-methylmorpholine-4-carboxylate (309 mg, 46% recovery). Chiral purity (method F): R_{T}=1.45 min, 100% ee. The first eluted material was arbitrarily assigned as tert-butyl (3S,5R)-3-(4-(4,6-dichloro-7H-pyrrolo[2,3-d]pyrimidin-7-yl)phenyl)-5-methylmorpholine-4-carboxylate (318.8 mg, 47% recovery). Chiral purity (method F): R_{T}=1.70 min, 99.4% ee.

### Step 2: tert-Butyl (3R,5S)-3-(4-(6-chloro-4-oxo-3,4-dihydro-7H-pyrrolo[2,3-d]pyrimidin-7-yl)phenyl)-5-methylmorpholine-4-carboxylate

General procedure 5 using tert-butyl (3R,5S)-3-(4-(4,6-dichloro-7H-pyrrolo[2,3-d]pyrimidin-7-yl)phenyl)-5-methylmorpholine-4-carboxylate (309 mg, 0.667 mmol), aqueous sodium hydroxide (4 M, 5 mL, 20.0 mmol) and 1,4-dioxane (5 mL) after 18 h at 100° C. using 1 M aqueous potassium bisulfate for acidification gave the title compound (337 mg, >100%) as a yellow glass. LCMS (method B): R_{T}=1.32 min, m/z=445, 447 [M+H]⁺.

### Step 3: tert-Butyl (3R,5S)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-5-methylmorpholine-4-carboxylate

General procedure 6 using tert-butyl (3R,5S)-3-(4-(6-chloro-4-oxo-3,4-dihydro-7H-pyrrolo[2,3-d]pyrimidin-7-yl)phenyl)-5-methylmorpholine-4-carboxylate (80 mg, 0.180 mmol), Epoxide 2 (68 mg, 0.270 mmol), Cs₂CO₃ (176 mg, 0.539 mmol) and DMF (3 mL) after 18 h at 80° C. gave the title compound (69 mg, 55%) as a colourless glass. LCMS (method B): R_{T}=1.50 min, m/z=640, 642 [M−butene+H]⁺.

### Step 4: 6-Chloro-3-((1-(4-chlorobenzoyl)-4-hydroxypiperidin-4-yl)methyl)-7-(4-((3R,5S)-5-methylmorpholin-3-yl)phenyl)-3,7-dihydro-4H-pyrrolo[2,3-d]pyrimidin-4-one

General procedure 7 using tert-butyl (3R,5S)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-5-methylmorpholine-4-carboxylate (69 mg, 0.0990 mmol), TFA (2 mL) and DCM (4 mL) gave, after freeze-drying, the title compound (54 mg, 91%) as an off-white solid. LCMS (method A): R_{T}=0.76 min, m/z=596, 598 [M+H]⁺. ¹H NMR (500 MHz, DMSO-d₆) δ 8.07 (s, 1H), 7.64-7.60 (m, 2H), 7.54-7.48 (m, 2H), 7.46-7.37 (m, 4H), 6.80 (s, 1H), 4.96 (s, 1H), 4.29-4.09 (m, 1H), 4.03 (s, 2H), 4.00-3.88 (m, 1H), 3.85-3.64 (m, 2H), 3.49-3.34 (m, 1H), 3.31-3.20 (m, 1H), 3.21-3.07 (m, 2H), 3.08-2.78 (m, 3H), 1.76-1.27 (m, 4H), 0.97 (d, J=6.0 Hz, 3H).

### Example 27: 6-Chloro-3-((1-(4-chlorobenzoyl)-4-hydroxypiperidin-4-yl)methyl)-7-(4-((3S,5R)-5-methylmorpholin-3-yl)phenyl)-3,7-dihydro-4H-pyrrolo[2,3-d]pyrimidin-4-one

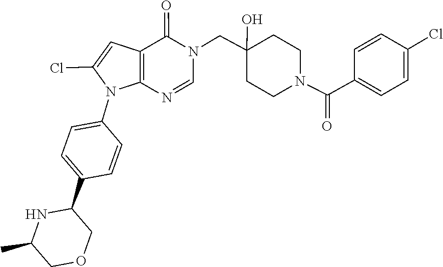 [SMILES: C[C@@H]1COC[C@H](c2ccc(-n3c(Cl)cc4c(=O)n(CC5(O)CCN(C(=O)c6ccc(Cl)cc6)CC5)cnc43)cc2)N1]

### Step 1: tert-Butyl (3S,5R)-3-(4-(6-chloro-4-oxo-3,4-dihydro-7H-pyrrolo[2,3-d]pyrimidin-7-yl)phenyl)-5-methylmorpholine-4-carboxylate

General procedure 5 using tert-butyl (3S,5R)-3-(4-(4,6-dichloro-7H-pyrrolo[2,3-d]pyrimidin-7-yl)phenyl)-5-methylmorpholine-4-carboxylate (319 mg, 0.688 mmol), aqueous sodium hydroxide (4 M, 5.16 mL, 20.7 mmol) and 1,4-dioxane (5.2 mL) after 18 h at 100° C. using 1 M aqueous potassium bisulfate for acidification gave the title compound (360 mg, >100%) as a yellow glass. LCMS (method B): R_{T}=1.32 min, m/z=445, 447 [M+H]⁺.

### Step 2: tert-Butyl (3S,5R)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-5-methylmorpholine-4-carboxylate

General procedure 6 using tert-butyl (3S,5R)-3-(4-(6-chloro-4-oxo-3,4-dihydro-7H-pyrrolo[2,3-d]pyrimidin-7-yl)phenyl)-5-methylmorpholine-4-carboxylate (80 mg, 0.180 mmol), Epoxide 2 (68 mg, 0.270 mmol), Cs₂CO₃ (176 mg, 0.539 mmol) and DMF (3 mL) after 18 h at 80° C. gave the title compound (78 mg, 62%) as a colourless glass. LCMS (method A): R_{T}=1.64 min, m/z=696, 698 [M+H]⁺.

### Step 3: 6-Chloro-3-((1-(4-chlorobenzoyl)-4-hydroxypiperidin-4-yl)methyl)-7-(4-((3S,5R)-5-methylmorpholin-3-yl)phenyl)-3,7-dihydro-4H-pyrrolo[2,3-d]pyrimidin-4-one

General procedure 7 using tert-butyl (3S,5R)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-5-methylmorpholine-4-carboxylate (69 mg, 0.0990 mmol), TFA (2 mL) and DCM (4 mL) gave, after freeze-drying, the title compound (59 mg, 99%) as a white solid. LCMS (method B): R_{T}10=1.08 min, m/z=596, 598 [M+H]⁺. ¹H NMR (500 MHz, DMSO-d₆) δ 8.06 (s, 1H), 7.64-7.60 (m, 2H), 7.53-7.47 (m, 2H), 7.44-7.36 (m, 4H), 6.79 (s, 1H), 4.95 (s, 1H), 4.25-4.08 (m, 1H), 4.02 (s, 2H), 3.96 (dd, J=10.2, 3.1 Hz, 1H), 3.78 (dd, J=10.5, 3.2 Hz, 1H), 3.71 (dd, J=10.3, 2.6 Hz, 1H), 3.44-3.32 (m, 1H), 3.30-3.19 (m, 1H), 3.19-3.06 (m, 2H), 3.05-2.83 (m, 3H), 1.73-1.26 (m, 4H), 0.95 (d, J=6.0 Hz, 3H).

### Example 28: (S)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one

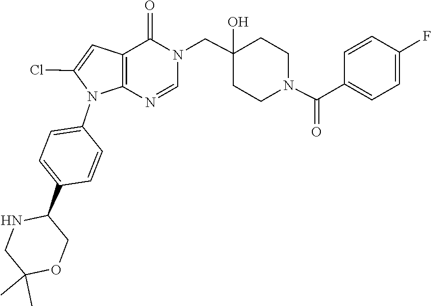 [SMILES: CC1(C)CN[C@@H](c2ccc(-n3c(Cl)cc4c(=O)n(CC5(O)CCN(C(=O)c6ccc(F)cc6)CC5)cnc43)cc2)CO1]

### Step 1: tert-Butyl (S)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate

General procedure 6 using tert-butyl (S)-5-(4-(6-chloro-4-oxo-3,4-dihydro-7H-pyrrolo[2,3-d]pyrimidin-7-yl)phenyl)-2,2-dimethylmorpholine-4-carboxylate (40 mg, 0.0872 mmol), Epoxide 5 (30.8 mg, 0.131 mmol), Cs₂CO₃ (85.2 mg, 0.262 mmol) and DMF (3 mL) after 18 h at 80° C. gave the title compound (45 mg, 74%) as a colourless glass. LCMS (method A): R_{T}=1.61 min, m/z=638, 640 [M−butene+H]⁺.

### Step 2: (S)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one

General procedure 7 using tert-butyl (S)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate (45 mg, 0.0648 mmol), TFA (1 mL) and DCM (2 mL) gave, after freeze-drying, the title compound (38 mg, 98%) as a white solid. LCMS (method A): R_{T}=0.74 min, m/z=594, 596 [M+H]⁺. ¹H NMR (500 MHz, DMSO-d₆) δ 8.06 (s, 1H), 7.67-7.59 (m, 2H), 7.49-7.43 (m, 2H), 7.43-7.37 (m, 2H), 7.30-7.23 (m, 2H), 6.79 (s, 1H), 4.95 (s, 1H), 4.33-4.07 (m, 1H), 4.02 (s, 2H), 3.80 (dd, J=10.3, 3.6 Hz, 1H), 3.57 (dd, J=11.1, 3.6 Hz, 1H), 3.46 (t, J=10.7 Hz, 1H), 3.43-3.33 (m, 1H), 3.30-2.97 (m, 2H), 2.93-2.72 (m, 2H), 2.68 (d, J=11.6 Hz, 1H), 1.74-1.25 (m, 7H), 1.13 (s, 3H).

### Example 29: (R)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one

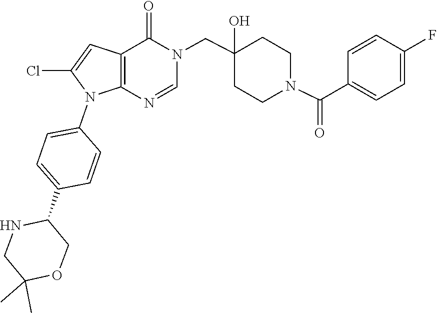 [SMILES: CC1(C)CN[C@H](c2ccc(-n3c(Cl)cc4c(=O)n(CC5(O)CCN(C(=O)c6ccc(F)cc6)CC5)cnc43)cc2)CO1]

### Step 1: tert-Butyl (R)-5-(4-(6-chloro-4-oxo-3,4-dihydro-7H-pyrrolo[2,3-d]pyrimidin-7-yl)phenyl)-2,2-dimethylmorpholine-4-carboxylate

General procedure 5 using tert-butyl (R)-5-(4-(4,6-dichloro-7H-pyrrolo[2,3-d]pyrimidin-7-yl)phenyl)-2,2-dimethylmorpholine-4-carboxylate (321 mg, 0.672 mmol), aqueous sodium hydroxide (4 M, 5.04 mL, 20.2 mmol) and 1,4-dioxane (5.1 mL) after 18 h at 100° C. using 1 M aqueous potassium bisulfate for acidification gave the title compound (321 mg, >100%) as a yellow glass. LCMS (method B): R_{T}=1.40 min, m/z=459, 461 [M+H]⁺.

### Step 2: tert-Butyl (R)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate

General procedure 6 using tert-butyl (R)-5-(4-(6-chloro-4-oxo-3,4-dihydro-7H-pyrrolo[2,3-d]pyrimidin-7-yl)phenyl)-2,2-dimethylmorpholine-4-carboxylate (40 mg, 0.0872 mmol), Epoxide 5 (30.8 mg, 0.131 mmol), Cs₂CO₃ (85.2 mg, 0.262 mmol) and DMF (3 mL) after 18 h at 80° C. gave the title compound (29 mg, 47%) as a colourless glass. LCMS (method B): R_{T}=1.64 min, m/z=638, 640 [M−butene+H]⁺.

### Step 3: (R)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one

General procedure 7 using tert-butyl (R)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate (29 mg, 0.0418 mmol), TFA (1 mL) and DCM (2 mL) gave, after freeze-drying, the title compound (24 mg, 96%) as a white solid. LCMS (method A): R_{T}=0.75 min, m/z=594, 596 [M+H]⁺. ¹H NMR (500 MHz, DMSO-d₆) δ 8.06 (s, 1H), 7.67-7.59 (m, 2H), 7.51-7.42 (m, 2H), 7.42-7.37 (m, 2H), 7.31-7.21 (m, 2H), 6.79 (s, 1H), 4.95 (s, 1H), 4.30-4.08 (m, 1H), 4.02 (s, 2H), 3.80 (dd, J=10.3, 3.6 Hz, 1H), 3.57 (dd, J=11.1, 3.6 Hz, 1H), 3.51-3.43 (m, 1H), 3.43-3.33 (m, 1H), 3.29-3.01 (m, 2H), 2.96-2.72 (m, 2H), 2.68 (d, J=11.6 Hz, 1H), 1.72-1.26 (m, 7H), 1.13 (s, 3H).

### Example 30: 6-Chloro-3-((1-(4-chloro-2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

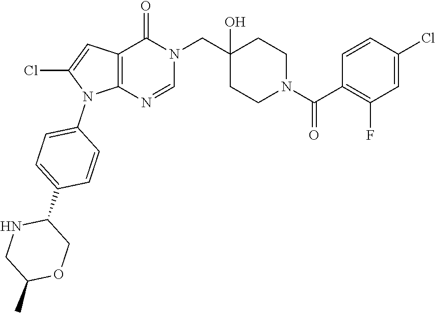 [SMILES: C[C@H]1CN[C@H](c2ccc(-n3c(Cl)cc4c(=O)n(CC5(O)CCN(C(=O)c6ccc(Cl)cc6F)CC5)cnc43)cc2)CO1]

### Step 1: tert-Butyl (2S,5R)-5-(4-(6-chloro-3-((1-(4-chloro-2-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 7 (36.4 mg, 0.135 mmol), Cs₂CO₃ (88 mg, 0.270 mmol) and DMF (2 mL) after 18 h at 80° C. gave the title compound (32 mg, 49%) as a colourless glass. LCMS (method A): R_{T}=1.69 min, m/z=658, 660 [M−butene+H]⁺.

### Step 2: 6-Chloro-3-((1-(4-chloro-2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-chloro-2-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (32 mg, 0.0448 mmol), TFA (1 mL) and DCM (2 mL) gave, after freeze-drying, the title compound (23 mg, 83%) as a white solid. LCMS (method A): R_{T}=0.79 min, m/z=614, 616 [M+H]⁺. ¹H NMR (500 MHz, DMSO-d₆) δ 8.06 (s, 1H), 7.63-7.58 (m, 2H), 7.57-7.53 (m, 1H), 7.50-7.42 (m, 1H), 7.41-7.34 (m, 3H), 6.79 (s, 1H), 4.98 (s, 1H), 4.26-4.15 (m, 1H), 4.11-3.95 (m, 2H), 3.89-3.75 (m, 2H), 3.61-3.49 (m, 1H), 3.30-3.19 (m, 4H), 3.19-3.10 (m, 1H), 3.00-2.91 (m, 1H), 2.58-2.52 (m, 1H), 1.69-1.42 (m, 3H), 1.37-1.29 (m, 1H), 1.09 (d, J=6.2 Hz, 3H).

### Example 31: 6-Chloro-3-((1-(2-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

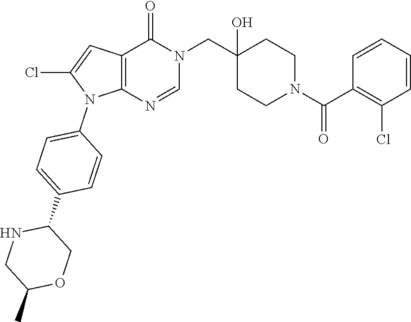 [SMILES: C[C@H]1CN[C@H](c2ccc(-n3c(Cl)cc4c(=O)n(CC5(O)CCN(C(=O)c6ccccc6Cl)CC5)cnc43)cc2)CO1]

### Step 1: tert-Butyl (2S,5R)-5-(4-(6-chloro-3-((1-(2-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 8 (34 mg, 0.135 mmol), Cs₂CO₃ (88 mg, 0.270 mmol) and DMF (2 mL) after 18 h at 80° C. gave the title compound (45 mg, 71%) as a colourless glass. LCMS (method A): R_{T}=1.62 min, m/z=640, 642 [M−butene+H]⁺.

### Step 2: 6-Chloro-3-((1-(2-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(2-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (45 mg, 0.0646 mmol), TFA (1 mL) and DCM (2 mL) gave, after freeze-drying, the title compound (33 mg, 85%) as a white solid. LCMS (method A): R_{T}=0.71 min, m/z=596, 598 [M+H]⁺. ¹H NMR (500 MHz, DMSO-d₆) δ 8.06 (d, J=1.8 Hz, 1H), 7.63-7.56 (m, 2H), 7.56-7.48 (m, 1H), 7.47-7.27 (m, 5H), 6.78 (d, J=2.2 Hz, 1H), 4.97 (d, J=2.3 Hz, 1H), 4.28-4.17 (m, 1H), 4.10-3.92 (m, 2H), 3.87-3.75 (m, 2H), 3.60-3.48 (m, 1H), 3.29-3.03 (m, 4H), 3.01-2.81 (m, 2H), 2.59-2.52 (m, 1H), 1.72-1.43 (m, 3H), 1.37-1.27 (m, 1H), 1.09 (d, J=6.2 Hz, 3H).

### Example 32: 6-Chloro-3-((4-hydroxy-1-((1S,2S)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one or 6-chloro-3-((4-hydroxy-1-((1R,2R)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

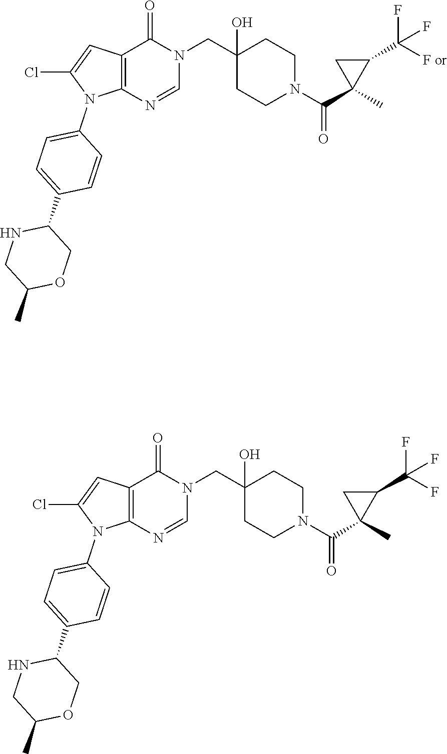

### Step 1: tert-Butyl (2S,5R)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate or tert-butyl (2S,5R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (100 mg, 0.225 mmol), Epoxide 9 (71 mg, 0.270 mmol), Cs₂CO₃ (161 mg, 0.495 mmol) and DMF (2 mL) after 18 h at 80° C. gave, after purification by prep-HPLC, the title compound (72.9 mg, 45%) as a white solid. LCMS (method B): R_{T}=1.48 min, m/z=652, 654 [M−butene+H]⁺.

### Step 2: 6-Chloro-3-((4-hydroxy-1-((1S,2S)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one or 6-chloro-3-((4-hydroxy-1-((1R,2R)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((4-hydroxy-1-((1 S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate or tert-butyl (2S,5R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (72.9 mg, 0.103 mmol), TFA (1 mL) and DCM (2 mL) gave, after purification by flash chromatography and freeze-drying, the title compound (45.6 mg, 72%) as a white solid. LCMS (method A): R_{T}=0.69 min, m/z=608, 610 [M+H]⁺. ¹H NMR (500 MHz, DMSO-d₆) δ 8.06 (s, 1H), 7.63-7.56 (m, 2H), 7.42-7.36 (m, 2H), 6.81 (s, 1H), 4.93 (s, 1H), 4.08-3.77 (m, 6H), 3.60-3.50 (m, 1H), 3.48-3.33 (m, 1H), 3.25 (t, J=10.3 Hz, 1H), 3.12-2.76 (m, 3H), 2.57-2.52 (m, 1H), 2.19-2.03 (m, 1H), 1.74-1.35 (m, 5H), 1.34 (s, 3H), 1.15-1.11 (m, 1H), 1.09 (d, J=6.3 Hz, 3H).

### Example 33: 6-Chloro-3-((4-hydroxy-1-((1R,2R)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one or 6-chloro-3-((4-hydroxy-1-((1S,2S)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

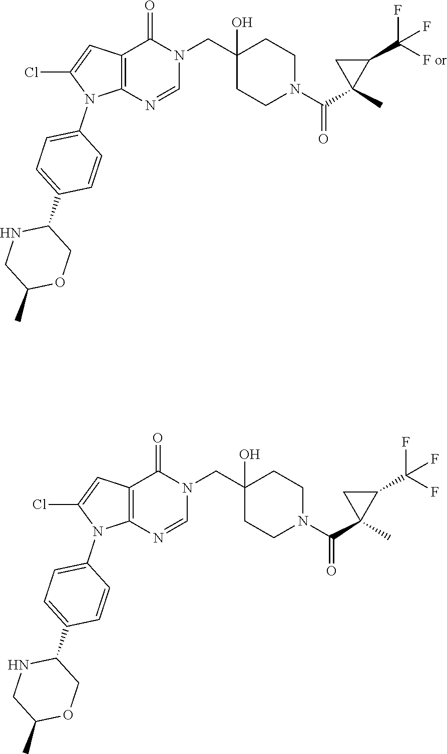

### Step 1: tert-Butyl (2S,5R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate or tert-butyl (2S,5R)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (100 mg, 0.225 mmol), Epoxide 10 (71 mg, 0.270 mmol), Cs₂CO₃ (161 mg, 0.495 mmol) and DMF (2 mL) after 18 h at 80° C. gave, after purification by prep-HPLC, the title compound (111 mg, 69%) as a white solid. LCMS (method B): R_{T}=1.48 min, m/z=652, 654 [M−butene+H]⁺.

### Step 2: 6-Chloro-3-((4-hydroxy-1-((1R,2R)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one or 6-chloro-3-((4-hydroxy-1-((1S,2S)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate or tert-butyl (2S,5R)-5-(4-(6-chloro-3-((4-hydroxy-1-((1 S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (111 mg, 0.157 mmol), TFA (1.5 mL) and DCM (3 mL) gave, after purification by flash chromatography and freeze-drying, the title compound (73 mg, 75%) as a very pale yellow solid. LCMS (method A): R_{T}=0.71 min, m/z=608, 610 [M+H]⁺. ¹H NMR (500 MHz, DMSO-d₆) δ 8.06 (s, 1H), 7.63-7.58 (m, 2H), 7.41-7.36 (m, 2H), 6.81 (s, 1H), 4.93 (s, 1H), 4.06-3.78 (m, 6H), 3.59-3.51 (m, 1H), 3.47-3.33 (m, 1H), 3.25 (t, J=10.3 Hz, 1H), 3.08-2.79 (m, 3H), 2.57-2.52 (m, 1H), 2.18-2.05 (m, 1H), 1.73-1.36 (m, 5H), 1.34 (s, 3H), 1.14-1.10 (m, 1H), 1.09 (d, J=6.3 Hz, 3H).

### Example 34: (R)-6-Chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one

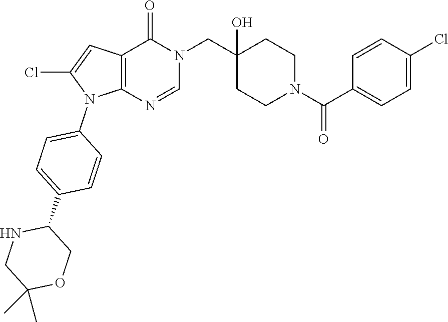 [SMILES: CC1(C)CN[C@H](c2ccc(-n3c(Cl)cc4c(=O)n(CC5(O)CCN(C(=O)c6ccc(Cl)cc6)CC5)cnc43)cc2)CO1]

### Step 1: tert-Butyl (R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate

General procedure 6 using tert-butyl (R)-5-(4-(6-chloro-4-oxo-3,4-dihydro-7H-pyrrolo[2,3-d]pyrimidin-7-yl)phenyl)-2,2-dimethylmorpholine-4-carboxylate (80 mg, 0.174 mmol), Epoxide 2 (52.7 mg, 0.209 mmol), Cs₂CO₃ (125 mg, 0.384 mmol) and DMF (1.5 mL) after 18 h at 80° C. gave, after purification by prep-HPLC, the title compound (87 mg, 70%) as a white solid. LCMS (method B): R_{T}=1.55 min, m/z=654, 656 [M−butene+H]⁺.

### Step 2: (R)-6-Chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one

General procedure 7 using tert-butyl (R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2,2-dimethylmorpholine-4-carboxylate (87 mg, 0.122 mmol), TFA (1.5 mL) and DCM (3 mL) gave, after purification by flash chromatography and freeze-drying, the title compound (45.8 mg, 61%) as an off-white solid. LCMS (method B): R_{T}=0.78 min, m/z=610, 612 [M+H]⁺. ¹H NMR (500 MHz, DMSO-d₆) δ 8.06 (s, 1H), 7.66-7.61 (m, 2H), 7.53-7.47 (m, 2H), 7.45-7.37 (m, 4H), 6.79 (s, 1H), 4.95 (s, 1H), 4.25-4.09 (m, 1H), 4.02 (s, 2H), 3.80 (dd, J=10.3, 3.6 Hz, 1H), 3.57 (dd, J=11.1, 3.6 Hz, 1H), 3.46 (t, J=10.7 Hz, 1H), 3.40-3.33 (m, 1H), 3.30-3.20 (m, 1H), 3.19-3.07 (m, 1H), 2.80 (s, 1H), 2.77 (d, J=11.6 Hz, 1H), 2.68 (d, J=11.6 Hz, 1H), 1.68-1.55 (m, 2H), 1.52-1.41 (m, 1H), 1.40-1.27 (m, 1H), 1.35 (s, 3H), 1.13 (s, 3H).

### Example 35: 6-Chloro-3-((1-(3-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

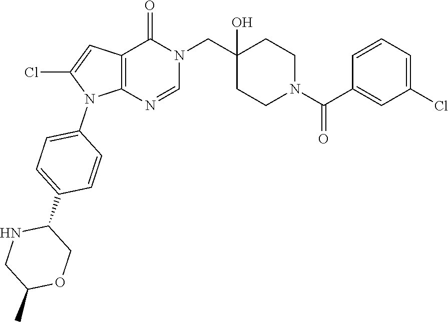 [SMILES: C[C@H]1CN[C@H](c2ccc(-n3c(Cl)cc4c(=O)n(CC5(O)CCN(C(=O)c6cccc(Cl)c6)CC5)cnc43)cc2)CO1]

### Step 1: tert-Butyl (2S,5R)-5-(4-(6-chloro-3-((1-(3-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (50 mg, 0.112 mmol), Epoxide 11 (34 mg, 0.135 mmol), Cs₂CO₃ (80.6 mg, 0.247 mmol) and DMF (1 mL) after 18 h at 80° C. gave the title compound (55 mg, 70%) as a colourless gum. LCMS (method A): R_{T}=1.61 min, m/z=640, 642 [M−butene+H]⁺.

### Step 2: 6-Chloro-3-((1-(3-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(3-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (55 mg, 0.0790 mmol), TFA (0.74 mL) and DCM (1.5 mL) gave, after purification by flash chromatography and freeze-drying, the title compound (35 mg, 73%) as a white solid. LCMS (method B): R_{T}=0.73 min, m/z=596, 598 [M+H]⁺. ¹H NMR (500 MHz, DMSO-d₆) δ 8.06 (s, 1H), 7.63-7.57 (m, 2H), 7.53-7.44 (m, 3H), 7.41-7.37 (m, 2H), 7.37-7.32 (m, 1H), 6.79 (s, 1H), 4.95 (s, 1H), 4.23-4.12 (m, 1H), 4.07-3.96 (m, 2H), 3.87-3.77 (m, 2H), 3.60-3.51 (m, 1H), 3.39-3.20 (m, 3H (signals overlap with HDO)), 3.17-3.07 (m, 1H), 3.00-2.92 (m, 1H), 2.89 (s, 1H), 2.58-2.53 (m, 1H), 1.69-1.56 (m, 2H), 1.51-1.41 (m, 1H), 1.37-1.29 (m, 1H), 1.09 (d, J=6.2 Hz, 3H).

### Example 36: 6-Chloro-3-((1-(2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

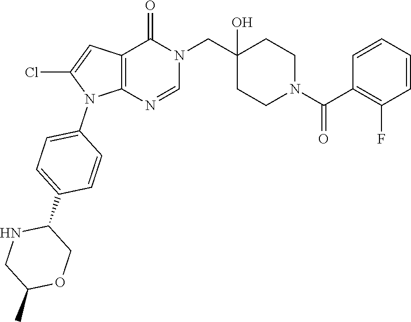 [SMILES: C[C@H]1CN[C@H](c2ccc(-n3c(Cl)cc4c(=O)n(CC5(O)CCN(C(=O)c6ccccc6F)CC5)cnc43)cc2)CO1]

### Step 1: tert-Butyl (2S,5R)-5-(4-(6-chloro-3-((1-(2-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (50 mg, 0.112 mmol), Epoxide 12 (31.7 mg, 0.135 mmol), Cs₂CO₃ (80.6 mg, 0.247 mmol) and DMF (1 mL) after 18 h at 80° C. gave the title compound (42 mg, 54%) as a colourless gum. LCMS (method A): R_{T}=1.52 min, m/z=624, 626 [M−butene+H]⁺.

### Step 2: 6-Chloro-3-((1-(2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(2-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (42 mg, 0.0617 mmol), TFA (0.75 mL) and DCM (1.5 mL) gave, after purification by flash chromatography and freeze-drying, the title compound (30.1 mg, 83%) as a white solid. LCMS (method A): R_{T}=0.66 min, m/z=580, 582 [M+H]⁺. ¹H NMR (500 MHz, DMSO-d₆) δ 8.06 (s, 1H), 7.63-7.57 (m, 2H), 7.52-7.46 (m, 1H), 7.44-7.35 (m, 3H), 7.32-7.25 (m, 2H), 6.79 (s, 1H), 4.98 (s, 1H), 4.22 (dt, J=12.6, 4.0 Hz, 1H), 4.10-3.94 (m, 2H), 3.87-3.77 (m, 2H), 3.55 (dqd, J=12.5, 6.1, 2.2 Hz, 1H), 3.29-3.19 (m, 3H), 3.19-3.11 (m, 1H), 2.99-2.92 (m, 1H), 2.89 (s, 1H), 2.57-2.52 (m, 1H), 1.67-1.45 (m, 3H), 1.38-1.29 (m, 1H), 1.09 (d, J=6.2 Hz, 3H).

### Example 37: 6-Chloro-3-((1-(3,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

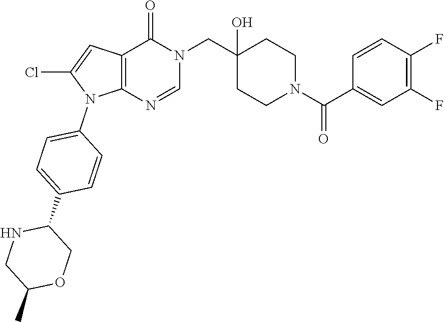 [SMILES: C[C@H]1CN[C@H](c2ccc(-n3c(Cl)cc4c(=O)n(CC5(O)CCN(C(=O)c6ccc(F)c(F)c6)CC5)cnc43)cc2)CO1]

### Step 1: tert-Butyl (2S,5R)-5-(4-(6-chloro-3-((1-(3,4-difluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (50 mg, 0.112 mmol), Epoxide 13 (34 mg, 0.135 mmol), Cs₂CO₃ (80.6 mg, 0.247 mmol) and DMF (1 mL) after 18 h at 80° C. gave the title compound (50 mg, 63%) as a colourless gum. LCMS (method A): R_{T}=1.57 min, m/z=642, 644 [M−butene+H]⁺.

### Step 2: 6-Chloro-3-((1-(3,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(3,4-difluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (50 mg, 0.0716 mmol), TFA (0.75 mL) and DCM (1.5 mL) gave, after purification by flash chromatography and freeze-drying, the title compound (35 mg, 80%) as a white solid. LCMS (method B): R_{T}=0.72 min, m/z=598, 600 [M+H]⁺. ¹H NMR (500 MHz, DMSO-d₆) δ 8.06 (s, 1H), 7.63-7.57 (m, 2H), 7.57-7.46 (m, 2H), 7.42-7.35 (m, 2H), 7.30-7.23 (m, 1H), 6.79 (s, 1H), 4.95 (s, 1H), 4.15 (br. s, 1H), 4.09-3.90 (m, 2H), 3.89-3.77 (m, 2H), 3.55 (dqd, J=12.5, 6.1, 2.2 Hz, 1H), 3.36 (br. s, 1H), 3.25 (t, J=10.3 Hz, 2H), 3.12 (br. s, 1H), 3.00-2.92 (m, 1H), 2.89 (br. s, 1H), 2.58-2.52 (m, 1H), 1.70-1.56 (m, 2H), 1.51-1.27 (m, 2H), 1.09 (d, J=6.2 Hz, 3H).

### Example 38: 6-Chloro-3-((1-(3-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

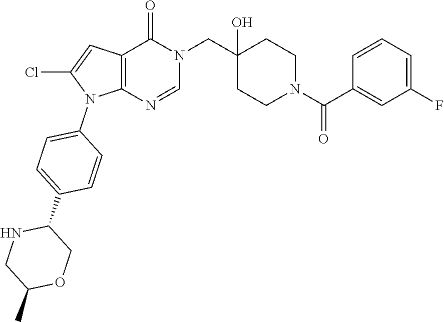 [SMILES: C[C@H]1CN[C@H](c2ccc(-n3c(Cl)cc4c(=O)n(CC5(O)CCN(C(=O)c6cccc(F)c6)CC5)cnc43)cc2)CO1]

### Step 1: tert-Butyl (2S,5R)-5-(4-(6-chloro-3-((1-(3-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 14 (25.4 mg, 0.108 mmol), Cs₂CO₃ (64.4 mg, 0.198 mmol) and DMF (1 mL) after 18 h at 80° C. gave the title compound (29.5 mg, 48%) as colourless gum. LCMS (method A): R_{T}=1.53 min, m/z=624, 626 [M−butene+H]⁺.

### Step 2: 6-Chloro-3-((1-(3-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(3-fluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (29 mg, 0.0426 mmol), TFA (0.75 mL) and DCM (1.5 mL) gave, after freeze-drying, the title compound (18 mg, 72%) as a white solid. LCMS (method A): R_{T}=0.69 min, m/z=580, 582 [M+H]⁺. ¹H NMR (500 MHz, DMSO-d₆) δ 8.06 (s, 1H), 7.64-7.56 (m, 2H), 7.49 (td, J=7.9, 5.8 Hz, 1H), 7.42-7.35 (m, 2H), 7.31-7.19 (m, 3H), 6.79 (s, 1H), 4.95 (s, 1H), 4.17 (s, 1H), 4.10-3.95 (m, 2H), 3.82 (ddd, J=19.6, 10.3, 3.2 Hz, 2H), 3.55 (dqd, J=12.4, 6.1, 2.2 Hz, 1H), 3.25 (t, J=10.3 Hz, 3H (signals overlap with HDO)), 3.12 (t, J=14.9 Hz, 1H), 2.99-2.79 (m, 2H), 2.54 (d, J=11.1 Hz, 1H), 1.61 (s, 2H), 1.47 (s, 1H), 1.33 (s, 1H), 1.09 (d, J=6.2 Hz, 3H).

### Example 39: 6-Chloro-3-((1-(2,3-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

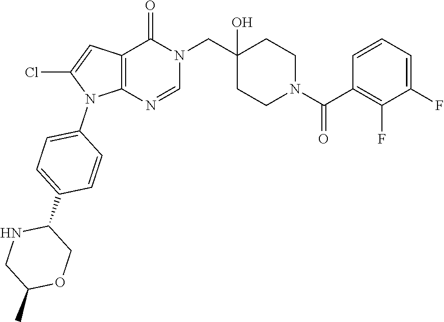 [SMILES: C[C@H]1CN[C@H](c2ccc(-n3c(Cl)cc4c(=O)n(CC5(O)CCN(C(=O)c6cccc(F)c6F)CC5)cnc43)cc2)CO1]

### Step 1: tert-Butyl (2S,5R)-5-(4-(6-chloro-3-((1-(2,3-difluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (50 mg, 0.112 mmol), Epoxide 15 (34.2 mg, 0.135 mmol), Cs₂CO₃ (80.6 mg, 0.247 mmol) and DMF (1 mL) after 18 h at 80° C. gave the title compound (30 mg, 38%) as a white solid. LCMS (method B): R_{T}=1.56 min, m/z=642, 644 [M−butene+H]⁺.

### Step 2: 6-Chloro-3-((1-(2,3-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(2,3-difluorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (30 mg, 0.0430 mmol), TFA (0.75 mL) and DCM (1.5 mL) gave, after purification by flash chromatography and freeze-drying, the title compound (22.3 mg, 86%) as a white solid. LCMS (method B): R_{T}=0.79 min, m/z=598, 600 [M+H]⁺. ¹H NMR (500 MHz, DMSO-d₆) δ 8.06 (s, 1H), 7.60 (d, J=8.5 Hz, 2H), 7.56-7.46 (m, 1H), 7.39 (d, J=8.5 Hz, 2H), 7.34-7.26 (m, 1H), 7.26-7.15 (m, 1H), 6.79 (s, 1H), 4.99 (s, 1H), 4.22 (dt, J=13.3, 4.2 Hz, 1H), 4.10-3.94 (m, 2H), 3.89-3.75 (m, 2H), 3.55 (dqd, J=12.5, 6.1, 2.2 Hz, 1H), 3.28-3.21 (m, 3H), 3.21-3.12 (m, 1H), 2.98-2.92 (m, 1H), 2.89 (s, 1H), 2.58-2.53 (m, 1H), 1.67-1.45 (m, 3H), 1.40-1.30 (m, 1H), 1.09 (d, J=6.2 Hz, 3H).

### Example 40: (S)-3-((1-([1,1′-bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one

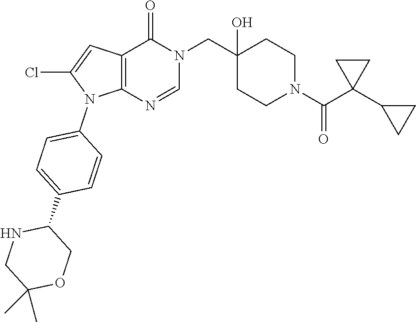 [SMILES: CC1(C)CN[C@H](c2ccc(-n3c(Cl)cc4c(=O)n(CC5(O)CCN(C(=O)C6(C7CC7)CC6)CC5)cnc43)cc2)CO1]

### Step 1: tert-Butyl (S)-5-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-2,2-dimethylmorpholine-4-carboxylate

General procedure 6 using tert-butyl (S)-5-(4-(6-chloro-4-oxo-3,4-dihydro-7H-pyrrolo[2,3-d]pyrimidin-7-yl)phenyl)-2,2-dimethylmorpholine-4-carboxylate (45 mg, 0.0981 mmol), Epoxide 16 (32.6 mg, 0.147 mmol), Cs₂CO₃ (95.8 mg, 0.294 mmol) and DMF (2 mL) after 18 h at 80° C. gave the title compound (55 mg, 82%) as a colourless glass. LCMS (method B): R_{T}=1.47 min, m/z=624, 626 [M−butene+H]⁺.

### Step 2: (S)-3-((1-([1,1′-Bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one

General procedure 7 using tert-butyl (S)-5-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-2,2-dimethylmorpholine-4-carboxylate (55 mg, 0.0809 mmol), TFA (1 mL) and DCM (2 mL) gave, after freeze-drying, the title compound (40 mg, 85%) as a white solid. LCMS (method B): R_{T}=0.78 min, m/z=580, 582 [M+H]⁺. ¹H NMR (500 MHz, DMSO-d₆) δ 8.07 (s, 1H), 7.67-7.60 (m, 2H), 7.44-7.35 (m, 2H), 6.81 (s, 1H), 4.92 (s, 1H), 4.19-3.89 (m, 4H), 3.81 (dd, J=10.4, 3.5 Hz, 1H), 3.57 (dd, J=11.1, 3.6 Hz, 1H), 3.46 (t, J=10.7 Hz, 1H), 3.38-2.89 (m, 2H), 2.88-2.65 (m, 3H), 1.69-1.38 (m, 4H), 1.36 (s, 3H), 1.17-1.09 (m, 4H), 0.73-0.64 (m, 2H), 0.52-0.42 (m, 2H), 0.42-0.33 (m, 2H), 0.14-0.05 (m, 2H).

### Example 41: (R)-3-((1-([1,1′-bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one

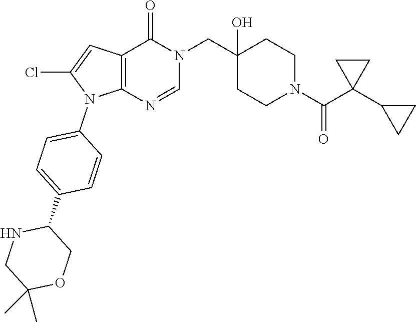 [SMILES: CC1(C)CN[C@H](c2ccc(-n3c(Cl)cc4c(=O)n(CC5(O)CCN(C(=O)C6(C7CC7)CC6)CC5)cnc43)cc2)CO1]

### Step 1: tert-Butyl (R)-5-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-2,2-dimethylmorpholine-4-carboxylate

General procedure 6 using tert-butyl (R)-5-(4-(6-chloro-4-oxo-3,4-dihydro-7H-pyrrolo[2,3-d]pyrimidin-7-yl)phenyl)-2,2-dimethylmorpholine-4-carboxylate (45 mg, 0.0981 mmol), Epoxide 16 (32.6 mg, 0.147 mmol), Cs₂CO₃ (95.8 mg, 0.294 mmol) and DMF (2 mL) after 18 h at 80° C. gave the title compound (46 mg, 69%) as a colourless solid. LCMS (method B): R_{T}=1.47 min, m/z=624, 626 [M−butene+H]⁺.

### Step 2: (R)-3-((1-([1,1′-Bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one

General procedure 7 using tert-butyl (R)-5-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-2,2-dimethylmorpholine-4-carboxylate (46 mg, 0.0676 mmol), TFA (1 mL) and DCM (2 mL) gave, after freeze-drying, the title compound (37 mg, 94%) as a white solid. LCMS (method B): R_{T}=0.77 min, m/z=580, 582 [M+H]⁺. ¹H NMR (500 MHz, DMSO-d₆) δ 8.07 (s, 1H), 7.68-7.60 (m, 2H), 7.44-7.37 (m, 2H), 6.81 (s, 1H), 4.92 (s, 1H), 4.08-3.93 (m, 4H), 3.81 (dd, J=10.3, 3.5 Hz, 1H), 3.57 (dd, J=11.1, 3.6 Hz, 1H), 3.46 (t, J=10.7 Hz, 1H), 3.37-2.91 (m, 2H), 2.89-2.65 (m, 3H), 1.66-1.38 (m, 4H), 1.36 (s, 3H), 1.20-1.03 (m, 4H), 0.72-0.62 (m, 2H), 0.50-0.43 (m, 2H), 0.42-0.32 (m, 2H), 0.15-0.04 (m, 2H).

### Example 42: 3-((1-([1,1′-Bi(cyclopropane)]-1-carbonyl)-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one

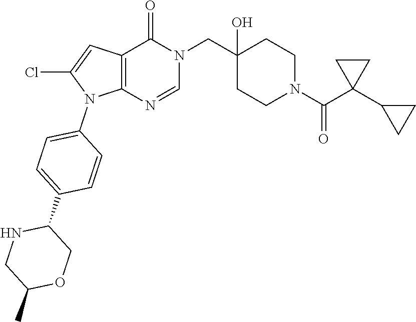 [SMILES: C[C@H]1CN[C@H](c2ccc(-n3c(Cl)cc4c(=O)n(CC5(O)CCN(C(=O)C6(C7CC7)CC6)CC5)cnc43)cc2)CO1]

### Step 1: tert-Butyl (2S,5R)-5-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (45 mg, 0.101 mmol), Epoxide 16 (34 mg, 0.152 mmol), Cs₂CO₃ (98.9 mg, 0.303 mmol) and DMF (2 mL) after 18 h at 80° C. gave the title compound (47 mg, 69%) as a colourless glass. LCMS (method B): R_{T}=1.41 min, m/z=610, 612 [M−butene+H]⁺.

### Step 2: 3-((1-([1,1′-Bi(cyclopropane)]-1-carbonyl)-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate (47 mg, 0.0705 mmol), TFA (1 mL) and DCM (2 mL) gave, after freeze-drying, the title compound (36 mg, 90%) as a white solid. LCMS (method B): R_{T}=0.76 min, m/z=566, 568 [M+H]⁺. ¹H NMR (500 MHz, DMSO-d₆) δ 8.07 (s, 1H), 7.64-7.57 (m, 2H), 7.43-7.36 (m, 2H), 6.80 (s, 1H), 4.91 (s, 1H), 4.12-3.91 (m, 4H), 3.90-3.77 (m, 2H), 3.61-3.51 (m, 1H), 3.46-2.75 (m, 5H (signals overlap HDO)), 2.59-2.53 (m, 1H), 1.62-1.46 (m, 2H), 1.46-1.35 (m, 2H), 1.17-1.11 (m, 1H), 1.09 (d, J=6.2 Hz, 3H), 0.71-0.62 (m, 2H), 0.50-0.41 (m, 2H), 0.41-0.34 (m, 2H), 0.13-0.04 (m, 2H).

### Example 43: 7-(4-(5-Oxa-8-azaspiro[3.5]nonan-7-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one

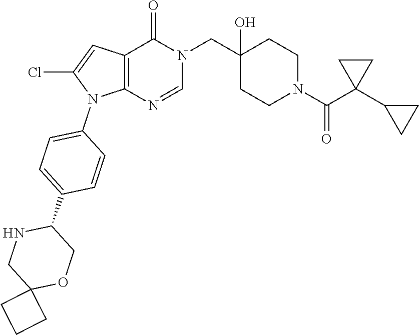 [SMILES: O=C(N1CCC(O)(Cn2cnc3c(cc(Cl)n3-c3ccc([C@@H]4COC5(CCC5)CN4)cc3)c2=O)CC1)C1(C2CC2)CC1]

### Step 1: 8-(tert-Butyl) 7-(4,5,6,7-tetrachloro-1,3-dioxoisoindolin-2-yl) 5-oxa-8-azaspiro[3.5]nonane-7,8-dicarboxylate

General procedure 11 using 8-(tert-butoxycarbonyl)-5-oxa-8-azaspiro[3.5]nonane-7-carboxylic acid (2.60 g, 9.59 mmol), 4,5,6,7-tetrachloro-2-hydroxyisoindoline-1,3-dione (2.89 g, 9.59 mmol), EDC (2.21 g, 11.5 mmol) and DMAP (117 mg, 0.959 mmol) gave the title compound (2.34 g, 46%). ¹H NMR (500 MHz, Chloroform-d) δ 5.11-4.62 (m, 1H), 4.34-4.18 (m, 1H), 4.05-3.81 (m, 2H), 3.24-3.02 (m, 1H), 2.23-2.05 (m, 2H), 2.04-1.90 (m, 2H), 1.89-1.79 (m, 1H), 1.70-1.61 (m, 1H), 1.58-1.40 (m, 9H).

### Step 2: tert-Butyl 7-(4-bromophenyl)-5-oxa-8-azaspiro[3.5]nonane-8-carboxylate

To 8-(tert-butyl) 7-(4,5,6,7-tetrachloro-1,3-dioxoisoindolin-2-yl) 5-oxa-8-azaspiro[3.5]nonane-7,8-dicarboxylate (710 mg, 1.28 mmol), (4-bromophenyl)boronic acid (772 mg, 3.84 mmol) in a 100 mL RBF under nitrogen was added 1,4-dioxane (48.5 mL). The reaction mixture was heated to 75-80° C. (internal temperature) before triethylamine (1.79 mL, 12.8 mmol) and NiCl₂·6H₂O: bathophenanthroline complex in DMF (0.05 M, 4.85 mL, 0.243 mmol, (Angew. Chem. Int. Ed. 2016, 55, 9676-9679)) were sequentially added. The resulting mixture was stirred at 75° C. overnight. Upon cooling to rt the reaction mixture was diluted with EtOAc (250 mL) and washed with brine. The organic layer was dried over MgSO₄, filtered and concentrated under reduced pressure. The residue was purified by flash chromatography to give the title compound (366 mg, 74%) as a colourless glass. LCMS (method B): R_{T}=1.75 min, m/z=282, 284 [M−Boc+H]⁺. This material was used without further purification.

### Step 3: tert-Butyl 7-(4-(4,4,5,5-tetramethyl-1,3,2-dioxaborolan-2-yl)phenyl)-5-oxa-8-azaspiro[3.5]nonane-8-carboxylate

General procedure 3 (Work-up A) using tert-butyl 7-(4-bromophenyl)-5-oxa-8-azaspiro[3.5]nonane-8-carboxylate (415 mg, 1.09 mmol), B₂Pin₂ (414 mg, 1.63 mmol), Pd(dppf)Cl₂·CH₂Cl₂ (91.9 mg, 0.109 mmol), KOAc (320 mg, 3.26 mmol) and 1,4-dioxane (11 mL) after stirring at 100° C. (thermal) for 4 h gave the title compound (460 mg, 98%) as a yellow solid. LCMS (method B): R_{T}=1.84 min, m/z=330 [M−Boc+H]⁺. This material was used without further purification.

### Step 4: tert-Butyl 7-(4-(4,6-dichloro-7H-pyrrolo[2,3-d]pyrimidin-7-yl)phenyl)-5-oxa-8-azaspiro[3.5]nonane-8-carboxylate

General procedure 4 (Work-up A) using 4,6-dichloro-7H-pyrrolo[2,3-d]pyrimidine (200 mg, 1.06 mmol), tert-butyl 7-(4-(4,4,5,5-tetramethyl-1,3,2-dioxaborolan-2-yl)phenyl)-5-oxa-8-azaspiro[3.5]nonane-8-carboxylate (457 mg, 1.06 mmol), copper(II) trifluoroacetate hydrate (616 mg, 2.13 mmol), 1,10-phenanthroline (383 mg, 2.13 mmol), boric acid (263 mg, 4.26 mmol) and DMF (20 mL) after 3 days at 50° C. gave the title compound (127 mg, 24%) as a colourless glass. LCMS (method B): R_{T}=1.80 min, m/z=489, 491 [M+H]⁺.

### Step 5: tert-Butyl 7-(4-(6-chloro-4-oxo-3,4-dihydro-7H-pyrrolo[2,3-d]pyrimidin-7-yl)phenyl)-5-oxa-8-azaspiro[3.5]nonane-8-carboxylate

General procedure 5 using tert-butyl 7-(4-(4,6-dichloro-7H-pyrrolo[2,3-d]pyrimidin-7-yl)phenyl)-5-oxa-8-azaspiro[3.5]nonane-8-carboxylate (20 mg, 0.0409 mmol), aqueous sodium hydroxide (4 M, 0.31 mL, 1.23 mmol) and 1,4-dioxane (0.3 mL) after 18 h at 100° C. using 1 M aqueous potassium bisulfate for acidification gave the title compound (19 mg, quant.) as a yellow glass. LCMS (method B): R_{T}=1.38 min, m/z=471, 473 [M+H]⁺.

### Step 6: tert-Butyl 7-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-5-oxa-8-azaspiro[3.5]nonane-8-carboxylate

General procedure 6 using tert-butyl 7-(4-(6-chloro-4-oxo-3,4-dihydro-7H-pyrrolo[2,3-d]pyrimidin-7-yl)phenyl)-5-oxa-8-azaspiro[3.5]nonane-8-carboxylate (10 mg, 0.0212 mmol), Epoxide 16 (7.1 mg, 0.0319 mmol), Cs₂CO₃ (20.8 mg, 0.0637 mmol) and DMF (0.5 mL) after 18 h at 80° C. gave the title compound (7.5 mg, 51%) as a colourless glass. LCMS (method B): R_{T}=1.54 min, m/z=636, 638 [M−butene+H]⁺.

### Step 7: 7-(4-(5-oxa-8-azaspiro[3.5]nonan-7-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one

General procedure 7 using tert-butyl 7-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-5-oxa-8-azaspiro[3.5]nonane-8-carboxylate (7.5 mg, 0.0108 mmol), TFA (1 mL) and DCM (2 mL) gave, after freeze-drying, the title compound (6 mg, 93%) as a white solid. LCMS (method B): R_{T}=0.81 min, m/z=592, 594 [M+H]⁺. ¹H NMR (500 MHz, DMSO-d₆) δ 8.07 (s, 1H), 7.67-7.56 (m, 2H), 7.45-7.33 (m, 2H), 6.80 (s, 1H), 4.91 (s, 1H), 4.12-3.93 (m, 4H), 3.84 (dd, J=10.1, 3.2 Hz, 1H), 3.60 (dd, J=10.9, 3.3 Hz, 1H), 3.47-2.75 (m, 5H), 2.70 (dd, J=11.7, 1.9 Hz, 1H), 2.35-2.27 (m, 1H), 2.08-1.69 (m, 4H), 1.70-1.32 (m, 5H), 1.18-1.08 (m, 1H), 0.67 (s, 2H), 0.52-0.41 (m, 2H), 0.41-0.32 (m, 2H), 0.12-0.07 (m, 1H).

### Example 44: 7-(4-(5-Oxa-8-azaspiro[3.5]nonan-7-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

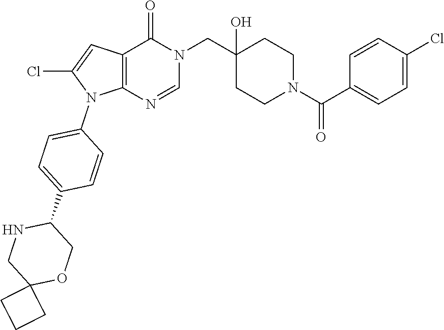 [SMILES: O=C(c1ccc(Cl)cc1)N1CCC(O)(Cn2cnc3c(cc(Cl)n3-c3ccc([C@@H]4COC5(CCC5)CN4)cc3)c2=O)CC1]

### Step 1: tert-Butyl 7-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-5-oxa-8-azaspiro[3.5]nonane-8-carboxylate

General procedure 6 using tert-butyl 7-(4-(6-chloro-4-oxo-3,4-dihydro-7H-pyrrolo[2,3-d]pyrimidin-7-yl)phenyl)-5-oxa-8-azaspiro[3.5]nonane-8-carboxylate (10 mg, 0.0212 mmol), Epoxide 2 (8.02 mg, 0.0319 mmol), Cs₂CO₃ (20.8 mg, 0.0637 mmol) and DMF (0.5 mL) after 18 h at 80° C. gave the title compound (6 mg, 39%) as a colourless glass. LCMS (method B): R_{T}=1.61 min, m/z=666, 668 [M−butene+H]⁺.

### Step 2: 7-(4-(5-Oxa-8-azaspiro[3.5]nonan-7-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

General procedure 7 using tert-butyl 7-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-5-oxa-8-azaspiro[3.5]nonane-8-carboxylate (6 mg, 0.0083 mmol), TFA (1 mL) and DCM (2 mL) gave, after freeze-drying, the title compound (5 mg, 96%) as a white solid. LCMS (method B): R_{T}=0.88 min, m/z=622, 624 [M+H]⁺. ¹H NMR (500 MHz, DMSO-d₆) δ 8.06 (s, 1H), 7.63-7.57 (m, 2H), 7.53-7.47 (m, 2H), 7.46-7.36 (m, 4H), 6.79 (s, 1H), 4.95 (s, 1H), 4.27-4.07 (m, 1H), 4.02 (s, 2H), 3.84 (dd, J=10.2, 3.2 Hz, 1H), 3.59 (dd, J=10.9, 3.3 Hz, 1H), 3.42-3.33 (m, 1H), 3.29-2.96 (m, 4H), 2.96-2.76 (m, 1H), 2.70 (dd, J=11.7, 1.9 Hz, 1H), 2.35-2.22 (m, 1H), 2.01-1.70 (m, 4H), 1.69-1.27 (m, 5H).

### Example 45: 6-Chloro-7-(4-((3R,6S)-6-ethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one

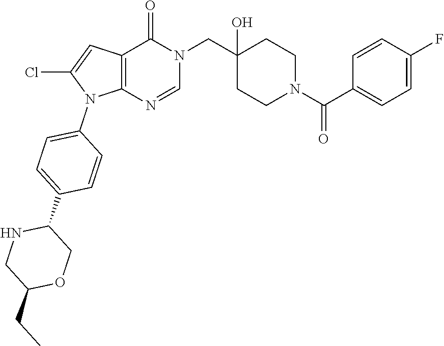 [SMILES: CC[C@H]1CN[C@H](c2ccc(-n3c(Cl)cc4c(=O)n(CC5(O)CCN(C(=O)c6ccc(F)cc6)CC5)cnc43)cc2)CO1]

### Step 1: (2S,5R)-5-(4-Bromophenyl)-2-ethylmorpholine

General procedure 8 using 4-bromobenzaldehyde (2.36 g, 12.8 mmol), (S)-2-((tributylstannyl)methoxy)butan-1-amine (made according to the procedure outlined in Org. Lett. 2014, 16 (4), 1236-1239 starting from (S)-1-aminobutan-2-ol) (5.00 g, 12.8 mmol), Cu(OTf)₂ (4.63 g, 12.8 mmol), 2,6-dimethylpyridine (1.37 g, 12.8 mmol), HFIP (51 mL), 4 Å molecular sieves (ca. 1.28 g) and DCM (205 mL) gave the title compound (1.93 g, 56%). LCMS (method C): R_{T}=0.93 min, m/z=270, 272 [M+H]⁺. ¹H NMR (400 MHz, Chloroform-d) δ 0.97 (t, J=7.5 Hz, 3H), 1.40-1.66 (m, 2H), 2.72 (dd, J=11.5, 10.2 Hz, 1H), 3.08 (dd, J=11.5, 2.3 Hz, 1H), 3.33-3.50 (m, 2H), 3.76-3.93 (m, 2H), 6.96 (d, J=7.7 Hz, 2H), 7.46 (d, J=7.7 Hz, 2H).

### Step 2: tert-Butyl (2S,5R)-5-(4-bromophenyl)-2-ethylmorpholine-4-carboxylate

General procedure 9 using (2S,5R)-5-(4-bromophenyl)-2-ethylmorpholine (1.93 g, 7.15 mmol) and Boc₂O (1.87 g, 8.58 mmol) gave the title compound (1.77 g, 67%). LCMS (method C): R_{T}=1.40 min, m/z=270, 272 [M−Boc+H]⁺. ¹H NMR (400 MHz, Chloroform-d) δ 0.95 (t, J=7.4 Hz, 3H), 1.35 (s, 9H), 1.48-1.61 (m, 1H), 1.65-1.79 (m, 1H), 3.38-3.52 (m, 2H), 3.58-3.67 (m, 1H), 3.73 (dd, J=12.3, 5.4 Hz, 1H), 3.96 (dd, J=12.2, 4.5 Hz, 1H), 4.79 (t, J=5.1 Hz, 1H), 7.24 (d, J=8.1 Hz, 2H), 7.43 (d, J=8.1 Hz, 2H).

### Step 3: tert-Butyl (2S,5R)-2-ethyl-5-(4-(4,4,5,5-tetramethyl-1,3,2-dioxaborolan-2-yl)phenyl)morpholine-4-carboxylate

General procedure 3 (Work-up B) using tert-butyl (2S,5R)-5-(4-bromophenyl)-2-ethylmorpholine-4-carboxylate (741 mg, 2.00 mmol), B₂Pin₂ (762 mg, 3.00 mmol), Pd(dppf)Cl₂·CH₂Cl₂ (169 mg, 0.200 mmol), KOAc (589 mg, 6.00 mmol) and 1,4-dioxane (20 mL) after stirring at 100° C. (thermal) for 70 min gave the title compound (914 mg, >100%) as a yellow oil. LCMS (method A): R_{T}=2.07 min, m/z=362 [M−butene+H]⁺. ¹H NMR (500 MHz, DMSO-d₆) δ 7.65 (d, J=8.1 Hz, 2H), 7.32 (d, J=7.8 Hz, 2H), 4.80 (t, J=5.4 Hz, 1H), 3.91 (dd, J=12.2, 4.9 Hz, 1H), 3.69 (dd, J=12.2, 6.0 Hz, 1H), 3.66-3.60 (m, 1H), 3.49 (dd, J=13.7, 3.8 Hz, 1H), 3.41 (dd, J=13.7, 4.4 Hz, 1H), 1.65-1.55 (m, 1H), 1.53-1.44 (m, 1H), 1.29 (s, 9H), 1.28 (s, 6H), 1.17 (s, 6H), 0.88 (t, J=7.4 Hz, 3H). This material was used without further purification.

### Step 4: tert-Butyl (2S,5R)-5-(4-(4,6-dichloro-7H-pyrrolo[2,3-d]pyrimidin-7-yl)phenyl)-2-ethylmorpholine-4-carboxylate

General procedure 4 (Work-up B) using 4,6-dichloro-7H-pyrrolo[2,3-d]pyrimidine (289 mg, 1.54 mmol), tert-butyl (2S,5R)-2-ethyl-5-(4-(4,4,5,5-tetramethyl-1,3,2-dioxaborolan-2-yl)phenyl)morpholine-4-carboxylate (834 mg, 2.00 mmol), copper(II) trifluoroacetate hydrate (890 mg, 3.07 mmol), 1,10-phenanthroline (554 mg, 3.07 mmol), boric acid (247 mg, 4.00 mmol) and DMF (15.4 mL) after 3 days at 50° C. gave the title compound (469 mg, 63%) as a colourless glass. LCMS (method A): R_{T}=2.02 min, m/z=477, 479 [M+H]⁺. ¹H NMR (500 MHz, DMSO-d₆) δ 8.62 (s, 1H), 7.58-7.54 (m, 2H), 7.54-7.50 (m, 2H), 7.10 (s, 1H), 4.92 (t, J=5.4 Hz, 1H), 4.01 (dd, J=12.2, 4.8 Hz, 1H), 3.79 (dd, J=12.2, 6.0 Hz, 1H), 3.70 (tt, J=7.3, 4.1 Hz, 1H), 3.51 (d, J=4.2 Hz, 2H), 1.69-1.58 (m, 1H), 1.58-1.47 (m, 1H), 1.32 (s, 9H), 0.91 (t, J=7.4 Hz, 3H).

### Step 5: tert-Butyl (2S,5R)-5-(4-(6-chloro-4-oxo-3,4-dihydro-7H-pyrrolo[2,3-d]pyrimidin-7-yl)phenyl)-2-ethylmorpholine-4-carboxylate

General procedure 5 using tert-butyl (2S,5R)-5-(4-(4,6-dichloro-7H-pyrrolo[2,3-d]pyrimidin-7-yl)phenyl)-2-ethylmorpholine-4-carboxylate (467 mg, 0.978 mmol), aqueous sodium hydroxide (4 M, 2.45 mL, 9.78 mmol) and 1,4-dioxane (4.9 mL) after 26 h 20 min at 100° C. using 2 M aqueous hydrochloric acid for acidification gave the title compound (525 mg, >100%) as a brown solid. LCMS (method A): R_{T}=1.43 min, m/z=459, 461 [M+H]⁺. ¹H NMR (500 MHz, DMSO-d₆) δ 12.21 (s, 1H), 7.82 (s, 1H), 7.52-7.47 (m, 2H), 7.44-7.37 (m, 2H), 6.65 (s, 1H), 4.90 (t, J=5.4 Hz, 1H), 3.99 (dd, J=12.2, 4.9 Hz, 1H), 3.79 (dd, J=12.2, 5.9 Hz, 1H), 3.69 (tt, J=7.3, 4.2 Hz, 1H), 3.54-3.45 (m, 2H), 1.68-1.60 (m, 1H), 1.56-1.46 (m, 1H), 1.31 (s, 9H), 0.91 (t, J=7.4 Hz, 3H).

### Step 6: tert-Butyl (2S,5R)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-ethylmorpholine-4-carboxylate (60 mg, 0.131 mmol), Epoxide 5 (36.9 mg, 0.157 mmol), Cs₂CO₃ (93.7 mg, 0.288 mmol) and DMF (1 mL) after 18 h at 80° C. gave the title compound (43 mg, 47%) as a colourless gum. LCMS (method B): R_{T}=1.50 min, m/z=638, 640 [M−butene+H]⁺.

### Step 7: 6-Chloro-7-(4-((3R,6S)-6-ethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one

General procedure 7 using tert-butyl (2S,5R)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate (43 mg, 0.0619 mmol), TFA (0.75 mL) and DCM (1.5 mL) gave, after purification by flash chromatography and freeze-drying, the title compound (26.9 mg, 73%) as a white solid. LCMS (method B): R_{T}=0.82 min, m/z=594, 596 [M+H]⁺. ¹H NMR (500 MHz, DMSO-d₆) δ 8.06 (s, 1H), 7.60 (d, J=8.5 Hz, 2H), 7.53-7.42 (m, 2H), 7.39 (d, J=8.4 Hz, 2H), 7.31-7.22 (m, 2H), 6.79 (s, 1H), 4.95 (s, 1H), 4.16 (s, 1H), 4.07-3.95 (m, 2H), 3.92-3.78 (m, 2H), 3.56-2.76 (m, 7H (signals overlap with HDO)), 2.59-2.53 (m, 1H), 1.70-1.53 (m, 2H), 1.53-1.25 (m, 4H), 0.91 (t, J=7.5 Hz, 3H).

### Example 46: 6-Chloro-3-((1-(4-chlorobenzoyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one

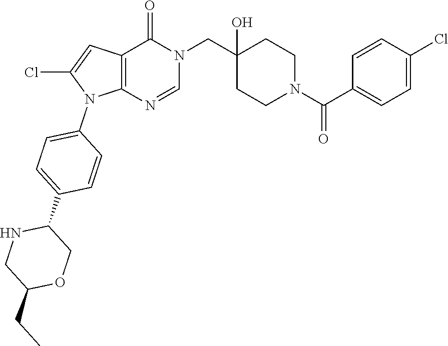 [SMILES: CC[C@H]1CN[C@H](c2ccc(-n3c(Cl)cc4c(=O)n(CC5(O)CCN(C(=O)c6ccc(Cl)cc6)CC5)cnc43)cc2)CO1]

### Step 1: tert-Butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-ethylmorpholine-4-carboxylate (60 mg, 0.131 mmol), Epoxide 2 (39.5 mg, 0.157 mmol), Cs₂CO₃ (93.7 mg, 0.288 mmol) and DMF (1 mL) after 18 h at 80° C. gave the title compound (43 mg, 46%) as a colourless gum. LCMS (method B): R_{T}=1.57 min, m/z=654, 656 [M−butene+H]⁺.

### Step 2: 6-Chloro-3-((1-(4-chlorobenzoyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate (32.0 mg, 0.0450 mmol), TFA (0.17 mL) and DCM (1.5 mL) gave, after purification by flash chromatography and freeze-drying, the title compound (18.5 mg, 67%) as a white solid. LCMS (method B): R_{T}=0.87 min, m/z=610, 612 [M+H]⁺. ¹H NMR (500 MHz, DMSO-d₆) δ 8.06 (s, 1H), 7.63-7.57 (m, 2H), 7.53-7.47 (m, 2H), 7.46-7.34 (m, 4H), 6.79 (s, 1H), 4.95 (s, 1H), 4.24-4.11 (m, 1H), 4.02 (s, 2H), 3.88-3.79 (m, 2H), 3.43-3.20 (m, 4H (signals overlap with HDO)), 3.18-3.08 (m, 1H), 2.97 (dd, J=11.5, 2.3 Hz, 1H), 2.89 (s, 1H), 2.58-2.53 (m, 1H), 1.66-1.53 (m, 2H), 1.51-1.38 (m, 3H), 1.38-1.27 (m, 1H), 0.91 (t, J=7.5 Hz, 3H).

### Example 47: 6-Chloro-3-((1-(4-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

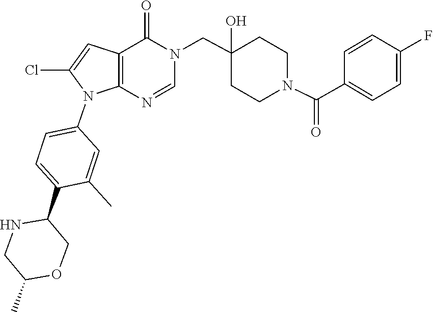 [SMILES: Cc1cc(-n2c(Cl)cc3c(=O)n(CC4(O)CCN(C(=O)c5ccc(F)cc5)CC4)cnc32)ccc1[C@H]1CO[C@H](C)CN1]

### Step 1: tert-Butyl (2R,5S)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (60 mg, 0.131 mmol), Epoxide 5 (36.9 mg, 0.157 mmol), Cs₂CO₃ (93.7 mg, 0.288 mmol) and DMF (1 mL) after 18 h at 80° C. gave the title compound (57 mg, 62%) as a light brown gum. LCMS (method B): R_{T}=1.46 min, m/z=638, 640 [M−butene+H]⁺.

### Step 2: 6-Chloro-3-((1-(4-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-fluorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (57 mg, 0.0821 mmol), TFA (0.75 mL) and DCM (1.5 mL) gave, after purification by flash chromatography and freeze-drying, the title compound (34.3 mg, 70%) as a white solid. LCMS (method B): R_{T}=0.81 min, m/z=594, 596 [M+H]⁺. ¹H NMR (500 MHz, DMSO-d₆) δ 8.06 (s, 1H), 7.73 (d, J=8.0 Hz, 1H), 7.50-7.42 (m, 2H), 7.35-7.17 (m, 4H), 6.78 (s, 1H), 4.95 (s, 1H), 4.16 (br. s, 1H), 4.06-3.93 (m, 3H), 3.80 (dd, J=10.9, 3.0 Hz, 1H), 3.63-3.53 (m, 1H), 3.39 (br. s, 1H), 3.34-3.06 (m, 3H (signals overlap with HDO)), 3.03-2.94 (m, 1H), 2.83 (br. s, 1H), 2.61-2.54 (m, 1H), 2.41 (s, 3H), 1.69-1.53 (m, 2H), 1.52-1.40 (m, 1H), 1.39-1.27 (m, 1H), 1.10 (d, J=6.2 Hz, 3H).

### Example 48: 7-(4-((1R,2S,5S)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

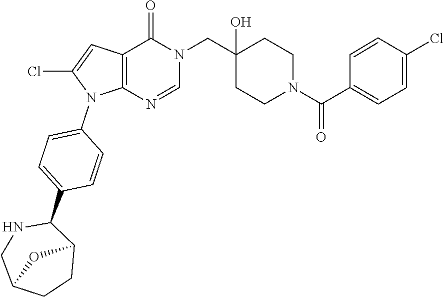 [SMILES: O=C(c1ccc(Cl)cc1)N1CCC(O)(Cn2cnc3c(cc(Cl)n3-c3ccc([C@@H]4NC[C@@H]5CC[C@H]4O5)cc3)c2=O)CC1]

### Step 1: (S)-5-(((tert-Butyldiphenylsilyl)oxy)methyl)dihydrofuran-2(3H)-one

To (S)-5-(hydroxymethyl)dihydrofuran-2(3H)-one (2.90 g, 25.0 mmol) and imidazole (3.40 g, 49.9 mmol) in DMF (29 mL) at 0° C. was added tert-butylchlorodiphenylsilane (8.44 mL, 32.5 mmol). The reaction mixture was allowed to warm to rt and stirred overnight before EtOAc (150 mL) was added. The resulting mixture was washed with 1:1 brine/water (3×100 mL). The organic phase was dried over MgSO₄, filtered and concentrated under reduced pressure. The residue was purified by flash chromatography to give title compound (7.56 g, 85%) as colourless oil that solidified upon storage. LCMS (method B): R_{T}=1.70 min, m/z=277 [M−Ph]⁺. ¹H NMR (500 MHz, Chloroform-d) δ 7.69-7.61 (m, 4H), 7.48-7.33 (m, 6H), 4.65-4.55 (m, 1H), 3.88 (ddd, J=11.2, 3.4, 1.0 Hz, 1H), 3.69 (ddd, J=11.2, 3.3, 1.0 Hz, 1H), 2.73-2.60 (m, 1H), 2.58-2.44 (m, 1H), 2.36-2.12 (m, 2H), 1.06 (s, 9H).

### Step 2: (5S)-5-(((tert-Butyldiphenylsilyl)oxy)methyl)tetrahydrofuran-2-ol

To a solution of (S)-5-(((tert-butyldiphenylsilyl)oxy)methyl)dihydrofuran-2(3H)-one (7.56 g, 21.3 mmol) dry diethyl ether (100 mL) under nitrogen and at −78° C. was added diisobutylaluminium hydride (1 M in DCM, 32.0 mL, 32.0 mmol). The reaction was stirred at −78° C. for 5 h before being quenched by the addition of MeOH (15 mL). After stirring at −78° C. for 15 min the reaction mixture was warmed to rt and diluted with diethyl ether (50 mL). The resulting mixture was washed with 0.2 M aqueous sodium tartrate (2×150 mL) and brine. The organic phase was dried over MgSO₄, filtered, and concentrated under reduced pressure to give the title compound (7.61 g, quant.) as a colourless oil. LCMS (method B): R_{T}=1.69 min, m/z=339 [M−OH]⁺.

### Step 3: (S)-tert-Butyl((2,3-dihydrofuran-2-yl)methoxy)diphenylsilane

To a solution of (5S)-5-(((tert-butyldiphenylsilyl)oxy)methyl)tetrahydrofuran-2-ol (7.61 g, 21.3 mmol) in DCM (140 mL) at −50° C. under N₂, were added triethylamine (11.9 mL, 85.4 mmol) followed by methanesulfonyl chloride (2.32 mL, 29.9 mmol). The reaction mixture was stirred at −50° C. for 3 h until all the starting material had been consumed at which point the reaction was warmed up to rt and subsequently stirred at reflux for a further 18 h. Upon cooling to rt the reaction mixture was washed with 1:1 brine/water solution (2×100 mL). The organic phase was dried over MgSO₄, filtered and concentrated under reduced pressure. The residue was purified by flash chromatography to give title compound (4.82 g, 66%) as a colourless oil. LCMS (method A): R_{T}=2.24 min, mass ion not observed. ¹H NMR (500 MHz, Chloroform-d) δ 7.71-7.65 (m, 4H), 7.45-7.35 (m, 6H), 6.27 (q, J=2.4 Hz, 1H), 4.85 (q, J=2.6 Hz, 1H), 4.71-4.58 (m, 1H), 3.79-3.65 (m, 2H), 2.69-2.59 (m, 1H), 2.54-2.40 (m, 1H), 1.06 (s, 9H).

### Step 4: (S)-(2,3-Dihydrofuran-2-yl)methyl 4-methylbenzenesulfonate

To (S)-tert-butyl((2,3-dihydrofuran-2-yl)methoxy)diphenylsilane (1.00 g, 2.95 mmol) was added TBAF (1 M in THF, 2.95 mL, 2.95 mmol) and the resulting mixture was stirred at rt for 1 h. The reaction mixture was diluted with DCM (20 mL) and cooled to 0° C. before triethylamine (3.29 mL, 23.6 mmol) and 4-methylbenzenesulfonyl chloride (2.25 g, 11.8 mmol) were added. After stirring at rt for 19 h the reaction mixture was concentrated under reduced pressure, the residue was dissolved in EtOAc (50 mL) and washed with 1:1 brine/water (2×50 mL). The organic layer was dried over MgSO₄, filtered and concentrated under reduced pressure. The residue was purified by flash chromatography to give title compound (683 mg, 90%) as a colourless oil. LCMS (method A): R_{T}=1.38 min, m/z=255 [M+H]⁺. ¹H NMR (500 MHz, Chloroform-d) δ 7.85-7.73 (m, 2H), 7.37-7.29 (m, 2H), 5.34-5.16 (m, 1H), 4.30-4.15 (m, 1H), 4.11-3.89 (m, 2H), 2.45 (d, J=1.8 Hz, 3H), 2.16-1.58 (m, 3H).

### Step 5: (S)-2-((2,3-Dihydrofuran-2-yl)methyl)isoindoline-1,3-dione

A mixture of (S)-(2,3-dihydrofuran-2-yl)methyl 4-methylbenzenesulfonate (683 mg, 2.69 mmol) and potassium 1,3-dioxoisoindolin-2-ide (746 mg, 4.03 mmol) in DMF (10 mL) under nitrogen was stirred at 75° C. for 19 h. Upon cooling at rt the reaction mixture was diluted with EtOAc (50 mL) and washed with 1:1 brine/water (3×50 mL). The organic layer was dried over MgSO₄, filtered and concentrated under reduced pressure. The residue was purified by flash chromatography to give title compound (465 mg, 75%) as a white solid. LCMS (method A): R_{T}=1.18 min, m/z=230 [M+H]⁺. ¹H NMR (500 MHz, Chloroform-d) δ 7.86 (dd, J=5.4, 3.0 Hz, 2H), 7.72 (dd, J=5.5, 3.0 Hz, 2H), 6.25 (q, J=2.5 Hz, 1H), 4.98-4.82 (m, 2H), 3.97 (dd, J=13.9, 8.4 Hz, 1H), 3.67 (dd, J=13.9, 4.5 Hz, 1H), 2.77 (ddt, J=15.1, 10.1, 2.4 Hz, 1H), 2.40 (ddt, J=15.3, 6.0, 2.4 Hz, 1H).

### Step 6: (S)-(2,3-Dihydrofuran-2-yl)methanamine

To (S)-2-((2,3-dihydrofuran-2-yl)methyl)isoindoline-1,3-dione (230 mg, 1.00 mmol) in MeOH (4 mL) was added hydrazine monohydrate (0.19 mL, 6.02 mmol). The reaction mixture was heated at 60° C. for 2h. Upon cooling to rt aqueous sodium hydroxide (2 M, 7.5 mL) was added and the resulting mixture was extracted with DCM (3×15 mL). The combined organic layers were washed with brine (50 mL), dried over MgSO₄, filtered and concentrated at reduced pressure (41° C., 200 mbar) to give the title compound (150 mg, >100%). ¹H NMR (500 MHz, Chloroform-d) δ 6.28 (q, J=2.5 Hz, 1H), 4.88 (q, J=2.6 Hz, 1H), 4.55 (ddt, J=10.7, 7.4, 5.5 Hz, 1H), 2.83 (d, J=5.5 Hz, 2H), 2.69 (ddt, J=15.1, 10.3, 2.4 Hz, 1H), 2.30 (ddt, J=15.2, 7.4, 2.4 Hz, 1H), 1.37-1.19 (m, 2H). This material was used without purification in the next step.

### Step 7: (1R,2S,5S)-2-(4-Bromophenyl)-8-oxa-3-azabicyclo[3.2.1]octane

A mixture of (S)-(2,3-dihydrofuran-2-yl)methanamine (100 mg, 1.01 mmol), 4-bromobenzaldehyde (187 mg, 1.01 mmol) and 3 Å molecular sieves (200 mg) in DCM (1 mL) was stirred at rt for 19 h. The reaction mixture was filtered through a syringe filter and the volatiles were evaporated under reduced pressure (200 mbar, 40° C.). The residue was dissolved in 2-propanol (10 mL) and transferred to in a 25 mL pressure vial before tris(2,2,6,6-tetramethyl-3,5-heptanedionato)manganese(III) (30.4 mg, 0.0504 mmol) was added and the vial was capped with a septa. To the resulting mixture was then added via a syringe through the septa PhSiH₃ (0.248 μL, 2.01 mmol) and the reaction was heated at 85° C. for 5 h. Further PhSiH₃ (0.248 μL, 2.01 mmol) was added, the pressure inside the vial was released and the reaction was heated at 85° C. for 19 h. Further PhSiH₃ (0.248 μL, 2.01 mmol) was added, the pressure inside the vial was released and the reaction was heated at 85° C. for 4 h. Upon cooling to rt the reaction mixture was concentrated under reduced pressure and the residue was purified by flash chromatography to give the title compound (112 mg, 41%) as a colourless glass. LCMS (method B): R_{T}=0.50 min, m/z=268, 270 [M+H]⁺.

### Step 8: tert-Butyl (1R,2S,5S)-2-(4-bromophenyl)-8-oxa-3-azabicyclo[3.2.1]octane-3-carboxylate

To a solution of (1R,2S,5S)-2-(4-bromophenyl)-8-oxa-3-azabicyclo[3.2.1]octane (164 mg, 0.612 mmol) and DIPEA (160 μL, 0.917 mmol) in DCM (6 mL) at rt was added dropwise a solution of Boc₂O (160 mg, 0.734 mmol) in DCM (2 mL). The reaction mixture was stirred at rt for 18 h before the volatiles were removed under reduced pressure and the residue was purified by flash chromatography to give the title compound (173 mg, 76%) as a colourless glass. LCMS (method B): R_{T}=1.58 min, m/z=268, 270 [M−Boc+H]⁺.

### Step 9: tert-Butyl (1R,2S,5S)-2-(4-(4,4,5,5-tetramethyl-1,3,2-dioxaborolan-2-yl)phenyl)-8-oxa-3-azabicyclo[3.2.1]octane-3-carboxylate

General procedure 3 (Work-up A) using tert-butyl (1R,2S,5S)-2-(4-bromophenyl)-8-oxa-3-azabicyclo[3.2.1]octane-3-carboxylate (173 mg, 0.470 mmol), B₂Pin₂ (179 mg, 0.705 mmol), Pd(dppf)Cl₂·CH₂Cl₂ (39.8 mg, 0.0470 mmol), KOAc (138 mg, 1.41 mmol) and 1,4-dioxane (5 mL) after stirring at 100° C. (thermal) for 4 h gave the title compound (195 mg, 95%) as a colourless glass. LCMS (method B): R_{T}=1.71 min, m/z=315 [M−Boc+H]⁺.

### Step 10: tert-Butyl (1R,2S,5S)-2-(4-(4,6-dichloro-7H-pyrrolo[2,3-d]pyrimidin-7-yl)phenyl)-8-oxa-3-azabicyclo[3.2.1]octane-3-carboxylate

General procedure 4 (Work-up A) using 4,6-dichloro-7H-pyrrolo[2,3-d]pyrimidine (68 mg, 0.362 mmol), tert-butyl (1R,2S,5S)-2-(4-(4,4,5,5-tetramethyl-1,3,2-dioxaborolan-2-yl)phenyl)-8-oxa-3-azabicyclo[3.2.1]octane-3-carboxylate (195 mg, 0.470 mmol), copper(II) trifluoroacetate hydrate (209 mg, 0.723 mmol), 1,10-phenanthroline (130 mg, 0.723 mmol), boric acid (89.5 mg, 1.45 mmol) and DMF (6.8 mL) after 3 days at 65° C. gave the title compound (105 mg, 61%) as a colourless glass. LCMS (method B): R_{T}=1.67 min, m/z=475, 477 [M+H]⁺.

### Step 11: tert-Butyl (1R,2S,5S)-2-(4-(6-chloro-4-oxo-3,4-dihydro-7H-pyrrolo[2,3-d]pyrimidin-7-yl)phenyl)-8-oxa-3-azabicyclo[3.2.1]octane-3-carboxylate

General procedure 5 using tert-butyl (1R,2S,5S)-2-(4-(4,6-dichloro-7H-pyrrolo[2,3-d]pyrimidin-7-yl)phenyl)-8-oxa-3-azabicyclo[3.2.1]octane-3-carboxylate (105 mg, 0.221 mmol), aqueous sodium hydroxide (4 M, 1.66 mL, 6.63 mmol) and 1,4-dioxane (1.65 mL) after 18 h at 100° C. using 1 M aqueous potassium bisulfate for acidification gave the title compound (154 mg, >100%) as a yellow glass. LCMS (method A): R_{T}=1.24 min, m/z=455, 456 [M−H]⁻. This material was used without further purification.

### Step 12: tert-Butyl (1R,2S,5S)-2-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-8-oxa-3-azabicyclo[3.2.1]octane-3-carboxylate

General procedure 6 using tert-butyl (1R,2S,5S)-2-(4-(6-chloro-4-oxo-3,4-dihydro-7H-pyrrolo[2,3-d]pyrimidin-7-yl)phenyl)-8-oxa-3-azabicyclo[3.2.1]octane-3-carboxylate (50 mg, 0.109 mmol), Epoxide 2 (41.3 mg, 0.164 mmol), Cs₂CO₃ (107 mg, 0.328 mmol) and DMF (2 mL) after 18 h at 80° C. gave the title compound (47 mg, 60%) as a colourless glass. LCMS (method B): R_{T}=1.46 min, m/z=652, 654 [M−butene+H]⁺.

### Step 13: 7-(4-((1R,2S,5S)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

General procedure 7 using tert-butyl (1R,2S,5S)-2-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-8-oxa-3-azabicyclo[3.2.1]octane-3-carboxylate (47 mg, 0.0663 mmol), TFA (1 mL) and DCM (2 mL) gave, after freeze-drying, the title compound (36 mg, 89%) as a white solid. LCMS (method B): R_{T}=0.82 min, m/z=608, 610 [M+H]⁺. ¹H NMR (500 MHz, DMSO-d₆) δ 8.07 (s, 1H), 7.66-7.57 (m, 2H), 7.54-7.48 (m, 2H), 7.46-7.37 (m, 4H), 6.80 (s, 1H), 4.96 (s, 1H), 4.30-4.22 (m, 2H), 4.22-4.08 (m, 2H), 4.08-3.96 (m, 2H), 3.51-3.34 (m, 1H), 3.30-3.06 (m, 2H), 3.06-2.96 (m, 1H), 2.83-2.59 (m, 2H), 1.98-1.88 (m, 1H), 1.84-1.72 (m, 2H), 1.70-1.24 (m, 5H).

### Example 49: 7-(4-((1R,2S,5S)-8-Oxa-3-azabicyclo[3.2.1]octan-2-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one

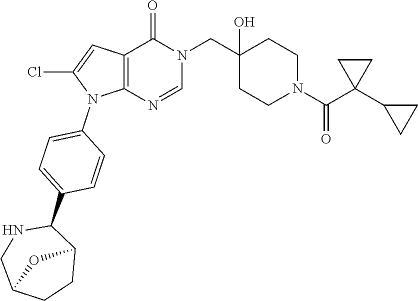 [SMILES: O=C(N1CCC(O)(Cn2cnc3c(cc(Cl)n3-c3ccc([C@@H]4NC[C@@H]5CC[C@H]4O5)cc3)c2=O)CC1)C1(C2CC2)CC1]

### Step 1: tert-Butyl (1R,2S,5S)-2-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-8-oxa-3-azabicyclo[3.2.1]octane-3-carboxylate

General procedure 6 using tert-butyl (1R,2S,5S)-2-(4-(6-chloro-4-oxo-3,4-dihydro-7H-pyrrolo[2,3-d]pyrimidin-7-yl)phenyl)-8-oxa-3-azabicyclo[3.2.1]octane-3-carboxylate (50 mg, 0.109 mmol), Epoxide 16 (36.3 mg, 0.164 mmol), Cs₂CO₃ (107 mg, 0.328 mmol) and DMF (2 mL) after 18 h at 80° C. gave the title compound (32 mg, 43%) as a colourless glass. LCMS (method B): R_{T}=1.38 min, m/z=622, 624 [M−butene+H]⁺.

### Step 2: 7-(4-((1R,2S,5S)-8-Oxa-3-azabicyclo[3.2.1]octan-2-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one

General procedure 7 using tert-butyl (1R,2S,5S)-2-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)-8-oxa-3-azabicyclo[3.2.1]octane-3-carboxylate (32 mg, 0.0472 mmol), TFA (1 mL) and DCM (2 mL) gave, after freeze-drying, the title compound (26 mg, 95%) as a white solid. LCMS (method B): R_{T}=0.75 min, m/z=578, 580 [M+H]⁺. ¹H NMR (500 MHz, DMSO-d₆) δ 8.08 (s, 1H), 7.64-7.57 (m, 2H), 7.44-7.36 (m, 2H), 6.81 (s, 1H), 4.92 (s, 1H), 4.31-4.19 (m, 2H), 4.13 (s, 1H), 4.08-3.87 (m, 4H), 3.42-2.83 (m, 3H), 2.82-2.66 (m, 2H), 1.98-1.85 (m, 1H), 1.86-1.70 (m, 2H), 1.68-1.29 (m, 5H), 1.19-1.08 (m, 1H), 0.76-0.59 (m, 2H), 0.52-0.41 (m, 2H), 0.42-0.29 (m, 2H), 0.12-0.06 (m, 2H).

### Example 50: 6-Chloro-3-((1-(4-chloro-2-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

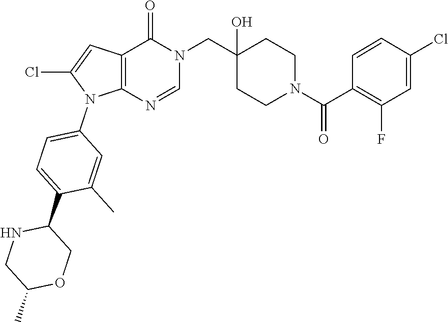 [SMILES: Cc1cc(-n2c(Cl)cc3c(=O)n(CC4(O)CCN(C(=O)c5ccc(Cl)cc5F)CC4)cnc32)ccc1[C@H]1CO[C@H](C)CN1]

### Step 1: tert-Butyl (2R,5S)-5-(4-(6-chloro-3-((1-(4-chloro-2-fluorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 7 (44 mg, 0.163 mmol), Cs₂CO₃ (106 mg, 0.327 mmol) and DMF (2 mL) after 18 h at 80° C. gave the title compound (39 mg, 49%) as a colourless glass. LCMS (method B): R_{T}=1.56 min, m/z=672, 674 [M−butene+H]⁺.

### Step 2: 6-Chloro-3-((1-(4-chloro-2-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-chloro-2-fluorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (39 mg, 0.0535 mmol), TFA (1 mL) and DCM (2 mL) gave, after freeze-drying, the title compound (33 mg, 98%) as a white solid. LCMS (method B): R_{T}=0.87 min, m/z=628, 630 [M+H]⁺. ¹H NMR (500 MHz, DMSO-d₆) δ 8.06 (s, 1H), 7.73 (d, J=8.0 Hz, 1H), 7.56 (dd, J=9.4, 2.0 Hz, 1H), 7.46 (s, 1H), 7.39 (dd, J=8.2, 1.9 Hz, 1H), 7.28-7.20 (m, 2H), 6.78 (s, 1H), 4.99 (s, 1H), 4.29-4.13 (m, 1H), 4.13-3.95 (m, 3H), 3.80 (dd, J=10.8, 3.0 Hz, 1H), 3.65-3.52 (m, 1H), 3.29-3.10 (m, 4H), 2.97 (dd, J=11.6, 2.3 Hz, 1H), 2.87-2.70 (m, 1H), 2.62-2.54 (m, 1H), 2.42 (s, 3H), 1.67-1.44 (m, 3H), 1.40-1.30 (m, 1H), 1.10 (d, J=6.2 Hz, 3H).

### Example 51: 6-Chloro-3-((1-(3-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

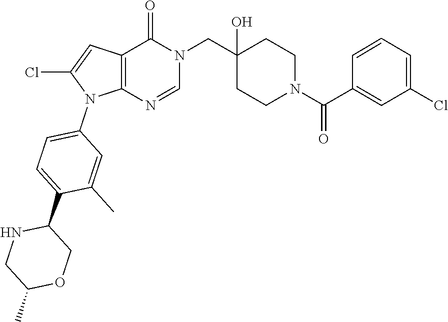 [SMILES: Cc1cc(-n2c(Cl)cc3c(=O)n(CC4(O)CCN(C(=O)c5cccc(Cl)c5)CC4)cnc32)ccc1[C@H]1CO[C@H](C)CN1]

### Step 1: tert-Butyl (2R,5S)-5-(4-(6-chloro-3-((1-(3-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 11 (41 mg, 0.163 mmol), Cs₂CO₃ (106 mg, 0.327 mmol) and DMF (2 mL) after 18 h at 80° C. gave the title compound (46 mg, 59%) as a colourless glass. LCMS (method A): R_{T}=1.68 min, m/z=654, 656 [M−butene+H]⁺.

### Step 2: 6-Chloro-3-((1-(3-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(3-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (46 mg, 0.0647 mmol), TFA (1 mL) and DCM (2 mL) gave, after freeze-drying, the title compound (37 mg, 93%) as a white solid. LCMS (method B): R_{T}=0.86 min, m/z=610, 612 [M+H]⁺. ¹H NMR (500 MHz, DMSO-d₆) δ 8.06 (s, 1H), 7.73 (d, J=7.9 Hz, 1H), 7.55-7.45 (m, 3H), 7.38-7.33 (m, 1H), 7.28-7.20 (m, 2H), 6.79 (s, 1H), 4.96 (s, 1H), 4.27-4.11 (m, 1H), 4.09-3.94 (m, 3H), 3.80 (dd, J=10.9, 3.0 Hz, 1H), 3.64-3.51 (m, 1H), 3.44-3.34 (m, 1H), 3.30-3.04 (m, 3H), 2.97 (dd, J=11.6, 2.3 Hz, 1H), 2.88-2.68 (m, 1H), 2.62-2.54 (m, 1H), 2.42 (s, 3H), 1.72-1.55 (m, 2H), 1.52-1.26 (m, 2H), 1.10 (d, J=6.2 Hz, 3H).

### Example 52: 6-Chloro-3-((1-(2-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

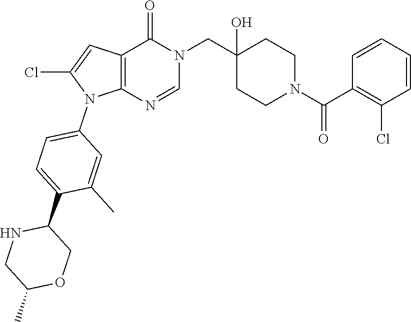 [SMILES: Cc1cc(-n2c(Cl)cc3c(=O)n(CC4(O)CCN(C(=O)c5ccccc5Cl)CC4)cnc32)ccc1[C@H]1CO[C@H](C)CN1]

### Step 1: tert-Butyl (2R,5S)-5-(4-(6-chloro-3-((1-(2-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 8 (41.1 mg, 0.163 mmol), Cs₂CO₃ (106 mg, 0.327 mmol) and DMF (2 mL) after 18 h at 80° C. gave the title compound (47 mg, 60%) as colourless glass. LCMS (method A): R_{T}=1.62 min, m/z=654, 656 [M−butene+H]⁺.

### Step 2: 6-Chloro-3-((1-(2-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(2-chlorobenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (47 mg, 0.0661 mmol), TFA (1 mL) and DCM (2 mL) gave, after freeze-drying, the title compound (34 mg, 84%) as a white solid. LCMS (method B): R_{T}=0.82 min, m/z=610, 612 [M+H]⁺. ¹H NMR (500 MHz, DMSO-d₆) δ 8.06 (d, J=1.8 Hz, 1H), 7.73 (d, J=7.9 Hz, 1H), 7.57-7.48 (m, 1H), 7.48-7.29 (m, 3H), 7.27-7.19 (m, 2H), 6.78 (d, J=2.3 Hz, 1H), 4.98 (d, J=2.1 Hz, 1H), 4.28-4.16 (m, 1H), 4.10-3.92 (m, 3H), 3.86-3.75 (m, 1H), 3.66-3.53 (m, 1H), 3.28-3.05 (m, 4H), 3.05-2.94 (m, 1H), 2.91-2.69 (m, 1H), 2.62-2.55 (m, 1H), 2.42 (s, 3H), 1.69-1.44 (m, 3H), 1.36-1.28 (m, 1H), 1.10 (d, J=6.2 Hz, 3H).

### Example 53: 6-Chloro-3-((1-(4-chlorobenzoyl)-4-hydroxypiperidin-4-yl)methyl)-7-(4-(octahydro-2H-benzo[b][1,4]oxazin-3-yl)phenyl)-3,7-dihydro-4H-pyrrolo[2,3-d]pyrimidin-4-one

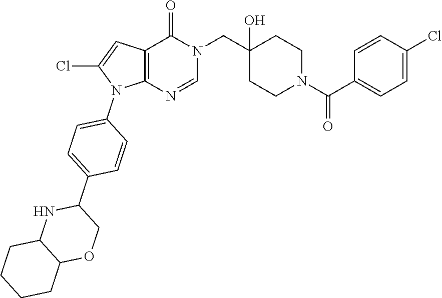 [SMILES: O=C(c1ccc(Cl)cc1)N1CCC(O)(Cn2cnc3c(cc(Cl)n3-c3ccc(C4COC5CCCCC5N4)cc3)c2=O)CC1]

### Step 1: 3-(4-Bromophenyl)octahydro-2H-benzo[b][1,4]oxazine

General procedure 8 using 4-bromobenzaldehyde (1.02 g, 5.53 mmol), 2-((tributylstannyl)methoxy)cyclohexan-1-amine (2.32 g, 5.53 mmol), Cu(OTf)₂ (2.00 g, 5.53 mmol), 2,6-dimethylpyridine (592 mg, 5.53 mmol), HFIP (22 mL), 4 Å molecular sieves (ca. 0.5 g) and DCM (49 mL) gave the title compound (0.97 g, 59%). LCMS (method C): R_{T}=0.58 min, m/z=296, 298 [M+H]⁺.

### Step 2: tert-Butyl 3-(4-bromophenyl)octahydro-4H-benzo[b][1,4]oxazine-4-carboxylate

General procedure 9 using 3-(4-bromophenyl)octahydro-2H-benzo[b][1,4]oxazine (0.97 g, 3.26 mmol) and Boc₂O (854 mg, 3.91 mmol) gave the title compound (852 mg, 66%). LCMS (method C): R_{T}=1.67 min, m/z=296, 298 [M−Boc+H]⁺. ¹H NMR (500 MHz, Chloroform-d) δ 1.21-1.34 (m, 3H), 1.44 (s, 9H), 1.56-1.70 (m, 3H), 1.79-1.89 (m, 1H), 2.57-2.67 (m, 1H), 3.27 (td, J=10.6, 3.9 Hz, 1H), 3.46 (td, J=10.5, 3.2 Hz, 1H), 4.29 (dd, J=11.2, 5.2 Hz, 1H), 4.38 (dd, J=11.2, 3.2 Hz, 1H), 5.05-5.14 (m, 1H), 7.27 (d, J=8.7 Hz, 2H), 7.48 (d, J=8.1 Hz, 2H).

### Step 3: tert-Butyl 3-(4-(4,4,5,5-tetramethyl-1,3,2-dioxaborolan-2-yl)phenyl)octahydro-4H-benzo[b][1,4]oxazine-4-carboxylate

General procedure 3 (Work-up B) using tert-butyl 3-(4-bromophenyl)octahydro-4H-benzo[b][1,4]oxazine-4-carboxylate (400 mg, 1.01 mmol), B₂Pin₂ (384 mg, 1.51 mmol), Pd(dppf)Cl₂·CH₂Cl₂ (85.5 mg, 0.101 mmol), KOAc (297 mg, 3.03 mmol) and 1,4-dioxane (10.1 mL) after stirring at 90° C. (thermal) for 1 h gave the title compound (408 mg, 91%) as a colourless gum. LCMS (method B): R_{T}=1.90 min, m/z=344 [M−Boc+H]⁺.

### Step 4: tert-Butyl 3-(4-(4,6-dichloro-7H-pyrrolo[2,3-d]pyrimidin-7-yl)phenyl)octahydro-4H-benzo[b][1,4]oxazine-4-carboxylate

General procedure 4 (Work-up B) using 4,6-dichloro-7H-pyrrolo[2,3-d]pyrimidine (259.5 mg, 1.38 mmol), tert-butyl 3-(4-(4,4,5,5-tetramethyl-1,3,2-dioxaborolan-2-yl)phenyl)octahydro-4H-benzo[b][1,4]oxazine-4-carboxylate (408 mg, 0.920 mmol), copper(II) trifluoroacetate hydrate (533 mg, 1.84 mmol), 1,10-phenanthroline (332 mg, 1.84 mmol), boric acid (171 mg, 2.76 mmol) and DMF (9.2 mL) after 5 days at 50° C. gave the title compound (190 mg, 41%) as a white foam. LCMS (method B): R_{T}=1.85 min, m/z=503, 505 [M+H]⁺.

### Step 5: tert-Butyl 3-(4-(6-chloro-4-oxo-3,4-dihydro-7H-pyrrolo[2,3-d]pyrimidin-7-yl)phenyl)octahydro-4H-benzo[b][1,4]oxazine-4-carboxylate

General procedure 5 using tert-butyl 3-(4-(4,6-dichloro-7H-pyrrolo[2,3-d]pyrimidin-7-yl)phenyl)octahydro-4H-benzo[b][1,4]oxazine-4-carboxylate (190 mg, 0.377 mmol), aqueous sodium hydroxide (4 M, 0.94 mL, 3.77 mmol) and 1,4-dioxane (2 mL) after 26 h at 100° C. using 2 M aqueous hydrochloric acid for acidification gave the title compound (181 mg, 90%) as a brown solid. LCMS (method B): R_{T}=1.42 min, m/z=485, 487 [M+H]⁺. This compound was used without further purification.

### Step 6: tert-Butyl 3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)octahydro-4H-benzo[b][1,4]oxazine-4-carboxylate

General procedure 6 using tert-butyl 3-(4-(6-chloro-4-oxo-3,4-dihydro-7H-pyrrolo[2,3-d]pyrimidin-7-yl)phenyl)octahydro-4H-benzo[b][1,4]oxazine-4-carboxylate (181 mg, 0.373 mmol), Epoxide 2 (113 mg, 0.448 mmol), Cs₂CO₃ (268 mg, 0.821 mmol) and DMF (2 mL) after 18 h at 80° C. gave the title compound (72 mg, 26%) as a colourless gum. LCMS (method B): R_{T}=1.65 min, m/z=636, 638 [M−Boc+H]+.

### Step 7: 6-Chloro-3-((1-(4-chlorobenzoyl)-4-hydroxypiperidin-4-yl)methyl)-7-(4-(octahydro-2H-benzo[b][1,4]oxazin-3-yl)phenyl)-3,7-dihydro-4H-pyrrolo[2,3-d]pyrimidin-4-one

General procedure 7 using tert-butyl 3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)octahydro-4H-benzo[b][1,4]oxazine-4-carboxylate (10 mg, 0.0136 mmol), TFA (0.5 mL) and DCM (1 mL) gave, after freeze-drying, the title compound (7 mg, 81%) as a white solid. LCMS (method B): R_{T}=0.88 min, m/z=636, 638 [M+H]⁺. ¹H NMR (500 MHz, DMSO-d₆) δ 8.06 (s, 1H), 7.63-7.58 (m, 2H), 7.53-7.48 (m, 2H), 7.45-7.35 (m, 4H), 6.79 (s, 1H), 4.95 (s, 1H), 4.25-4.10 (m, 1H), 4.06-3.97 (m, 3H), 3.82 (dd, J=10.7, 3.3 Hz, 1H), 3.43-3.33 (m, 1H), 3.30-3.22 (m, 2H), 3.19-3.08 (m, 1H), 3.06-2.99 (m, 1H), 2.84 (s, 1H), 2.47-2.41 (m, 1H), 1.82-1.75 (m, 1H), 1.74-1.68 (m, 2H), 1.67-1.54 (m, 3H), 1.51-1.41 (m, 1H), 1.37-1.18 (m, 5H).

### Example 54: 6-Chloro-3-((1-(4-chlorobenzoyl)-4-hydroxypiperidin-4-yl)methyl)-7-(4-(octahydrocyclopenta[b][1,4]oxazin-3-yl)phenyl)-3,7-dihydro-4H-pyrrolo[2,3-d]pyrimidin-4-one

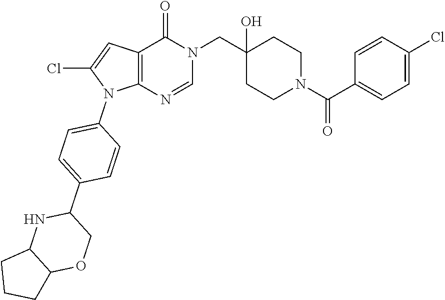 [SMILES: O=C(c1ccc(Cl)cc1)N1CCC(O)(Cn2cnc3c(cc(Cl)n3-c3ccc(C4COC5CCCC5N4)cc3)c2=O)CC1]

### Step 1: 3-(4-Bromophenyl)octahydrocyclopenta[b][1,4]oxazine

General procedure 8 using 4-bromobenzaldehyde (1.87 g, 10.1 mmol), 2-((tributylstannyl)methoxy)cyclopentan-1-amine (4.08 g, 10.1 mmol), Cu(OTf)₂ (3.66 g, 10.1 mmol), 2,6-dimethylpyridine (1.08 g, 10.1 mmol), HFIP (40 mL), 4 Å molecular sieves (ca. 1 g) and DCM (160 mL) gave the title compound (1.45 g, 51%). LCMS (method C): R_{T}=0.58 min, m/z=282, 284 [M+H]⁺.

### Step 2: tert-Butyl 3-(4-bromophenyl)hexahydrocyclopenta[b][1,4]oxazine-4(4aH)-carboxylate

General procedure 9 using 3-(4-bromophenyl)octahydrocyclopenta[b][1,4]oxazine (1.45 g, 5.14 mmol) and Boc₂O (1.35 g, 6.17 mmol) gave the title compound (1.24 g, 63%). LCMS (method C): R_{T}=1.62 min, m/z=282, 284 [M−Boc+H]⁺. ¹H NMR (500 MHz, Chloroform-d) δ 1.42 (s, 9H), 1.44-1.64 (m, 3H), 1.66-1.75 (m, 1H), 1.77-1.85 (m, 1H), 2.48-2.61 (m, 1H), 3.51 (td, J=10.5, 6.9 Hz, 1H), 3.64 (td, J=10.6, 7.0 Hz, 1H), 4.22-4.33 (m, 2H), 4.89 (t, J=3.7 Hz, 1H), 7.28 (dd, J=85.9, 8.2 Hz, 2H), 7.46 (d, J=8.3 Hz, 2H).

### Step 3: tert-Butyl 3-(4-(4,4,5,5-tetramethyl-1,3,2-dioxaborolan-2-yl)phenyl)hexahydrocyclopenta[b][1,4]oxazine-4(4aH)-carboxylate

General procedure 3 (Work-up B) using tert-butyl 3-(4-bromophenyl)hexahydrocyclopenta[b][1,4]oxazine-4(4aH)-carboxylate (500 mg, 1.31 mmol), B₂Pin₂ (498 mg, 1.96 mmol), Pd(dppf)Cl₂·CH₂Cl₂ (111 mg, 0.131 mmol), KOAc (385 mg, 3.92 mmol) and 1,4-dioxane (13 mL) after stirring at 90° C. (thermal) for 1 h gave the title compound (555 mg, 98%) as a colourless gum. LCMS (method B): R_{T}=1.82 min, m/z=330 [M−Boc+H]⁺.

### Step 4: tert-Butyl 3-(4-(4,6-dichloro-7H-pyrrolo[2,3-d]pyrimidin-7-yl)phenyl)hexahydrocyclopenta[b][1,4]oxazine-4(4aH)-carboxylate

General procedure 4 (Work-up B) using 4,6-dichloro-7H-pyrrolo[2,3-d]pyrimidine (364.5 mg, 1.94 mmol), tert-butyl 3-(4-(4,4,5,5-tetramethyl-1,3,2-dioxaborolan-2-yl)phenyl)hexahydrocyclopenta[b][1,4]oxazine-4(4aH)-carboxylate (555 mg, 1.29 mmol), copper(II) trifluoroacetate hydrate (749 mg, 2.59 mmol), 1,10-phenanthroline (466 mg, 2.59 mmol), boric acid (240 mg, 3.88 mmol) and DMF (12.9 mL) after 5 days at 50° C. gave the title compound (265 mg, 41%) as a white foam. LCMS (method B): R_{T}=1.77 min, m/z=489, 491 [M+H]⁺.

### Step 5: tert-Butyl 3-(4-(6-chloro-4-oxo-3,4-dihydro-7H-pyrrolo[2,3-d]pyrimidin-7-yl)phenyl)hexahydrocyclopenta[b][1,4]oxazine-4(4aH)-carboxylate

General procedure 5 using tert-butyl 3-(4-(4,6-dichloro-7H-pyrrolo[2,3-d]pyrimidin-7-yl)phenyl)hexahydrocyclopenta[b][1,4]oxazine-4(4aH)-carboxylate (265 mg, 0.542 mmol), aqueous sodium hydroxide (4 M, 1.35 mL, 5.41 mmol) and 1,4-dioxane (3 mL) after 26 h at 100° C. using 2 M aqueous hydrochloric acid for acidification gave the title compound (254 mg, 91%) as a brown solid. LCMS (method B): R_{T}=1.34 min, m/z=471, 473 [M+H]⁺.

### Step 6: tert-Butyl 3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)hexahydrocyclopenta[b][1,4]oxazine-4(4aH)-carboxylate

General procedure 6 using tert-butyl 3-(4-(6-chloro-4-oxo-3,4-dihydro-7H-pyrrolo[2,3-d]pyrimidin-7-yl)phenyl)hexahydrocyclopenta[b][1,4]oxazine-4(4aH)-carboxylate (254 mg, 0.539 mmol), Epoxide 2 (163 mg, 0.647 mmol), Cs₂CO₃ (387 mg, 1.19 mmol) and DMF (3 mL) after 18 h at 80° C. gave the title compound (43 mg, 11%) as colourless gum. LCMS (method B): R_{T}=1.58 min, m/z=622, 624 [M−Boc+H]+.

### Step 7: 6-Chloro-3-((1-(4-chlorobenzoyl)-4-hydroxypiperidin-4-yl)methyl)-7-(4-(octahydrocyclopenta[b][1,4]oxazin-3-yl)phenyl)-3,7-dihydro-4H-pyrrolo[2,3-d]pyrimidin-4-one

General procedure 7 using tert-butyl 3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)hexahydrocyclopenta[b][1,4]oxazine-4(4aH)-carboxylate (20 mg, 0.0277 mmol), TFA (1.5 mL) and DCM (3 mL) gave, after freeze-drying, the title compound (12 mg, 69%) as a white solid. LCMS (method B): R_{T}=0.85 min, m/z=622, 624 [M+H]⁺. ¹H NMR (500 MHz, DMSO-d₆) δ 8.06 (s, 1H), 7.66-7.57 (m, 2H), 7.53-7.47 (m, 2H), 7.45-7.32 (m, 4H), 6.79 (s, 1H), 4.95 (s, 1H), 4.22-4.12 (m, 1H), 4.02 (s, 2H), 3.98 (dd, J=10.4, 3.3 Hz, 2H), 3.86 (dd, J=11.0, 3.4 Hz, 1H), 3.42-3.33 (m, 1H), 3.29-3.02 (m, 5H), 1.85-1.65 (m, 4H), 1.64-1.53 (m, 2H), 1.51-1.41 (m, 2H), 1.41-1.29 (m, 2H).

### Example 55: 6-Chloro-3-((4-hydroxy-1-(4-methylbenzyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

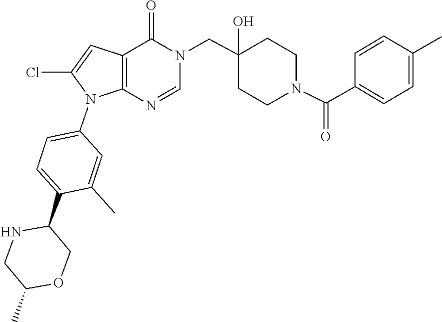 [SMILES: Cc1ccc(C(=O)N2CCC(O)(Cn3cnc4c(cc(Cl)n4-c4ccc([C@H]5CO[C@H](C)CN5)c(C)c4)c3=O)CC2)cc1]

### Step 1: tert-Butyl (2R,5S)-5-(4-(6-chloro-3-((4-hydroxy-1-(4-methylbenzoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 17 (27.7 mg, 0.120 mmol), Cs₂CO₃ (71 mg, 0.218 mmol) and DMF (1.1 mL) after 19 h at 80° C. gave the title compound (61 mg, 81%) as a viscous yellow gum. LCMS (method A): R_{T}=1.64 min, m/z=634, 636 [M−butene+H]⁺, 690, 692 [M+H]⁺.

### Step 2: 6-Chloro-3-((4-hydroxy-1-(4-methylbenzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-(4-methylbenzoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate (61 mg, 0.0884 mmol), TFA (0.44 mL) and DCM (0.88 mL) gave, after purification by flash chromatography and freeze-drying, the title compound (28.7 mg, 54%) as an off-white solid. LCMS (method B): R_{T}=0.86 min, m/z=590, 592 [M+H]⁺. ¹H NMR (500 MHz, DMSO-d₆) δ 8.06 (s, 1H), 7.73 (d, J=7.9 Hz, 1H), 7.41-7.10 (m, 6H), 6.78 (s, 1H), 4.94 (s, 1H), 4.15 (br. s, 1H), 4.06-3.94 (m, 3H), 3.80 (dd, J=10.9, 3.0 Hz, 1H), 3.57 (ddt, J=13.7, 7.5, 3.8 Hz, 1H), 3.42 (br. s, 1H), 3.29-3.18 (m, 2H), 3.14 (br. s, 1H), 2.97 (dd, J=11.7, 2.3 Hz, 1H), 2.57 (t, J=10.9 Hz, 1H), 2.41 (s, 3H), 2.33 (s, 3H), 1.58 (br. s, 2H), 1.44 (br. s, 1H), 1.33 (br. s, 1H), 1.10 (d, J=6.2 Hz, 3H). NH not visible.

### Example 56: 6-Chloro-3-((4-hydroxy-1-(4-(trifluoromethyl)benzol)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

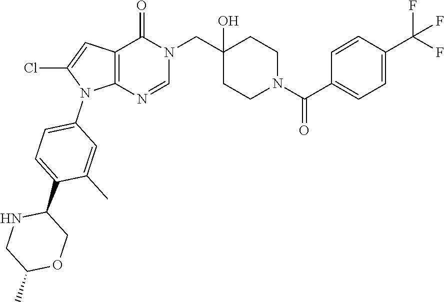 [SMILES: Cc1cc(-n2c(Cl)cc3c(=O)n(CC4(O)CCN(C(=O)c5ccc(C(F)(F)F)cc5)CC4)cnc32)ccc1[C@H]1CO[C@H](C)CN1]

### Step 1: tert-Butyl (2R,5S)-5-(4-(6-chloro-3-((4-hydroxy-1-(4-(trifluoromethyl)benzoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 18 (34 mg, 0.120 mmol), Cs₂CO₃ (71 mg, 0.218 mmol) and DMF (1.1 mL) after 19 h at 80° C. gave the title compound (60 mg, 74%) as a viscous yellow gum. LCMS (method A): R_{T}=1.72 min, m/z=688, 690 [M−butene+H]⁺.

### Step 2: 6-Chloro-3-((4-hydroxy-1-(4-(trifluoromethyl)benzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-(4-(trifluoromethyl)benzoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate (60 mg, 0.0806 mmol), TFA (0.41 mL) and DCM (0.82 mL) gave, after purification by flash chromatography and freeze-drying, the title compound (33.3 mg, 62%) as a white solid. LCMS (method B): R_{T}=0.92 min, m/z=644, 646 [M+H]⁺. ¹H NMR (500 MHz, DMSO-d₆) δ 8.06 (s, 1H), 7.81 (d, J=8.0 Hz, 2H), 7.73 (d, J=8.0 Hz, 1H), 7.61 (d, J=8.0 Hz, 2H), 7.27-7.19 (m, 2H), 6.77 (s, 1H), 4.97 (s, 1H), 4.26-4.15 (m, 1H), 4.06-3.95 (m, 3H), 3.80 (dd, J=10.9, 3.0 Hz, 1H), 3.61-3.53 (m, 1H), 3.30-3.25 (m, 2H), 3.22 (t, J=10.5 Hz, 1H), 3.19-3.10 (m, 1H), 2.97 (dd, J=11.7, 2.3 Hz, 1H), 2.82 (br. s, 1H), 2.58 (t, J=10.9 Hz, 1H), 2.41 (s, 3H), 1.72-1.55 (m, 2H), 1.52-1.42 (m, 1H), 1.38-1.28 (m, 1H), 1.10 (d, J=6.2 Hz, 3H).

### Example 57: 6-Chloro-3-((1-(2-chloro-4-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

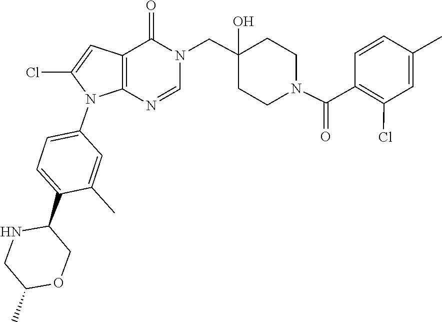 [SMILES: Cc1ccc(C(=O)N2CCC(O)(Cn3cnc4c(cc(Cl)n4-c4ccc([C@H]5CO[C@H](C)CN5)c(C)c4)c3=O)CC2)c(Cl)c1]

### Step 1: tert-Butyl (2R,5S)-5-(4-(6-chloro-3-((1-(2-chloro-4-methylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 19 (31.9 mg, 0.120 mmol), Cs₂CO₃ (71 mg, 0.218 mmol) and DMF (1.1 mL) after 19 h at 80° C. gave the title compound (54.6 mg, 69%) as a yellow glass. LCMS (method A): R_{T}=1.71 min, m/z=668, 670 [M−butene+H]⁺.

### Step 2: 6-Chloro-3-((1-(2-chloro-4-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(2-chloro-4-methylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (54.6 mg, 0.0753 mmol), TFA (0.38 mL) and DCM (0.75 mL) gave, after purification by flash chromatography and freeze-drying, the title compound (30.7 mg, 63%) as an off-white solid. LCMS (method B): R_{T}=0.89 min, m/z=624, 626 [M+H]⁺. ¹H NMR (500 MHz, DMSO-d₆) δ 8.05 (s, 1H), 7.73 (d, J=8.0 Hz, 1H), 7.35 (d, J=10.2 Hz, 1H), 7.32-7.12 (m, 4H), 6.77 (s, 0.5H), 6.77 (s, 0.5H), 4.97 (s, 1H), 4.26-4.16 (m, 1H), 4.09-3.92 (m, 3H), 3.79 (dd, J=10.9, 3.0 Hz, 1H), 3.62-3.53 (m, 1H), 3.29-3.05 (m, 4H), 2.97 (dd, J=11.6, 2.3 Hz, 1H), 2.79 (s, 1H), 2.57 (dd, J=11.7, 10.1 Hz, 1H), 2.41 (s, 3H), 2.33 (s, 3H), 1.66-1.44 (m, 3H), 1.35-1.28 (m, 1H), 1.10 (d, J=6.2 Hz, 3H).

### Example 58: 6-Chloro-3-((1-(2,4-dimethylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

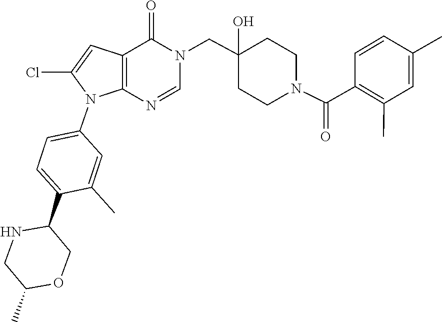 [SMILES: Cc1ccc(C(=O)N2CCC(O)(Cn3cnc4c(cc(Cl)n4-c4ccc([C@H]5CO[C@H](C)CN5)c(C)c4)c3=O)CC2)c(C)c1]

### Step 1: tert-Butyl (2R,5S)-5-(4-(6-chloro-3-((1-(2,4-dimethylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 20 (40.1 mg, 0.163 mmol), Cs₂CO₃ (106 mg, 0.327 mmol) and DMF (2 mL) after 18 h at 80° C. gave the title compound (47 mg, 61%). LCMS (method A): R_{T}=1.55 min, m/z=648, 650 [M−butene+H]⁺.

### Step 2: 6-Chloro-3-((1-(2,4-dimethylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(2,4-dimethylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (47 mg, 0.067 mmol), TFA (1 mL) and DCM (2 mL) gave, after freeze-drying, the title compound (35 mg, 87%). LCMS (method B): R_{T}=0.87 min, m/z=604, 606 [M+H]⁺. ¹H NMR (500 MHz, DMSO-d₆) δ 8.05 (s, 1H), 7.72 (d, J=7.9 Hz, 1H), 7.30-7.18 (m, 2H), 7.14-6.92 (m, 3H), 6.77 (s, 1H), 4.94 (s, 1H), 4.27-4.15 (m, 1H), 4.09-3.90 (m, 3H), 3.80 (dd, J=10.9, 3.0 Hz, 1H), 3.64-3.53 (m, 1H), 3.29-3.07 (m, 4H), 3.03-2.91 (m, 1H), 2.58 (t, J=10.8 Hz, 1H), 2.41 (s, 3H), 2.28 (s, 3H), 2.23-2.08 (m, 3H), 1.66-1.36 (m, 3H), 1.34-1.22 (m, 1H), 1.10 (d, J=6.2 Hz, 3H). NH not visible.

### Example 59: 6-Chloro-3-((1-(2,4-dimethylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

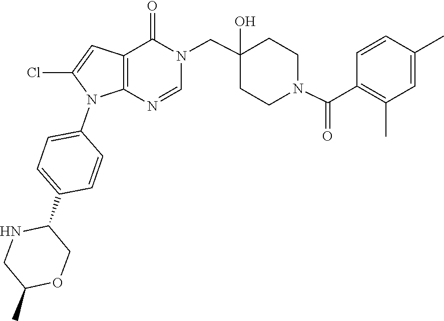 [SMILES: Cc1ccc(C(=O)N2CCC(O)(Cn3cnc4c(cc(Cl)n4-c4ccc([C@@H]5CO[C@@H](C)CN5)cc4)c3=O)CC2)c(C)c1]

### Step 1: tert-Butyl (2S,5R)-5-(4-(6-chloro-3-((1-(2,4-dimethylbenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (50 mg, 0.112 mmol), Epoxide 20 (41.4 mg, 0.169 mmol), Cs₂CO₃ (110 mg, 0.337 mmol) and DMF (2 mL) after 18 h at 80° C. gave the title compound (50 mg, 65%) as white solid. LCMS (method B): R_{T}=1.50 min, m/z=634, 636 [M−butene+H]⁺.

### Step 2: 6-Chloro-3-((1-(2,4-dimethylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(2,4-dimethylbenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (50 mg, 0.0724 mmol), TFA (1 mL) and DCM (2 mL) gave, after freeze-drying, the title compound (29 mg, 67%) as a white solid. LCMS (method B): R_{T}=0.83 min, m/z=590, 592 [M+H]⁺. ¹H NMR (500 MHz, DMSO-d₆) δ 8.05 (s, 1H), 7.64-7.56 (m, 2H), 7.43-7.35 (m, 2H), 7.14-6.95 (m, 3H), 6.78 (s, 1H), 4.94 (s, 1H), 4.27-4.16 (m, 1H), 4.01 (s, 2H), 3.88-3.76 (m, 2H), 3.59-3.50 (m, 1H), 3.25 (t, J=10.3 Hz, 1H), 3.20-3.06 (m, 3H), 3.02-2.81 (m, 2H), 2.57-2.53 (m, 1H), 2.28 (s, 3H), 2.22-2.03 (m, 3H), 1.66-1.40 (m, 3H), 1.32-1.23 (m, 1H), 1.09 (d, J=6.3 Hz, 3H).

### Example 60: 7-(4-(2-Oxa-5-azabicyclo[4.1.0]heptan-4-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

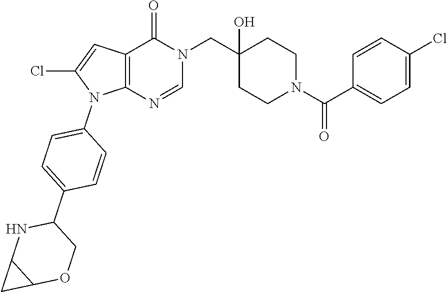 [SMILES: O=C(c1ccc(Cl)cc1)N1CCC(O)(Cn2cnc3c(cc(Cl)n3-c3ccc(C4COC5CC5N4)cc3)c2=O)CC1]

### Step 1: 5-(tert-Butyl) 4-methyl 2-oxa-5-azabicyclo[4.1.0]heptane-4,5-dicarboxylate

A solution of 4-(tert-butyl) 3-methyl 2,3-dihydro-4H-1,4-oxazine-3,4-dicarboxylate (5 g, 20.6 mmol) and diiodomethane (9.91 mL, 123 mmol) in toluene (150 mL) was cooled to −25° C. (bath temperature) before diethylzinc (1 M solution in hexane, 62.0 mL, 62.0 mmol) was added dropwise over 15 min. The reaction was stirred at −25° C. for 8 h before being allowed to warm up slowly overnight. The reaction was quenched with saturated aqueous sodium hydrogen carbonate, the precipitate was removed by filtration, and the layers were separated. The organic phase was washed with brine, dried over Na₂SO₄, filtered, and concentrated under reduced pressure. The residue was purified by flash chromatography (0-100% chloroform:hexane) to give the title compound (2.50 g, 47%) as a mixture of diastereoisomers. LCMS (method C): R_{T}=0.96 min, m/z=202 [M−butene+H]⁺.

### Step 2: 5-(tert-Butoxycarbonyl)-2-oxa-5-azabicyclo[4.1.0]heptane-4-carboxylic acid

To a solution of 5-(tert-butyl) 4-methyl 2-oxa-5-azabicyclo[4.1.0]heptane-4,5-dicarboxylate (1.54 g, 5.98 mmol) in MeOH (50 mL) and water (10 mL) was added sodium hydroxide (717 mg, 17.9 mmol). The resulting mixture was stirred at rt overnight, water (˜40 mL) was added and the mixture was extracted once with MTBE (organic phase was discarded). The aqueous phase was cooled to 0° C. and acidified carefully with 5% aqueous hydrochloric acid before the product was extracted with EtOAc (3×75 mL). The combined organic phases were washed with brine, dried over Na₂SO₄, filtered, and concentrated under reduced pressure to give the title compound (1.05 g, 72%). LCMS (method C): R_{T}=0.96 min, m/z=242 [M−H]⁻. ¹H NMR (400 MHz, Chloroform-d) δ 0.54-0.65 (m, 1H), 0.74-0.85 (m, 1H), 1.44 (s, 9H), 2.57 (s, 1H), 3.54-3.63 (m, 1H), 3.63-3.73 (m, 1H), 3.88-3.99 (m, 1H), 4.05-4.16 (m, 1H).

### Step 3: 5-(tert-Butyl) 4-(4,5,6,7-tetrachloro-1,3-dioxoisoindolin-2-yl) 2-oxa-5-azabicyclo[4.1.0]heptane-4,5-dicarboxylate

General procedure 11 using 5-(tert-butoxycarbonyl)-2-oxa-5-azabicyclo[4.1.0]heptane-4-carboxylic acid (1.05 g, 4.31 mmol), 4,5,6,7-tetrachloro-2-hydroxyisoindoline-1,3-dione (1.43 g, 4.31 mmol), EDC (991 mg, 5.17 mmol) and DMAP (53 mg, 0.431 mmol) gave the title compound (1.91 g, 44%). LCMS (method C): R_{T}=1.39 min, m/z=427, 425 [M−Boc+H]⁺.

### Step 4: tert-Butyl 4-(4-bromophenyl)-2-oxa-5-azabicyclo[4.1.0]heptane-5-carboxylate

To 5-(tert-butyl) 4-(4,5,6,7-tetrachloro-1,3-dioxoisoindolin-2-yl) 2-oxa-5-azabicyclo[4.1.0]heptane-4,5-dicarboxylate (807 mg, 1.53 mmol), (4-bromophenyl)boronic acid (924 mg, 4.60 mmol) in a 100 mL RBF under nitrogen was added 1,4-dioxane (58 mL). The reaction mixture was heated to 75° C. (internal temperature) before triethylamine (2.14 mL, 15.3 mmol) and NiCl₂ 6H₂O: bathophenanthroline complex in DMF (0.05 M, 5.8 mL, 0.290 mmol (Angew. Chem. Int. Ed. 2016, 55, 9676-9679)) were sequentially added. The resulting mixture was stirred at 75° C. overnight. Upon cooling to rt the reaction mixture was diluted with EtOAc (250 mL) and washed with brine. The organic layer was dried over MgSO₄, filtered and concentrated under reduced pressure. The residue was purified by flash chromatography to give the title compound (360 mg, 66%) as a colourless glass. LCMS (method B): R_{T}=1.52 min, m/z=254, 256 [M−Boc+H]⁺.

### Step 5: tert-Butyl 4-(4-(4,4,5,5-tetramethyl-1,3,2-dioxaborolan-2-yl)phenyl)-2-oxa-5-azabicyclo[4.1.0]heptane-5-carboxylate

General procedure 3 (Work-up A) using tert-butyl 4-(4-bromophenyl)-2-oxa-5-azabicyclo[4.1.0]heptane-5-carboxylate (360 mg, 1.02 mmol), B₂Pin₂ (387 mg, 1.52 mmol), Pd(dppf)Cl₂·CH₂Cl₂ (86 mg, 0.102 mmol), KOAc (299 mg, 3.05 mmol) and 1,4-dioxane (10 mL) after stirring at 100° C. (thermal) for 4 h gave the title compound (352 mg, 86%) as a yellow solid. LCMS (method B): R_{T}=1.65-1.68 min (mixture of diastereoisomers), m/z=302 [M−Boc+H]⁺.

### Step 6: tert-Butyl 4-(4-(4,6-dichloro-7H-pyrrolo[2,3-d]pyrimidin-7-yl)phenyl)-2-oxa-5-azabicyclo[4.1.0]heptane-5-carboxylate

General procedure 4 (Work-up A) using 4,6-dichloro-7H-pyrrolo[2,3-d]pyrimidine (165 mg, 0.878 mmol), tert-butyl 4-(4-(4,4,5,5-tetramethyl-1,3,2-dioxaborolan-2-yl)phenyl)-2-oxa-5-azabicyclo[4.1.0]heptane-5-carboxylate (352 mg, 0.878 mmol), copper(II) trifluoroacetate hydrate (508 mg, 1.76 mmol), 1,10-phenanthroline (316 mg, 1.76 mmol), boric acid (217 mg, 3.51 mmol) and DMF (16.5 mL) after 3 days at 50° C. gave the title compound (153 mg, 37%) as a colourless glass. LCMS (method B): R_{T}=1.60 min, m/z=461, 463 [M+H]⁺.

### Step 7: tert-Butyl 4-(4-(6-chloro-4-oxo-3,4-dihydro-7H-pyrrolo[2,3-d]pyrimidin-7-yl)phenyl)-2-oxa-5-azabicyclo[4.1.0]heptane-5-carboxylate

General procedure 5 using tert-butyl 4-(4-(4,6-dichloro-7H-pyrrolo[2,3-d]pyrimidin-7-yl)phenyl)-2-oxa-5-azabicyclo[4.1.0]heptane-5-carboxylate (20 mg, 0.0434 mmol), aqueous sodium hydroxide (4 M, 0.33 mL, 1.30 mmol) and 1,4-dioxane (0.3 mL) after 19 h at 100° C. using 1 M aqueous potassium bisulfate for acidification gave the title compound (26 mg, >100%) as a yellow glass. LCMS (method B): R_{T}=1.17 min, m/z=443, 445 [M+H]⁺. This material was used without further purification.

### Step 8: tert-Butyl 4-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-oxa-5-azabicyclo[4.1.0]heptane-5-carboxylate

General procedure 6 using tert-butyl 4-(4-(6-chloro-4-oxo-3,4-dihydro-7H-pyrrolo[2,3-d]pyrimidin-7-yl)phenyl)-2-oxa-5-azabicyclo[4.1.0]heptane-5-carboxylate (19.2 mg, 0.0434 mmol), Epoxide 2 (16.4 mg, 0.0650 mmol), Cs₂CO₃ (42.4 mg, 0.130 mmol) and DMF (1 mL) after 18 h at 80° C. gave the title compound (14 mg, 46%) as a colourless glass. LCMS (method B): R_{T}=1.44 min, m/z=638, 640 [M−butene+H]⁺.

### Step 9: 7-(4-(2-Oxa-5-azabicyclo[4.1.0]heptan-4-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

General procedure 7 using tert-butyl 4-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-oxa-5-azabicyclo[4.1.0]heptane-5-carboxylate (14 mg, 0.0202 mmol), TFA (1 mL) and DCM (2 mL) gave, after freeze-drying, the title compound (11 mg, 91%) as a white solid. LCMS (method B): R_{T}=0.84 min, m/z=594, 596 [M+H]⁺. ¹H NMR (500 MHz, DMSO-d₆) δ 8.06 (s, 1H), 7.64-7.58 (m, 2H), 7.55-7.49 (m, 2H), 7.45-7.41 (m, 2H), 7.41-7.35 (m, 2H), 6.79 (s, 1H), 4.95 (s, 1H), 4.26-4.11 (m, 1H), 4.10-3.98 (m, 3H), 3.63-3.49 (m, 2H), 3.46-3.33 (m, 1H), 3.30-2.93 (m, 4H), 2.58-2.53 (m, 1H), 1.70-1.28 (m, 4H), 0.81-0.73 (m, 1H), 0.59 (q, J=6.3 Hz, 1H).

### Example 61: 6-Chloro-3-((1-(4-chlorobenzoyl)-4-hydroxypiperidin-4-yl)methyl)-7-(4-((3S,6R)-6-cyclopropylmorpholin-3-yl)phenyl)-3,7-dihydro-4H-pyrrolo[2,3-d]pyrimidin-4-one

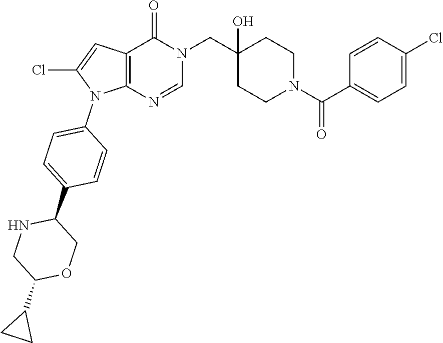 [SMILES: O=C(c1ccc(Cl)cc1)N1CCC(O)(Cn2cnc3c(cc(Cl)n3-c3ccc([C@H]4CO[C@H](C5CC5)CN4)cc3)c2=O)CC1]

### Step 1: rac-(2R,5S)-5-(4-Bromophenyl)-2-cyclopropylmorpholine

General procedure 8 using 4-bromobenzaldehyde (1.37 g, 7.43 mmol), 2-cyclopropyl-2-((tributylstannyl)methoxy)ethan-1-amine (made according to the procedure outlined in Org. Lett. 2014, 16 (4), 1236-1239 starting from 2-amino-1-cyclopropylethan-1-ol) (3.00 g, 7.43 mmol), Cu(OTf)₂ (2.69 g, 7.43 mmol), 2,6-dimethylpyridine (795 mg, 7.43 mmol), HFIP (30 mL), 4 Å molecular sieves (ca. 0.75 g) and DCM (119 mL) gave the title compound (1.23 g, 62%). LCMS (method C): R_{T}=0.93 min, m/z=282, 284 [M+H]⁺. ¹H NMR (500 MHz, Chloroform-d) δ 0.20-0.32 (m, 1H), 0.36-0.45 (m, 1H), 0.47-0.63 (m, 2H), 0.81-0.94 (m, 1H), 2.77-2.86 (m, 1H), 2.87-2.96 (m, 1H), 3.10-3.22 (m, 1H), 3.29-3.39 (m, 1H), 3.79-3.91 (m, 2H), 7.25 (d, J=8.1 Hz, 2H), 7.44 (d, J=8.3 Hz, 2H).

### Step 2: rac-tert-Butyl (2R,5S)-5-(4-bromophenyl)-2-cyclopropylmorpholine-4-carboxylate

General procedure 9 using rac-(2R,5S)-5-(4-Bromophenyl)-2-cyclopropylmorpholine (1.23 g, 4.61 mmol) and Boc₂O (1.21 g, 5.53 mmol) gave the title compound (1.46 g, 83%). LCMS (method C): R_{T}=1.63 min, m/z=282, 284 [M−Boc+H]⁺. ¹H NMR (500 MHz, Chloroform-d) δ 0.27 (dt, J=9.7, 4.6 Hz, 1H), 0.41 (dt, J=8.7, 4.9 Hz, 1H), 0.60 (ddt, J=13.6, 8.8, 4.4 Hz, 2H), 1.17-1.24 (m, 1H), 1.35 (s, 9H), 2.94-3.02 (m, 1H), 3.47-3.54 (m, 1H), 3.64-3.71 (m, 1H), 3.73-3.81 (m, 1H), 4.09-4.18 (m, 1H), 4.81-4.87 (m, 1H), 7.25 (d, J=10.1 Hz, 2H), 7.44 (d, J=8.4 Hz, 2H).

### Step 3: rac-tert-Butyl (2R,5S)-2-cyclopropyl-5-(4-(4,4,5,5-tetramethyl-1,3,2-dioxaborolan-2-yl)phenyl)morpholine-4-carboxylate

General procedure 3 (Work-up B) using rac-tert-butyl (2R,5S)-5-(4-bromophenyl)-2-cyclopropylmorpholine-4-carboxylate (765 mg, 2.00 mmol), B₂Pin₂ (762 mg, 3.00 mmol), Pd(dppf)Cl₂·CH₂Cl₂ (169 mg, 0.200 mmol), KOAc (589 mg, 6.00 mmol) and 1,4-dioxane (20 mL) after stirring at 90° C. (thermal) for 1 h gave the title compound (839 mg, 97%) as a yellow semi-solid. LCMS (method A): R_{T}=2.03 min, m/z=374 [M−Butene+H]⁺. ¹H NMR (500 MHz, DMSO-d₆) δ 7.64 (d, J=8.1 Hz, 2H), 7.32 (d, J=7.9 Hz, 2H), 4.83 (t, J=5.2 Hz, 1H), 4.03 (dd, J=12.2, 4.7 Hz, 1H), 3.72 (dd, J=12.2, 5.6 Hz, 1H), 3.62 (dd, J=13.5, 4.0 Hz, 1H), 3.44 (dd, J=13.5, 4.3 Hz, 1H), 3.05 (dt, J=8.4, 4.1 Hz, 1H), 1.29 (s, 15H), 1.17 (s, 6H), 1.15-1.09 (m, 1H), 0.55-0.45 (m, 2H), 0.31-0.22 (m, 2H).

### Step 4: rac-tert-Butyl (2R,5S)-2-cyclopropyl-5-(4-(4,6-dichloro-7H-pyrrolo[2,3-d]pyrimidin-7-yl)phenyl)morpholine-4-carboxylate

General procedure 4 (Work-up B) using 4,6-dichloro-7H-pyrrolo[2,3-d]pyrimidine (283 mg, 1.51 mmol), rac-tert-butyl (2R,5S)-2-cyclopropyl-5-(4-(4,4,5,5-tetramethyl-1,3,2-dioxaborolan-2-yl)phenyl)morpholine-4-carboxylate (840 mg, 1.96 mmol), copper(II) trifluoroacetate hydrate (872 mg, 3.01 mmol), 1,10-phenanthroline (543 mg, 3.01 mmol), boric acid (242 mg, 3.91 mmol) and DMF (15.1 mL) after 3 days at 50° C. gave the title compound (383 mg, 51%) as a white foam. LCMS (method A): R_{T}=1.98 min, m/z=489, 491 [M+H]⁺. ¹H NMR (500 MHz, DMSO-d₆) δ 8.62 (s, 1H), 7.58-7.54 (m, 2H), 7.54-7.49 (m, 2H), 7.10 (s, 1H), 4.95 (t, J=5.2 Hz, 1H), 4.13 (dd, J=12.2, 4.7 Hz, 1H), 3.81 (dd, J=12.2, 5.7 Hz, 1H), 3.65 (dd, J=13.6, 4.3 Hz, 1H), 3.55 (dd, J=13.6, 4.3 Hz, 1H), 3.13 (dt, J=8.5, 4.2 Hz, 1H), 1.32 (s, 9H), 1.20-1.11 (m, 1H), 0.57-0.47 (m, 2H), 0.35-0.26 (m, 2H).

### Step 5: tert-Butyl (2S,5R)-2-cyclopropyl-5-(4-(4,6-dichloro-7H-pyrrolo[2,3-d]pyrimidin-7-yl)phenyl)morpholine-4-carboxylate and tert-butyl (2R,5S)-2-cyclopropyl-5-(4-(4,6-dichloro-7H-pyrrolo[2,3-d]pyrimidin-7-yl)phenyl)morpholine-4-carboxylate

rac-tert-Butyl (2R,5S)-2-cyclopropyl-5-(4-(4,6-dichloro-7H-pyrrolo[2,3-d]pyrimidin-7-yl)phenyl)morpholine-4-carboxylate (641 mg, 1.31 mmol) was resolved into the single stereoisomers by chiral HPLC using a Lux iC5 (21.2 mm×250 mm, 5 μm) column with isocratic solvent conditions: 3:2 hexane/EtOH (0.2% v/v NH₃). The first eluted material was arbitrarily assigned as tert-butyl (2S,5R)-2-cyclopropyl-5-(4-(4,6-dichloro-7H-pyrrolo[2,3-d]pyrimidin-7-yl)phenyl)morpholine-4-carboxylate (226 mg, 35% recovery). Chiral purity (method G): R_{T}=3.83 min, 100% ee. The second eluted material was arbitrarily assigned as tert-butyl (2R,5S)-2-cyclopropyl-5-(4-(4,6-dichloro-7H-pyrrolo[2,3-d]pyrimidin-7-yl)phenyl)morpholine-4-carboxylate (265.6 mg, 41% recovery). Chiral purity (method G): R_{T}=2.61 min, 97.8% ee.

### Step 6: tert-Butyl (2R,5S)-5-(4-(6-chloro-4-oxo-3,4-dihydro-7H-pyrrolo[2,3-d]pyrimidin-7-yl)phenyl)-2-cyclopropylmorpholine-4-carboxylate

General procedure 5 using tert-butyl (2R,5S)-2-cyclopropyl-5-(4-(4,6-dichloro-7H-pyrrolo[2,3-d]pyrimidin-7-yl)phenyl)morpholine-4-carboxylate (40 mg, 0.0817 mmol), aqueous sodium hydroxide (4 M, 0.61 mL, 2.45 mmol) and 1,4-dioxane (0.61 mL) after 18 h at 100° C. using 1 M aqueous potassium bisulfate for acidification gave the title compound (38 mg, 98%) as a yellow glass. LCMS (method B): R_{T}=1.32 min, m/z=471, 473 [M+H]⁺.

### Step 7: tert-Butyl (2R,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-cyclopropylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-cyclopropylmorpholine-4-carboxylate (38.5 mg, 0.0817 mmol), Epoxide 2 (30.9 mg, 0.123 mmol), Cs₂CO₃ (79.9 mg, 0.245 mmol) and DMF (2 mL) after 18 h at 80° C. gave the title compound (31 mg, 52%) as a colourless glass. LCMS (method B): R_{T}=1.56 min, m/z=666, 668 [M−butene+H]⁺.

### Step 8: 6-Chloro-3-((1-(4-chlorobenzoyl)-4-hydroxypiperidin-4-yl)methyl)-7-(4-((3S,6R)-6-cyclopropylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-cyclopropylmorpholine-4-carboxylate (31 mg, 0.0429 mmol), TFA (1 mL) and DCM (2 mL) gave, after freeze-drying, the title compound (16 mg, 97%) as a white solid. LCMS (method B): R_{T}=0.88 min, m/z=622, 624 [M+H]⁺. ¹H NMR (500 MHz, DMSO-d₆) δ 8.07 (s, 1H), 7.64-7.57 (m, 2H), 7.55-7.48 (m, 2H), 7.46-7.35 (m, 4H), 6.80 (s, 1H), 4.95 (s, 1H), 4.28-4.12 (m, 1H), 4.03 (s, 2H), 3.97-3.77 (m, 2H), 3.46-3.34 (m, 1H), 3.31-2.97 (m, 5H), 2.90-2.80 (m, 1H), 2.80-2.68 (m, 1H), 1.73-1.25 (m, 4H), 0.91-0.77 (m, 1H), 0.51-0.38 (m, 2H), 0.37-0.16 (m, 2H).

### Example 62: 6-Chloro-3-((4-hydroxy-1-((1 S,2S)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one or 6-chloro-3-((4-hydroxy-1-((1R,2R)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

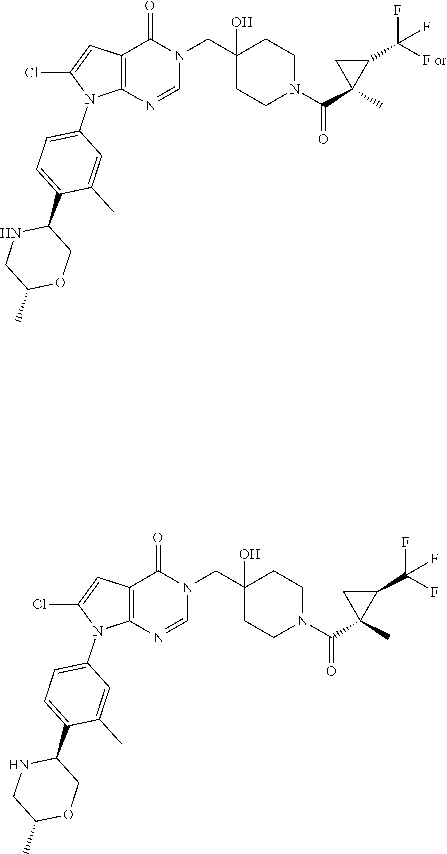

### Step 1: tert-Butyl (2R,5S)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)-2-methylphenyl)-2-methylmorpholine-4-carboxylate or tert-butyl (2R,5S)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (40 mg, 0.0872 mmol), Epoxide 9 (34.4 mg, 0.131 mmol), Cs₂CO₃ (85.2 mg, 0.262 mmol) and DMF (1 mL) after 18 h at 80° C. gave the title compound (43 mg, 68%) as a colourless glass. LCMS (method B): R_{T}=1.52 min, m/z=666, 668 [M−butene+H]⁺.

### Step 2: 6-Chloro-3-((4-hydroxy-1-((1S,2S)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one or 6-chloro-3-((4-hydroxy-1-((1R,2R)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-((1 S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)-2-methylphenyl)-2-methylmorpholine-4-carboxylate or tert-butyl (2R,5S)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)-2-methylphenyl)-2-methylmorpholine-4-carboxylate (43 mg, 0.0595 mmol), TFA (1 mL) and DCM (2 mL) gave, after freeze-drying, the title compound (37 mg, 99%) as a white solid. LCMS (method B): R_{T}=0.90 min, m/z=622, 624 [M+H]⁺. ¹H NMR (500 MHz, DMSO-d₆) δ 8.07 (s, 1H), 7.74 (d, J=8.0 Hz, 1H), 7.26 (d, J=9.7 Hz, 2H), 6.80 (s, 1H), 4.94 (s, 1H), 4.18-3.75 (m, 6H), 3.68-3.52 (m, 1H), 3.50-3.33 (m, 1H), 3.30-2.73 (m, 4H), 2.64-2.56 (m, 1H), 2.43 (s, 3H), 2.21-2.03 (m, 1H), 1.79-1.28 (m, 8H), 1.19-1.04 (m, 4H).

### Example 63: 6-Chloro-3-((1-cinnamoyl-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

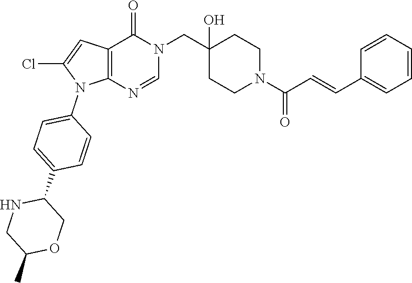 [SMILES: C[C@H]1CN[C@H](c2ccc(-n3c(Cl)cc4c(=O)n(CC5(O)CCN(C(=O)/C=C/c6ccccc6)CC5)cnc43)cc2)CO1]

### Step 1: tert-Butyl (2S,5R)-5-(4-(6-chloro-3-((4-hydroxy-1-((4-nitrophenyl)sulfonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (40 mg, 0.0899 mmol), Epoxide 21 (40.2 mg, 0.135 mmol), Cs₂CO₃ (88 mg, 0.270 mmol) and DMF (1.5 mL) after 18 h at 80° C. gave the title compound (36 mg, 53%) as a yellow glass. LCMS (method A): R_{T}=1.53 min, m/z=743, 745 [M+H]⁺.

### Step 2: tert-Butyl (2S,5R)-5-(4-(6-chloro-3-((4-hydroxypiperidin-4-yl)methyl)-4-oxo-3,4-dihydro-7H-pyrrolo[2,3-d]pyrimidin-7-yl)phenyl)-2-methylmorpholine-4-carboxylate

To tert-butyl (2S,5R)-5-(4-(6-chloro-3-((4-hydroxy-1-((4-nitrophenyl)sulfonyl)piperidin-4-yl)methyl)-4-oxo-3,4-dihydro-7H-pyrrolo[2,3-d]pyrimidin-7-yl)phenyl)-2-methylmorpholine-4-carboxylate (36 mg, 0.0484 mmol) in DMF (2 mL) was added thiophenol (25 μL, 0.242 mmol) and K₂CO₃ (33.5 mg, 0.242 mmol). The reaction mixture was stirred at rt for 19 h and at 50° C. for 6 h. Additional thiophenol (25 μL, 0.242 mmol) and K₂CO₃ (33.5 mg, 0.242 mmol) were added and the reaction mixture was stirred at 50° C. for 19 h. Upon cooling to rt reaction mixture was diluted with DCM (5 mL) and injected onto a 5 g SCX-2 cartridge pre-equilibrated with DCM. The cartridge was washed with a 4:1 mixture of DCM/MeOH and the basic compound was eluted using a 4:1 mixture of DCM/7 M NH₃ in MeOH. The ammoniacal fractions were concentrated under reduced pressure to give the title compound (20 mg, 74%) as a yellow glass. LCMS (method B): R_{T}=1.00 min, m/z=558, 560 [M+H]⁺.

### Step 3: tert-Butyl (2S,5R)-5-(4-(6-chloro-3-((1-cinnamoyl-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 1 using tert-butyl (2S,5R)-5-(4-(6-chloro-3-((4-hydroxypiperidin-4-yl)methyl)-4-oxo-3,4-dihydro-7H-pyrrolo[2,3-d]pyrimidin-7-yl)phenyl)-2-methylmorpholine-4-carboxylate (20 mg, 0.0358 mmol), cinnamic acid (8 mg, 0.0538 mmol), HATU (20.4 mg, 0.0538 mmol), DIPEA (19 μL, 0.108 mmol) and DCM (2 mL) after 19 h gave the title compound (16 mg, 64%) as a colourless oil. LCMS (method b): R_{T}=1.48 min, m/z=632, 634 [M−butene+H]⁺.

### Step 4: 6-Chloro-3-((1-cinnamoyl-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-cinnamoyl-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (16 mg, 0.0232 mmol), TFA (1 mL) and DCM (2 mL) gave, after freeze-drying, the title compound (13 mg, 95%) as a white solid. LCMS (method B): R_{T}=0.88 min, m/z=588, 590 [M+H]⁺. ¹H NMR (500 MHz, DMSO-d₆) δ 8.08 (s, 1H), 7.74-7.69 (m, 2H), 7.65-7.57 (m, 2H), 7.47 (d, J=15.4 Hz, 1H), 7.44-7.35 (m, 5H), 7.28 (d, J=15.4 Hz, 1H), 6.80 (s, 1H), 4.96 (s, 1H), 4.24-3.93 (m, 4H), 3.91-3.76 (m, 2H), 3.62-3.51 (m, 1H), 3.47-3.34 (m, 1H), 3.30-2.77 (m, 4H), 2.59-2.53 (m, 1H), 1.67-1.37 (m, 4H), 1.10 (d, J=6.2 Hz, 3H).

### Example 64: 6-Chloro-3-((1-(4-chlorobenzoyl)-4-hydroxypiperidin-4-yl)methyl)-7-(4-((3R,6S)-6-cyclopropylmorpholin-3-yl)phenyl)-3,7-dihydro-4H-pyrrolo[2,3-d]pyrimidin-4-one

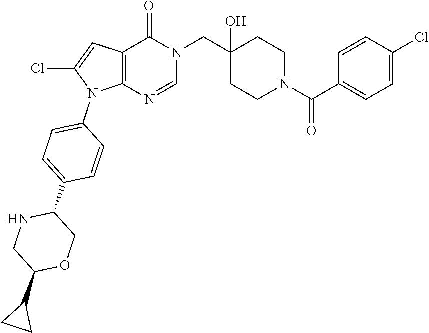 [SMILES: O=C(c1ccc(Cl)cc1)N1CCC(O)(Cn2cnc3c(cc(Cl)n3-c3ccc([C@@H]4CO[C@@H](C5CC5)CN4)cc3)c2=O)CC1]

### Step 1: tert-Butyl (2S,5R)-5-(4-(6-chloro-4-oxo-3,4-dihydro-7H-pyrrolo[2,3-d]pyrimidin-7-yl)phenyl)-2-cyclopropylmorpholine-4-carboxylate

General procedure 5 using tert-butyl (2S,5R)-2-cyclopropyl-5-(4-(4,6-dichloro-7H-pyrrolo[2,3-d]pyrimidin-7-yl)phenyl)morpholine-4-carboxylate (50 mg, 0.102 mmol), aqueous sodium hydroxide (4 M, 0.77 mL, 3.07 mmol) and 1,4-dioxane (0.61 mL) after 18 h at 100° C. using 1 M aqueous potassium bisulfate for acidification gave the title compound (38 mg, 79%) as a yellow glass. LCMS (method B): R_{T}=1.32 min, m/z=471, 473 [M+H]⁺.

### Step 2: tert-Butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-cyclopropylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-cyclopropylmorpholine-4-carboxylate (38 mg, 0.081 mmol), Epoxide 2 (30.5 mg, 0.121 mmol), Cs₂CO₃ (79 mg, 0.242 mmol) and DMF (2 mL) after 18 h at 80° C. gave the title compound (39 mg, 67%) as a colourless glass. LCMS (method A): R_{T}=1.56 min, m/z=666, 668 [M−butene+H]⁺.

### Step 3: 6-Chloro-3-((1-(4-chlorobenzoyl)-4-hydroxypiperidin-4-yl)methyl)-7-(4-((3R,6S)-6-cyclopropylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-cyclopropylmorpholine-4-carboxylate (39.0 mg, 0.0540 mmol), TFA (1 mL) and DCM (2 mL) gave, after purification by flash chromatography and freeze-drying, the title compound (20 mg, 59%) as a white solid. LCMS (method B): R_{T}=0.91 min, m/z=622, 624 [M+H]⁺. ¹H NMR (500 MHz, DMSO-d₆) δ 8.06 (s, 1H), 7.62-7.54 (m, 2H), 7.54-7.45 (m, 2H), 7.45-7.31 (m, 4H), 6.79 (s, 1H), 4.95 (s, 1H), 4.22-4.12 (m, 1H), 4.02 (s, 2H), 3.89-3.77 (m, 2H), 3.43-3.07 (m, 5H (signals overlap with HDO)), 3.03 (dd, J=11.5, 2.2 Hz, 1H), 2.82 (ddd, J=10.1, 7.8, 2.1 Hz, 1H), 2.71 (t, J=10.9 Hz, 1H), 1.66-1.55 (m, 2H), 1.51-1.43 (m, 1H), 1.37-1.28 (m, 1H), 0.87-0.78 (m, 1H), 0.48-0.39 (m, 2H), 0.34-0.21 (m, 2H).

### Example 65: 6-Chloro-3-((4-hydroxy-1-(4-(trifluoromethyl)benzoyl)Piperidin-4-yl)methyl)-7-(4-((3R,6S)-6-methylmorpholin-3-yl)phenyl)-3,7-dihydro-4H-pyrrolo[2,3-d]pyrimidin-4-one

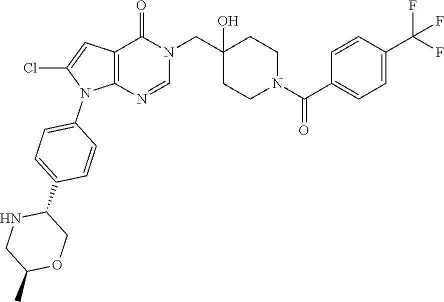 [SMILES: C[C@H]1CN[C@H](c2ccc(-n3c(Cl)cc4c(=O)n(CC5(O)CCN(C(=O)c6ccc(C(F)(F)F)cc6)CC5)cnc43)cc2)CO1]

### Step 1: tert-Butyl (2S,5R)-5-(4-(3-((1-(tert-butoxycarbonyl)-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (25 mg, 0.0562 mmol), tert-butyl 1-oxa-6-azaspiro[2.5]octane-6-carboxylate (14.4 mg, 0.0674 mmol), Cs₂CO₃ (36.6 mg, 0.112 mmol) and DMF (0.56 mL) after 18 h at 80° C. gave the title compound (40 mg, >100%) as a viscous yellow gum. LCMS (method A): R_{T}=1.71 min, m/z=658, 660 [M+H]⁺.

### Step 2: 6-Chloro-3-((4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(3-((1-(tert-butoxycarbonyl)-4-hydroxypiperidin-4-yl)methyl)-6-chloro-4-oxo-3,4-dihydro-7H-pyrrolo[2,3-d]pyrimidin-7-yl)phenyl)-2-methylmorpholine-4-carboxylate (40 mg, 0.0608 mmol), TFA (0.3 mL) and DCM (0.6 mL) gave the title compound (22.9 mg, 82%) as a yellow glass. LCMS (method B): R_{T}=0.47 min, m/z=458, 460 [M+H]⁺.

### Step 3: 6-Chloro-3-((4-hydroxy-1-(4-(trifluoromethyl)benzoyl)piperidin-4-yl)methyl)-7-(4-((3R,6S)-6-methylmorpholin-3-yl)phenyl)-3,7-dihydro-4H-pyrrolo[2,3-d]pyrimidin-4-one

To a mixture of 4-(trifluoromethyl)benzoic acid (9.5 mg, 0.0500 mmol) and HATU (19 mg, 0.0500 mmol) in DMF (1 mL) at rt in a 5 mL RBF was added DIPEA (26.1 μL, 0.150 mmol). After stirring at rt for 10 min the reaction mixture taken up in a syringe and added dropwise to a solution of 6-chloro-3-((4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one (22.9 mg, 0.0500 mmol) in DMF (0.5 mL) at 0° C. After stirring at 0° C. for 20 min the reaction was quenched by the addition of saturated aqueous sodium hydrogen carbonate (5 mL). Upon warming to rt the reaction mixture was further diluted with saturated aqueous sodium hydrogen carbonate (10 mL) and the resulting mixture was extracted with DCM (3×10 mL) using an ISOLUTE® phase separator. The combined organic layers were concentrated under reduced pressure and the residue was purified by flash chromatography to give the title compound (10 mg, 30%) as a white solid after lyophilisation. LCMS (method B): R_{T}=0.93 min, m/z=630, 632 [M+H]⁺. ¹H NMR (500 MHz, DMSO-d₆) δ 8.06 (s, 1H), 7.81 (d, J=8.1 Hz, 2H), 7.67-7.52 (m, 4H), 7.39 (d, J=8.1 Hz, 2H), 6.79 (s, 1H), 4.96 (s, 1H), 4.27-4.15 (m, 1H), 4.02 (s, 2H), 3.89-3.75 (m, 2H), 3.60-3.49 (m, 1H), 3.30-3.22 (m, 3H (signals overlap with HDO)), 3.19-3.08 (m, 1H), 2.96 (d, J=11.7 Hz, 1H), 2.89 (br. s, 1H), 2.59-2.53 (m, 1H (signals overlap with DMSO)), 1.71-1.54 (m, 2H), 1.54-1.44 (m, 1H), 1.37-1.29 (m, 1H), 1.09 (d, J=6.1 Hz, 3H).

### Example 66: 6-Chloro-7-(4-((3R,6S)-6-ethylmorpholin-3-yl)phenyl)-3-((4-hydroxy-1-((1 S,2S)-1-methyl-2-(trifluoromethyl)cyclopropane-1-carbonyl)piperidin-4-yl)methyl)-3,7-dihydro-4H-pyrrolo[2,3-d]pyrimidin-4-one or 6-chloro-7-(4-((3R,6S)-6-ethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one

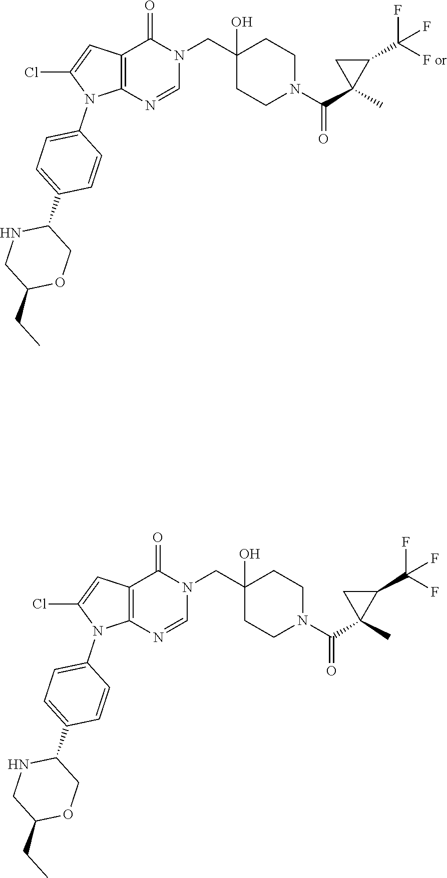

### Step 1: tert-Butyl (2S,5R)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate or tert-butyl (2S,5R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-ethylmorpholine-4-carboxylate (73 mg, 0.159 mmol), Epoxide 9 (46.1 mg, 0.175 mmol), Cs₂CO₃ (104 mg, 0.318 mmol) and DMF (1.6 mL) after 18 h at 80° C. gave the title compound (91.1 mg, 79.3%) as a viscous yellow oil. LCMS (method B): R_{T}=1.56 min, m/z=666, 668 [M−butene+H]⁺.

### Step 2: 6-Chloro-7-(4-((3R,6S)-6-ethylmorpholin-3-yl)phenyl)-3-((4-hydroxy-1-((1S,2S)-1-methyl-2-(trifluoromethyl)cyclopropane-1-carbonyl)piperidin-4-yl)methyl)-3,7-dihydro-4H-pyrrolo[2,3-d]pyrimidin-4-one or 6-chloro-7-(4-((3R,6S)-6-ethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one

General procedure 7 using tert-butyl (2S,5R)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate or tert-butyl (2S,5R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate (91.1 mg, 0.126 mmol), TFA (0.63 mL) and DCM (1.26 mL) gave, after purification by flash chromatography and freeze-drying, the title compound (48.3 mg, 60%) as a very slightly off-white solid. LCMS (method B): R_{T}=0.88 min, m/z=622, 624 [M+H]⁺. ¹H NMR (500 MHz, DMSO-d₆) δ 8.06 (s, 1H), 7.63-7.58 (m, 2H), 7.42-7.36 (m, 2H), 6.81 (s, 1H), 4.93 (s, 1H), 4.20-3.64 (m, 7H), 3.51-3.21 (m, 2H (signals overlap with HDO)), 3.14-2.80 (m, 2H), 2.58-2.52 (m, 1H), 2.18-2.05 (m, 1H), 1.79-1.35 (m, 8H), 1.34 (s, 3H), 1.16-1.05 (m, 1H), 0.91 (t, J=7.5 Hz, 3H).

### Example 67: 6-Chloro-7-(4-((R)-6,6-dimethylmorpholin-3-yl)phenyl)-3-((4-hydroxy-1-((1 S,2S)-1-methyl-2-(trifluoromethyl)cyclopropane-1-carbonyl)piperidin-4-yl)methyl)-3,7-dihydro-4H-pyrrolo[2,3-d]pyrimidin-4-one, 6-chloro-7-(4-((S)-6,6-dimethylmorpholin-3-yl)phenyl)-3-((4-hydroxy-1-((1 S,2S)-1-methyl-2-(trifluoromethyl)cyclopropane-1-carbonyl)piperidin-4-yl)methyl)-3,7-dihydro-4H-pyrrolo[2,3-d]pyrimidin-4-one, 6-chloro-7-(4-((R)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one or 6-chloro-7-(4-((S)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one

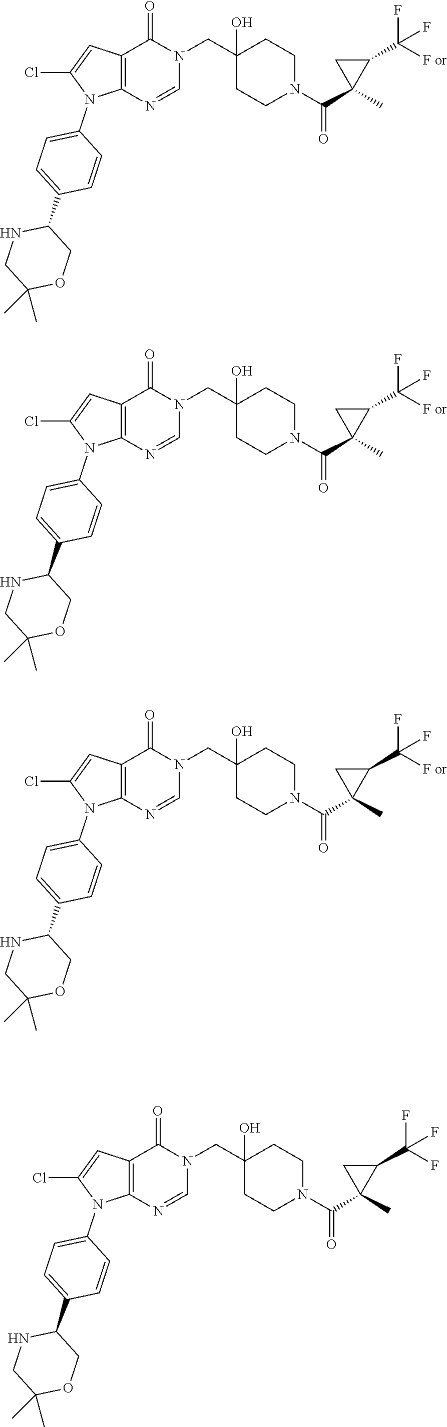

### Step 1: tert-Butyl (R)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate, tert-butyl (S)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate, tert-butyl (R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate or tert-butyl (S)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate

General procedure 6 using tert-butyl (R)-5-(4-(6-chloro-4-oxo-3,4-dihydro-7H-pyrrolo[2,3-d]pyrimidin-7-yl)phenyl)-2,2-dimethylmorpholine-4-carboxylate (67 mg, 0.146 mmol), Epoxide 9 (42.3 mg, 0.161 mmol), Cs₂CO₃ (95.1 mg, 0.292 mmol) and DMF (1.46 mL) after 18 h at 80° C. gave the title compound (113 mg, >100%) as a viscous yellow oil. LCMS (method B): R_{T}=1.54 min, m/z=666, 668 [M−butene+H]⁺.

### Step: 2: 6-Chloro-7-(4-((R)-6,6-dimethylmorpholin-3-yl)phenyl)-3-((4-hydroxy-1-((1S,2S)-1-methyl-2-(trifluoromethyl)cyclopropane-1-carbonyl)piperidin-4-yl)methyl)-3,7-dihydro-4H-pyrrolo[2,3-d]pyrimidin-4-one, 6-chloro-7-(4-((S)-6,6-dimethylmorpholin-3-yl)phenyl)-3-((4-hydroxy-1-((1S,2S)-1-methyl-2-(trifluoromethyl)cyclopropane-1-carbonyl)piperidin-4-yl)methyl)-3,7-dihydro-4H-pyrrolo[2,3-d]pyrimidin-4-one, 6-chloro-7-(4-((R)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one or 6-chloro-7-(4-((S)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one

General procedure 7 using tert-butyl (R)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate, tert-butyl (S)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate, tert-butyl (R)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate or tert-butyl (S)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate (113 mg, 0.157 mmol), TFA (0.78 mL) and DCM (1.56 mL) gave, after purification by flash chromatography and freeze-drying, the title compound (49.7 mg, 49%) as an off-white solid. LCMS (method B): R_{T}=0.85 min, m/z=622, 624 [M+H]⁺. ¹H NMR (500 MHz, DMSO-d₆) δ 8.07 (s, 1H), 7.64 (d, J=8.4 Hz, 2H), 7.40 (d, J=8.4 Hz, 2H), 6.81 (s, 1H), 4.93 (s, 1H), 4.16-3.66 (m, 6H), 3.57 (dd, J=11.1, 3.6 Hz, 1H), 3.46 (t, J=10.7 Hz, 1H), 3.41-3.32 (m, 1H (signals overlap with HDO)), 2.96 (s, 1H), 2.77 (d, J=11.6 Hz, 1H), 2.68 (d, J=11.6 Hz, 1H), 2.19-2.04 (m, 1H), 1.74-1.37 (m, 5H), 1.36 (s, 3H), 1.34 (s, 3H), 1.13 (s, 3H), 1.12-1.07 (m, 1H).

### Example 68: 6-Chloro-3-((1-(4-chlorobenzoyl)-4-hydroxypiperidin-4-yl)methyl)-7-(4-((3S,6S)-6-methylmorpholin-3-yl)phenyl)-3,7-dihydro-4H-pyrrolo[2,3-d]pyrimidin-4-one

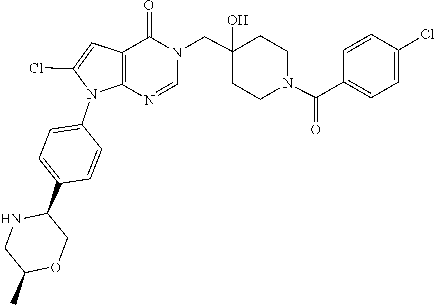 [SMILES: C[C@H]1CN[C@@H](c2ccc(-n3c(Cl)cc4c(=O)n(CC5(O)CCN(C(=O)c6ccc(Cl)cc6)CC5)cnc43)cc2)CO1]

### Step 1: N-(1-(4-Bromophenyl)-2-hydroxyethyl)-2-chloropropanamide

To a stirred suspension of 2-amino-2-(4-bromophenyl)ethan-1-ol hydrochloride (15.0 g, 59.4 mmol) in 1,4-dioxane (200 mL) was added NEt₃ (18.0 mL, 131 mmol) and the resulting mixture was cooled to 0° C. before 2-chloropropanoyl chloride (7.54 g, 59.4 mmol) was added dropwise over 20 min. The reaction mixture was stirred at rt 2 h before the volatiles were removed under reduced pressure. The residue was transferred to a separating funnel using water and EtOAc and the resulting mixture was extracted with EtOAc (3×100 mL). The combined organic layers were dried over Na₂SO₄, filtered and concentrated under reduced pressure. The residue was purified by flash chromatography (gradient 0-50% MTBE in chloroform) to give the title compound (11.9 g, 65%). LCMS (method C): R_{T}=1.09 min, m/z=306, 308 [M+H]⁺. ¹H NMR (400 MHz, DMSO-d₆) δ 1.46-1.55 (m, 3H), 3.54-3.60 (m, 2H), 4.53-4.66 (m, 1H), 4.74-4.82 (m, 1H), 7.21-7.29 (m, 2H), 7.45-7.54 (m, 2H), 8.56-8.68 (m, 1H).

### Step 2: rac-(2R,5R)-5-(4-Bromophenyl)-2-methylmorpholin-3-one

To a stirred solution of N-(1-(4-bromophenyl)-2-hydroxyethyl)-2-chloropropanamide (9.92 g, 32.4 mmol) in 2-methyl-2-propanol (90 mL) at rt was dropwise added a solution of potassium tert-butoxide (7.26 g, 64.7 mmol) in 2-methyl-2-propanol (150 mL). After stirring at rt for 1 h the reaction mixture was acidified to ˜pH 3 by the addition of 1 M aqueous hydrochloric acid and the resulting solution was concentrated under reduced pressure to remove most of 2-methyl-2-propanol. The resulting mixture was extracted with EtOAc (3×100 mL), the combined organic layers were dried over Na₂SO₄, filtered, and concentrated under reduced pressure to give the title compound (8.20 g, 94%). LCMS (method C): R_{T}=1.11 min, m/z=270, 272 [M+H]⁺. ¹H NMR (400 MHz, DMSO-d₆) δ 1.32 (d, J=6.9 Hz, 3H), 3.72 (d, J=11.8 Hz, 1H), 3.96 (dt, J=11.9, 2.9 Hz, 1H), 4.15 (d, J=7.3 Hz, 1H), 4.53 (s, 1H), 7.26 (d, J=7.5 Hz, 2H), 7.56 (d, J=7.8 Hz, 2H), 8.35 (s, 1H).

### Step 3: rac-(2R,5R)-5-(4-Bromophenyl)-2-methylmorpholine

A solution of rac-(2R,5R)-5-(4-bromophenyl)-2-methylmorpholin-3-one (6.20 g, 23.0 mmol) in THE (155 mL) was added dropwise to sodium bis(2-methoxyethoxy)aluminium hydride (63% in toluene, 33.12 g of solution, 103 mmol) at 0° C. Upon warming to rt the reaction was stirred at rt for 19 h before being quenched by the addition of 2 M aqueous sodium hydroxide (200 mL). The resulting mixture was extracted with MTBE (3×200 mL), the combined organic layers were dried over Na₂SO₄, filtered, and concentrated under reduced pressure. The residue was purified by flash chromatography (5:1 chloroform:MTBE) to give title compound (1 g, 17%). LCMS (method C): R_{T}=0.84 min, m/z=256, 258 [M+H]⁺.

### Step 4: rac-tert-Butyl (2R,5R)-5-(4-bromophenyl)-2-methylmorpholine-4-carboxylate

General procedure 9 using rac-(2R,5R)-5-(4-Bromophenyl)-2-methylmorpholine (1.54 g, 6.02 mmol) and Boc₂O (1.58 g, 7.22 mmol) gave the title compound (2.44 g, 67%). LCMS (method C): R_{T}=1.55 min, m/z=300, 302 [M−butene+H]⁺. ¹H NMR (500 MHz, Chloroform-d) 6 1.14 (d, J=6.2 Hz, 3H), 1.46 (s, 9H), 3.48-3.78 (m, 2H), 3.82-3.98 (m, 2H), 4.25-4.39 (m, 1H), 4.75-5.18 (m, 1H), 7.33 (d, J=7.3 Hz, 2H), 7.44 (d, J=7.3 Hz, 2H).

### Step 5: rac-tert-Butyl (2R,5R)-2-methyl-5-(4-(4,4,5,5-tetramethyl-1,3,2-dioxaborolan-2-yl)phenyl)morpholine-4-carboxylate

General procedure 3 (Work-up A) using rac-tert-butyl (2R,5R)-5-(4-bromophenyl)-2-methylmorpholine-4-carboxylate (3.89 g, 10.9 mmol), B₂Pin₂ (4.16 g, 16.4 mmol), Pd(dppf)Cl₂·CH₂Cl₂ (925 mg, 1.09 mmol), KOAc (3.21 g, 32.8 mmol) and 1,4-dioxane (110 mL) after stirring at 100° C. (thermal) for 4 h gave the title compound (2.97 g, 67%) as a yellow oil. LCMS (method B): R_{T}=1.73 min, m/z=304 [M−Boc+H]⁺.

### Step 6: tert-Butyl (2S,5S)-5-(4-(4,6-dichloro-7H-pyrrolo[2,3-d]pyrimidin-7-yl)phenyl)-2-methylmorpholine-4-carboxylate and tert-butyl (2R,5R)-5-(4-(4,6-dichloro-7H-pyrrolo[2,3-d]pyrimidin-7-yl)phenyl)-2-methylmorpholine-4-carboxylate

General procedure 4 (Work-up A) using 4,6-dichloro-7H-pyrrolo[2,3-d]pyrimidine (1.07 g, 5.66 mmol), rac-tert-butyl (2R,5R)-2-methyl-5-(4-(4,4,5,5-tetramethyl-1,3,2-dioxaborolan-2-yl)phenyl)morpholine-4-carboxylate (2.97 g, 7.36 mmol), copper(II) trifluoroacetate hydrate (3.28 g, 11.3 mmol), 1,10-phenanthroline (2.04 g, 11.3 mmol), boric acid (1.40 g, 22.7 mmol) and DMF (143 mL) after 24 h at 75-80° C. gave rac-tert-butyl (2R,5R)-5-(4-(4,6-dichloro-7H-pyrrolo[2,3-d]pyrimidin-7-yl)phenyl)-2-methylmorpholine-4-carboxylate (1.23 g, 46%) as a white foam. LCMS (method B): R_{T}=1.68 min, m/z=463, 465 [M+H]⁺. rac-tert-Butyl (2R,5R)-5-(4-(4,6-dichloro-7H-pyrrolo[2,3-d]pyrimidin-7-yl)phenyl)-2-methylmorpholine-4-carboxylate (1.23 g) was resolved into the single stereoisomers by chiral SFC using a Chiralpak IG (21 mm×250 mm, 5 μm) column with isocratic solvent conditions: 30:70 MeOH/CO₂. The first eluted material was arbitrarily assigned as tert-butyl (2S,5S)-5-(4-(4,6-dichloro-7H-pyrrolo[2,3-d]pyrimidin-7-yl)phenyl)-2-methylmorpholine-4-carboxylate (457.6 mg, 37% recovery). Chiral purity (method H): R_{T}=2.86 min, 100% ee. The second eluted material was arbitrarily assigned as tert-butyl (2R,5R)-5-(4-(4,6-dichloro-7H-pyrrolo[2,3-d]pyrimidin-7-yl)phenyl)-2-methylmorpholine-4-carboxylate (439.5 mg, 35% recovery). Chiral purity (method H): R_{T}=3.40 min, 99.4% ee.

### Step 7: tert-Butyl (2S,5S)-5-(4-(6-chloro-4-oxo-3,4-dihydro-7H-pyrrolo[2,3-d]pyrimidin-7-yl)phenyl)-2-methylmorpholine-4-carboxylate

General procedure 5 using tert-butyl (2S,5S)-5-(4-(4,6-dichloro-7H-pyrrolo[2,3-d]pyrimidin-7-yl)phenyl)-2-methylmorpholine-4-carboxylate (458 mg, 0.988 mmol), aqueous sodium hydroxide (4 M, 7.41 mL, 29.7 mmol) and 1,4-dioxane (7.4 mL) after 18 h at 100° C. using 1 M aqueous potassium bisulfate for acidification gave the title compound (485 mg, >100%) as a yellow glass. LCMS (method B): R_{T}=1.25 min, m/z=445, 447 [M+H]⁺. This material was used without further purification.

### Step 8: tert-Butyl (2S,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5S)-5-(4-(6-chloro-4-oxo-3,4-dihydro-7H-pyrrolo[2,3-d]pyrimidin-7-yl)phenyl)-2-methylmorpholine-4-carboxylate (45 mg, 0.101 mmol), Epoxide 2 (38.2 mg, 0.152 mmol), Cs₂CO₃ (99 mg, 0.303 mmol) and DMF (2 mL) after 18 h at 80° C. gave the title compound (48 mg, 68%) as colourless glass. LCMS (method B): R_{T}=1.50 min, m/z=640, 642 [M−butene+H]⁺.

### Step 9: 6-Chloro-3-((1-(4-chlorobenzoyl)-4-hydroxypiperidin-4-yl)methyl)-7-(4-((3S,6S)-6-methylmorpholin-3-yl)phenyl)-3,7-dihydro-4H-pyrrolo[2,3-d]pyrimidin-4-one

General procedure 7 using tert-butyl (2S,5S)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (48 mg, 0.0689 mmol), TFA (1 mL) and DCM (2 mL) gave, after freeze-drying, the title compound (40 mg, 97%) as a white solid. LCMS (method B): R_{T}=0.89 min, m/z=596, 598 [M+H]⁺. ¹H NMR (500 MHz, DMSO-d₆) δ 8.07 (s, 1H), 7.74-7.66 (m, 2H), 7.55-7.49 (m, 2H), 7.46-7.37 (m, 4H), 6.80 (s, 1H), 4.96 (s, 1H), 4.30-4.09 (m, 1H), 4.03 (s, 2H), 3.97 (dd, J=11.4, 5.1 Hz, 1H), 3.93-3.83 (m, 1H), 3.81-3.66 (m, 2H), 3.46-3.33 (m, 1H), 3.30-2.95 (m, 3H), 2.82 (dd, J=12.3, 3.2 Hz, 1H), 2.58 (dd, J=12.3, 6.3 Hz, 1H), 1.73-1.27 (m, 4H), 1.18 (d, J=6.4 Hz, 3H).

### Example 69: 6-Chloro-3-((1-(4-chlorobenzoyl)-4-hydroxypiperidin-4-yl)methyl)-7-(4-((3R,6R)-6-methylmorpholin-3-yl)phenyl)-3,7-dihydro-4H-pyrrolo[2,3-d]pyrimidin-4-one

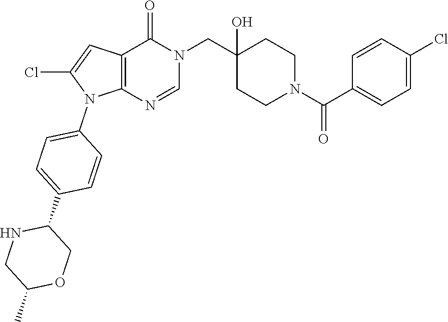 [SMILES: C[C@@H]1CN[C@H](c2ccc(-n3c(Cl)cc4c(=O)n(CC5(O)CCN(C(=O)c6ccc(Cl)cc6)CC5)cnc43)cc2)CO1]

### Step 1: tert-Butyl (2R,5R)-5-(4-(6-chloro-4-oxo-3,4-dihydro-7H-pyrrolo[2,3-d]pyrimidin-7-yl)phenyl)-2-methylmorpholine-4-carboxylate

General procedure 5 using tert-butyl (2R,5R)-5-(4-(4,6-dichloro-7H-pyrrolo[2,3-d]pyrimidin-7-yl)phenyl)-2-methylmorpholine-4-carboxylate (440 mg, 0.950 mmol), aqueous sodium hydroxide (4 M, 7.12 mL, 28.5 mmol) and 1,4-dioxane (7.1 mL) after 18 h at 100° C. using 1 M aqueous potassium bisulfate for acidification gave the title compound (466 mg, >100%) as a yellow glass. LCMS (method B): R_{T}=1.25 min, m/z=445, 447 [M+H]⁺. This material was used without further purification.

### Step 2: tert-Butyl (2R,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,5R)-5-(4-(6-chloro-4-oxo-3,4-dihydro-7H-pyrrolo[2,3-d]pyrimidin-7-yl)phenyl)-2-methylmorpholine-4-carboxylate (45 mg, 0.101 mmol), Epoxide 2 (38.2 mg, 0.152 mmol), Cs₂CO₃ (99 mg, 0.303 mmol) and DMF (2 mL) after 18 h at 80° C. gave the title compound (38 mg, 53%) as colourless glass. LCMS (method B): R_{T}=1.50 min, m/z=640, 642 [M−butene+H]⁺.

### Step 3: 6-Chloro-3-((1-(4-chlorobenzoyl)-4-hydroxypiperidin-4-yl)methyl)-7-(4-((3R,6R)-6-methylmorpholin-3-yl)phenyl)-3,7-dihydro-4H-pyrrolo[2,3-d]pyrimidin-4-one

General procedure 7 using tert-butyl (2R,5R)-5-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (38 mg, 0.0545 mmol), TFA (1 mL) and DCM (2 mL) gave, after freeze-drying, the title compound (31 mg, 95%) as a white solid. LCMS (method B): R_{T}=0.89 min, m/z=596, 598 [M+H]⁺. ¹H NMR (500 MHz, DMSO-d₆) δ 8.07 (s, 1H), 7.72-7.66 (m, 2H), 7.55-7.48 (m, 2H), 7.46-7.36 (m, 4H), 6.80 (s, 1H), 4.96 (s, 1H), 4.30-4.09 (m, 1H), 4.03 (s, 2H), 3.97 (dd, J=11.3, 5.1 Hz, 1H), 3.92-3.85 (m, 1H), 3.82-3.67 (m, 2H), 3.47-3.33 (m, 1H), 3.30-3.02 (m, 3H), 2.82 (dd, J=12.2, 3.2 Hz, 1H), 2.58 (dd, J=12.3, 6.3 Hz, 1H), 1.66-1.29 (m, 4H), 1.18 (d, J=6.4 Hz, 3H).

### Example 70: (S)-6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one

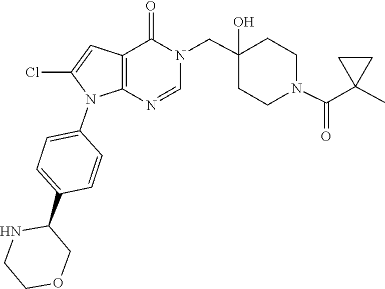 [SMILES: CC1(C(=O)N2CCC(O)(Cn3cnc4c(cc(Cl)n4-c4ccc([C@H]5COCCN5)cc4)c3=O)CC2)CC1]

### Step 1: tert-Butyl 3-(4-(4,4,5,5-tetramethyl-1,3,2-dioxaborolan-2-yl)phenyl)morpholine-4-carboxylate

General procedure 3 (Work-up A) using tert-butyl 3-(4-bromophenyl)morpholine-4-carboxylate (3.19 g, 9.33 mmol), B₂Pin₂ (3.56 g, 14.0 mmol), Pd(dppf)Cl₂·CH₂Cl₂ (790 mg, 0.933 mmol), KOAc (2.75 g, 28.0 mmol) and 1,4-dioxane (60 mL) after stirring at 90° C. (thermal) for 4 h gave the title compound (3.54 g, 97%) as brownish oil that solidified upon standing. LCMS (method B): R_{T}=1.63 min, m/z=334 [M−butene+H]⁺.

### Step 2: tert-Butyl 3-(4-(4,6-dichloro-7H-pyrrolo[2,3-d]pyrimidin-7-yl)phenyl)morpholine-4-carboxylate

General procedure 4 (Work-up A) using 4,6-dichloro-7H-pyrrolo[2,3-d]pyrimidine (1.00 g, 5.32 mmol), tert-butyl 3-(4-(4,4,5,5-tetramethyl-1,3,2-dioxaborolan-2-yl)phenyl)morpholine-4-carboxylate (4.14 g, 10.6 mmol), copper(II) acetate (1.93 g, 10.6 mmol), 1,10-phenanthroline (1.92 g, 10.6 mmol), boric acid (1.32 g, 21.3 mmol) and DMF (100 mL) after 4 days gave the title compound (1.31 g, 54%) as a white foam. LCMS (method B): R_{T}=1.58 min, m/z=449, 451 [M+H]⁺.

### Step 3: tert-Butyl 3-(4-(6-chloro-4-oxo-3,4-dihydro-7H-pyrrolo[2,3-d]pyrimidin-7-yl)phenyl)morpholine-4-carboxylate

General procedure 5 using tert-butyl 3-(4-(4,6-dichloro-7H-pyrrolo[2,3-d]pyrimidin-7-yl)phenyl)morpholine-4-carboxylate (910 mg, 2.03 mmol), aqueous sodium hydroxide (4 M, 10.1 mL, 40.5 mmol) and 1,4-dioxane (10 mL) after 18 h at 100° C. using AcOH for acidification gave the title compound (880 mg, quant.) as yellowish oil that solidified upon standing. LCMS (method B): R_{T}=1.16 min, m/z=431, 433 [M+H]⁺.

### Step 4: tert-Butyl (S)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate and tert-butyl (R)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate

General procedure 6 using tert-butyl 3-(4-(6-chloro-4-oxo-3,4-dihydro-7H-pyrrolo[2,3-d]pyrimidin-7-yl)phenyl)morpholine-4-carboxylate (873 mg, 2.03 mmol), Epoxide 1 (396 mg, 2.03 mmol), Cs₂CO₃ (1.98 g, 6.08 mmol) and DMF (4 mL) after 18 h at 80° C. gave the racemic product (250 mg, 19%). LCMS (method B): R_{T}=1.27 min, m/z=570 [M−butene+H]⁺. This material was resolved into the single stereoisomers by chiral SFC using a Chiralpak IG (30 mm×250 mm, 5 μm) column with isocratic solvent conditions: 45:55 EtOH:CO₂ (0.2% v/v NH₃). The first eluted material was arbitrarily assigned as tert-butyl (S)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (97.4 mg, 39% recovery) as an off-white solid. Chiral purity (method I): R_{T}=3.23 min, 100% ee. The second eluted material was arbitrarily assigned as tert-butyl (R)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (99.8 mg, 40% recovery) as an off-white solid. Chiral purity (method I): R_{T}=4.57 min, 99.7% ee.

### Step 5: (S)-6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one

General procedure 7 using tert-butyl (S)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (97 mg, 0.155 mmol), TFA (2 mL) and DCM (4 mL) gave, after freeze-drying, the title compound (72 mg, 88%) as white solid. LCMS (method B), R_{T}=0.68 min, m/z=526, 528 [M+H]⁺. ¹H NMR (500 MHz, DMSO-d₆) δ 8.08 (s, 1H), 7.62 (d, J=8.4 Hz, 2H), 7.41 (d, J=8.4 Hz, 2H), 6.81 (s, 1H), 4.91 (s, 1H), 4.01 (s, 2H), 3.99-3.88 (m, 3H), 3.83-3.74 (m, 2H), 3.53-3.45 (m, 1H), 3.26-3.05 (m, 3H), 3.01-2.84 (m, 3H), 1.60-1.34 (m, 4H), 1.21 (s, 3H), 0.81-0.72 (m, 2H), 0.57-0.48 (m, 2H).

### Example 71: (R)-6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one

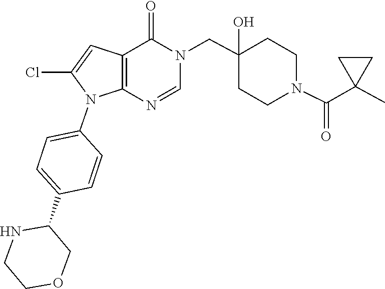 [SMILES: CC1(C(=O)N2CCC(O)(Cn3cnc4c(cc(Cl)n4-c4ccc([C@@H]5COCCN5)cc4)c3=O)CC2)CC1]

General procedure 7 using tert-butyl (R)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (99.8 mg, 0.160 mmol), TFA (2 mL) and DCM (4 mL) gave, after freeze-drying, the title compound (71 mg, 84%) as white solid. LCMS (method B), R_{T}=0.68 min, m/z=526, 528 [M+H]⁺. ¹H NMR (500 MHz, DMSO-d₆) δ 8.08 (s, 1H), 7.62 (d, J=8.4 Hz, 2H), 7.41 (d, J=8.4 Hz, 2H), 6.81 (s, 1H), 4.91 (s, 1H), 4.01 (s, 2H), 4.00-3.87 (m, 3H), 3.84-3.73 (m, 2H), 3.53-3.44 (m, 1H), 3.28-3.06 (m, 3H), 2.98-2.85 (m, 3H), 1.60-1.35 (m, 4H), 1.21 (s, 3H), 0.82-0.72 (m, 2H), 0.56-0.47 (m, 2H).

### Example 72: 6-Chloro-7-(3-fluoro-4-(morpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

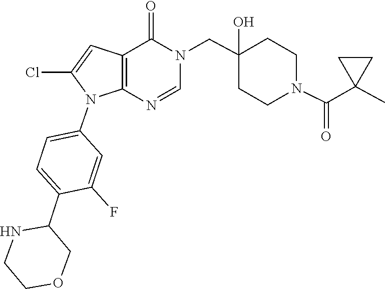 [SMILES: CC1(C(=O)N2CCC(O)(Cn3cnc4c(cc(Cl)n4-c4ccc(C5COCCN5)c(F)c4)c3=O)CC2)CC1]

### Step 1: 3-(4-Bromo-2-fluorophenyl)morpholine

General procedure 8 using 4-bromo-2-fluorobenzaldehyde (2.03 g, 10.0 mmol), 2-((tributylstannyl)methoxy)ethan-1-amine (3.64 g, 10.0 mmol), Cu(OTf)₂ (3.62 g, 10 mmol), 2,6-dimethylpyridine (1.07 g, 10.0 mmol), HFIP (40 mL), 4 Å molecular sieves (ca. 1 g) and DCM (160 mL) gave the title compound (1 g, 39%). LCMS (method C), R_{T}=0.64 min, m/z=260, 262 [M+H]⁺.

### Step 2: tert-Butyl 3-(4-bromo-2-fluorophenyl)morpholine-4-carboxylate

General procedure 9 using 3-(4-Bromo-2-fluorophenyl)morpholine (1 g, 3.90 mmol) and Boc₂O (1.02 g, 4.68 mmol) gave the title compound (1.39 g, quant.). LCMS (method C), R_{T}=1.50 min, m/z=382, 384 [M+Na]*; 260, 262 [M−Boc+H]⁺. ¹H NMR (500 MHz, Chloroform-d) δ 7.39-7.34 (m, 1H), 7.28-7.22 (m, 2H), 5.25-5.19 (m, 1H), 4.16 (d, J=12.0 Hz, 1H), 3.94 (dd, J=11.5, 3.9 Hz, 1H), 3.90-3.82 (m, 2H), 3.60 (td, J=11.6, 3.2 Hz, 1H), 3.34-3.25 (m, 1H), 1.41 (s, 9H).

### Step 3: tert-Butyl 3-(2-fluoro-4-(4,4,5,5-tetramethyl-1,3,2-dioxaborolan-2-yl)phenyl)morpholine-4-carboxylate

General procedure 3 (Work-up A) using tert-butyl 3-(4-bromo-2-fluorophenyl)morpholine-4-carboxylate (600 mg, 1.67 mmol), B₂Pin₂ (634 mg, 2.50 mmol), Pd(dppf)Cl₂·CH₂Cl₂ (141 mg, 0.167 mmol), KOAc (490 mg, 5.00 mmol) and 1,4-dioxane (17 mL) after stirring at 120° C. (microwave irradiation) for 30 min gave the title compound (546 mg, 80%) as a yellow oil that solidified upon standing. LCMS (method B): R_{T}=1.70 min, m/z=308 [M−Boc+H]⁺, 352 [M−butene+H]⁺.

### Step 4: tert-Butyl 3-(4-(4,6-dichloro-7H-pyrrolo[2,3-d]pyrimidin-7-yl)-2-fluorophenyl)morpholine-4-carboxylate

General procedure 4 (Work-up A) using 4,6-dichloro-7H-pyrrolo[2,3-d]pyrimidine (63 mg, 0.335 mmol), tert-butyl 3-(2-fluoro-4-(4,4,5,5-tetramethyl-1,3,2-dioxaborolan-2-yl)phenyl)morpholine-4-carboxylate (273 mg, 0.670 mmol), copper(II) acetate (122 mg, 0.670 mmol), 1,10-phenanthroline (121 mg, 0.670 mmol), boric acid (83 mg, 1.34 mmol) and DMF (6.3 mL) after 4 days gave the title compound (36 mg, 23%) as a colourless glass. LCMS (method B): R_{T}=1.61 min, m/z=467, 469 [M+H]⁺.

### Step 5: tert-Butyl 3-(4-(6-chloro-4-oxo-3,4-dihydro-7H-pyrrolo[2,3-d]pyrimidin-7-yl)-2-fluorophenyl)morpholine-4-carboxylate

General procedure 5 using tert-butyl 3-(4-(4,6-dichloro-7H-pyrrolo[2,3-d]pyrimidin-7-yl)-2-fluorophenyl)morpholine-4-carboxylate (36 mg, 0.0770 mmol), aqueous sodium hydroxide (4 M, 0.39 mL, 1.54 mmol) and 1,4-dioxane (0.4 mL) after 18 h at 100° C. using AcOH for acidification gave the title compound (35 mg, quant.) as a yellowish oil that solidified upon standing. LCMS (method B): R_{T}=1.20 min, m/z=449, 451 [M+H]⁺.

### Step 6: tert-Butyl 3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)-2-fluorophenyl)morpholine-4-carboxylate

General procedure 6 using tert-butyl 3-(4-(6-chloro-4-oxo-3,4-dihydro-7H-pyrrolo[2,3-d]pyrimidin-7-yl)-2-fluorophenyl)morpholine-4-carboxylate (35 mg, 0.0780 mmol), Epoxide 1 (15.2 mg, 0.0780 mmol), Cs₂CO₃ (76.2 mg, 0.234 mmol) and DMF (1 mL) after 18 h at 80° C. gave the title compound (12 mg, 23%) as a colourless glass. LCMS (method A): R_{T}=1.40 min, m/z=644, 646 [M+H]⁺.

### Step 7: 6-Chloro-7-(3-fluoro-4-(morpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

General procedure 7 using tert-butyl 3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)-2-fluorophenyl)morpholine-4-carboxylate (12 mg, 0.0186 mmol), TFA (1 mL) and DCM (2 mL) gave, after freeze-drying, the title compound (7 mg, 69%) as a white solid. LCMS (method B): R_{T}=0.68 min, m/z=544, 546 [M+H]⁺. ¹H NMR (500 MHz, DMSO-d₆) δ 8.10 (s, 1H), 7.80 (t, J=8.1 Hz, 1H), 7.44 (dd, J=10.7, 2.1 Hz, 1H), 7.34 (dd, J=8.2, 2.0 Hz, 1H), 6.84 (s, 1H), 4.92 (s, 1H), 4.19-4.12 (m, 1H), 4.02 (s, 2H), 4.00-3.90 (m, 2H), 3.84-3.75 (m, 2H), 3.54-3.47 (m, 1H), 3.28-3.04 (m, 3H), 3.01-2.87 (m, 3H), 1.59-1.34 (m, 4H), 1.21 (s, 3H), 0.81-0.73 (m, 2H), 0.57-0.46 (m, 2H).

### Example 73: (S)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one

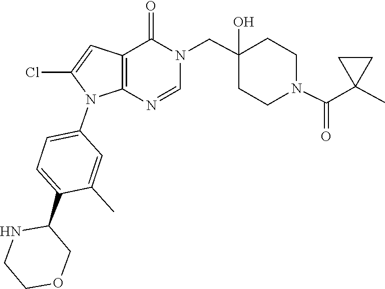 [SMILES: Cc1cc(-n2c(Cl)cc3c(=O)n(CC4(O)CCN(C(=O)C5(C)CC5)CC4)cnc32)ccc1[C@H]1COCCN1]

### Step 1: 3-(4-Bromo-2-methylphenyl)morpholine

General procedure 8 using 4-bromo-2-methylbenzaldehyde (5.97 g, 30.0 mmol), 2-((tributylstannyl)methoxy)ethan-1-amine (10.9 g, 30.0 mmol), Cu(OTf)₂ (10.9 g, 30.0 mmol), 2,6-dimethylpyridine (3.21 g, 30.0 mmol), HFIP (120 mL), 4 Å molecular sieves (ca. 3 g) and DCM (480 mL) gave the title compound which was used directly in the step.

### Step 2: tert-Butyl 3-(4-bromo-2-methylphenyl)morpholine-4-carboxylate

General procedure 9 using 3-(4-bromo-2-methylphenyl)morpholine (7.68 g, 30.0 mmol) and Boc₂O (7.85 g, 36.0 mmol) gave the title compound (2.4 g, 22% (2 steps)). LCMS (method C): R_{T}=1.46 min, m/z=256, 258 [M−Boc+H]⁺. ¹H NMR (500 MHz, Chloroform-d) δ 7.52 (d, J=8.1 Hz, 1H), 7.34-7.28 (m, 2H), 5.06 (dd, J=4.4, 2.2 Hz, 1H), 4.04 (dd, J=12.1, 2.2 Hz, 1H), 3.92 (ddd, J=11.3, 3.9, 1.9 Hz, 1H), 3.85 (dd, J=11.9, 4.2 Hz, 1H), 3.77 (dt, J=13.7, 2.6 Hz, 1H), 3.64 (td, J=11.4, 3.2 Hz, 1H), 3.29 (ddd, J=13.7, 11.4, 3.8 Hz, 1H), 2.31 (s, 3H), 1.40 (s, 9H).

### Step 3: tert-Butyl 3-(2-methyl-4-(4,4,5,5-tetramethyl-1,3,2-dioxaborolan-2-yl)phenyl)morpholine-4-carboxylate

General procedure 3 (Work-up A) using tert-butyl 3-(4-bromo-2-methylphenyl)morpholine-4-carboxylate (1.80 g, 5.05 mmol), B₂Pin₂ (1.92 g, 7.58 mmol), Pd(dppf)Cl₂·CH₂Cl₂ (428 mg, 0.505 mmol), KOAc (1.49 g, 15.2 mmol) and 1,4-dioxane (51 mL) after stirring at 90° C. (thermal) for 3 h gave the title compound (622 mg, 91%) as yellow oil. LCMS (method B): R_{T}=1.69 min, m/z=304 [M−Boc+H]⁺.

### Step 4: tert-Butyl (S)-3-(4-(4,6-dichloro-7H-pyrrolo[2,3-d]pyrimidin-7-yl)-2-methylphenyl)morpholine-4-carboxylate and tert-butyl (R)-3-(4-(4,6-dichloro-7H-pyrrolo[2,3-d]pyrimidin-7-yl)-2-methylphenyl)morpholine-4-carboxylate

General procedure 4 (Work-up A) using 4,6-dichloro-7H-pyrrolo[2,3-d]pyrimidine (450 mg, 2.39 mmol), tert-butyl 3-(2-methyl-4-(4,4,5,5-tetramethyl-1,3,2-dioxaborolan-2-yl)phenyl)morpholine-4-carboxylate (1.93 g, 4.79 mmol), copper(II) acetate (869 mg, 4.79 mmol), 1,10-phenanthroline (863 mg, 4.79 mmol), boric acid (592 mg, 9.57 mmol) and DMF (45 mL) after 3 days gave the racemic product (374 mg, 33%) as a white foam. LCMS (method B): R_{T}=1.66 min, m/z=463, 465 [M+H]⁺. This material was resolved into the single stereoisomers by chiral HPLC using a Lux C₄ (21.2 mm×250 mm, 5 μm) column with isocratic solvent conditions: EtOH. The first eluted material was arbitrarily assigned as tert-butyl (S)-3-(4-(4,6-dichloro-7H-pyrrolo[2,3-d]pyrimidin-7-yl)-2-methylphenyl)morpholine-4-carboxylate (155 mg, 41% recovery) as an off-white solid. Chiral purity (method J): R_{T}=1.95 min, 97.2% ee. The second eluted material was arbitrarily assigned as tert-butyl (R)-3-(4-(4,6-dichloro-7H-pyrrolo[2,3-d]pyrimidin-7-yl)-2-methylphenyl)morpholine-4-carboxylate (121 mg, 32% recovery) as an off-white solid. Chiral purity (method J): R_{T}=2.44 min, 99.2% ee.

### Step 5: tert-Butyl (S)-3-(4-(6-chloro-4-oxo-3,4-dihydro-7H-pyrrolo[2,3-d]pyrimidin-7-yl)-2-methylphenyl)morpholine-4-carboxylate

General procedure 5 using tert-butyl (S)-3-(4-(4,6-dichloro-7H-pyrrolo[2,3-d]pyrimidin-7-yl)-2-methylphenyl)morpholine-4-carboxylate (120 mg, 0.259 mmol), aqueous sodium hydroxide (4 M, 1.29 mL, 5.18 mmol) and 1,4-dioxane (1.3 mL) after 18 h at 100° C. and then 24 h at 90° C. using AcOH for acidification gave the title compound (120 mg, >100%) as a yellow solid. This material was used without further purification. LCMS (method B): R_{T}=1.20 min, m/z=445, 447 [M+H]⁺.

### Step 6: tert-Butyl (S)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)-2-methylphenyl)morpholine-4-carboxylate

General procedure 6 using tert-butyl (S)-3-(4-(6-chloro-4-oxo-3,4-dihydro-7H-pyrrolo[2,3-d]pyrimidin-7-yl)-2-methylphenyl)morpholine-4-carboxylate (115 mg, 0.259 mmol), Epoxide 1 (50.5 mg, 0.259 mmol), Cs₂CO₃ (253 mg, 0.775 mmol) and DMF (3 mL) after 18 h at 80° C. gave the title compound (61 mg, 36%) as a colourless glass. LCMS (method A): R_{T}=1.43 min, m/z=640, 642 [M+H]⁺.

### Step 7: (S)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one

General procedure 7 using tert-butyl (S)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)-2-methylphenyl)morpholine-4-carboxylate (61 mg, 0.0953 mmol), TFA (1 mL) and DCM (2 mL) gave, after freeze-drying, the title compound (47 mg, 91%) as a white solid. LCMS (method B): R_{T}=0.71 min, m/z=540, 542 [M+H]⁺. ¹H NMR (500 MHz, DMSO-d₆) δ 8.07 (s, 1H), 7.76 (d, J=8.0 Hz, 1H), 7.32-7.20 (m, 2H), 6.80 (s, 1H), 4.92 (s, 1H), 4.14-4.04 (m, 1H), 4.01 (s, 2H), 3.99-3.89 (m, 2H), 3.84-3.74 (m, 2H), 3.58-3.48 (m, 1H), 3.29-2.69 (m, 6H), 2.43 (s, 3H), 1.60-1.35 (m, 4H), 1.21 (s, 3H), 0.81-0.72 (m, 2H), 0.59-0.45 (m, 2H).

### Example 74: (R)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one

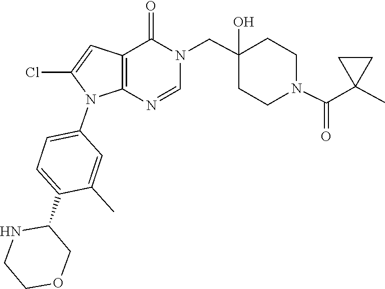 [SMILES: Cc1cc(-n2c(Cl)cc3c(=O)n(CC4(O)CCN(C(=O)C5(C)CC5)CC4)cnc32)ccc1[C@@H]1COCCN1]

### Step 1: tert-Butyl (R)-3-(4-(6-chloro-4-oxo-3,4-dihydro-7H-pyrrolo[2,3-d]pyrimidin-7-yl)-2-methylphenyl)morpholine-4-carboxylate

General procedure 5 using tert-butyl (R)-3-(4-(4,6-dichloro-7H-pyrrolo[2,3-d]pyrimidin-7-yl)-2-methylphenyl)morpholine-4-carboxylate (120 mg, 0.259 mmol), aqueous sodium hydroxide (4 M, 1.29 mL, 5.18 mmol) and 1,4-dioxane (1.3 mL) after 18 h at 100° C. and then 24 h at 90° C. using AcOH for acidification gave the title compound (130 mg, >100%) as a yellow solid. This material was used without further purification. LCMS (method B): R_{T}=1.21 min, m/z=445, 447 [M+H]⁺.

### Step 2: tert-Butyl (R)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)-2-methylphenyl)morpholine-4-carboxylate

General procedure 6 using tert-butyl (R)-3-(4-(6-chloro-4-oxo-3,4-dihydro-7H-pyrrolo[2,3-d]pyrimidin-7-yl)-2-methylphenyl)morpholine-4-carboxylate (115 mg, 0.259 mmol), Epoxide 1 (50.5 mg, 0.259 mmol), Cs₂CO₃ (253 mg, 0.775 mmol) and DMF (3 mL) after 18 h at 80° C. gave the title compound (61 mg, 36%) as a colourless glass. LCMS (method A): R_{T}=1.42 min, m/z=640, 642 [M+H]⁺.

### Step 3: (R)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one

General procedure 7 using tert-butyl (R)-3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)-2-methylphenyl)morpholine-4-carboxylate (61 mg, 0.0953 mmol), TFA (1 mL) and DCM (2 mL) gave, after freeze-drying, the title compound (51 mg, 99%) as a white solid. LCMS (method B), R_{T}=0.71 min, m/z=540, 542 [M+H]⁺. ¹H NMR (500 MHz, DMSO-d₆) δ 8.07 (s, 1H), 7.76 (d, J=8.0 Hz, 1H), 7.25 (s, 2H), 6.80 (s, 1H), 4.92 (s, 1H), 4.13-4.05 (m, 1H), 4.01 (s, 2H), 3.99-3.92 (m, 2H), 3.84-3.76 (m, 2H), 3.57-3.50 (m, 1H), 3.26-2.76 (m, 6H), 2.43 (s, 3H), 1.58-1.35 (m, 4H), 1.21 (s, 3H), 0.80-0.73 (m, 2H), 0.55-0.47 (m, 2H).

### Example 75: 6-Chloro-3-((4-hydroxy-1-(1-methylcyclopropane-1-carbonyl)piperidin-4-yl)methyl)-7-(4-(4-methylmorpholin-3-yl)phenyl)-3,7-dihydro-4H-pyrrolo[2,3-d]pyrimidin-4-one

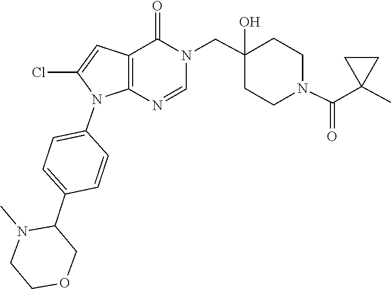

### Step 1: 6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one

General procedure 7 using tert-butyl 3-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (30 mg, 0.0479 mmol), TFA (1 mL) and DCM (2 mL) gave, after freeze-drying, the title compound (23 mg, 91%) as a white solid. LCMS (method A), R_{T}=0.57 min, m/z=526, 528 [M+H]⁺. ¹H NMR (500 MHz, DMSO-d₆) δ 8.07 (s, 1H), 7.61 (d, J=8.4 Hz, 2H), 7.40 (d, J=8.4 Hz, 2H), 6.80 (s, 1H), 4.89 (s, 1H), 4.01 (s, 2H), 3.98-3.88 (m, 3H), 3.82-3.74 (m, 2H), 3.52-3.45 (m, 1H), 3.27-3.10 (m, 3H), 2.97-2.83 (m, 2H), 1.57-1.45 (m, 2H), 1.44-1.34 (m, 2H), 1.21 (s, 3H), 0.80-0.73 (m, 2H), 0.54-0.48 (m, 2H).

### Step 2: 6-Chloro-3-((4-hydroxy-1-(1-methylcyclopropane-1-carbonyl)piperidin-4-yl)methyl)-7-(4-(4-methylmorpholin-3-yl)phenyl)-3,7-dihydro-4H-pyrrolo[2,3-d]pyrimidin-4-one

To a solution of 6-chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one (16 mg, 0.0304 mmol) in 1:1 MeCN/MeOH (2 mL) at rt were added 37% aqueous formaldehyde (23 μL, 0.304 mmol) and sodium triacetoxyborohydride (64 mg, 0.304 mmol). The reaction was stirred at rt for 18 h and concentrated under reduced pressure before 1 M aqueous sodium hydroxide (2 mL) was added. The resulting mixture was extracted with DCM (2×3 mL) using an ISOLUTE® phase separator. The combined organic phases were concentrated under reduced pressure and the residue was purified by flash chromatography to give, after freeze-drying, the title compound (14 mg, 85%) as a white solid. LCMS (method B), R_{T}=0.69 min, m/z=540, 542 [M+H]⁺. ¹H NMR (500 MHz, DMSO-d₆) δ 8.09 (s, 1H), 7.60-7.54 (m, 2H), 7.49-7.41 (m, 2H), 6.82 (s, 1H), 4.92 (s, 1H), 4.02 (s, 2H), 4.00-3.92 (m, 2H), 3.92-3.83 (m, 1H), 3.73-3.61 (m, 2H), 3.30-3.25 (m, 1H), 3.23-3.02 (m, 3H), 2.90-2.83 (m, 1H), 2.36-2.29 (m, 1H), 2.03 (s, 3H), 1.56-1.47 (m, 2H), 1.43-1.37 (m, 2H), 1.21 (s, 3H), 0.80-0.74 (m, 2H), 0.55-0.49 (m, 2H).

### Example 76: (S)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one

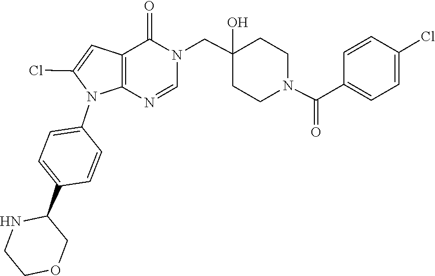 [SMILES: O=C(c1ccc(Cl)cc1)N1CCC(O)(Cn2cnc3c(cc(Cl)n3-c3ccc([C@H]4COCCN4)cc3)c2=O)CC1]

### Step 1: tert-Butyl (S)-3-(4-(4,6-dichloro-7H-pyrrolo[2,3-d]pyrimidin-7-yl)phenyl)morpholine-4-carboxylate and tert-butyl (R)-3-(4-(4,6-dichloro-7H-pyrrolo[2,3-d]pyrimidin-7-yl)phenyl)morpholine-4-carboxylate

tert-Butyl 3-(4-(4,6-dichloro-7H-pyrrolo[2,3-d]pyrimidin-7-yl)phenyl)morpholine-4-carboxylate (1.31 g) was resolved into the single stereoisomers by chiral HPLC using a Lux A1 (20 mm×250 mm, 5 μm) column with isocratic solvent conditions: MeOH. The first eluted material was arbitrarily assigned as tert-butyl (S)-3-(4-(4,6-dichloro-7H-pyrrolo[2,3-d]pyrimidin-7-yl)phenyl)morpholine-4-carboxylate (552 mg, 42% recovery) as a white solid. Chiral purity (method K): R_{T}=1.40 min, 100% ee. The second eluted material was arbitrarily assigned as tert-butyl (R)-3-(4-(4,6-dichloro-7H-pyrrolo[2,3-d]pyrimidin-7-yl)phenyl)morpholine-4-carboxylate (580 mg, 44% recovery) as a white solid. Chiral purity (method K): R_{T}=2.09 min, 99.6% ee.

### Step 2: tert-Butyl (S)-3-(4-(6-chloro-4-oxo-3,4-dihydro-7H-pyrrolo[2,3-d]pyrimidin-7-yl)phenyl)morpholine-4-carboxylate

General procedure 5 using tert-butyl (S)-3-(4-(4,6-dichloro-7H-pyrrolo[2,3-d]pyrimidin-7-yl)phenyl)morpholine-4-carboxylate (552 mg, 1.23 mmol), aqueous sodium hydroxide (4 M, 6.14 mL, 24.6 mmol) and 1,4-dioxane (6.2 mL) after 18 h at 100° C. using AcOH for acidification gave the title compound (550 mg, >100%) as a pale yellow glass. LCMS (method B): R_{T}=1.18 min, m/z=431, 433 [M+H]⁺.

### Step 3: tert-Butyl (S)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (S)-3-(4-(6-chloro-4-oxo-3,4-dihydro-7H-pyrrolo[2,3-d]pyrimidin-7-yl)phenyl)morpholine-4-carboxylate (50 mg, 0.116 mmol), Epoxide 2 (44 mg, 0.174 mmol), Cs₂CO₃ (113 mg, 0.348 mmol) and DMF (2 mL) after 18 h at 80° C. gave the title compound (50 mg, 63%) as a colourless glass. LCMS (method B): R_{T}=1.46 min, m/z=626, 628 [M−butene+H]⁺.

### Step 4: (S)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one

General procedure 7 using tert-butyl (S)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)morpholine-4-carboxylate (50 mg, 0.0733 mmol), TFA (1 mL) and DCM (2 mL) gave, after freeze-drying, the title compound (36 mg, 84%) as a white solid. LCMS (method B): R_{T}=0.84 min, m/z=582, 584 [M+H]⁺. ¹H NMR (500 MHz, DMSO-d₆) δ 8.07 (s, 1H), 7.63 (d, J=8.2 Hz, 2H), 7.51 (d, J=8.5 Hz, 2H), 7.46-7.37 (m, 4H), 6.80 (s, 1H), 4.96 (s, 1H), 4.24-4.11 (m, 1H), 4.03 (s, 2H), 3.98-3.88 (m, 1H), 3.87-3.75 (m, 2H), 3.57-3.47 (m, 1H), 3.43-3.34 (m, 1H), 3.29-3.21 (m, 2H), 3.19-3.08 (m, 1H), 3.03-2.81 (m, 3H), 1.70-1.53 (m, 2H), 1.51-1.27 (m, 2H).

### Example 77: (R)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one

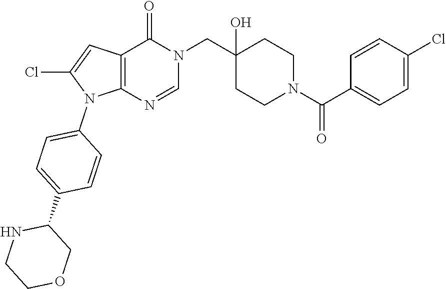 [SMILES: O=C(c1ccc(Cl)cc1)N1CCC(O)(Cn2cnc3c(cc(Cl)n3-c3ccc([C@@H]4COCCN4)cc3)c2=O)CC1]

### Step 1: tert-Butyl (R)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (R)-3-(4-(6-chloro-4-oxo-3,4-dihydro-7H-pyrrolo[2,3-d]pyrimidin-7-yl)phenyl)morpholine-4-carboxylate (50 mg, 0.116 mmol), Epoxide 2 (44 mg, 0.174 mmol), Cs₂CO₃ (113 mg, 0.348 mmol) and DMF (2 mL) after 18 h at 80° C. gave the title compound (50 mg, 63%) as a colourless glass. LCMS (method B): R_{T}=1.46 min, m/z=626, 628 [M−Boc+H]⁺.

### Step 4: (R)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one

General procedure 7 using tert-butyl (R)-3-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)morpholine-4-carboxylate (50 mg, 0.0733 mmol), TFA (1 mL) and DCM (2 mL) gave, after freeze-drying, the title compound (42 mg, 98%) as a white solid. LCMS (method B): R_{T}=0.84 min, m/z=582, 584 [M+H]⁺. ¹H NMR (500 MHz, DMSO-d₆) δ 8.07 (s, 1H), 7.66-7.59 (m, 2H), 7.53-7.48 (m, 2H), 7.46-7.37 (m, 4H), 6.80 (s, 1H), 4.96 (s, 1H), 4.23-4.11 (m, 1H), 4.03 (s, 2H), 3.99-3.90 (m, 1H), 3.86-3.75 (m, 2H), 3.56-3.46 (m, 1H), 3.41-3.33 (m, 1H), 3.30-3.19 (m, 2H), 3.19-3.09 (m, 1H), 3.07-2.84 (m, 3H), 1.72-1.53 (m, 2H), 1.51-1.29 (m, 2H).

### Example 78: 6-Chloro-3-((4-hydroxy-1-((R)-4,4,4-trifluoro-3-phenylbutanoyl)piperidin-4-yl)methyl)-7-(4-((S)-morpholin-3-yl)phenyl)-3,7-dihydro-4H-pyrrolo[2,3-d]pyrimidin-4-one

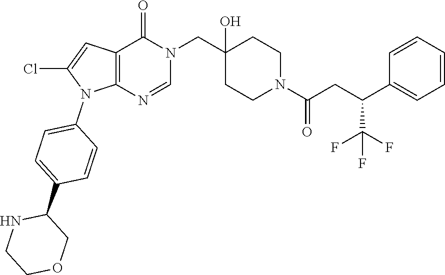 [SMILES: O=C(C[C@H](c1ccccc1)C(F)(F)F)N1CCC(O)(Cn2cnc3c(cc(Cl)n3-c3ccc([C@H]4COCCN4)cc3)c2=O)CC1]

### Step 1: tert-Butyl (S)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (S)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 3 (52 mg, 0.167 mmol), Cs₂CO₃ (100 mg, 0.306 mmol) and DMF (1.4 mL) after 18 h at 80° C. gave, after purification by prep-HPLC, the title compound (63 mg, 61%) as an orange oil. LCMS (method B): R_{T}=1.54 min, m/z=688, 690 [M−butene+H]⁺.

### Step 2: 6-Chloro-3-((4-hydroxy-1-((R)-4,4,4-trifluoro-3-phenylbutanoyl)piperidin-4-yl)methyl)-7-(4-((S)-morpholin-3-yl)phenyl)-3,7-dihydro-4H-pyrrolo[2,3-d]pyrimidin-4-one

General procedure 7 using tert-butyl (S)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate (63 mg, 0.0847 mmol), TFA (0.43 mL) and DCM (0.86 mL) gave, after purification by prep-HPLC and freeze-drying, the title compound (33 mg, 58%) as a white solid. LCMS (method B): R_{T}=0.91 min, m/z=644, 646 [M+H]⁺. ¹H NMR (500 MHz, DMSO-d₆) δ 8.04 (d, J=10.7 Hz, 1H), 7.61 (d, J=8.4 Hz, 2H), 7.43-7.29 (m, 7H), 6.79 (s, 1H), 4.88 (s, 1H), 4.16-4.05 (m, 1H), 4.04-3.84 (m, 4H), 3.82-3.71 (m, 3H), 3.48 (td, J=10.6, 3.9 Hz, 1H), 3.30-3.10 (m, 3H (signal overlaps with HDO)), 3.01-2.79 (m, 5H), 1.65-1.54 (m, 0.5H), 1.48-1.15 (m, 3.5H).

### Example 79: (S)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one

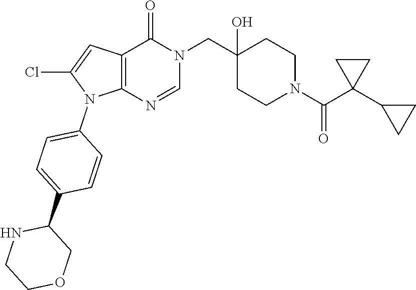 [SMILES: O=C(N1CCC(O)(Cn2cnc3c(cc(Cl)n3-c3ccc([C@H]4COCCN4)cc3)c2=O)CC1)C1(C2CC2)CC1]

### Step 1: tert-Butyl (S)-3-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)morpholine-4-carboxylate

General procedure 6 using tert-butyl (S)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 16 (37 mg, 0.167 mmol), Cs₂CO₃ (100 mg, 0.306 mmol) and DMF (1.4 mL) after 18 h at 80° C. gave the title compound (51 mg, 56%) as a white solid. LCMS (method B): R_{T}=1.38 min, m/z=596, 598 [M−butene+H]⁺.

### Step 2: (S)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one

General procedure 7 using tert-butyl (S)-3-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)morpholine-4-carboxylate (51 mg, 0.0782 mmol), TFA (0.4 mL) and DCM (0.8 mL) gave, after purification by prep-HPLC and freeze-drying, the title compound (31.5 mg, 71%) as a white solid. LCMS (method B): R_{T}=0.74 min, m/z=552, 554 [M+H]⁺. ¹H NMR (500 MHz, DMSO-d₆) δ 8.08 (s, 1H), 7.61 (d, J=8.5 Hz, 2H), 7.40 (d, J=8.5 Hz, 2H), 6.81 (s, 1H), 4.92 (s, 1H), 4.05-3.95 (m, 4H), 3.90 (dd, J=9.9, 2.8 Hz, 1H), 3.81-3.74 (m, 2H), 3.48 (td, J=10.7, 4.0 Hz, 1H), 3.41-3.00 (m, 3H), 2.97-2.85 (m, 3H), 1.63-1.44 (m, 4H), 1.17-1.09 (m, 1H), 0.67 (s, 2H), 0.46 (s, 2H), 0.41-0.34 (m, 2H), 0.14-0.04 (m, 2H).

### Example 80: (R)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one

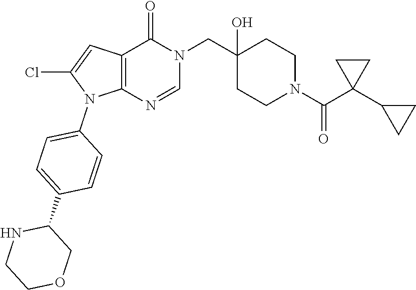 [SMILES: O=C(N1CCC(O)(Cn2cnc3c(cc(Cl)n3-c3ccc([C@@H]4COCCN4)cc3)c2=O)CC1)C1(C2CC2)CC1]

### Step 1: tert-Butyl (R)-3-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)morpholine-4-carboxylate

General procedure 6 using tert-butyl (R)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 16 (37 mg, 0.167 mmol), Cs₂CO₃ (100 mg, 0.306 mmol) and DMF (1.4 mL) after 18 h at 80° C. gave the title compound (51 mg, 56%) as a white solid. LCMS (method B): R_{T}=1.38 min, m/z=596, 598 [M−butene+H]⁺.

### Step 2: (R)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one

General procedure 7 using tert-butyl (R)-3-(4-(3-((1-([1,1′-bi(cyclopropane)]-1-carbonyl)-4-hydroxypiperidin-4-yl)methyl)-6-chloro-4-oxo-3,4-dihydro-7H-pyrrolo[2,3-d]pyrimidin-7-yl)phenyl)morpholine-4-carboxylate (49 mg, 0.0751 mmol), TFA (0.4 mL) and DCM (0.8 mL) gave, after purification by prep-HPLC and freeze-drying, the title compound (31.5 mg, 71%) as a white solid. LCMS (method B): R_{T}=0.75 min, m/z=552, 554 [M+H]⁺. ¹H NMR (500 MHz, DMSO-d₆) δ 8.07 (s, 1H), 7.65-7.59 (m, 2H), 7.43-7.37 (m, 2H), 6.81 (s, 1H), 4.92 (s, 1H), 4.08-3.95 (m, 4H), 3.95-3.87 (m, 1H), 3.84-3.74 (m, 2H), 3.50 (td, J=10.5, 4.1 Hz, 1H), 3.45-2.99 (m, 4H (signals overlap with HDO)), 2.98-2.87 (m, 2H), 1.60-1.48 (m, 2H), 1.45-1.36 (m, 2H), 1.13 (tt, J=8.2, 5.1 Hz, 1H), 0.73-0.62 (m, 2H), 0.51-0.42 (m, 2H), 0.41-0.34 (m, 2H), 0.13-0.03 (m, 2H).

### Example 81: 6-Chloro-3-((4-hydroxy-1-((R)-4,4,4-trifluoro-3-phenylbutanoyl)piperidin-4-yl)methyl)-7-(4-((R)-morpholin-3-yl)phenyl)-3,7-dihydro-4H-pyrrolo[2,3-d]pyrimidin-4-one

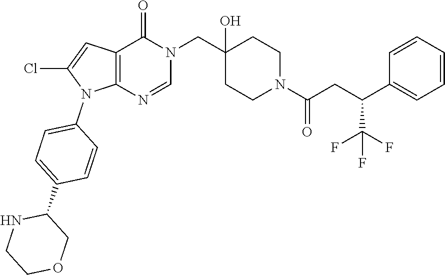 [SMILES: O=C(C[C@H](c1ccccc1)C(F)(F)F)N1CCC(O)(Cn2cnc3c(cc(Cl)n3-c3ccc([C@@H]4COCCN4)cc3)c2=O)CC1]

### Step 1: tert-Butyl (R)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (R)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 3 (52 mg, 0.167 mmol), Cs₂CO₃ (100 mg, 0.306 mmol) and DMF (1.4 mL) after 18 h at 80° C. gave, after purification by prep-HPLC, the title compound (42 mg, 40%) as an orange oil. LCMS (method B): R_{T}=1.54 min, m/z=688, 690 [M−butene+H]⁺.

### Step 2: 6-Chloro-3-((4-hydroxy-1-((R)-4,4,4-trifluoro-3-phenylbutanoyl)piperidin-4-yl)methyl)-7-(4-((R)-morpholin-3-yl)phenyl)-3,7-dihydro-4H-pyrrolo[2,3-d]pyrimidin-4-one

General procedure 7 using tert-butyl (R)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate (42 mg, 0.0564 mmol), TFA (0.35 mL) and DCM (0.7 mL) gave, after purification by prep-HPLC and freeze-drying, the title compound (33 mg, 58%) as a white solid. LCMS (method B): R_{T}=0.86 min, m/z=644, 646 [M+H]⁺. ¹H NMR (500 MHz, DMSO-d₆) δ 8.05 (d, J=10.5 Hz, 1H), 7.64-7.59 (m, 2H), 7.44-7.38 (m, 4H), 7.38-7.30 (m, 3H), 6.80 (d, J=1.0 Hz, 1H), 4.89 (s, 1H), 4.11 (td, J=9.8, 3.9 Hz, 1H), 4.03-3.85 (m, 4H), 3.82-3.73 (m, 3H), 3.49 (td, J=10.5, 3.9 Hz, 1H), 3.30-3.12 (m, 3H), 3.02-2.79 (m, 5H), 1.47-1.16 (m, 4H).

### Example 82: 6-Chloro-3-((1-((R)-4,4-difluoro-3-phenylbutanoyl)-4-hydroxypiperidin-4-yl)methyl)-7-(4-((S)-morpholin-3-yl)phenyl)-3,7-dihydro-4H-pyrrolo[2,3-d]pyrimidin-4-one

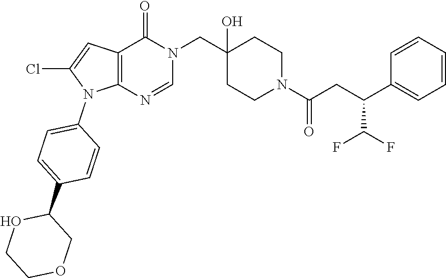 [SMILES: O=C(C[C@H](c1ccccc1)C(F)F)N1CCC(O)(Cn2cnc3c(cc(Cl)n3-c3ccc([C@H]4COCCO4)cc3)c2=O)CC1]

### Step 1: tert-Butyl (S)-3-(4-(6-chloro-3-((1-((R)-4,4-difluoro-3-phenylbutanoyl)-4-hydroxypiperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (S)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 22 (49 mg, 0.167 mmol), Cs₂CO₃ (100 mg, 0.306 mmol) and DMF (1.4 mL) after 18 h at 80° C. gave the title compound (32 mg, 32%) as a white solid. LCMS (method B): R_{T}=1.48 min, m/z=670, 672 [M−butene+H]⁺.

### Step 2: 6-Chloro-3-((1-((R)-4,4-difluoro-3-phenylbutanoyl)-4-hydroxypiperidin-4-yl)methyl)-7-(4-((S)-morpholin-3-yl)phenyl)-3,7-dihydro-4H-pyrrolo[2,3-d]pyrimidin-4-one

General procedure 7 using tert-butyl (S)-3-(4-(6-chloro-3-((1-((R)-4,4-difluoro-3-phenylbutanoyl)-4-hydroxypiperidin-4-yl)methyl)-4-oxo-3,4-dihydro-7H-pyrrolo[2,3-d]pyrimidin-7-yl)phenyl)morpholine-4-carboxylate (32.8 mg, 0.0452 mmol), TFA (0.45 mL) and DCM (0.45 mL) gave, after freeze-drying, the title compound (24.1 mg, 81%) as a white solid. LCMS (method B): R_{T}=0.81 min, m/z=626, 628 [M+H]⁺. ¹H NMR (500 MHz, DMSO-d₆) δ 8.05 (s, 0.5H), 8.03 (s, 0.5H), 7.68-7.54 (m, 2H), 7.44-7.36 (m, 2H), 7.36-7.29 (m, 4H), 7.29-7.22 (m, 1H), 6.80 (s, 0.5H), 6.79 (s, 0.5H), 6.36-6.10 (m, 1H), 4.88 (s, 1H), 4.07-3.87 (m, 4H), 3.84-3.74 (m, 2H), 3.74-3.57 (m, 2H), 3.49 (td, J=10.5, 4.0 Hz, 1H), 3.28-3.17 (m, 2H), 3.13-2.77 (m, 6H), 1.59-1.50 (m, 0.5H), 1.45-1.20 (m, 3.5H).

### Example 83: 6-Chloro-3-((4-hydroxy-1-(1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one

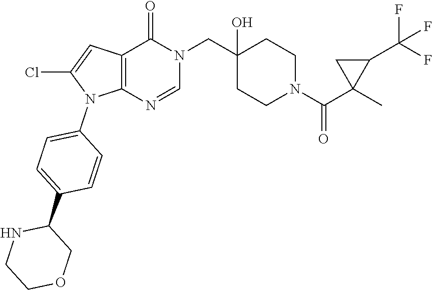 [SMILES: CC1(C(=O)N2CCC(O)(Cn3cnc4c(cc(Cl)n4-c4ccc([C@H]5COCCN5)cc4)c3=O)CC2)CC1C(F)(F)F]

### Step 1: tert-Butyl (3S)-3-(4-(6-chloro-3-((4-hydroxy-1-(1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (S)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 23 (44 mg, 0.167 mmol), Cs₂CO₃ (100 mg, 0.306 mmol) and DMF (1.4 mL) after 18 h at 80° C. gave the title compound (4:1 mixture of diastereoisomers, 54 mg, 55%) as a yellow oil. LCMS (method B): R_{T}=1.46 min, m/z=638, 640 [M−butene+H]⁺.

### Step 2: 6-Chloro-3-((4-hydroxy-1-(1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one

General procedure 7 using tert-butyl (3S)-3-(4-(6-chloro-3-((4-hydroxy-1-(1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (54 mg, 0.0778 mmol), TFA (0.8 mL) and DCM (0.8 mL) gave, after flash chromatography and freeze-drying, the title compound (4:1 mixture of diastereoisomers, 24.1 mg, 50%) as a white solid. LCMS (method B): R_{T}=0.79 min, m/z=594, 596 [M+H]⁺. ¹H NMR (500 MHz, DMSO-d₆) δ 8.07 (s, 1H), 7.61 (d, J=8.4 Hz, 2H), 7.40 (d, J=8.4 Hz, 2H), 6.81 (s, 1H), 4.93 (s, 1H), 4.08-3.73 (m, 7H), 3.53-3.45 (m, 1H), 3.43-3.27 (m, 1H (signal overlaps HDO)), 3.26-2.85 (m, 5H), 2.19-2.04 (m, 1H), 1.81-1.29 (m, 8H), 1.15-1.07 (m, 1H).

### Example 84: 6-Chloro-3-((4-hydroxy-1-(1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one

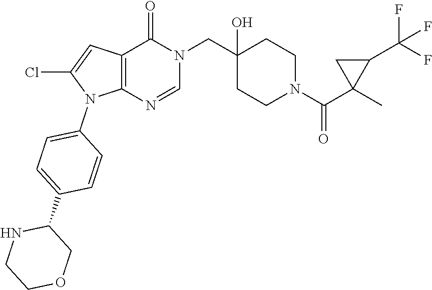 [SMILES: CC1(C(=O)N2CCC(O)(Cn3cnc4c(cc(Cl)n4-c4ccc([C@@H]5COCCN5)cc4)c3=O)CC2)CC1C(F)(F)F]

### Step 1: tert-Butyl (3R)-3-(4-(6-chloro-3-((4-hydroxy-1-(1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (R)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 23 (44 mg, 0.167 mmol), Cs₂CO₃ (100 mg, 0.306 mmol) and DMF (1.4 mL) after 18 h at 80° C. gave the title compound (4:1 mixture of diastereoisomers, 43 mg, 44%) as a yellow oil. LCMS (method B): R_{T}=1.44 min, m/z=638, 640 [M−butene+H]⁺.

### Step 2: 6-Chloro-3-((4-hydroxy-1-(1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one

General procedure 7 using tert-butyl (3R)-3-(4-(6-chloro-3-((4-hydroxy-1-(1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (43 mg, 0.0619 mmol), TFA (0.35 mL) and DCM (0.7 mL) gave, after freeze-drying, the title compound (4:1 mixture of diastereoisomers, 28 mg, 75%) as a white solid. LCMS (method B): R_{T}=0.78 min, m/z=594, 596 [M+H]⁺. ¹H NMR (500 MHz, DMSO-d₆) δ 8.07 (s, 1H), 7.66-7.60 (m, 2H), 7.43 (d, J=8.3 Hz, 2H), 6.82 (s, 1H), 4.94 (s, 1H), 4.09-3.77 (m, 7H), 3.52 (dd, J=12.1, 5.6 Hz, 1H), 3.42-3.32 (br. m, 1H, partially under HDO), 3.30-3.20 (br. m, 1H, partially under HDO), 2.96 (s, 3H), 2.12 (s, 1H), 1.56 (s, 2H), 1.40 (dt, J=14.9, 7.7 Hz, 3H), 1.36-1.33 (m, 3H), 1.12 (t, J=5.6 Hz, 1H).

### Example 85: 6-Chloro-3-((1-((R)-4,4-difluoro-3-phenylbutanoyl)-4-hydroxypiperidin-4-yl)methyl)-7-(4-((R)-morpholin-3-yl)phenyl)-3,7-dihydro-4H-pyrrolo[2,3-d]pyrimidin-4-one

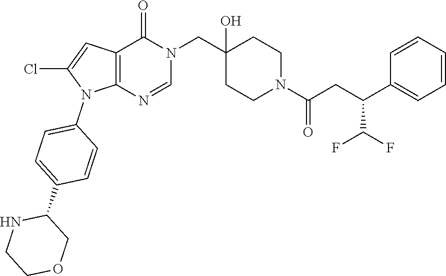 [SMILES: O=C(C[C@H](c1ccccc1)C(F)F)N1CCC(O)(Cn2cnc3c(cc(Cl)n3-c3ccc([C@@H]4COCCN4)cc3)c2=O)CC1]

### Step 1: tert-Butyl (R)-3-(4-(6-chloro-3-((1-((R)-4,4-difluoro-3-phenylbutanoyl)-4-hydroxypiperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (R)-3-(4-(6-chloro-4-oxo-3,4-dihydro-7H-pyrrolo[2,3-d]pyrimidin-7-yl)phenyl)morpholine-4-carboxylate (60 mg, 0.139 mmol), Epoxide 22 (49 mg, 0.167 mmol), Cs₂CO₃ (100 mg, 0.306 mmol) and DMF (1.4 mL) after 18 h at 80° C. gave the title compound (21 mg, 20%) as a white solid. LCMS (method B): R_{T}=1.47 min, m/z=670, 672 [M−butene+H]⁺.

### Step 2: 6-Chloro-3-((1-((R)-4,4-difluoro-3-phenylbutanoyl)-4-hydroxypiperidin-4-yl)methyl)-7-(4-((R)-morpholin-3-yl)phenyl)-3,7-dihydro-4H-pyrrolo[2,3-d]pyrimidin-4-one

General procedure 7 using tert-butyl (R)-3-(4-(6-chloro-3-((1-((R)-4,4-difluoro-3-phenylbutanoyl)-4-hydroxypiperidin-4-yl)methyl)-4-oxo-3,4-dihydro-7H-pyrrolo[2,3-d]pyrimidin-7-yl)phenyl)morpholine-4-carboxylate (21 mg, 0.0289 mmol), TFA (0.35 mL) and DCM (0.7 mL) gave, after freeze-drying, the title compound (14.8 mg, 78%) as a white solid. LCMS (method B): R_{T}=0.83 min, m/z=626, 628 [M+H]⁺. ¹H NMR (500 MHz, DMSO-d₆) δ 8.04 (d, J=10.9 Hz, 1H), 7.63-7.59 (m, 2H), 7.41 (d, J=8.0 Hz, 2H), 7.34-7.29 (m, 4H), 7.26 (q, J=4.4 Hz, 1H), 6.80 (d, J=2.3 Hz, 1H), 6.23 (td, J=56.4, 2.8 Hz, 1H), 4.87 (s, 1H), 4.03-3.87 (m, 4H), 3.79 (t, J=10.2 Hz, 2H), 3.74-3.56 (m, 2H), 3.50 (td, J=10.5, 4.1 Hz, 1H), 3.29-3.17 (m, 2H), 2.96-2.77 (m, 5H), 1.55 (s, 1H), 1.46-1.25 (m, 4H).

### Example 86: 6-Chloro-7-(3-chloro-4-((S)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one and 6-chloro-7-(3-chloro-4-((R)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one (1:1)

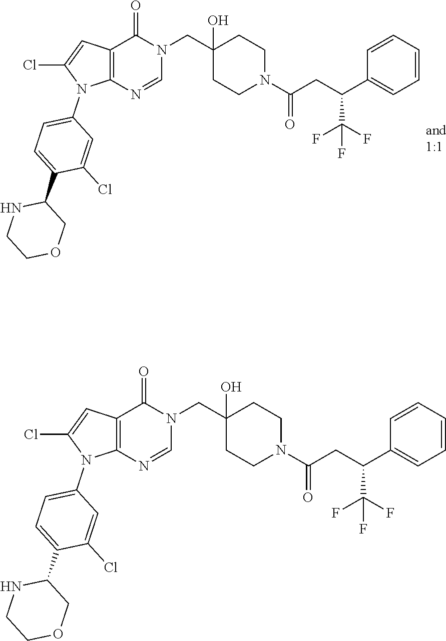 [SMILES: O=C(C[C@H](c1ccccc1)C(F)(F)F)N1CCC(O)(Cn2cnc3c(cc(Cl)n3-c3ccc([C@H]4COCCN4)c(Cl)c3)c2=O)CC1 ; O=C(C[C@H](c1ccccc1)C(F)(F)F)N1CCC(O)(Cn2cnc3c(cc(Cl)n3-c3ccc([C@@H]4COCCN4)c(Cl)c3)c2=O)CC1]

### Step 1: tert-Butyl 3-(4-bromo-2-chlorophenyl)morpholine-4-carboxylate

To a solution 2-((tributylstannyl)methoxy)ethan-1-amine (7.09 g, 19.5 mmol) in DCM (90 mL) at rt was added 4-bromo-2-chlorobenzaldehyde (4.27 g, 19.5 mmol) and 4 Å molecular sieves (ca. 2 g). The reaction mixture was stirred at rt overnight before being filtered through a short layer of Celite (DCM rinse) to give a solution of the imine in DCM. In a separate flask, 2,6-lutidine (2.26 mL, 19.5 mmol) was added in one portion to a suspension of anhydrous Cu(OTf)₂ (7.04 g, 19.5 mmol) in HFIP (78 mL) and the resulting mixture was stirred at rt overnight to give a blue suspension. The preformed solution of the imine in DCM was added in one portion and the resulting mixture was stirred at rt for 18 h. The reaction was quenched by the addition 10% aqueous ammonium hydroxide (230 mL) and, after stirring vigorously for 15 min, the layers were separated and the aqueous layer was further extracted with DCM (2×40 mL). The combined organic layers were washed with water (3×80 mL), dried over Na₂SO₄, filtered, and concentrated under reduced pressure. The crude product (containing tin compounds) was dissolved in THE (50 mL) and triethylamine (3.25 mL, 23.3 mmol), Boc₂O (5.36 mL, 23.3 mmol) and DMAP (c.a.) were added. The resulted solution was stirred at rt for 16 h before Boc₂O (5.36 mL, 23.3 mmol) was added. After the starting material was consumed, trifluoroethanol (4 mL) was added to decompose an excess of Boc₂O. After 5 h the reaction mixture was concentrated under reduced pressure and the residue was purified by flash chromatography to give the title compound (5.41 g, 74%). LCMS (method C): R_{T}=1.39 min, m/z=320, 322 [M−butene+H]⁺. ¹H NMR (400 MHz, Chloroform-d) δ 7.52 (d, J=1.8 Hz, 1H), 7.35 (dd, J=8.6, 1.8 Hz, 1H), 7.30 (d, J=8.6 Hz, 1H), 5.20 (d, J=2.4 Hz, 1H), 4.04 (d, J=11.8 Hz, 1H), 3.96 (dd, J=11.2, 2.8 Hz, 1H), 3.84 (dd, J=12.3, 4.6 Hz, 2H), 3.62 (dt, J=11.5, 3.4 Hz, 1H), 3.45 (dt, J=13.1, 4.3 Hz, 1H), 1.32 (s, 9H).

### Step 2: tert-Butyl 3-(2-chloro-4-(4,4,5,5-tetramethyl-1,3,2-dioxaborolan-2-yl)phenyl)morpholine-4-carboxylate

General procedure 3 (Work-up A) using tert-butyl 3-(4-bromo-2-chlorophenyl)morpholine-4-carboxylate (660 mg, 1.75 mmol), B₂Pin₂ (668 mg, 2.63 mmol), Pd(dppf)Cl₂·CH₂Cl₂ (149 mg, 0.175 mmol), KOAc (516 mg, 5.26 mmol) and 1,4-dioxane (17 mL) after stirring at 120° C. (microwave irradiation) for 30 min gave the title compound (554 mg, 74%) as pale yellow oil. LCMS (method B): R_{T}=1.88 min, m/z=324, 326 [M−Boc+H]⁺.

### Step 3: tert-Butyl 3-(2-chloro-4-(4,6-dichloro-7H-pyrrolo[2,3-d]pyrimidin-7-yl)phenyl)morpholine-4-carboxylate

General procedure 4 (Work-up A) using 4,6-dichloro-7H-pyrrolo[2,3-d]pyrimidine (100 mg, 0.532 mmol), tert-butyl 3-(2-chloro-4-(4,4,5,5-tetramethyl-1,3,2-dioxaborolan-2-yl)phenyl)morpholine-4-carboxylate (451 mg, 1.06 mmol), copper(II) acetate (193 mg, 1.06 mmol), 1,10-phenanthroline (192 mg, 1.06 mmol), boric acid (132 mg, 2.13 mmol) and DMF (10 mL) after 6 days at 50° C. gave the title compound (52 mg, 20%) as a colourless glass. LCMS (method B): R_{T}=1.72 min, m/z=483, 485 [M+H]⁺.

### Step 4: tert-Butyl 3-(2-chloro-4-(6-chloro-4-oxo-3,4-dihydro-7H-pyrrolo[2,3-d]pyrimidin-7-yl)phenyl)morpholine-4-carboxylate

General procedure 5 using tert-butyl 3-(2-chloro-4-(4,6-dichloro-7H-pyrrolo[2,3-d]pyrimidin-7-yl)phenyl)morpholine-4-carboxylate (52 mg, 0.108 mmol), aqueous sodium hydroxide (4 M, 0.81 mL, 3.22 mmol) and 1,4-dioxane (0.8 mL) after 18 h at 100° C. using AcOH for acidification gave the title compound (51 mg, >100%) as a yellow glass. LCMS (method B): R_{T}=1.29 min, m/z=465, 467 [M+H]⁺.

### Step 5: tert-Butyl (S)-3-(2-chloro-4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate and tert-butyl (R)-3-(2-chloro-4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate (1:1)

General procedure 6 using tert-butyl 3-(2-chloro-4-(6-chloro-4-oxo-3,4-dihydro-7H-pyrrolo[2,3-d]pyrimidin-7-yl)phenyl)morpholine-4-carboxylate (25 mg, 0.0537 mmol), Epoxide 3 (25.3 mg, 0.0806 mmol), Cs₂CO₃ (52.5 mg, 0.161 mmol) and DMF (2 mL) after 18 h at 80° C. gave the title compound (15 mg, 21%) as a colourless glass. LCMS (method B): R_{T}=1.61 min, m/z=722, 724 [M−butene+H]⁺.

### Step 6: 6-Chloro-7-(3-chloro-4-((S)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one and 6-chloro-7-(3-chloro-4-((R)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one (1:1)

General procedure 7 using tert-butyl (S)-3-(2-chloro-4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate and tert-butyl (R)-3-(2-chloro-4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)phenyl)morpholine-4-carboxylate (1:1) (22 mg, 0.0283 mmol), TFA (1 mL) and DCM (2 mL) gave, after freeze-drying, the title compound (18 mg, 93%) as a white solid. LCMS (method B): R_{T}=0.94 min, m/z=678, 680 [M+H]⁺. ¹H NMR (500 MHz, DMSO-d₆) δ 8.13-8.03 (m, 1H), 7.91 (d, J=8.3 Hz, 1H), 7.77-7.47 (m, 2H), 7.46-7.27 (m, 5H), 6.84 (s, 1H), 4.89 (s, 1H), 4.59-4.17 (m, 1H), 4.19-3.71 (m, 7H), 3.69-3.44 (m, 1H), 3.30-2.77 (m, 7H), 1.66-1.15 (m, 4H). NH not visible.

### Example 87: 6-Chloro-3-((4-hydroxy-1-((R)-4,4,4-trifluoro-3-phenylbutanoyl)piperidin-4-yl)methyl)-7-(4-((S)-morpholin-3-yl)-3-(trifluoromethyl)phenyl)-3,7-dihydro-4H-pyrrolo[2,3-d]pyrimidin-4-one and 6-chloro-3-((4-hydroxy-1-((R)-4,4,4-trifluoro-3-phenylbutanoyl)piperidin-4-yl)methyl)-7-(4-((R)-morpholin-3-yl)-3-(trifluoromethyl)phenyl)-3,7-dihydro-4H-pyrrolo[2,3-d]pyrimidin-4-one (1:1)

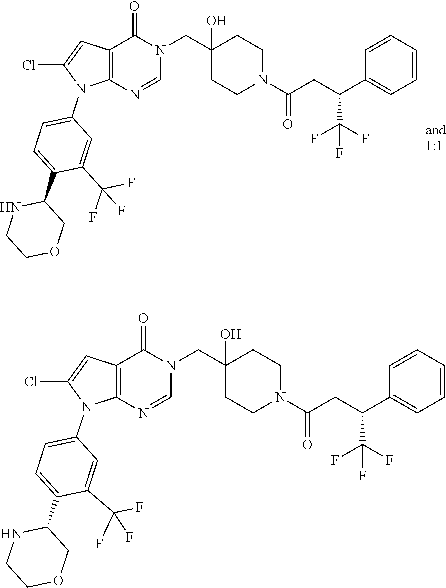 [SMILES: O=C(C[C@H](c1ccccc1)C(F)(F)F)N1CCC(O)(Cn2cnc3c(cc(Cl)n3-c3ccc([C@H]4COCCN4)c(C(F)(F)F)c3)c2=O)CC1 ; O=C(C[C@H](c1ccccc1)C(F)(F)F)N1CCC(O)(Cn2cnc3c(cc(Cl)n3-c3ccc([C@@H]4COCCN4)c(C(F)(F)F)c3)c2=O)CC1]

### Step 1: 3-(4-Bromo-2-(trifluoromethyl)phenyl)morpholine

General procedure 8 using 4-bromo-2-(trifluoromethyl)benzaldehyde (2.53 g, 10.0 mmol), 2-((tributylstannyl)methoxy)ethan-1-amine (3.64 g, 10.0 mmol), Cu(OTf)₂ (3.62 g, 10.0 mmol), 2,6-dimethylpyridine (1.07 g, 10.0 mmol), HFIP (120 mL), 4 Å molecular sieves (ca. 3 g) and DCM (480 mL) gave the title compound (1.74 g, 56%). LCMS (method C): R_{T}=0.89 min, m/z=310, 312 [M+H]⁺.

### Step 2: tert-Butyl 3-(4-bromo-2-(trifluoromethyl)phenyl)morpholine-4-carboxylate

General procedure 9 using 3-(4-bromo-2-(trifluoromethyl)phenyl)morpholine (1.74 g, 5.60 mmol) and Boc₂O (1.47 g, 6.72 mmol) gave the title compound (1.32 g, 57%). LCMS (method C): R_{T}=1.65 min, m/z=310, 312 [M−Boc+H]⁺. ¹H NMR (500 MHz, Chloroform-d) δ 7.78 (d, J=2.1 Hz, 1H), 7.62 (dd, J=8.3, 2.2 Hz, 1H), 7.46 (d, J=8.4 Hz, 1H), 5.08 (t, J=4.5 Hz, 1H), 4.02-3.93 (m, 1H), 3.87 (dd, J=12.1, 4.8 Hz, 1H), 3.80-3.63 (m, 4H), 1.22 (s, 9H).

### Step 3: tert-Butyl 3-(4-(4,4,5,5-tetramethyl-1,3,2-dioxaborolan-2-yl)-2-(trifluoromethyl)phenyl)morpholine-4-carboxylate

General procedure 3 (Work-up A) using tert-butyl 3-(4-bromo-2-(trifluoromethyl)phenyl)morpholine-4-carboxylate (500 mg, 1.22 mmol), B₂Pin₂ (464 mg, 1.83 mmol), Pd(dppf)Cl₂·CH₂Cl₂ (105 mg, 0.122 mmol), KOAc (359 mg, 3.66 mmol) and 1,4-dioxane (8 mL) after stirring at 120° C. (microwave irradiation) for 30 min gave the title compound (486 mg, 87%) as pale yellow oil. LCMS (method B): R_{T}=1.83 min, m/z=358 [M−Boc+H]⁺.

### Step 4: tert-Butyl 3-(4-(4,6-dichloro-7H-pyrrolo[2,3-d]pyrimidin-7-yl)-2-(trifluoromethyl)phenyl)morpholine-4-carboxylate

General procedure 4 (Work-up B) using 4,6-dichloro-7H-pyrrolo[2,3-d]pyrimidine (100 mg, 0.532 mmol), tert-butyl 3-(4-bromo-2-(trifluoromethyl)phenyl)morpholine-4-carboxylate (486 mg, 1.06 mmol), copper(II) acetate (193 mg, 1.06 mmol), 1,10-phenanthroline (192 mg, 1.06 mmol), boric acid (197 mg, 3.19 mmol) and DMF (4 mL) after 6 days at 50° C. gave the title compound (21 mg, 7%) as a colourless oil that solidified upon standing. LCMS (method B): R_{T}=1.74 min, m/z=517, 519 [M+H]⁺.

### Step 5: tert-Butyl 3-(4-(6-chloro-4-oxo-3,4-dihydro-7H-pyrrolo[2,3-d]pyrimidin-7-yl)-2-(trifluoromethyl)phenyl)morpholine-4-carboxylate

General procedure 5 using tert-butyl 3-(4-(4,6-dichloro-7H-pyrrolo[2,3-d]pyrimidin-7-yl)-2-(trifluoromethyl)phenyl)morpholine-4-carboxylate (21 mg, 0.0406 mmol), aqueous sodium hydroxide (2 M, 0.30 mL, 0.609 mmol) and 1,4-dioxane (0.7 mL) after 18 h at 100° C. gave the title compound (15.8 mg, 78%). LCMS (method B): R_{T}=1.33 min, m/z=499, 501 [M+H]⁺.

### Step 6: tert-Butyl (S)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)-2-(trifluoromethyl)phenyl)morpholine-4-carboxylate and tert-butyl (R)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)-2-(trifluoromethyl)phenyl)morpholine-4-carboxylate (1:1)

General procedure 6 using tert-butyl 3-(4-(6-chloro-4-oxo-3,4-dihydro-7H-pyrrolo[2,3-d]pyrimidin-7-yl)-2-(trifluoromethyl)phenyl)morpholine-4-carboxylate (15.8 mg, 0.0317 mmol), Epoxide 3 (12 mg, 0.0380 mmol), Cs₂CO₃ (22.7 mg, 0.0697 mmol) and DMF (0.7 mL) after 18 h at 80° C. gave, after purification by prep-HPLC, the title compound (9.6 mg, 37%) as a white solid. LCMS (method B): R_{T}=1.62 min, m/z=712, 714 [M−Boc+H]⁺.

### Step 7: 6-Chloro-3-((4-hydroxy-1-((R)-4,4,4-trifluoro-3-phenylbutanoyl)piperidin-4-yl)methyl)-7-(4-((S)-morpholin-3-yl)-3-(trifluoromethyl)phenyl)-3,7-dihydro-4H-pyrrolo[2,3-d]pyrimidin-4-one and 6-chloro-3-((4-hydroxy-1-((R)-4,4,4-trifluoro-3-phenylbutanoyl)piperidin-4-yl)methyl)-7-(4-((R)-morpholin-3-yl)-3-(trifluoromethyl)phenyl)-3,7-dihydro-4H-pyrrolo[2,3-d]pyrimidin-4-one (1:1)

General procedure 7 using tert-butyl (S)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)-2-(trifluoromethyl)phenyl)morpholine-4-carboxylate and tert-butyl (R)-3-(4-(6-chloro-3-((4-hydroxy-1-((R)-4,4,4-trifluoro-3-phenylbutanoyl)piperidin-4-yl)methyl)-4-oxo-3,4-dihydro-7H-pyrrolo[2,3-d]pyrimidin-7-yl)-2-(trifluoromethyl)phenyl)morpholine-4-carboxylate (1:1) (9.6 mg, 0.0118 mmol), TFA (0.35 mL) and DCM (0.7 mL) gave, after freeze-drying, the title compound (6.8 mg, 74%) as a white solid. LCMS (method B): R_{T}=0.97 min, m/z=712, 714 [M+H]⁺. ¹H NMR (500 MHz, DMSO-d₆) δ 8.19 (d, J=8.3 Hz, 1H), 8.07 (d, J=10.4 Hz, 1H), 7.89-7.78 (m, 2H), 7.49-7.25 (m, 5H), 6.88-6.81 (m, 1H), 4.89 (s, 1H), 4.18-4.06 (m, 2H), 4.04-3.87 (m, 3H), 3.83-3.69 (m, 3H), 3.59-3.50 (m, 1H), 3.28-3.11 (m, 3H), 3.02-2.79 (m, 4H), 1.64-1.55 (m, 0.5H), 1.49-1.26 (m, 3.5H). NH not visible.

### Example 88: 6-Chloro-3-((4-hydroxy-1-(1-methylcyclopropane-1-carbonyl)piperidin-4-yl)methyl)-7-(4-(morpholin-2-yl)phenyl)-3,7-dihydro-4H-pyrrolo[2,3-d]pyrimidin-4-one

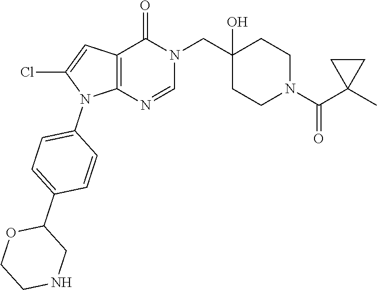 [SMILES: CC1(C(=O)N2CCC(O)(Cn3cnc4c(cc(Cl)n4-c4ccc(C5CNCCO5)cc4)c3=O)CC2)CC1]

### Step 1: tert-Butyl 2-(4-(4,6-dichloro-7H-pyrrolo[2,3-d]pyrimidin-7-yl)phenyl)morpholine-4-carboxylate

General procedure 4 (Work-up A) using 4,6-dichloro-7H-pyrrolo[2,3-d]pyrimidine (100 mg, 0.532 mmol), tert-butyl 2-(4-(4,4,5,5-tetramethyl-1,3,2-dioxaborolan-2-yl)phenyl)morpholine-4-carboxylate (414 mg, 1.06 mmol), copper(II) acetate (193 mg, 1.06 mmol), 1,10-phenanthroline (192 mg, 1.06 mmol), boric acid (132 mg, 2.13 mmol) and DMF (10 mL) after 3 days gave the title compound (107 mg. 44%) as a colourless oil. LCMS (method B): R_{T}=1.69 min, m/z=449, 451 [M+H]⁺.

### Step 2: tert-Butyl 2-(4-(6-chloro-4-oxo-3,4-dihydro-7H-pyrrolo[2,3-d]pyrimidin-7-yl)phenyl)morpholine-4-carboxylate

General procedure 5 using tert-butyl 2-(4-(4,6-dichloro-7H-pyrrolo[2,3-d]pyrimidin-7-yl)phenyl)morpholine-4-carboxylate (107 mg, 0.238 mmol), aqueous sodium hydroxide (4 M, 1.19 mL, 4.76 mmol) and 1,4-dioxane (1.2 mL) after 18 h at 100° C. using AcOH for acidification gave the title compound (103 mg, quant.) as a yellowish oil that solidified upon standing. LCMS (method A): R_{T}=1.30 min, m/z=430, 432 [M+H]⁺.

### Step 3: tert-Butyl 2-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate

General procedure 6 using tert-butyl 2-(4-(6-chloro-4-oxo-3,4-dihydro-7H-pyrrolo[2,3-d]pyrimidin-7-yl)phenyl)morpholine-4-carboxylate (103 mg, 0.239 mmol), Epoxide 1 (47 mg, 0.239 mmol), Cs₂CO₃ (234 mg, 0.717 mmol) and DMF (3 mL) after 18 h at 80° C. gave the title compound (24 mg, 16%) as a colourless glass. LCMS (method A): R_{T}=1.16 min, m/z=626, 628 [M+H]⁺.

### Step 4: 6-Chloro-3-((4-hydroxy-1-(1-methylcyclopropane-1-carbonyl)piperidin-4-yl)methyl)-7-(4-(morpholin-2-yl)phenyl)-3,7-dihydro-4H-pyrrolo[2,3-d]pyrimidin-4-one

General procedure 7 using tert-butyl 2-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (24 mg, 0.0383 mmol), TFA (1 mL) and DCM (2 mL) gave, after freeze-drying, the title compound (20 mg, 99%) as a white solid. LCMS (method B), R_{T}=0.69 min, m/z=526 [M+H]⁺. ¹H NMR (500 MHz, DMSO-d₆) δ 8.08 (s, 1H), 7.55 (d, J=8.4 Hz, 2H), 7.43 (d, J=8.4 Hz, 2H), 6.82 (s, 1H), 4.90 (s, 1H), 4.56 (dd, J=10.4, 2.4 Hz, 1H), 4.02 (s, 2H), 3.99-3.91 (m, 3H), 3.73-3.65 (m, 1H), 3.25-3.05 (m, 3H), 2.89-2.79 (m, 2H), 2.69-2.61 (m, 1H), 1.58-1.46 (m, 2H), 1.45-1.36 (m, 2H), 1.22 (s, 3H), 0.82-0.72 (m, 2H), 0.57-0.47 (m, 2H).

### Example 89: 6-Chloro-3-((1-(4-chloro-2-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

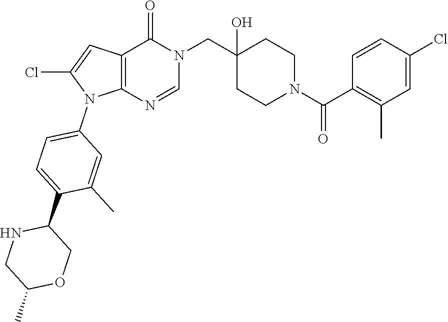 [SMILES: Cc1cc(Cl)ccc1C(=O)N1CCC(O)(Cn2cnc3c(cc(Cl)n3-c3ccc([C@H]4CO[C@H](C)CN4)c(C)c3)c2=O)CC1]

### Step 1: tert-Butyl (2R,5S)-5-(4-(6-chloro-3-((1-(4-chloro-2-methylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 24 (43.4 mg, 0.163 mmol), Cs₂CO₃ (106 mg, 0.327 mmol) and DMF (2 mL) after 18 h at 80° C. gave the title compound (52 mg, 65%) as a colourless gum. LCMS (method B): R_{T}=1.58 min, m/z=688, 690 [M−butene+H]⁺.

### Step 2: 6-Chloro-3-((1-(4-chloro-2-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-chloro-2-methylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (52 mg, 0.0718 mmol), TFA (0.5 mL) and DCM (2 mL) gave, after purification by flash chromatography and freeze-drying, the title compound (33 mg, 73%) as a white solid. LCMS (method B): R_{T}=0.92 min, m/z=624, 626 [M+H]⁺. ¹H NMR (500 MHz, DMSO-d₆) δ 8.05 (s, 1H), 7.72 (d, J=8.0 Hz, 1H), 7.44-7.34 (m, 1H), 7.33-7.27 (m, 1H), 7.27-7.20 (m, 2H), 7.20-7.10 (m, 1H), 6.77 (s, 1H), 4.95 (s, 1H), 4.24-4.16 (m, 1H), 4.07-4.00 (m, 2H), 3.98 (dd, J=10.0, 3.0 Hz, 1H), 3.79 (dd, J=10.8, 3.0 Hz, 1H), 3.63-3.52 (m, 1H), 3.26-3.04 (m, 4H), 2.97 (dd, J=11.7, 2.3 Hz, 1H), 2.77 (s, 1H), 2.57 (t, J=10.9 Hz, 1H), 2.41 (s, 3H), 2.28-2.11 (m, 3H), 1.69-1.41 (m, 3H), 1.36-1.23 (m, 1H), 1.10 (d, J=6.2 Hz, 3H).

### Example 90: 6-Chloro-3-((1-(4-chloro-2-methylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

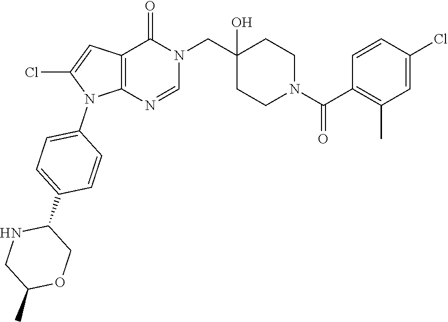 [SMILES: Cc1cc(Cl)ccc1C(=O)N1CCC(O)(Cn2cnc3c(cc(Cl)n3-c3ccc([C@@H]4CO[C@@H](C)CN4)cc3)c2=O)CC1]

### Step 1: tert-Butyl (2S,5R)-5-(4-(6-chloro-3-((1-(4-chloro-2-methylbenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (50 mg, 0.112 mmol), Epoxide 24 (44.8 mg, 0.169 mmol), Cs₂CO₃ (110 mg, 0.337 mmol) and DMF (2 mL) after 18 h at 80° C. gave the title compound (57 mg, 71%) as a colourless gum. LCMS (method B): R_{T}=1.53 min, m/z=654, 656 [M−butene+H]⁺.

### Step 2: 6-Chloro-3-((1-(4-chloro-2-methylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-chloro-2-methylbenzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (57 mg, 0.0802 mmol), TFA (1 mL) and DCM (2 mL) gave, after purification by flash chromatography and freeze-drying, the title compound (36 mg, 73%) as a white solid. LCMS (method B): R_{T}=0.87 min, m/z=610, 612 [M+H]⁺. ¹H NMR (500 MHz, DMSO-d₆) δ 8.06 (s, 1H), 7.64-7.57 (m, 2H), 7.43-7.35 (m, 3H), 7.35-7.12 (m, 2H), 6.79 (s, 1H), 4.96 (s, 1H), 4.29-4.16 (m, 1H), 4.02 (s, 2H), 3.89-3.76 (m, 2H), 3.61-3.50 (m, 1H), 3.29-3.04 (m, 4H), 3.01-2.81 (m, 2H), 2.59-2.52 (m, 1H (overlapping DMSO)), 2.31-2.10 (m, 3H), 1.73-1.39 (m, 3H), 1.38-1.18 (m, 1H), 1.10 (d, 3H).

### Example 91: 6-Chloro-3-((1-(4-(difluoromethyl)benzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

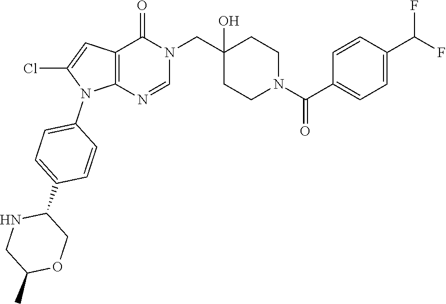 [SMILES: C[C@H]1CN[C@H](c2ccc(-n3c(Cl)cc4c(=O)n(CC5(O)CCN(C(=O)c6ccc(C(F)F)cc6)CC5)cnc43)cc2)CO1]

### Step 1: tert-Butyl (2S,5R)-5-(4-(6-chloro-3-((1-(4-(difluoromethyl)benzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (50 mg, 0.112 mmol), Epoxide 25 (45 mg, 0.169 mmol), Cs₂CO₃ (110 mg, 0.337 mmol) and DMF (2 mL) after 18 h at 80° C. gave the title compound (40 mg, 50%) as a colourless gum. LCMS (method B): R_{T}=1.44 min, m/z=656, 658 [M−butene+H]⁺.

### Step 2: 6-Chloro-3-((1-(4-(difluoromethyl)benzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(4-(difluoromethyl)benzoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (40 mg, 0.0562 mmol), TFA (0.5 mL) and DCM (2 mL) gave, after purification by flash chromatography and freeze-drying, the title compound (20 mg, 58%) as a white solid. LCMS (method B): R_{T}=0.83 min, m/z=612, 614 [M+H]⁺. ¹H NMR (500 MHz, DMSO-d₆) δ 8.06 (s, 1H), 7.64 (d, J=8.0 Hz, 2H), 7.63-7.57 (m, 2H), 7.53 (d, J=8.0 Hz, 2H), 7.43-7.35 (m, 2H), 7.08 (t, J=55.8 Hz, 1H), 6.79 (s, 1H), 4.96 (s, 1H), 4.27-4.11 (m, 1H), 4.10-3.96 (m, 2H), 3.88-3.75 (m, 2H), 3.55 (dqd, J=12.4, 6.1, 2.2 Hz, 1H), 3.38-3.21 (m, 3H (signals overlap HDO)), 3.20-3.08 (m, 1H), 2.95 (dd, J=11.6, 2.3 Hz, 1H), 2.90 (s, 1H), 2.57-2.52 (m, 1H (signal overlap DMSO)), 1.71-1.53 (m, 2H), 1.53-1.41 (m, 1H), 1.40-1.27 (m, 1H), 1.09 (d, J=6.2 Hz, 3H).

### Example 92: 6-Chloro-3-((1-(4-(difluoromethyl)benzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

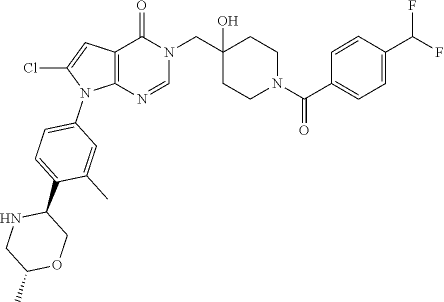 [SMILES: Cc1cc(-n2c(Cl)cc3c(=O)n(CC4(O)CCN(C(=O)c5ccc(C(F)F)cc5)CC4)cnc32)ccc1[C@H]1CO[C@H](C)CN1]

### Step 1: tert-Butyl (2R,5S)-5-(4-(6-chloro-3-((1-(4-(difluoromethyl)benzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 25 (43.7 mg, 0.163 mmol), Cs₂CO₃ (106 mg, 0.327 mmol) and DMF (2 mL) after 18 h at 80° C. gave the title compound (31 mg, 39%) as a colourless gum. LCMS (method B): R_{T}=1.48 min, m/z=670, 672 [M−butene+H]⁺.

### Step 2: 6-Chloro-3-((1-(4-(difluoromethyl)benzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-(difluoromethyl)benzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (31 mg, 0.0427 mmol), TFA (0.5 mL) and DCM (2 mL) gave, after purification by flash chromatography and freeze-drying, the title compound (19 mg, 71%) as a white solid. LCMS (method B): R_{T}=0.85 min, m/z=626, 628 [M+H]⁺. ¹H NMR (500 MHz, DMSO-d₆) δ 8.09-8.01 (m, 1H), 7.72 (d, J=8.2 Hz, 1H), 7.64 (d, J=7.9 Hz, 2H), 7.52 (d, J=7.7 Hz, 2H), 7.28-7.20 (m, 2H), 7.08 (t, J=55.8 Hz, 1H), 6.81-6.71 (m, 1H), 4.96 (s, 1H), 4.29-4.10 (m, 1H), 4.08-4.00 (m, 2H), 4.00-3.91 (m, 1H), 3.84-3.74 (m, 1H), 3.63-3.52 (m, 1H), 3.42-3.17 (m, 3H (signals overlap with HDO)), 3.17-3.06 (m, 1H), 2.96 (d, J=11.7 Hz, 1H), 2.77 (s, 1H), 2.57 (t, J=10.8 Hz, 1H), 2.41 (s, 3H), 1.70-1.53 (m, 2H), 1.52-1.40 (m, 1H), 1.40-1.26 (m, 1H), 1.09 (d, J=6.1 Hz, 3H).

### Example 93: 6-Chloro-3-((1-(4-cyclopropylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

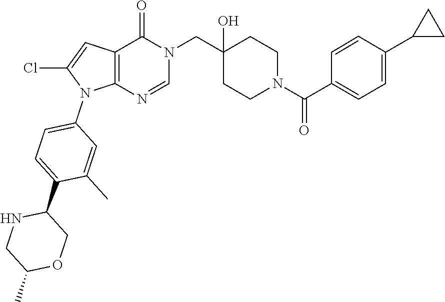 [SMILES: Cc1cc(-n2c(Cl)cc3c(=O)n(CC4(O)CCN(C(=O)c5ccc(C6CC6)cc5)CC4)cnc32)ccc1[C@H]1CO[C@H](C)CN1]

### Step 1: tert-Butyl (2R,5S)-5-(4-(6-chloro-3-((1-(4-cyclopropylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 26 (42.1 mg, 0.163 mmol), Cs₂CO₃ (106 mg, 0.327 mmol) and DMF (2 mL) after 18 h at 80° C. gave the title compound (56 mg, 71%) as a colourless gum. LCMS (method B): R_{T}=1.58 min, m/z=660, 662 [M−butene+H]⁺.

### Step 2: 6-Chloro-3-((1-(4-cyclopropylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((1-(4-cyclopropylbenzoyl)-4-hydroxypiperidin-4-yl)methyl)-4-oxo-3,4-dihydro-7H-pyrrolo[2,3-d]pyrimidin-7-yl)-2-methylphenyl)-2-methylmorpholine-4-carboxylate (56 mg, 0.0782 mmol), TFA (1 mL) and DCM (2 mL) gave, after purification by flash chromatography and freeze-drying, the title compound (35 mg, 72%) as a white solid. LCMS (method B): R_{T}=0.90 min, m/z=616, 618 [M+H]⁺. ¹H NMR (500 MHz, DMSO-d₆) δ 8.06 (s, 1H), 7.73 (d, J=7.9 Hz, 1H), 7.30-7.19 (m, 4H), 7.15-7.08 (m, 2H), 6.77 (s, 1H), 4.94 (s, 1H), 4.13 (br. s, 1H), 4.01 (s, 2H), 3.98 (dd, J=10.0, 3.0 Hz, 1H), 3.79 (dd, J=10.9, 3.0 Hz, 1H), 3.62-3.51 (m, 1H), 3.43 (br. s, 1H), 3.28-3.04 (m, 3H), 2.97 (dd, J=11.6, 2.3 Hz, 1H), 2.77 (br. s, 1H), 2.57 (t, J=10.9 Hz, 1H), 2.41 (s, 3H), 1.95 (tt, J=8.4, 5.0 Hz, 1H), 1.66-1.50 (m, 2H), 1.50-1.27 (m, 2H), 1.10 (d, J=6.2 Hz, 3H), 1.04-0.92 (m, 2H), 0.76-0.63 (m, 2H).

### Example 94: 6-Chloro-3-((4-hydroxy-1-(4-(1-methylcyclopropyl)benzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

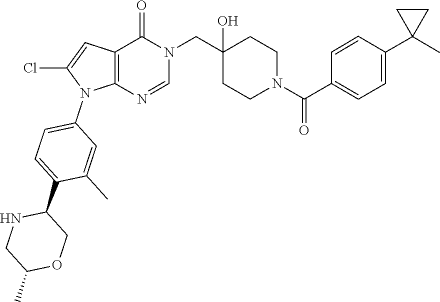 [SMILES: Cc1cc(-n2c(Cl)cc3c(=O)n(CC4(O)CCN(C(=O)c5ccc(C6(C)CC6)cc5)CC4)cnc32)ccc1[C@H]1CO[C@H](C)CN1]

### Step 1: tert-Butyl (2R,5S)-5-(4-(6-chloro-3-((4-hydroxy-1-(4-(1-methylcyclopropyl)benzoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)-2-methylphenyl)-2-methylmorpholine-4-carboxylate (50 mg, 0.109 mmol), Epoxide 27 (44.3 mg, 0.163 mmol), Cs₂CO₃ (106 mg, 0.327 mmol) and DMF (2 mL) after 18 h at 80° C. gave the title compound (53 mg, 66%) as a colourless gum. LCMS (method B): R_{T}=1.65 min, m/z=674, 676 [M−butene+H]⁺.

### Step 2: 6-Chloro-3-((4-hydroxy-1-(4-(1-methylcyclopropyl)benzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-(4-(1-methylcyclopropyl)benzoyl)piperidin-4-yl)methyl)-4-oxo-3,4-dihydro-7H-pyrrolo[2,3-d]pyrimidin-7-yl)-2-methylphenyl)-2-methylmorpholine-4-carboxylate (53 mg, 0.0726 mmol), TFA (1 mL) and DCM (2 mL) gave, after purification by flash chromatography and freeze-drying, the title compound (35 mg, 76%) as a white solid. LCMS (method B): R_{T}=0.96 min, m/z=630, 632 [M+H]⁺. ¹H NMR (500 MHz, DMSO-d₆) δ 8.06 (s, 1H), 7.73 (d, J=8.0 Hz, 1H), 7.34-7.20 (m, 6H), 6.77 (s, 1H), 4.94 (s, 1H), 4.15 (br. s, 1H), 4.01 (s, 2H), 3.98 (dd, J=10.0, 2.7 Hz, 1H), 3.79 (dd, J=10.9, 3.0 Hz, 1H), 3.62-3.53 (m, 1H), 3.43 (br. s, 1H), 3.28-3.04 (m, 3H), 2.97 (dd, J=11.6, 2.3 Hz, 1H), 2.77 (br. s, 1H), 2.56 (t, J=11.2 Hz, 1H), 2.41 (s, 3H), 1.58 (br. s, 2H), 1.45 (br. s, 1H), 1.39 (s, 3H), 1.30 (br. s, 1H), 1.10 (d, J=6.2 Hz, 3H), 0.91-0.83 (m, 2H), 0.83-0.75 (m, 2H).

### Example 95: 6-Chloro-3-((1-((1 S,2S)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one

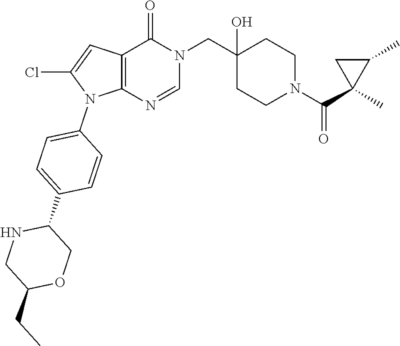 [SMILES: CC[C@H]1CN[C@H](c2ccc(-n3c(Cl)cc4c(=O)n(CC5(O)CCN(C(=O)[C@@]6(C)C[C@@H]6C)CC5)cnc43)cc2)CO1]

### Step 1: tert-Butyl (2S,5R)-5-(4-(6-chloro-3-((1-((1S,2S)-1,2-dimethylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-ethylmorpholine-4-carboxylate (67.9 mg, 0.148 mmol), Epoxide 28 (46.4 mg, 0.222 mmol), Cs₂CO₃ (145 mg, 0.444 mmol) and DMF (2 mL) after 18 h at 80° C. gave the title compound (76 mg, 76%) as a colourless gum. LCMS (method B): R_{T}=1.50 min, m/z=612, 614 [M−butene+H]⁺.

### Step 2: 6-Chloro-3-((1-((1S,2S)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-((1S,2S)-1,2-dimethylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate (76 mg, 0.114 mmol), TFA (1 mL) and DCM (2 mL) gave, after purification by flash chromatography and freeze-drying, the title compound (47 mg, 72%) as a white solid. LCMS (method B): R_{T}=0.82 min, m/z=568, 570 [M+H]⁺. ¹H NMR (500 MHz, DMSO-d₆) δ 8.07 (s, 1H), 7.65-7.56 (m, 2H), 7.43-7.35 (m, 2H), 6.80 (s, 1H), 4.90 (s, 1H), 4.03 (d, J=13.8 Hz, 1H), 3.98 (d, J=13.7 Hz, 1H), 3.92-3.73 (m, 4H), 3.37-3.32 (m, 1H), 3.25 (t, J=11.0 Hz, 1H), 3.12 (br. s, 2H), 2.97 (dd, J=11.7, 2.3 Hz, 1H), 2.92 (br. s, 1H), 2.58-2.52 (m, 1H), 1.57-1.33 (m, 6H), 1.15 (s, 3H), 1.09-1.01 (m, 3H), 1.00-0.87 (m, 5H), 0.18-0.11 (m, 1H).

### Example 96: 6-Chloro-3-((1-((1R,2R)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one

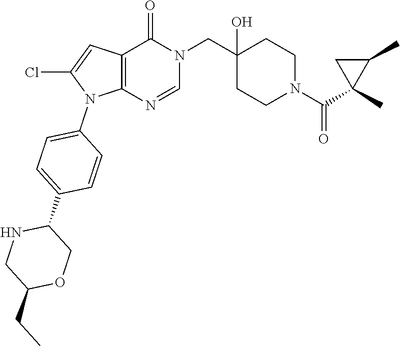 [SMILES: CC[C@H]1CN[C@H](c2ccc(-n3c(Cl)cc4c(=O)n(CC5(O)CCN(C(=O)[C@]6(C)C[C@H]6C)CC5)cnc43)cc2)CO1]

### Step 1: tert-Butyl (2S,5R)-5-(4-(6-chloro-3-((1-((1R,2R)-1,2-dimethylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-ethylmorpholine-4-carboxylate (67.9 mg, 0.148 mmol), Epoxide 29 (46.4 mg, 0.222 mmol), Cs₂CO₃ (145 mg, 0.444 mmol) and DMF (2 mL) after 18 h at 80° C. gave the title compound (75 mg, 75%) as a colourless gum. LCMS (method B): R_{T}=1.50 min, m/z=612, 614 [M−butene+H]⁺.

### Step 2: 6-Chloro-3-((1-((1R,2R)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-((1R,2R)-1,2-dimethylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-ethylmorpholine-4-carboxylate (75 mg, 0.112 mmol), TFA (1 mL) and DCM (2 mL) gave, after purification by flash chromatography and freeze-drying, the title compound (41 mg, 64%) as a white solid. LCMS (method B): R_{T}=0.82 min, m/z=568, 570 [M+H]⁺. ¹H NMR (500 MHz, DMSO-d₆) δ 8.07 (s, 1H), 7.63-7.57 (m, 2H), 7.43-7.36 (m, 2H), 6.80 (s, 1H), 4.90 (s, 1H), 4.03 (d, J=13.8 Hz, 1H), 3.98 (d, J=13.7 Hz, 1H), 3.92-3.73 (m, 4H), 3.38-3.32 (m, 1H), 3.25 (t, J=11.0 Hz, 1H), 3.13 (br. s, 2H), 2.98 (dd, J=11.7, 2.3 Hz, 1H), 2.92 (br. s, 1H), 2.55 (dd, J=11.6, 10.1 Hz, 1H), 1.56-1.34 (m, 6H), 1.15 (s, 3H), 1.10-1.02 (m, 3H), 0.99-0.87 (m, 5H), 0.17-0.11 (m, 1H).

### Example 97: 7-(4-((1 S,2R,5R)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

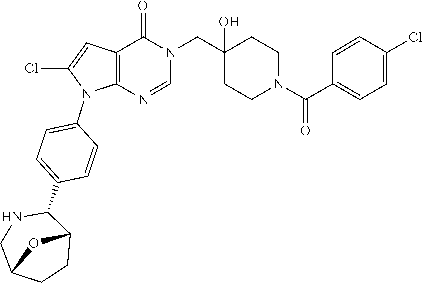 [SMILES: O=C(c1ccc(Cl)cc1)N1CCC(O)(Cn2cnc3c(cc(Cl)n3-c3ccc([C@H]4NC[C@H]5CC[C@@H]4O5)cc3)c2=O)CC1]

### Step 1: (R)-5-(((tert-Butyldiphenylsilyl)oxy)methyl)dihydrofuran-2(3H)-one

To (R)-5-(hydroxymethyl)dihydrofuran-2(3H)-one (1.00 g, 8.61 mmol) and imidazole (1.17 g, 17.2 mmol) in DMF (10 mL) at 0° C. was added tert-butylchlorodiphenylsilane (2.91 mL, 11.2 mmol). The reaction mixture was allowed to warm to rt and stirred overnight before EtOAc (50 mL) was added. The resulting mixture was washed with 1:1 brine/water (3×50 mL). The organic phase was dried over MgSO₄, filtered, and concentrated under reduced pressure. The residue was purified by flash chromatography to give title compound (2.74 g, 89%) as a colourless oil that solidified upon storage. LCMS (method B): R_{T}=1.70 min, m/z=277 [M−Ph]⁺. ¹H NMR (500 MHz, Chloroform-d) δ 7.70-7.61 (m, 4H), 7.48-7.34 (m, 6H), 4.66-4.56 (m, 1H), 3.88 (dd, J=11.4, 3.4 Hz, 1H), 3.69 (dd, J=11.3, 3.4 Hz, 1H), 2.67 (ddd, J=17.5, 10.2, 7.1 Hz, 1H), 2.51 (ddd, J=17.8, 10.1, 6.5 Hz, 1H), 2.37-2.15 (m, 2H), 1.06 (s, 9H).

### Step 2: (5R)-5-(((tert-Butyldiphenylsilyl)oxy)methyl)tetrahydrofuran-2-ol

To a solution of (R)-5-(((tert-butyldiphenylsilyl)oxy)methyl)dihydrofuran-2(3H)-one (2.74 g, 7.72 mmol) dry diethyl ether (40 mL) under nitrogen and at −78° C. was added diisobutylaluminium hydride (1 M in DCM, 11.6 mL, 11.6 mmol). The reaction was stirred at −78° C. for 5 h before being quenched by the addition of MeOH (5 mL). After stirring at −78° C. for 15 min the reaction mixture was warmed to rt and diluted with diethyl ether (150 mL). The resulting mixture was washed with 0.2 M aqueous sodium tartrate (3×100 mL) and brine. The organic phase was dried over MgSO₄, filtered, and concentrated under reduced pressure to give the title compound (2.75 g, quant.) as a colourless oil. LCMS (method B): R_{T}=1.69 min, m/z=339 [M−OH]⁺.

### Step 3: (R)-tert-Butyl((2,3-dihydrofuran-2-yl)methoxy)diphenylsilane

To a solution of (5R)-5-(((tert-butyldiphenylsilyl)oxy)methyl)tetrahydrofuran-2-ol (2.75 g, 7.71 mmol) in DCM (50 mL) at −50° C. under N₂, were added triethylamine (4.09 mL, 29.3 mmol) followed by methanesulfonyl chloride (748 μL, 9.64 mmol). The reaction mixture was stirred at −50° C. for 3 h until all the starting material had been consumed at which point the reaction was warmed up to rt and subsequently stirred at reflux for a further 18 h. Upon cooling to rt the reaction mixture was concentrated under reduced pressure and the residue was purified by flash chromatography to give title compound (1.37 g, 52%) as a colourless oil. ¹H NMR (500 MHz, Chloroform-d) δ 7.74-7.65 (m, 4H), 7.45-7.34 (m, 6H), 6.27 (q, J=2.5 Hz, 1H), 4.85 (q, J=2.6 Hz, 1H), 4.71-4.58 (m, 1H), 3.75 (dd, J=10.8, 5.6 Hz, 1H), 3.68 (dd, J=10.7, 4.9 Hz, 1H), 2.64 (ddt, J=15.2, 10.4, 2.4 Hz, 1H), 2.47 (ddt, J=15.1, 7.3, 2.4 Hz, 1H), 1.06 (s, 9H).

### Step 4: (R)-(2,3-Dihydrofuran-2-yl)methyl 4-methylbenzenesulfonate

To (R)-tert-butyl((2,3-dihydrofuran-2-yl)methoxy)diphenylsilane (1.37 g, 4.05 mmol) was added TBAF (1 M in THF, 4.05 mL, 4.05 mmol) and the resulting mixture was stirred at rt for 1 h. The reaction mixture was diluted with DCM (25 mL) and cooled to 0° C. before triethylamine (5.64 mL, 40.5 mmol) and 4-methylbenzenesulfonyl chloride (3.86 g, 20.3 mmol) were added. The reaction mixture was stirred for 19 h whilst slowly being allowed to warm to rt. The reaction mixture was concentrated under reduced pressure, the residue was dissolved in EtOAc (50 mL) and washed with 1:1 brine/water (2×50 mL). The organic layer was dried over MgSO₄, filtered, and concentrated under reduced pressure. The residue was purified by flash chromatography to give title compound (858 mg, 83%) as a colourless oil. LCMS (method A): R_{T}=1.40 min, m/z=255 [M+H]⁺.

### Step 5: (R)-2-((2,3-Dihydrofuran-2-yl)methyl)isoindoline-1,3-dione

A mixture of (R)-(2,3-dihydrofuran-2-yl)methyl 4-methylbenzenesulfonate (858 mg, 3.74 mmol) and potassium 1,3-dioxoisoindolin-2-ide (937 mg, 5.06 mmol) in DMF (13 mL) under nitrogen was stirred at 75° C. for 19 h. Upon cooling to rt the reaction mixture was diluted with EtOAc (50 mL) and washed with 1:1 brine/water (3×50 mL). The organic layer was dried over MgSO₄, filtered, and concentrated under reduced pressure. The residue was purified by flash chromatography to give title compound (623 mg, 80%) as a white solid. LCMS (method B): R_{T}=1.10 min, m/z=230 [M+H]⁺.

### Step 6: (R)-(2,3-Dihydrofuran-2-yl)methanamine

To (R)-2-((2,3-dihydrofuran-2-yl)methyl)isoindoline-1,3-dione (320 mg, 1.40 mmol) in MeOH (5.5 mL) was added hydrazine monohydrate (260 μL, 8.38 mmol) and the resulting mixture was heated at 60° C. for 2 h. Upon cooling to rt aqueous sodium hydroxide (2 M, 10 mL) was added and the resulting mixture was extracted with DCM (3×20 mL). The combined organic layers were washed with brine (50 mL), dried over MgSO₄, filtered, and concentrated at reduced pressure (41° C., 220 mbar) to give the title compound (184 mg, >100%). This material was used without purification in the next step.

### Step 7: (1S,2R,5R)-2-(4-Bromophenyl)-8-oxa-3-azabicyclo[3.2.1]octane

A mixture of crude (R)-(2,3-dihydrofuran-2-yl)methanamine (184 mg, 1.40 mmol), 4-bromobenzaldehyde (258 mg, 1.39 mmol) and 3 Å molecular sieves (300 mg) in DCM (1.4 mL) was stirred at rt for 19 h. The reaction mixture was filtered through a syringe filter and the volatiles were evaporated under reduced pressure (210 mbar, 40° C.). The residue was dissolved in 2-propanol (14 mL) and transferred to in a 25 mL pressure vial before tris(2,2,6,6-tetramethyl-3,5-heptanedionato)manganese(III) (42 mg, 0.0695 mmol) was added and the vial was capped with a septa. To the resulting mixture was then added via a syringe through the septa PhSiH₃ (342 μL, 2.78 mmol) and the reaction was heated at 85° C. for 5 h. Further PhSiH₃ (342 μL, 2.78 mmol) was added, the pressure inside the vial was released and the reaction was heated at 85° C. for 19 h. Further PhSiH₃ (342 μL, 2.78 mmol) was added, the pressure inside the vial was released and the reaction was heated at 85° C. for 4 h. Upon cooling to rt the reaction mixture was concentrated under reduced pressure and the residue was purified by flash chromatography to give the title compound (84 mg, 22%) as a colourless glass. LCMS (method B): R_{T}=0.59 min, m/z=268, 270 [M+H]⁺. ¹H NMR (500 MHz, Chloroform-d) δ 7.48-7.43 (m, 2H), 7.29-7.26 (m, 2H), 4.40-4.32 (m, 1H), 4.23-4.13 (m, 2H), 3.23 (dd, J=11.2, 2.3 Hz, 1H), 2.80 (dd, J=11.2, 1.7 Hz, 1H), 2.02-1.80 (m, 3H), 1.55-1.47 (m, 1H). NH not visible.

### Step 8: tert-Butyl (1S,2R,5R)-2-(4-bromophenyl)-8-oxa-3-azabicyclo[3.2.1]octane-3-carboxylate

To a solution of (1 S,2R,5R)-2-(4-bromophenyl)-8-oxa-3-azabicyclo[3.2.1]octane (82 mg, 0.306 mmol) and DIPEA (80 μL, 0.459 mmol) in DCM (5 mL) at rt was added dropwise a solution of Boc₂O (80 mg, 0.367 mmol) in DCM (2 mL). The reaction mixture was stirred at rt for 18 h before the volatiles were removed under reduced pressure and the residue was purified by flash chromatography to give the title compound (105 mg, 93%) as a colourless glass. LCMS (method B): R_{T}=1.59 min, m/z=268, 270 [M−Boc+H]⁺.

### Step 9: tert-Butyl (1S,2R,5R)-2-(4-(4,4,5,5-tetramethyl-1,3,2-dioxaborolan-2-yl)phenyl)-8-oxa-3-azabicyclo[3.2.1]octane-3-carboxylate

General procedure 3 (Work-up A) using tert-butyl (1S,2R,5R)-2-(4-bromophenyl)-8-oxa-3-azabicyclo[3.2.1]octane-3-carboxylate (105 mg, 0.285 mmol), B₂Pin₂ (109 mg, 0.428 mmol), Pd(dppf)Cl₂·CH₂Cl₂ (24.1 mg, 0.0285 mmol), KOAc (84 mg, 0.855 mmol) and 1,4-dioxane (5 mL) after stirring at 100° C. (thermal) for 4 h gave the title compound (112 mg, 94%) as a colourless glass. LCMS (method B): R_{T}=1.71 min, m/z=316 [M−Boc+H]⁺.

### Step 10: tert-Butyl (1S,2R,5R)-2-(4-(4,6-dichloro-7H-pyrrolo[2,3-d]pyrimidin-7-yl)phenyl)-8-oxa-3-azabicyclo[3.2.1]octane-3-carboxylate

General procedure 4 (Work-up A) using 4,6-dichloro-7H-pyrrolo[2,3-d]pyrimidine (39 mg, 0.207 mmol), tert-butyl (1S,2R,5R)-2-(4-(4,4,5,5-tetramethyl-1,3,2-dioxaborolan-2-yl)phenyl)-8-oxa-3-azabicyclo[3.2.1]octane-3-carboxylate (112 mg, 0.270 mmol), copper(II) trifluoroacetate hydrate (120 mg, 0.415 mmol), 1,10-phenanthroline (74.7 mg, 0.415 mmol), boric acid (51.3 mg, 0.830 mmol) and DMF (3.9 mL) after 4 days at 50° C. gave the title compound (60 mg, 60%) as a colourless glass. LCMS (method B): R_{T}=1.68 min, m/z=475, 477 [M+H]⁺.

### Step 11: tert-Butyl (1 S,2R,5R)-2-(4-(6-chloro-4-oxo-3,4-dihydro-7H-pyrrolo[2,3-d]pyrimidin-7-yl)phenyl)-8-oxa-3-azabicyclo[3.2.1]octane-3-carboxylate

General procedure 5 using tert-butyl (1S,2R,5R)-2-(4-(4,6-dichloro-7H-pyrrolo[2,3-d]pyrimidin-7-yl)phenyl)-8-oxa-3-azabicyclo[3.2.1]octane-3-carboxylate (60 mg, 0.126 mmol), aqueous sodium hydroxide (4 M, 947 μL, 3.79 mmol) and 1,4-dioxane (1 mL) after 18 h at 100° C. using 1 M aqueous potassium bisulfate for acidification gave the title compound (58 mg, quant.) as a yellow glass. LCMS (method A): R_{T}=1.20 min, m/z=455, 456 [M−H]⁻. This material was used without further purification.

### Step 12: tert-Butyl (1S,2R,5R)-2-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-8-oxa-3-azabicyclo[3.2.1]octane-3-carboxylate

General procedure 6 using tert-butyl (1 S,2R,5R)-2-(4-(6-chloro-4-oxo-3,4-dihydro-7H-pyrrolo[2,3-d]pyrimidin-7-yl)phenyl)-8-oxa-3-azabicyclo[3.2.1]octane-3-carboxylate (57 mg, 0.125 mmol), Epoxide 2 (47.1 mg, 0.187 mmol), Cs₂CO₃ (122 mg, 0.374 mmol) and DMF (2 mL) after 18 h at 80° C. gave the title compound (22 mg, 24%) as a colourless glass. LCMS (method B): R_{T}=1.47 min, m/z=652, 654 [M−butene+H]⁺.

### Step 13: 7-(4-((1S,2R,5R)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one

General procedure 7 using tert-butyl (1 S,2R,5R)-2-(4-(6-chloro-3-((1-(4-chlorobenzoyl)-4-hydroxypiperidin-4-yl)methyl)-4-oxo-3,4-dihydro-7H-pyrrolo[2,3-d]pyrimidin-7-yl)phenyl)-8-oxa-3-azabicyclo[3.2.1]octane-3-carboxylate (22 mg, 0.0311 mmol), TFA (1 mL) and DCM (2 mL) gave, after freeze-drying, the title compound (19 mg, quant.) as a white solid. LCMS (method B): R_{T}=0.80 min, m/z=608, 610 [M+H]⁺. ¹H NMR (500 MHz, DMSO-d₆) δ 8.07 (s, 1H), 7.62-7.57 (m, 2H), 7.54-7.48 (m, 2H), 7.47-7.38 (m, 4H), 6.80 (s, 1H), 4.96 (s, 1H), 4.31-4.21 (m, 2H), 4.23-4.09 (m, 2H), 4.09-3.96 (m, 2H), 3.46-3.33 (m, 1H), 3.29-3.08 (m, 2H), 3.06-2.97 (m, 1H), 2.82-2.59 (m, 2H), 1.98-1.87 (m, 1H), 1.86-1.70 (m, 2H), 1.70-1.26 (m, 5H).

### Example 98: (R)-6-Chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one or (S)-6-chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

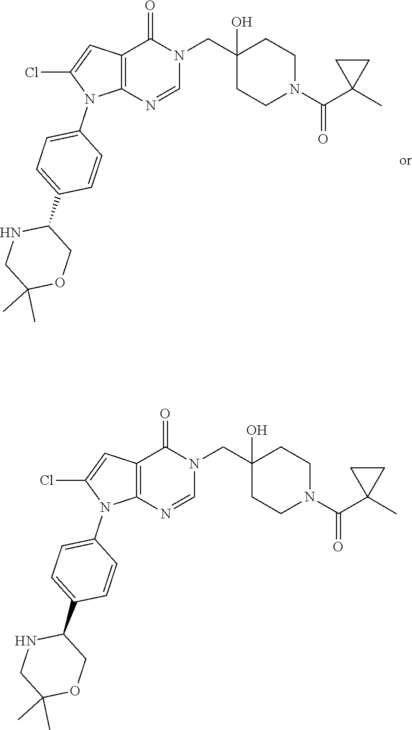

### Step 1: tert-Butyl (R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate or tert-butyl (S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate

General procedure 6 using tert-butyl (R)-5-(4-(6-chloro-4-oxo-3,4-dihydro-7H-pyrrolo[2,3-d]pyrimidin-7-yl)phenyl)-2,2-dimethylmorpholine-4-carboxylate (80 mg, 0.174 mmol), Epoxide 1 (51 mg, 0.262 mmol), Cs₂CO₃ (170 mg, 0.523 mmol) and DMF (2 mL) after 18 h at 80° C. gave the title compound (72 mg, 63%) as a colourless gum. LCMS (method B): R_{T}=1.39 min, m/z=598, 600 [M−butene+H]⁺.

### Step 2: (R)-6-Chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one or (S)-6-chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

General procedure 7 using tert-butyl (R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate or tert-butyl (S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2,2-dimethylmorpholine-4-carboxylate (72 mg, 0.110 mmol), TFA (1 mL) and DCM (2 mL) gave, after purification by flash chromatography and freeze-drying, the title compound (51 mg, 83%) as a white solid. LCMS (method D): R_{T}=0.73 min, m/z=554, 556 [M+H]⁺. ¹H NMR (500 MHz, DMSO-d₆) δ 8.07 (s, 1H), 7.67-7.59 (m, 2H), 7.44-7.37 (m, 2H), 6.80 (s, 1H), 4.91 (s, 1H), 4.01 (s, 2H), 3.95 (dt, J=13.2, 4.2 Hz, 2H), 3.80 (dd, J=10.3, 3.6 Hz, 1H), 3.57 (dd, J=11.1, 3.6 Hz, 1H), 3.46 (t, J=10.7 Hz, 1H), 3.16 (br. s, 2H), 2.80 (br. s, 1H), 2.77 (d, J=11.6 Hz, 1H), 2.68 (d, J=11.5 Hz, 1H), 1.58-1.45 (m, 2H), 1.44-1.36 (m, 2H), 1.35 (s, 3H), 1.21 (s, 3H), 1.13 (s, 3H), 0.82-0.71 (m, 2H), 0.58-0.46 (m, 2H).

### Example 99: 6-Chloro-3-((1-(2,2-dicyclopropylacetyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

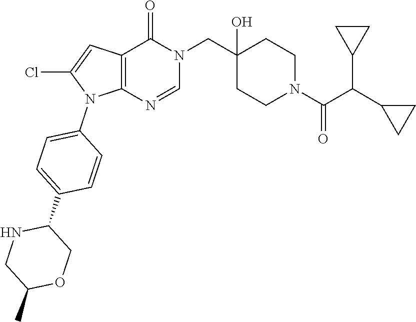 [SMILES: C[C@H]1CN[C@H](c2ccc(-n3c(Cl)cc4c(=O)n(CC5(O)CCN(C(=O)C(C6CC6)C6CC6)CC5)cnc43)cc2)CO1]

### Step 1: tert-Butyl (2S,5R)-5-(4-(6-chloro-3-((1-(2,2-dicyclopropylacetyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (70 mg, 0.157 mmol), Epoxide 30 (54 mg, 0.230 mmol), Cs₂CO₃ (154 mg, 0.472 mmol) and DMF (2 mL) after 18 h at 80° C. gave the title compound (70 mg, 65%) as a colourless gum. LCMS (method B): R_{T}=1.46 min, m/z=624, 626 [M−butene+H]⁺.

### Step 2: 6-Chloro-3-((1-(2,2-dicyclopropylacetyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(2,2-dicyclopropylacetyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (70 mg, 0.103 mmol), TFA (0.5 mL) and DCM (2 mL) gave, after purification by flash chromatography and freeze-drying, the title compound (40 mg, 67%) as a white solid. LCMS (method D): R_{T}=0.79 min, m/z=580, 582 [M+H]⁺. ¹H NMR (500 MHz, DMSO-d₆) δ 8.06 (s, 1H), 7.62-7.56 (m, 2H), 7.43-7.34 (m, 2H), 6.80 (s, 1H), 4.91 (s, 1H), 4.09-4.00 (m, 2H), 4.00-3.91 (m, 1H), 3.88-3.77 (m, 2H), 3.68-3.59 (m, 1H), 3.55 (dqd, J=12.4, 6.2, 2.2 Hz, 1H), 3.30-3.17 (m, 2H), 3.04-2.91 (m, 2H), 2.57-2.52 (m, 1H), 1.66 (t, J=8.8 Hz, 1H), 1.51-1.32 (m, 4H), 1.09 (d, J=6.2 Hz, 3H), 1.06-0.94 (m, 2H), 0.51-0.39 (m, 2H), 0.36-0.18 (m, 4H), 0.09-0.00 (m, 2H). NH not visible.

### Example 100: 6-Chloro-7-(4-((3R,6S)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

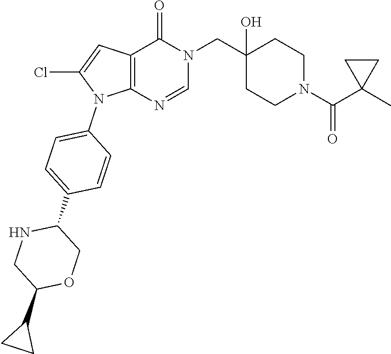 [SMILES: CC1(C(=O)N2CCC(O)(Cn3cnc4c(cc(Cl)n4-c4ccc([C@@H]5CO[C@@H](C6CC6)CN5)cc4)c3=O)CC2)CC1]

### Step 1: tert-Butyl (2S,5R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-cyclopropylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-cyclopropylmorpholine-4-carboxylate (75 mg, 0.159 mmol), Epoxide 1 (46.6 mg, 0.239 mmol), Cs₂CO₃ (156 mg, 0.478 mmol) and DMF (2 mL) after 18 h at 80° C. gave the title compound (73 mg, 68%) as a colourless glass. LCMS (method D): R_{T}=1.39 min, m/z=610, 612 [M−butene+H]⁺.

### Step 2: 6-Chloro-7-(4-((3R,6S)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

General procedure 7 using tert-butyl (2S,5R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-cyclopropylmorpholine-4-carboxylate (73 mg, 0.129 mmol), TFA (1 mL) and DCM (2 mL) gave, after purification by flash chromatography and freeze-drying, the title compound (39.4 mg, 63%) as a white solid. LCMS (method D): R_{T}=0.74 min, m/z=566, 568 [M+H]⁺. ¹H NMR (500 MHz, DMSO-d₆) δ 8.06 (s, 1H), 7.63-7.54 (m, 2H), 7.42-7.35 (m, 2H), 6.80 (s, 1H), 4.90 (s, 1H), 4.00 (s, 2H), 3.94 (dt, J=13.4, 4.2 Hz, 2H), 3.90-3.78 (m, 2H), 3.19 (t, J=10.2 Hz, 1H), 3.16 (br. s, 2H), 3.06-3.01 (m, 1H), 2.90 (br. s, 1H), 2.82 (ddd, J=10.0, 7.7, 2.2 Hz, 1H), 2.76-2.68 (m, 1H), 1.58-1.45 (m, 2H), 1.45-1.35 (m, 2H), 1.21 (s, 3H), 0.88-0.79 (m, 1H), 0.78-0.73 (m, 2H), 0.57-0.48 (m, 2H), 0.48-0.40 (m, 2H), 0.34-0.21 (m, 2H).

### Example 101: 6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

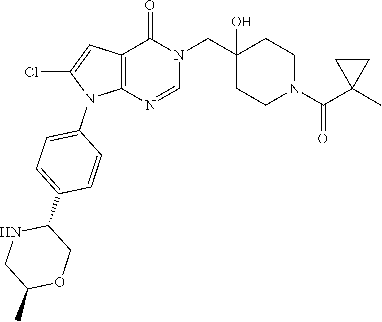 [SMILES: C[C@H]1CN[C@H](c2ccc(-n3c(Cl)cc4c(=O)n(CC5(O)CCN(C(=O)C6(C)CC6)CC5)cnc43)cc2)CO1]

### Step 1: tert-Butyl (2S,5R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (80 mg, 0.180 mmol), Epoxide 1 (52.7 mg, 0.270 mmol), Cs₂CO₃ (176 mg, 0.539 mmol) and DMF (2 mL) after 18 h at 80° C. gave the title compound (88 mg, 76%) as a colourless gum. LCMS (method D): R_{T}=1.39 min, m/z=584, 586 [M−butene+H]⁺.

### Step 2: 6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (88 mg, 0.138 mmol), TFA (0.5 mL) and DCM (2 mL) gave, after purification by flash chromatography and freeze-drying, the title compound (63 mg, 84%) as a white solid. LCMS (method D): R_{T}=0.65 min, m/z=540, 542 [M+H]⁺. ¹H NMR (500 MHz, DMSO-d₆) δ 8.07 (s, 1H), 7.63-7.57 (m, 2H), 7.43-7.36 (m, 2H), 6.80 (s, 1H), 4.91 (s, 1H), 4.01 (s, 2H), 3.94 (dt, J=13.3, 4.3 Hz, 2H), 3.88-3.77 (m, 2H), 3.55 (dqd, J=12.5, 6.1, 2.3 Hz, 1H), 3.25 (t, J=10.3 Hz, 1H), 3.15 (br. s, 2H), 3.00-2.92 (m, 1H), 2.90 (br. s, 1H), 2.57-2.52 (m, 1H), 1.58-1.45 (m, 2H), 1.44-1.34 (m, 2H), 1.21 (s, 3H), 1.09 (d, J=6.2 Hz, 3H), 0.82-0.70 (m, 2H), 0.57-0.45 (m, 2H).

### Example 102: 6-Chloro-3-((4-hydroxy-1-(1-methylcyclobutane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

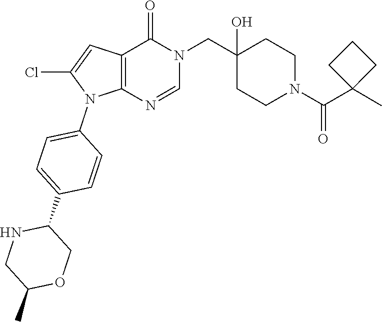 [SMILES: C[C@H]1CN[C@H](c2ccc(-n3c(Cl)cc4c(=O)n(CC5(O)CCN(C(=O)C6(C)CCC6)CC5)cnc43)cc2)CO1]

### Step 1: tert-Butyl (2S,5R)-5-(4-(6-chloro-3-((4-hydroxy-1-(1-methylcyclobutane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (80 mg, 0.180 mmol), Epoxide 31 (56.4 mg, 0.270 mmol), Cs₂CO₃ (176 mg, 0.539 mmol) and DMF (2 mL) after 18 h at 80° C. gave the title compound (54 mg, 45%) as a colourless glass. LCMS (method D): R_{T}=1.41 min, m/z=598, 600 [M−butene+H]⁺.

### Step 2: 6-Chloro-3-((4-hydroxy-1-(1-methylcyclobutane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((4-hydroxy-1-(1-methylcyclobutane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (54 mg, 0.0825 mmol), TFA (0.5 mL) and DCM (2 mL) gave, after purification by flash chromatography and freeze-drying, the title compound (44 mg, 96%) as a white solid. LCMS (method D): R_{T}=0.70 min, m/z=554, 556 [M+H]⁺. ¹H NMR (500 MHz, DMSO-d₆) δ 8.06 (s, 1H), 7.63-7.57 (m, 2H), 7.42-7.36 (m, 2H), 6.80 (s, 1H), 4.90 (s, 1H), 4.08-3.90 (m, 3H), 3.87-3.78 (m, 2H), 3.55 (dqd, J=12.5, 6.2, 2.2 Hz, 1H), 3.39-3.32 (m, 1H), 3.25 (t, J=10.3 Hz, 1H), 3.21-3.11 (m, 1H), 3.02-2.83 (m, 3H), 2.57-2.52 (m, 1H), 2.42-2.33 (m, 2H), 1.96-1.85 (m, 1H), 1.83-1.72 (m, 2H), 1.66-1.56 (m, 1H), 1.56-1.41 (m, 2H), 1.41-1.34 (m, 2H), 1.33 (s, 3H), 1.09 (d, J=6.2 Hz, 3H).

### Example 103: 6-Chloro-3-((1-(3-cyclopropylpropanoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

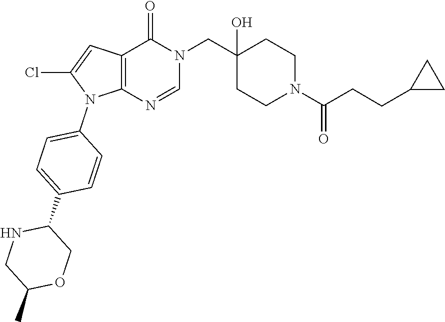 [SMILES: C[C@H]1CN[C@H](c2ccc(-n3c(Cl)cc4c(=O)n(CC5(O)CCN(C(=O)CCC6CC6)CC5)cnc43)cc2)CO1]

### Step 1: tert-Butyl (2S,5R)-5-(4-(6-chloro-3-((1-(3-cyclopropylpropanoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (80 mg, 0.180 mmol), Epoxide 32 (56.4 mg, 0.270 mmol), Cs₂CO₃ (176 mg, 0.539 mmol) and DMF (2 mL) after 18 h at 80° C. gave the title compound (50 mg, 42%) as a colourless glass. LCMS (method D): R_{T}=1.41 min, m/z=598, 600 [M−butene+H]⁺.

### Step 2: 6-Chloro-3-((1-(3-cyclopropylpropanoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-(3-cyclopropylpropanoyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (50 mg, 0.0764 mmol), TFA (0.5 mL) and DCM (2 mL) gave, after purification by flash chromatography and freeze-drying, the title compound (22 mg, 52%) as a white solid. LCMS (method D): R_{T}=0.71 min, m/z=554, 556 [M+H]⁺. ¹H NMR (500 MHz, DMSO-d₆) δ 8.04 (s, 1H), 7.62-7.54 (m, 2H), 7.41-7.34 (m, 2H), 6.77 (s, 1H), 4.87 (s, 1H), 4.06-3.90 (m, 3H), 3.85-3.73 (m, 2H), 3.66-3.59 (m, 1H), 3.57-3.47 (m, 1H), 3.27-3.20 (m, 2H), 3.07-2.75 (m, 3H), 2.55-2.49 (m, 1H), 2.39-2.28 (m, 2H), 1.56-1.46 (m, 1H), 1.44-1.29 (m, 5H), 1.06 (d, J=6.2 Hz, 3H), 0.74-0.62 (m, 1H), 0.37-0.29 (m, 2H), 0.08-−0.08 (m, 2H).

### Example 104: 6-Chloro-7-(4-((3S,6R)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

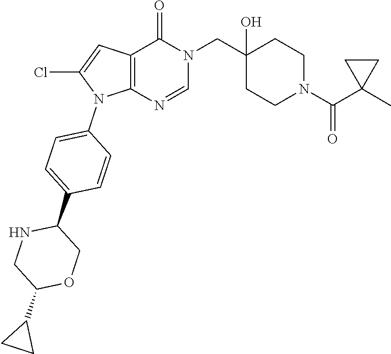 [SMILES: CC1(C(=O)N2CCC(O)(Cn3cnc4c(cc(Cl)n4-c4ccc([C@H]5CO[C@H](C6CC6)CN5)cc4)c3=O)CC2)CC1]

### Step 1: tert-Butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-cyclopropylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-cyclopropylmorpholine-4-carboxylate (80 mg, 0.170 mmol), Epoxide 1 (49.8 mg, 0.255 mmol), Cs₂CO₃ (166 mg, 0.510 mmol) and DMF (2 mL) after 18 h at 80° C. gave the title compound (77 mg, 68%) as a colourless glass. LCMS (method D): R_{T}=1.39 min, m/z=610, 612 [M−butene+H]⁺.

### Step 2: 6-Chloro-7-(4-((3S,6R)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

General procedure 7 using tert-butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-cyclopropylmorpholine-4-carboxylate (77 mg, 0.116 mmol), TFA (0.5 mL) and DCM (2 mL) gave, after purification by flash chromatography and freeze-drying, the title compound (50 mg, 76%) as a white solid. LCMS (method D): R_{T}=0.69 min, m/z=566, 568 [M+H]⁺. ¹H NMR (500 MHz, DMSO-d₆) δ 8.07 (s, 1H), 7.61-7.57 (m, 2H), 7.41-7.36 (m, 2H), 6.80 (s, 1H), 4.90 (s, 1H), 4.00 (s, 2H), 3.94 (dt, J=13.4, 4.1 Hz, 2H), 3.89-3.77 (m, 2H), 3.20 (t, J=10.2 Hz, 1H), 3.16 (br. s, 2H), 3.04 (dd, J=11.5, 2.2 Hz, 1H), 2.98 (br. s, 1H), 2.83 (ddd, J=10.1, 7.8, 2.2 Hz, 1H), 2.72 (dd, J=11.6, 10.2 Hz, 1H), 1.55-1.46 (m, 2H), 1.43-1.35 (m, 2H), 1.21 (s, 3H), 0.87-0.79 (m, 1H), 0.79-0.72 (m, 2H), 0.55-0.49 (m, 2H), 0.48-0.41 (m, 2H), 0.34-0.22 (m, 2H).

### Example 105: 6-Chloro-7-(4-((3S,6R)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

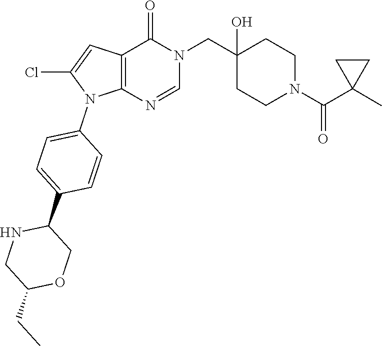 [SMILES: CC[C@@H]1CN[C@@H](c2ccc(-n3c(Cl)cc4c(=O)n(CC5(O)CCN(C(=O)C6(C)CC6)CC5)cnc43)cc2)CO1]

### Step 1: tert-Butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-ethylmorpholine-4-carboxylate (80 mg, 0.174 mmol), Epoxide 1 (51.1 mg, 0.262 mmol), Cs₂CO₃ (170 mg, 0.523 mmol) and DMF (2 mL) after 18 h at 80° C. gave the title compound (90 mg, 78%) as a colourless glass. LCMS (method D): R_{T}=1.42 min, m/z=598, 600 [M−butene+H]⁺.

### Step 2: 6-Chloro-7-(4-((3S,6R)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

General procedure 7 using tert-butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate (90 mg, 0.138 mmol), TFA (1 mL) and DCM (2 mL) gave, after purification by flash chromatography and freeze-drying, the title compound (49 mg, 64%) as a white solid. LCMS (method D): R_{T}=0.69 min, m/z=554, 556 [M+H]⁺. ¹H NMR (500 MHz, DMSO-d₆) δ 8.07 (s, 1H), 7.63-7.58 (m, 2H), 7.42-7.36 (m, 2H), 6.80 (s, 1H), 4.91 (s, 1H), 4.01 (s, 2H), 3.95 (dt, J=13.2, 4.2 Hz, 2H), 3.89-3.79 (m, 2H), 3.37-3.32 (m, 1H), 3.30-3.21 (m, 1H), 3.15 (br. s, 2H), 2.98 (dd, J=11.7, 2.3 Hz, 1H), 2.93 (br. s, 1H), 2.55 (dd, J=11.5, 10.1 Hz, 1H), 1.58-1.34 (m, 6H), 1.21 (s, 3H), 0.91 (t, J=7.5 Hz, 3H), 0.80-0.68 (m, 2H), 0.56-0.45 (m, 2H).

### Example 106: 6-Chloro-7-(4-((3R,6S)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

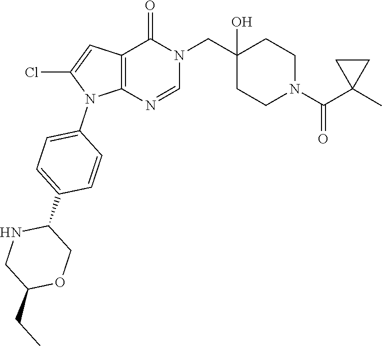 [SMILES: CC[C@H]1CN[C@H](c2ccc(-n3c(Cl)cc4c(=O)n(CC5(O)CCN(C(=O)C6(C)CC6)CC5)cnc43)cc2)CO1]

### Step 1: tert-Butyl (2S,5R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-ethylmorpholine-4-carboxylate (80 mg, 0.174 mmol), Epoxide 1 (51.1 mg, 0.262 mmol), Cs₂CO₃ (170 mg, 0.523 mmol) and DMF (2 mL) after 18 h at 80° C. gave the title compound (72 mg, 63%) as a colourless glass. LCMS (method D): R_{T}=1.41 min, m/z=598, 600 [M−butene+H]⁺.

### Step 2: 6-Chloro-7-(4-((3R,6S)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one

General procedure 7 using tert-butyl (2S,5R)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-ethylmorpholine-4-carboxylate (72 mg, 0.110 mmol), TFA (1 mL) and DCM (2 mL) gave, after purification by flash chromatography and freeze-drying, the title compound (42 mg, 68%) as a white solid. LCMS (method D): R_{T}=0.69 min, m/z=554, 556 [M+H]⁺. ¹H NMR (500 MHz, DMSO-d₆) δ 8.07 (s, 1H), 7.64-7.57 (m, 2H), 7.43-7.36 (m, 2H), 6.80 (s, 1H), 4.91 (s, 1H), 4.01 (s, 2H), 3.94 (dt, J=13.2, 4.3 Hz, 2H), 3.88-3.80 (m, 2H), 3.38-3.32 (m, 1H), 3.28-3.22 (m, 1H), 3.15 (br. s, 2H), 2.97 (dd, J=11.7, 2.3 Hz, 1H), 2.94 (br. s, 1H), 2.55 (dd, J=11.7, 10.2 Hz, 1H), 1.56-1.31 (m, 6H), 1.21 (s, 3H), 0.91 (t, J=7.5 Hz, 3H), 0.80-0.73 (m, 2H), 0.54-0.48 (m, 2H).

### Example 107: 6-Chloro-3-((4-hydroxy-1-(1-methylcyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one

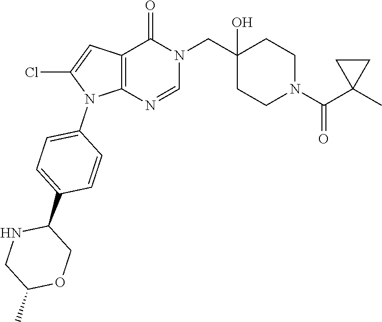 [SMILES: C[C@@H]1CN[C@@H](c2ccc(-n3c(Cl)cc4c(=O)n(CC5(O)CCN(C(=O)C6(C)CC6)CC5)cnc43)cc2)CO1]

### Step 1: tert-Butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (80 mg, 0.180 mmol), Epoxide 1 (52.7 mg, 0.270 mmol), Cs₂CO₃ (176 mg, 0.539 mmol) and DMF (2 mL) after 18 h at 80° C. gave the title compound (78 mg, 67%) as a colourless glass. LCMS (method D): R_{T}=1.39 min, m/z=584, 586 [M−butene+H]⁺.

### Step 2: 6-Chloro-3-((4-hydroxy-1-(1-methylcyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-(1-methylcyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (78 mg, 0.122 mmol), TFA (1 mL) and DCM (2 mL) gave, after purification by flash chromatography and freeze-drying, the title compound (56 mg, 85%) as a white solid. LCMS (method D): R_{T}=0.64 min, m/z=540, 542 [M+H]⁺. ¹H NMR (500 MHz, DMSO-d₆) δ 8.07 (s, 1H), 7.63-7.57 (m, 2H), 7.42-7.37 (m, 2H), 6.80 (s, 1H), 4.91 (s, 1H), 4.01 (s, 2H), 3.94 (dt, J=13.1, 4.3 Hz, 2H), 3.88-3.76 (m, 2H), 3.59-3.49 (m, 1H), 3.25 (t, J=10.3 Hz, 1H), 3.16 (br. s, 2H), 2.95 (dd, J=11.7, 2.3 Hz, 1H), 2.92 (br. s, 1H), 2.58-2.52 (m, 1H), 1.62-1.44 (m, 2H), 1.44-1.32 (m, 2H), 1.21 (s, 3H), 1.09 (d, J=6.2 Hz, 3H), 0.76 (q, J=4.1 Hz, 2H), 0.60-0.44 (m, 2H).

### Example 108: 6-Chloro-3-((4-hydroxy-1-((1R,2R)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one

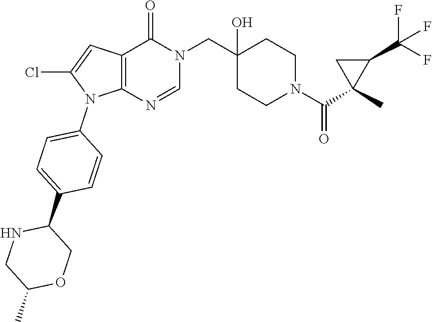 [SMILES: C[C@@H]1CN[C@@H](c2ccc(-n3c(Cl)cc4c(=O)n(CC5(O)CCN(C(=O)[C@]6(C)C[C@H]6C(F)(F)F)CC5)cnc43)cc2)CO1]

### Step 1: tert-Butyl (2R,5S)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (70 mg, 0.157 mmol), Epoxide 9 (62.1 mg, 0.236 mmol), Cs₂CO₃ (154 mg, 0.472 mmol) and DMF (2 mL) after 18 h at 80° C. gave the title compound (79 mg, 70%) as a colourless glass. LCMS (method D): R_{T}=1.49 min, m/z=652, 654 [M−butene+H]⁺.

### Step 2: 6-Chloro-3-((4-hydroxy-1-((1R,2R)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (79 mg, 0.112 mmol), TFA (1 mL) and DCM (2 mL) gave, after purification by flash chromatography and freeze-drying, the title compound (56 mg, 82%) as a white solid. LCMS (method D): R_{T}=0.76 min, m/z=608, 610 [M+H]⁺. ¹H NMR (500 MHz, DMSO-d₆) δ 8.06 (s, 1H), 7.63-7.56 (m, 2H), 7.43-7.36 (m, 2H), 6.81 (s, 1H), 4.93 (s, 1H), 4.08-3.74 (m, 6H), 3.55 (dqd, J=12.5, 6.1, 2.2 Hz, 1H), 3.38 (br. s, 1H), 3.25 (t, J=10.3 Hz, 1H), 2.99 (br. s, 2H), 2.95 (dd, J=11.7, 2.3 Hz, 1H), 2.57-2.52 (m, 1H), 2.20-2.03 (m, 1H), 1.80-1.47 (m, 2H), 1.47-1.35 (m, 3H), 1.34 (s, 3H), 1.15-1.10 (m, 1H), 1.09 (d, J=6.2 Hz, 3H).

### Example 109: 6-Chloro-3-((4-hydroxy-1-((1S,2S)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one

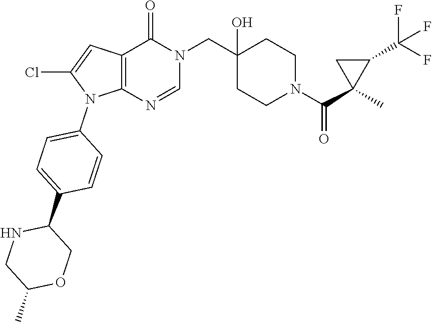 [SMILES: C[C@@H]1CN[C@@H](c2ccc(-n3c(Cl)cc4c(=O)n(CC5(O)CCN(C(=O)[C@@]6(C)C[C@@H]6C(F)(F)F)CC5)cnc43)cc2)CO1]

### Step 1: tert-Butyl (2R,5S)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2R,5S)-5-(4-(6-chloro-4-oxo-3,4-dihydro-7H-pyrrolo[2,3-d]pyrimidin-7-yl)phenyl)-2-methylmorpholine-4-carboxylate (70 mg, 0.157 mmol), Epoxide 10 (62.1 mg, 0.236 mmol), Cs₂CO₃ (154 mg, 0.472 mmol) and DMF (2 mL) after 18 h at 80° C. gave the title compound (82 mg, 73%) as a colourless glass. LCMS (method D): R_{T}=1.49 min, m/z=652, 654 [M−butene+H]⁺.

### Step 2: 6-Chloro-3-((4-hydroxy-1-((1S,2S)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one

General procedure 7 using tert-butyl (2R,5S)-5-(4-(6-chloro-3-((4-hydroxy-1-((1S,2S)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)-2-methylmorpholine-4-carboxylate (82 mg, 0.116 mmol), TFA (1 mL) and DCM (2 mL) gave, after purification by flash chromatography and freeze-drying, the title compound (45 mg, 63%) as a white solid. LCMS (method D): R_{T}=0.76 min, m/z=608, 610 [M+H]⁺. ¹H NMR (500 MHz, DMSO-d₆) δ 8.06 (s, 1H), 7.64-7.57 (m, 2H), 7.42-7.36 (m, 2H), 6.81 (s, 1H), 4.93 (s, 1H), 4.08-3.77 (m, 6H), 3.59-3.49 (m, 1H), 3.35 (br. s, 1H), 3.25 (t, J=10.3 Hz, 1H), 2.95 (dd, J=11.7, 2.4 Hz, 1H), 2.92 (br. s, 2H), 2.57-2.52 (m, 1H), 2.20-2.04 (m, 1H), 1.79-1.49 (m, 2H), 1.49-1.35 (m, 3H), 1.34 (s, 3H), 1.15-1.10 (m, 1H), 1.09 (d, J=6.3 Hz, 3H).

### Example 110: 6-Chloro-3-((4-hydroxy-1-((1R,2R)-1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one or 6-chloro-3-((4-hydroxy-1-((1R,2R)-1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one

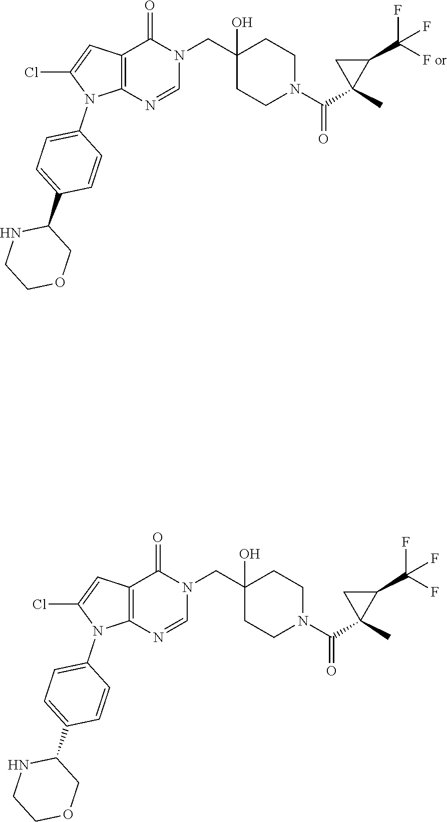

### Step 1: tert-Butyl (S)-3-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate or tert-butyl (R)-3-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate

General procedure 6 using tert-butyl (S)-3-(4-(6-chloro-4-oxo-3,4-dihydro-7H-pyrrolo[2,3-d]pyrimidin-7-yl)phenyl)morpholine-4-carboxylate (63 mg, 0.146 mmol), Epoxide 9 (42.3 mg, 0.161 mmol), Cs₂CO₃ (95.3 mg, 0.292 mmol) and DMF (2 mL) after 18 h at 80° C. gave the title compound (67.5 mg, 66%) as an off-white foam. LCMS (method A): R_{T}=1.53 min, m/z=638, 640 [M−butene+H]⁺.

### Step 2: 6-Chloro-3-((4-hydroxy-1-((1R,2R)-1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one or 6-chloro-3-((4-hydroxy-1-((1R,2R)-1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one

General procedure 7 using tert-butyl (S)-3-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate or tert-butyl (R)-3-(4-(6-chloro-3-((4-hydroxy-1-((1R,2R)-1-methyl-2-(trifluoromethyl)cyclopropane-1-carbonyl)piperidin-4-yl)methyl)-4-oxo-3,4-dihydro-7H-pyrrolo[2,3-d]pyrimidin-7-yl)phenyl)morpholine-4-carboxylate (67.5 mg, 0.0972 mmol), TFA (0.5 mL) and DCM (1 mL) gave, after freeze-drying, the title compound (47.3 mg, 81%) as an off-white solid. LCMS (method A): R_{T}=0.71 min, m/z=594, 596 [M+H]⁺. ¹H NMR (500 MHz, DMSO-d₆) δ 8.07 (s, 1H), 7.65-7.57 (m, 2H), 7.44-7.35 (m, 2H), 6.81 (s, 1H), 4.93 (s, 1H), 4.11-3.69 (m, 7H), 3.48 (td, J=10.6, 3.8 Hz, 1H), 3.35 (br. s, 1H (signal overlaps with HDO)), 3.22 (t, J=10.4 Hz, 1H), 3.13-2.79 (m, 4H), 2.19-2.04 (m, 1H), 1.83-1.35 (m, 5H), 1.35-1.26 (m, 3H), 1.11 (t, J=5.9 Hz, 1H).

### Example 111: 6-Chloro-3-((1-((1 S,2S)-2-(difluoromethyl)-1-methylcyclopropane-1-carbonyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

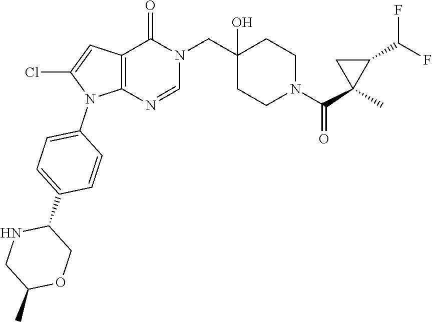 [SMILES: C[C@H]1CN[C@H](c2ccc(-n3c(Cl)cc4c(=O)n(CC5(O)CCN(C(=O)[C@@]6(C)C[C@@H]6C(F)F)CC5)cnc43)cc2)CO1]

### Step 1: tert-Butyl (2S,5R)-5-(4-(6-chloro-3-((1-((1S,2S)-2-(difluoromethyl)-1-methylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate and tert-butyl (2S,5R)-5-(4-(6-chloro-3-((1-((1R,2R)-2-(difluoromethyl)-1-methylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate

General procedure 6 using tert-butyl (2S,5R)-5-(4-(6-chloro-4-oxo-3,4-dihydro-7H-pyrrolo[2,3-d]pyrimidin-7-yl)phenyl)-2-methylmorpholine-4-carboxylate (245 mg, 0.551 mmol), Epoxide 33 (162 mg, 0.661 mmol), Cs₂CO₃ (359 mg, 1.10 mmol) and DMF (5.5 mL) after 18 h at 80° C. gave the product as a 1:1 mixture of diastereoisomers (284 mg, 74%). LCMS (method B): R_{T}=1.40 min, m/z=634, 636 [M−butene+H]⁺. A portion of this material (250 mg, 0.362 mmol) was resolved into the single stereoisomers by chiral SFC using a Chiralpak IH (21 mm×250 mm, 5 μm) column with isocratic solvent conditions: 15:85 MeOH:CO₂ (0.2% v/v NH₃). The first eluted material was tert-butyl (2S,5R)-5-(4-(6-chloro-3-((1-((1 S,2S)-2-(difluoromethyl)-1-methylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (74.5 mg, 30% recovery) obtained as an off-white solid. Chiral purity (method L): R_{T}=10.73 min, 100% ee. The second eluted material was tert-butyl (2S,5R)-5-(4-(6-chloro-3-((1-((1R,2R)-2-(difluoromethyl)-1-methylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (66.3 mg, 27% recovery) obtained as an off-white solid. Chiral purity (method L): R_{T}=12.18 min, 99.8% ee.

### Step 2: 6-Chloro-3-((1-((1S,2S)-2-(difluoromethyl)-1-methylcyclopropane-1-carbonyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

General procedure 7 using tert-butyl (2S,5R)-5-(4-(6-chloro-3-((1-((1 S,2S)-2-(difluoromethyl)-1-methylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (74.5 mg, 0.108 mmol), TFA (0.5 mL) and DCM (1 mL) gave, after freeze-drying, the title compound (61.7 mg, 96%) as a greyish-white solid. LCMS (method B): R_{T}=0.76 min, m/z=590, 592 [M+H]⁺. ¹H NMR (500 MHz, DMSO-d₆) δ 8.06 (s, 1H), 7.64-7.57 (m, 2H), 7.43-7.36 (m, 2H), 6.81 (s, 1H), 5.93 (td, J=55.5, 7.2 Hz, 1H), 4.92 (s, 1H), 4.03 (d, J=13.8 Hz, 1H), 3.98 (d, J=13.8 Hz, 1H), 3.95-3.75 (m, 4H), 3.56 (dqd, J=12.5, 6.1, 2.2 Hz, 1H), 3.45-2.75 (br. m, 3H (signals overlap with HDO)), 3.27 (t, J=10.4 Hz, 1H), 2.97 (dd, J=11.8, 2.3 Hz, 1H), 2.55 (dd, J=11.8, 10.1 Hz, 1H), 1.70-1.46 (m, 3H), 1.45-1.34 (m, 2H), 1.31 (s, 3H), 1.18 (ddd, J=8.5, 4.9, 2.8 Hz, 1H), 1.09 (d, J=6.2 Hz, 3H), 0.94-0.88 (m, 1H).

### Example 112: 6-Chloro-3-((1-((1R,2R)-2-(difluoromethyl)-1-methylcyclopropane-1-carbonyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one

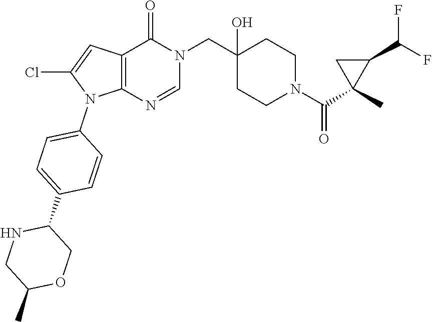 [SMILES: C[C@H]1CN[C@H](c2ccc(-n3c(Cl)cc4c(=O)n(CC5(O)CCN(C(=O)[C@]6(C)C[C@H]6C(F)F)CC5)cnc43)cc2)CO1]

General procedure 7 using tert-butyl (2S,5R)-5-(4-(6-chloro-3-((1-((1R,2R)-2-(difluoromethyl)-1-methylcyclopropane-1-carbonyl)-4-hydroxypiperidin-4-yl)methyl)-4-oxo-3,4-dihydro-7H-pyrrolo[2,3-d]pyrimidin-7-yl)phenyl)-2-methylmorpholine-4-carboxylate (66.3 mg, 0.0961 mmol), TFA (0.5 mL) and DCM (1 mL) gave, after freeze-drying, the title compound (55.7 mg, 98%) as a white solid. LCMS (method B): R_{T}=0.76 min, m/z=590, 592 [M+H]⁺. ¹H NMR (500 MHz, DMSO-d₆) δ 8.06 (s, 1H), 7.64-7.56 (m, 2H), 7.44-7.35 (m, 2H), 6.81 (s, 1H), 5.93 (td, J=55.5, 7.2 Hz, 1H), 4.91 (s, 1H), 4.03 (d, J=13.8 Hz, 1H), 3.99 (d, J=13.7 Hz, 1H), 3.96-3.76 (m, 4H), 3.56 (dqd, J=12.4, 6.1, 2.2 Hz, 1H), 3.43-2.73 (br. m, 3H), 3.27 (t, J=10.3 Hz, 1H), 2.96 (dd, J=11.7, 2.3 Hz, 1H), 2.58-2.51 (m, 1H), 1.70-1.45 (m, 3H), 1.43-1.35 (m, 2H), 1.31 (s, 3H), 1.18 (ddd, J=8.8, 5.1, 2.9 Hz, 1H), 1.09 (d, J=6.2 Hz, 3H), 0.94-0.88 (m, 1H).

### Co-Crystal Structure/Determination of Stereochemistry

The objective of this study was the crystal structure determination of USP7 in complex with an inhibitor according to the present invention.

### Experimental Setup

The construct used for crystallization comprises residues 207 to 560. The His-tag for purification was uncleaved. Crystals of USP7 in complex with the compound of Example 62 (hereafter C62) were obtained using sitting-drop vapour diffusion set-ups. USP7 at a concentration of 11.9 mg/ml (10 mM Tris/HCl, 100 mM NaCl, 4 mM TCEP, pH 8.0) was pre-incubated with 1.4 mM (4.95-fold molar excess) of C62 (150.0 mM in DMSO) for 1 h. 1 μl of the protein solution was then mixed with 0.1 μl of seeds and 0.9 μl of reservoir solution (0.03 M DL-Malic acid pH 7, 13% (w/v) PEG 3350) and equilibrated at 20° C. over 0.2 ml of reservoir solution. Well diffracting crystals were selected for data collection after 5 days.

### Data Collection

Crystals were cryo-protected by transferring them into a drop of Paratone-n before mounting. A complete 2.1 Å data set of a USP7/C62 crystal was collected in-house using a Bruker D8 Venture diffractometer equipped with an Excillum MetalJet D2+X-ray source and Photon III M28 detector and the data were integrated and analyzed by dials from the xia2 pipeline, and scaled by aimless from the ccp4 suite, respectively.

### Structure Determination and Refinement

Molecular replacement was done using a previously solved structure of USP7 in complex with a USP7 inhibitor as a starting model. Several rounds of alternating manual re-building and refinement with REFMAC5 and BUSTER resulted in the final model. Local non-crystallographic symmetry restraints were used. Atomic displacement factors were modelled with a single isotropic B-factor and a single TLS tensor per chain. The model has excellent stereochemistry with 97.9% and 2.1% of the residues in the favoured and allowed regions of the Ramachandran plot, respectively, and no outliers.

### Results

Crystals of USP7/C62 were produced that diffract to 2.1 Å resolution and determined its 3-dimensional structure. The final model contains two molecules of USP7 (residues 210-552 in chain A and residues 210-553 in chain B), two molecules of the compound C62 and 686 water molecules. The residues 459-462 and 501-509 in chain A and residues 502-509 in chain B are disordered and were omitted from the model. The overall structure is in accordance with the published USP7 structure in complex with inhibitor (PDB entry 6F5H) with r.m.s.d.s of 0.3 Å considering 517 and 515 Ca atoms respectively.

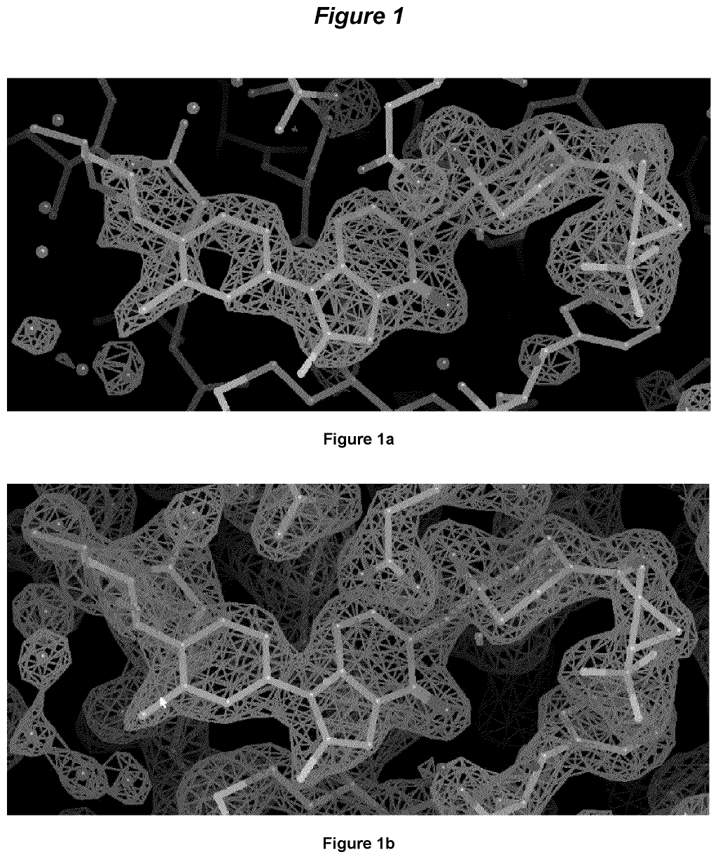

Clear electron density in the Fo-Fc omit map of the initial model at the compound binding site revealed the binding of the entire compound (FIG. 1a) and allowed an unambiguous placement of the ligand. Further refinement confirmed the initial placement (FIG. 1b).

C₆₂ binds in the catalytic cleft between the ‘palm’ and ‘thumb’, occupying the position of the C-terminus of the ubiquitin aldehyde in the published USP7/ubiquitin aldehyde complex (PDB accession code 1NBF).

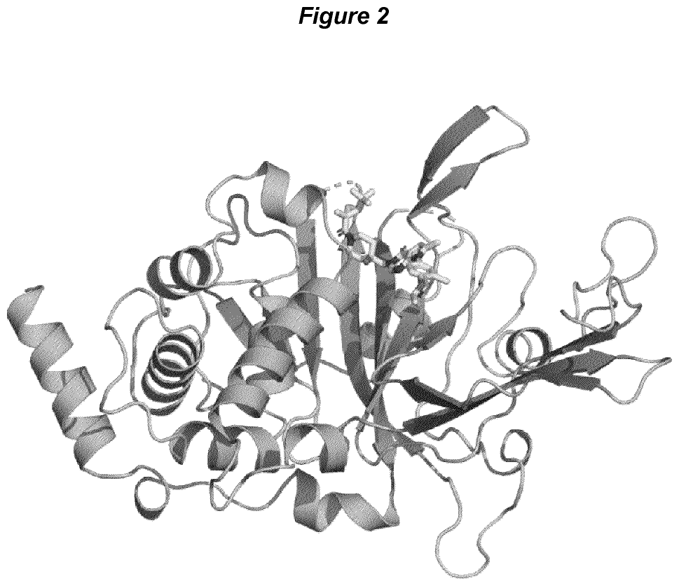

The trifluoromethyl head is interacting with the sidechain of Met292 via aliphatic interactions. The adjacent cyclopropyl moiety is stabilized by aliphatic-π-interactions with Phe409. The amide bond is forming a hydrogen bond from its carbonyl group to Tyr465. The piperidinol moiety itself is interacting with Tyr514 via aliphatic-π-interactions and forms hydrogen bonds from the hydroxyl group to Asp295 and to the peptide bond nitrogen of Val296. The adjacent chloro-pyrrolo-pyrimidinone is stabilized by r-r-stacking interactions with Met407. Hydrogen bonds are formed between the carbonyl oxygen 0 and the peptide bond nitrogen of Phe409, as well as between the pyrimidine nitrogen N and Gln297. Furthermore, atoms C2 and C17 are in hydrogen bonding distance to Asp295 and the peptide bond carbonyl of Phe409, respectively. The toluene linker moiety is stabilized by aliphatic-π-interactions with Gln351 and with Arg487 of a symmetry mate. The methylmorpholine tail group is forming hydrogen bonds from N4 to Gln351 and from 03 to a water molecule in chain A and to the peptide bond carbonyl of Arg487 of a symmetry mate (FIG. 2).

Analysis of the co-crystal structure allowed confirmation of the absolute stereochemistry of the compound of Example 62. The absolute stereochemistry of other, similar examples (e.g. examples 32, 33, 62, 67, 108, 109, 110) was determined by analogy. The following structures were confirmed:

### Example 32 Structure

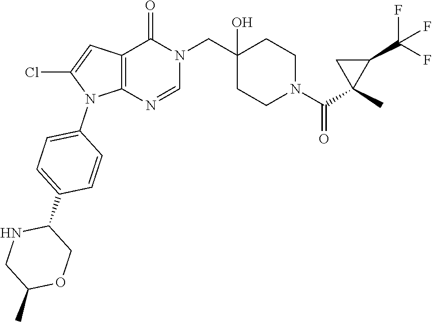 [SMILES: C[C@H]1CN[C@H](c2ccc(-n3c(Cl)cc4c(=O)n(CC5(O)CCN(C(=O)[C@]6(C)C[C@H]6C(F)(F)F)CC5)cnc43)cc2)CO1]

### Example 33 Structure

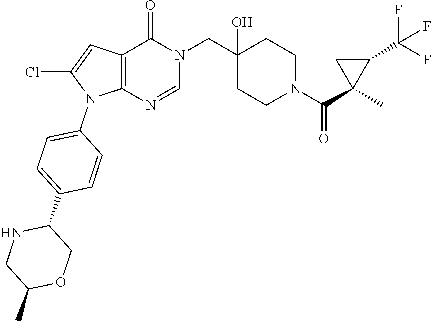 [SMILES: C[C@H]1CN[C@H](c2ccc(-n3c(Cl)cc4c(=O)n(CC5(O)CCN(C(=O)[C@@]6(C)C[C@@H]6C(F)(F)F)CC5)cnc43)cc2)CO1]

### Example 62 Structure

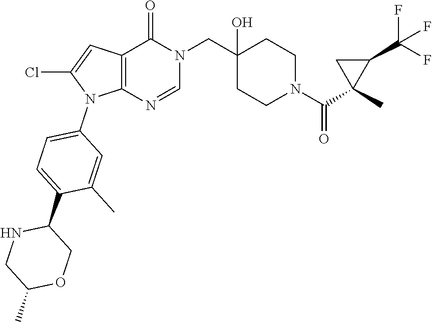 [SMILES: Cc1cc(-n2c(Cl)cc3c(=O)n(CC4(O)CCN(C(=O)[C@]5(C)C[C@H]5C(F)(F)F)CC4)cnc32)ccc1[C@H]1CO[C@H](C)CN1]

### Example 66 Structure

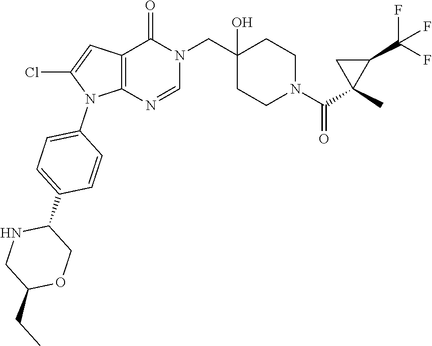 [SMILES: CC[C@H]1CN[C@H](c2ccc(-n3c(Cl)cc4c(=O)n(CC5(O)CCN(C(=O)[C@]6(C)C[C@H]6C(F)(F)F)CC5)cnc43)cc2)CO1]

### Example 67 structure

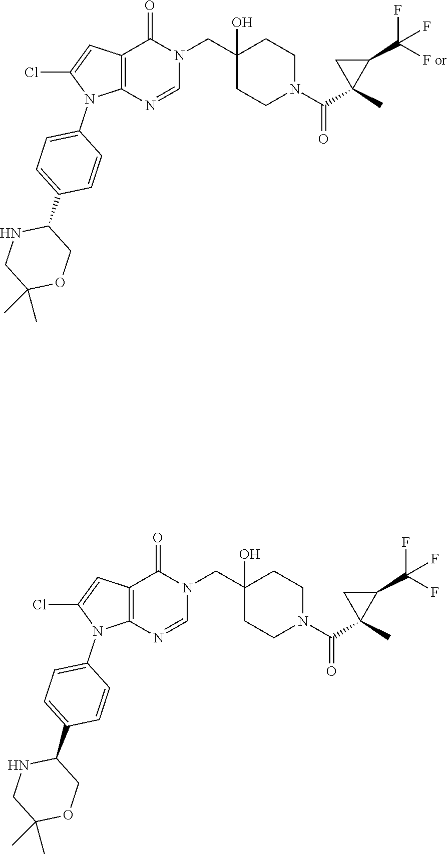

### Analysis of Activity and Pharmacological Properties

Measurement of USP7 inhibitory activity

USP7 activity was monitored in a fluorescence polarisation (FP) homogeneous assay using the isopeptide ubiquitin-Lys-TAMRA substrate (U-558, Boston Biochem). Full-length USP7 was purchased from Boston Biochem (His6-USP7FL, E-519). Unless otherwise stated, all other reagents were purchased from Sigma-Aldrich. Enzymatic reactions were conducted in black flat-bottom low volume polystyrene 384-well plates (Grenier) and 15 μL total volume. USP7 (2.5 nM, 5 μL) was incubated in assay buffer (50 mM HEPES (pH 7.2), 150 mM NaCl, 5 mM DTT, 0.05% BSA (w/v), 0.05% CHAPS) in the presence or absence of inhibitor (5 μL). Inhibitors were stored as 10 mM DMSO stocks in an inert environment (low humidity, dark, low oxygen, room temperature) using a Storage Pod System and serial dilutions were prepared in buffer just prior to the assay (from 200 to 0.003 μM, 11 dp curve). Following incubation at room temperature for 30 min, the enzymatic reactions were initiated by dispensing the Ub substrate (50 nM, 5 μL). FP was measured every 5 min over a period of 1.5 h (within the linear range of the assay) using a Pherastar FSX (BMG Labtech) exciting at 530 nm and measuring the amount of parallel and perpendicular light at 575 nm. The FP signal was subsequently normalised to the control without compound present. Data were plotted and fitted, and the concentrations resulting in 50% inhibition (IC₅₀) were calculated using the non-linear regression curve fitting model using GraphPad Prism. IC₅₀ values for the inhibitors of the invention are compiled in Table 3 above and represent the average of at least duplicate experiments.

### Target Engagement Assay-HTRF

A target engagement assay was conducted to demonstrate inhibition of USP7 by the exemplified compounds in cells by competition with a HA-tagged Ub-vinyl pentynyl sulfone probe (HA-Ub-VPS). HEK293 cells stably expressing Flag-tagged USP7 were treated with vehicle (DMSO) or compound (using 50 μM starting concentration and 11 dp using 1:3 serial dilution) for 2 h and subsequently lysed on ice and homogenised. Cell lysate and HA-Ub-VPS probe was then added to a white flat bottomed 384 well plate (Greiner) to a total volume of 10 μL and incubated for 40 min. 10 μL of D2 and Tb detection antibodies were subsequently added to give a final assay volume of 20 μL. Following incubation, HTRF were measured using a Pherastar FSX (BMG Labtech) exciting at 337 nm and measuring the emission at 620/665 nm. The HTRF ratiometric signal was derived and subsequently normalised to the lysate only controls. Data were plotted and fitted, and the concentrations resulting in 50% inhibition (IC₅₀) were calculated using the non-linear regression curve fitting model using GraphPad Prism. IC₅₀ values for the inhibitors of the invention are compiled in Table 1 above and represent the average of at least duplicate experiments.

### Determination of the Kinetic Solubility in PBS Buffer

Test compounds (5 μL; 10 mM DMSO stock) were added to 245 μL of PBS buffer pH 7.4 (Dulbecco A) in a Millipore MultiScreen® Solubility Filter plate and mixed at 300 rpm at room temperature on a plate shaker for 90 minutes. Meanwhile 5-points calibration curves for each compound were established in a mixture of acetonitrile/PBS buffer (top concentration 200 μM). After filtration and matrix match, the calibration and assay plates were analysed on a BMG Labtech CLARIOstar plate reader (240-400 nm). Final concentration of the test compound in the filtrate was calculated using the slope of the calibration curve.

### Determination of CLint Estimates Using Liver Microsomes

Test compounds (final concentration=1 μM; final DMSO concentration=0.1%) were incubated in 0.1M phosphate buffer pH 7.4 with liver microsomes (human, mouse, rat or dog; 0.5 mg of protein/mL) at 37° C. Reactions were started by addition of NADPH in 0.1M phosphate buffer pH 7.4 (final concentration 1 mM). 40 μL aliquots were removed at 3, 6, 10, 15, 20, 30, 40 and 50 minutes. Reactions were quenched in 80 μL of ice-cold methanol. Samples were subsequently frozen overnight then centrifuged at 3500 rpm for 20 minutes at 4° C. The supernatants were removed and transferred into analytical plates and analysed by LC/MS/MS.

### LC/MS/MS Method:

All samples were analysed on a Waters Acquity I-Class coupled to a Waters Xevo TQD mass spectrometer. A Waters BEH C18 2.1×50 mm 1.7 μm column was used and mobile phases were water and methanol containing 0.1% formic acid as modifier. Analysis was by multiple reaction monitoring and conditions were optimised for each test compound.

### Data Analyses:

From a plot of In peak area against time, the gradient of the line is determined.

Subsequently, half-life and intrinsic clearance are calculated using the equations below:

$$Eliminated rate constant ( k ) = ( - gradient )$$

$$Half life ( {t}_{1 / 2} ) ( min ) = \frac{0.693}{k}$$

$$Intrinsic Clearance ( {CL}_{int} ) ( μL / min / million cells ) = \frac{V \times 0.693}{{t}_{1 / 2}}$$

where V=Incubation volume (μL)/number of cells

The CLₕₑₚ was calculated as follows:

$${Cl}_{h e p} ( mL / min / kg ) = \frac{Clint ( mL / min / kg ) * Qh}{Clint ( mL / min / kg ) + Q h}$$

Where CLᵢₙₜ (mL/min/kg) is the intrinsic clearance scaled to a whole liver and Qh is hepatic liver blood flow.

### Pharmacokinetic (PK) Parameters

PK parameters for certain exemplary compounds are provided below.

### Example 21

BALB/c mouse IV 1 mg/kg: t_{1/2}=2.09 h, AUC_{inf}=479 hr*ng/mL, Vₛₛ=3.89 (L/kg), CL_{b}=35.1 (mL/min/kg)
BALB/c mouse PO 3 mg/kg: AUC_{inf}=593 hr*ng/mL, F=40.3%

### Example 32:

BALB/c mouse IV 1 mg/kg: t_{1/2}=3.5 h, AUC_{inf}=591 hr*ng/mL, Vₛₛ=5.49 (L/kg), CL_{b}=28.2 (mL/min/kg)
BALB/c mouse PO 3 mg/kg: AUC_{inf}=2096 hr*ng/mL, F=116%

### Cypre 30 Panel Spheroid Assay—30 Model Panel of PDX Tumorqraft-Derived Spheroids

### Study Description

The Cypre 30 Panel (30 PDX tumorgraft-derived in vitro spheroid models) was pre-grown in Cypre's VersaGel® hydrogel matrix for a period of 3-5 days, co-cultured with pre-activated PBMCs from a single donor and followed by treatment with a compound of the present invention (test article; hereafter TA), either alone or in combination with a known immune checkpoint inhibitor (ICI). On day 4 after PBMC addition and treatment, Hoechst and DRAQ7 were added to cell culture medium and images were captured using high content instrumentation. Average tumorsphere size and apoptosis were calculated using Cypre's proprietary high content image analysis algorithms.

### Materials and Methods

TA was shipped in DMSO on dry ice.

TA was stored at −20° C. ICI was stored at 4° C.

Other chemicals and enzymes needed to perform the in vitro assays are detailed in the table below:

TABLE 4a
chemicals and enzymes

| Product | Supplier |
|---|---|
| Hoechst 33342 | Thermofisher |
| 4% Paraforamaldehyde | Fisher |
| DRAQ7 | Abcam ab109202 |

Buffers and media needed to perform the in vitro assays are detailed in the table below:

TABLE 4b
buffers, media and solvents

| Product | Supplier |
|---|---|
| Fetal Bovine serum (FBS) | Thermo Fisher, 16-140-071 |
| Penicillin-Streptomycin | Thermo Fisher, 15140-148 |
| 10 × PBS, pH 7.4 | Thermo Fisher, 70011-069 |
| RPMI 1640-Medium with 25 mM HEPES | Thermo Fisher, 72400-120 |
| buffer, with GlutaMax | |
| Recombinant FGF2 | R&D Systems, 233-FB-025 |
| Insulin human, recombinant | Sigma-Aldrich, I2643 |
| Tryple Express Enzyme | Thermo Fisher, 12605-010 |
| rhIL-2 | StemCell |
| T Cell Transact ™ | Miltenyi Biotech |
| Activation Kit (CD3/CD28) | |

### Cell Model

The thirty PDX (patient-derived xenograft) tumorgraft models used in the study were CXF 94, CXF 269, CXF 1103, GXF 3023, GXA 3067, GXF 251, HNXF 1853, LIXAH 575, LXFA 289, LXFA 526, LXFA 586, LXFA 629, LXFA 923, LXFE 66, LXFL 1674, LXFL 430, MAXFTN 401, MEXF 1829, MEXF 622, OVXF 899, PAXF 1997, PAXF 2005, PXF 698, RXF 486, RXF 1781, RXF 2282, RXF 2516, SXFO 678, SXFS 1301, and UXF 1138, provided to Cypre by Charles River Laboratories Germany GmbH. The fibroblasts used in this study were foetal Human Dermal Fibroblastsl purchased from ScienCell Research Laboratories. The PBMCs used in the study were provided by Charles River's Cell Supply company, Hemacare.

### Cultivation of Cell Lines

Tumour cell lines were routinely passaged once or twice weekly and maintained in culture for up to 20 passages. Cells were grown at 37° C. in a humidified atmosphere with 5% CO2 in RPMI-1640 medium supplemented with 10% (v/v) fetal bovine serum and 100U/mL Penicillin-Streptomycin. HDF cells were grown in fibroblast media containing FGF. The percentage of viable cells was determined using a Thermo Countess II Cell Counter.

### Cultivation of PBMC Donor

PBMCs from a single donor were thawed in 6-well plates and grown at 37° C. in a humidified atmosphere with 5% CO₂ in RPMI-1640 medium supplemented with 10% (v/v) fetal bovine serum and 100U/mL Penicillin-Streptomycin with recombinant-human-IL2 and activated with CD3/CD28. Following two days of culture, cells were washed from the activation kit and replated in T Cell Media for another two days. The percentage of viable cells was determined using a Thermo Countess II Cell Counter.

### 3D Tumour Spheroid Model Setup

PDX and HDF cells were trypsinized and counted. 0.5×10⁶/mL of tumour cells and 0.4×106/mL HDFs were mixed with VersaGel® and polymerized into a hydrogel. In some cases for slower growing tumours, 1.0×10⁶/mL of tumour cells and 0.8×10⁶/mL HDFs were used. The fully constructed 3D cell model was then grown in RPMI-1640 medium supplemented with 10% (v/v) FBS, and 100U/mL penicillin-streptomycin. Medium with TA and PBMCs were added on days 3-5 (depending on the speed of growth for specific tumour lines). On day 4, following PBMC and TA addition, wells were stained with Hoechst nuclear dye and DRAQ7 cell death marker in the live culture assay, followed by fixation with 4% Paraformaldehyde.

### Imaging Acquisition and Data Analysis

The 3D tumour spheroid models were imaged and analysed using ImageXpress Micro Confocal High-Content imaging system (Molecular Devices)—tumorsphere size and tumour cell death was quantitated using the MX software package and proprietary image processing. A cell mask with a size threshold was used to identify multi-cellular tumorspheres from single cell PBMCs. Tumour Size=Average tumorsphere area (μm²) in the well as quantitated by proprietary high content image analysis.

Total Tumour Area=Tumour Size multiplied by tumorsphere count in the well.

DRAQ7%=DRAQ7 signal within tumorspheres/total tumour area.

### Results and Discussion

Cypre utilised its 3D hydrogel patterning technology to construct its signature Cypre 30 PDX Spheroid Panel, which encompasses thirty 3D PDX-derived tumorgraft in vitro spheroid models in 96-well plates comprising a range of tumour histotypes (CRC, Head & Neck, Liver, Triple Negative Breast Cancer (TNBC), Ovarian, Pancreatic, Osteo-Sarcoma, Soft Tissue Sarcoma, Uterus, Gastric, Melanoma, Non-Small Cell Lung Cancer (NSCLC), Pleuromesothelioma, and Renal Cell Carcinoma (RCC)).

Responder lines with TA alone included LXFA 526, MEXF 622, and RXF 2282; responders in combination with ICI included LXFA 923, LXFL 430, MEXF 1829, and OVXF 899. Historically, for 10 checkpoint inhibitors, Cypre applied a threshold of 15% reduction in size to categorise responder models. A more stringent threshold of 20% reduction in size was used to define responders in the work described herein.

### In Vivo Study—CT26 Murine CRC Model

An in vivo efficacy study of a USP7 inhibitor of the present invention (test compound: TC), alone and in combination with an immune checkpoint inhibitor (ICI), in the treatment of a subcutaneous CT26 murine colorectal cancer model in female BALB/c Mice was performed.

### Study Design:

| | | | Dose | Dosing | Dosing | | |
|---|---|---|---|---|---|---|---|
| | | | level | solution | volume | | Dosing frequency |
| Group | No. | Treatment | (mg/kg) | (mg/mL) | (μL/g) | ROA | and Duration |
| 1 | 10 | IgG2a + | 10 | 1 | 10 | i.p. + | n = 10, Q4D × 7 weeks |
| | | vehicle | | | | p.o. | (14 doses) + QD vehicle × |
| | | | | | | | 49 doses |
| | 10 | | | | | | n = 10, Q4D × 2 weeks |
| | | | | | | | (4 doses) + QD vehicle × |
| | | | | | | | 14 doses |
| 2 | 10 | TC | 30 | 3 | 10 | p.o. | n = 10, TC QD × 7 weeks |
| | | | | | | | (49 doses) |
| | 10 | | | | | | n = 10, TC QD × 2 weeks |
| | | | | | | | (14 doses) |
| 3 | 10 | TC | 75 | 7.5 | 10 | p.o. | n = 10, TC QD × 7 weeks |
| | | | | | | | (49 doses) |
| | 10 | | | | | | n = 10, TC QD × 2 weeks |
| | | | | | | | (14 doses) |
| 4 | 10 | ICI | 10 | 1 | 10 | i.p. | n = 10, ICI Q4D × 7 weeks |
| | | | | | | | (14 doses) |
| | 10 | | | | | | n = 10, ICI Q4D × 2 weeks |
| | | | | | | | (4 doses) |
| 5 | 10 | TC + | 30 + 10 | 3 + 1 | 10 | p.o | n = 10, TC QD × 7 weeks |
| | | ICI | | | 10 | i.p. | (49 doses) ICI Q4D × 7 |
| | | | | | | | weeks (14 doses) |
| | 10 | | | | | | n = 10, TC QD × 2 weeks |
| | | | | | | | (14 doses) ICI Q4D × 2 |
| | | | | | | | weeks (4 doses) |
| 6 | 10 | TC + | 75 + 10 | 7.5 + 1 | 10 | p.o. | n = 10, TC QD × 8 weeks |
| | | ICI | | | 10 | i.p. | (56 doses) ICI Q4D × 8 |
| | | | | | | | weeks (16 doses) |
| | 10 | | | | | | n = 10, TC QD × 2 weeks |
| | | | | | | | (14 doses) ICI Q4D × 2 |
| | | | | | | | weeks (4 doses) |

Notes:
1. No.: animal number per group
2. Randomisation when the mean tumour volume reached ~100 mm³ (designated as Day 0)
3. Treatment with test compound (TC) on same day as randomisation or no later than 24 h later. For combination groups (Groups 5 & 6), dosing with ICI agent started 24 h after start of dosing of the TC.
4. In combination groups (Groups 5 & 6) when dosing TC and ICI on same day, TC was given 30 min before ICI.

### Experimental Methods

The CT-26 tumour cells (SIBS) were maintained in vitro at 37° C. in an atmosphere of 5% CO2. The cells in exponential growth phase were harvested and quantitated by cell counter before tumour inoculation. (Detailed culture medium: RMPI-1640+10% FBS).

One mouse at a time was immobilized and the site of injection was disinfected with an alcohol swab. Each mouse was inoculated subcutaneously in the right flank region for tumour development. (Inoculation details: 5×10⁵ in 0.1 ml PBS).

The randomization was carried out when the mean tumour size reached ˜100 mm³ (p>0.05 among all 6 groups). A total of 120 mice were enrolled in the study and all animals were randomly allocated to 6 study groups (see experimental design, above). Randomization was performed based on “Stratified distribution” method (StudyDirector™ software, version 3.1.399.19)/randomized block design. The date of randomization was denoted as day 0.

After the tumour cell inoculation, animals were checked daily for morbidity and mortality. During routine monitoring, the animals were checked for any adverse effects of tumour growth and treatment on behaviour such as: mobility, food and water consumption, body weight gain/loss, eye/hair matting and any other abnormalities. Body weight measurement: daily. Mortality and any observed adverse clinical signs were recorded in detail for individual animals.

Tumour volumes measurement: 3 times per week, in two dimensions using a caliper, and the volume was expressed in mm³ using the formula: V=(L×W×W)/2, where V is tumour volume, L is tumour length (the longest tumour dimension) and W is tumour width (the longest tumour dimension perpendicular to L). Dosing, as well as tumour and body weight measurements, were conducted in a Laminar Flow Cabinet. All measurements were recorded electronically using StudyDirector™ software (version 3.1.399.19).

The treatment of all groups were initiated after randomization/grouping as per the study design, ICI agent was initiated 24 hours later (Day 1).

Body weight (BW) changes were calculated relative to the BW of the mouse on the first day of treatment. Mice with >10% BW loss were given a dosing holiday and/or provided with supplemental diet nutrient gel (Ready Dietech Jelly 76).

T/C was calculated for each group. T and C are the mean tumour volume of the treated and control groups, respectively, on a given day.

Tumour growth inhibition (TGI): TGI % is an indication of anti-tumour activity and expressed as: TGI (%)=100×(1−T/C).

### Study Endpoints

Humane surrogate endpoints for tumour volume, body weight loss and survival were applied consistently to animals in this experiment. Tumour volumes were not allowed to exceed 1800 mm³; body weight loss was not allowed to exceed 20% (from weight at start of dosing) and death was not permitted as an endpoint (surrogate survival endpoints based on the humane tumour volume endpoint were applied). Kaplan-Meier survival pairwise comparisons were performed using the Log-Rank test (GraphPad Prism).

A total of 10 mice per group were chosen at random as satellites and terminated on Day 13 for sample collection. The remaining animals were treated and observed until their study endpoint.

### Results Summary

In this study, the therapeutic effect of a novel small molecule inhibitor according to the present invention (test compound, TC) alone, or in combination with an immune checkpoint inhibitor (ICI) on the tumour microenvironment, tumour size, and survival benefit was evaluated in a subcutaneous CT26 model of mouse colon cancer. Tumour growth curves, tumour growth inhibition, anti-tumour responses (including regressions), and survival benefit were analysed. Tolerability was assessed by examining changes in mouse body weight, the onset of any adverse clinical signs, and moratlity.

Treatment with the novel compound, TC, was well tolerated by test animals at up to 75 mg/kg daily and also in combination with the ICI agent. No noteworthy losses in body weights were observed over the course of the study period.

In terms of survival benefit, the median survival time of the vehicle control group was calculated as 15 days, while TC single agent treatment at 30 and 75 mg/kg, and in combination with ICI, all demonstrated significant improvements in survival (all p<0.05), when compared to the vehicle group. The biggest survival benefit was provided by 75 mg/kg TC in combination with ICI treatment (Group 6), with a median survival time (MST) of 46 days.

The mean tumour size of the vehicle treated group (Group 1) reached 1494.88 mm³ on Day 12. Treatment with TC single agent at 30 mg/kg (Group 2) and in combination with ICI (Group 5) produced mild and non-significant anti-tumour efficacy, with TGI values of 19.29% and 34.05% (both p>0.05). However, TC single agent at 75 mg/kg (Group 3) and in combination with ICI (Group 6), improved anti-tumour activity, with TGIs of 47.21% and 50.47%, respectively (both p<0.05).

To evaluate and compare drug exposure of single dose and combined treatment, plasma samples were collected at 1 h, 4h, and 8h after the first and last dose for the groups dosed with TC alone at different doses (Group 2,3), and in combination at different doses (Group 5, 6). The TC drug exposure was similar in single dose groups and combined dose groups at different timepoints. Meanwhile, there was a positive correlation between drug exposure and dose at two different dose levels. Following multiple doses, no drug accumulation was observed.

### In Vivo Efficacy Studies—12 Panel Syngeneic Model Screen (MuScreen Platform)

The MuScreen™ platform (Crown Bioscience) is a high throughput in vivo screen to evaluate immune modulating agents across preselected panels of well-characterised syngeneic tumour models. In vivo efficacy of a selected compound of the present invention was tested across a panel of 12 syngeneic models (see Table 5 below). Response to the compound alone and in combination with immune checkpoint inhibitors was assessed by reference to absolute mean tumour volume (TV), relative individual TV, and changes to body weight (BW).

TABLE 5
MuScreen ™ study results

| | | | Single agent | Combo | Combo |
|---|---|---|---|---|---|
| Model | Cancer type | P53 status | response | response | enhancement |
| CT26 | Colon | WT | R | R | R |
| MC-38 | Colon | Mut (Het) | R | R | R |
| EMT6 | Breast | WT | R | R | Eq |
| H22 | Liver | WT | R | R | NR |
| Hepa 1-6 | Liver | WT | R | R | Eq |
| Renca | Kidney | Mut (Het) | NR | NR | NR |
| Pan02 | Pancreatic | WT | R | R | NR |
| A20 | Lymphoma | Mut (Hom) | NR | R | R |
| B16-F10 | Melanoma | Mut (Het) | R | R | NR |
| LL/2 (LLC1) | Lung | WT | R | R | R |
| RM-1 | Prostate | Mut (Het) | R | R | Eq |
| B16/BL6 | Melanoma | WT | Eq | Eq | NR |

Note:
R—responders;
NR—non-responders;
Eq—equivocal response

### MuScreen Results—Summary

In 12 syngeneic models:
At least 9 out of 12 models showed single agent response.
At least 4 out of 12 models showed combination enhancement.
Four ICI-insensitive models (Pan02, A20, B16-F10, and RM-1) showed a response to combination with the agent of the invention.
CT26 data with the TC was in line with previous data; in fact, suggests earlier and stronger anti-tumour benefit.
Improved combination benefit was indicated in additional models, for example, see MC-38, A20, and LL/2 (LLC1).
Cases of complete tumour responses (to the extent that tumour material could not be measured, TV=0 mm³) were observed in CT26, EMT6, and A20 models with combination treatment; additionally, a three-week follow up period after dosing had stopped in EMT6 and A20 models, tumours that measured TV=0 mm³ remained ‘cured’).
Some anti-tumour benefit was observed in B16F10 models, which have been identified as being immunologically ‘cold’.

The cancer cachexia characteristics of Renca and B16/BL6 models complicated their dosing phase wherein transient dosing holidays were given for humane/welfare reasons, which may lead to an underestimation of the anti-tumour effect.

1. A compound according to formula (I), including stereoisomers thereof: 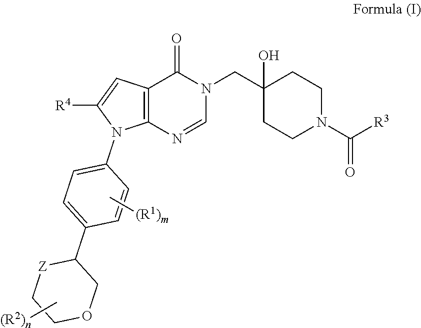
wherein:
“m” is 0, 1 or 2, and wherein each R¹ is independently selected from: halo; C₁-C₆ alkyl optionally substituted with halo; and C₃-C₆ cycloalkyl;
“n” is 0, 1 or 2, and wherein each R² is independently selected from optionally substituted C₁-C₆ alkyl, optionally substituted C₃-C₆ cycloalkyl, wherein each of the one or more optional substituents are independently selected from C₁-C₆ alkyl, and halo,
or wherein “n” is 2 and each R² together with the carbon ring atom or atoms to which they are attached combine to form a C₃-C₆ fused cycloalkyl ring, a C₃-C₆ spirocyclic cycloalkyl ring, or C₃-C₄ bridge;
R³ is selected from optionally substituted C₁-C₆ alkyl, optionally substituted C₃-C₆ cycloalkyl, optionally substituted phenyl, optionally substituted C₇-C₁₀ arylalkyl, optionally substituted C₈-C₁₀ arylalkenyl, and optionally substituted C₃-C₆ heteroaryl; wherein each of the one or more optional substituents is independently selected from: halo; C₁-C₆ alkyl optionally substituted with halo; and C₃-C₆ cycloalkyl optionally substituted with methyl or CF₃;
R⁴ is H, Cl, C₁-C₆ alkyl;
wherein one of X and Z is O and one of X and Z is NR′, such that the ring comprising X and Z is a morpholine ring, wherein R′ is H or C₁-C₆ alkyl, optionally H;
or a pharmaceutically acceptable salt or solvate thereof.

2. A compound of claim 1 according to formula (Ia), including stereoisomers thereof: 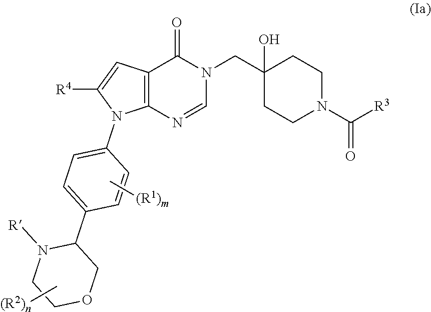
wherein:
“m”, “n”, R¹-R⁴ and R′ are as defined in claim 1,
or a pharmaceutically acceptable salt or solvate thereof.

3. A compound of claim 1, which is a compound according to formula (Ic), including stereoisomers thereof: 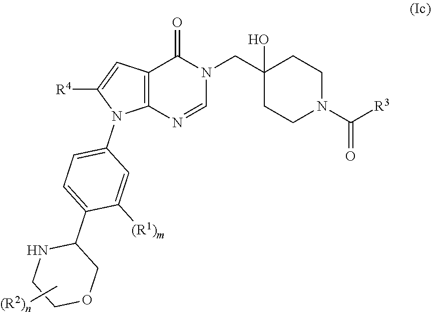
wherein:
“m”, “n”, R¹-R⁴ and R′ are as defined in claim 1,
or a pharmaceutically acceptable salt or solvate thereof.

4. A compound according to claim 1, or a pharmaceutically acceptable salt or solvate thereof, wherein X is NR′ and Z is O;
and/or wherein “m” is 0 or 1;
and/or wherein “n” is 1 or 2;
and/or wherein each R¹ is independently selected from Cl; F; methyl optionally substituted with F, optionally CF₃; ethyl optionally substituted with F, propyl, and cyclopropyl;
and/or wherein each R² is independently selected from: methyl optionally substituted with F;
ethyl; and cyclopropyl.

5. (canceled)

6. (canceled)

7. (canceled)

8. (canceled)

9. A compound according to claim 1, or a pharmaceutically acceptable salt or solvate thereof, wherein each R¹ is independently methyl, optionally substituted with F;
and/or wherein each R² is independently selected from: methyl; ethyl; and cyclopropyl;
and/or wherein “m” is 0;
and/or wherein “n” is 1.

10. (canceled)

11. (canceled)

12. (canceled)

13. A compound according to claim 1, or a pharmaceutically acceptable salt or solvate thereof, wherein “n” is 1 and R² is methyl, or wherein “n” is 2 and each R² is methyl.

14. (canceled)

15. A compound according to claim 1, or a pharmaceutically acceptable salt or solvate thereof, wherein “n” is 2 and each R² together with the carbon ring atoms to which they are attached combine to form: a C₃ or C₄ spirocyclic cycloalkyl ring; or a C₃-C₆ fused cycloalkyl ring; or a C₄ bridge.

16. (canceled)

17. (canceled)

18. (canceled)

19. A compound according to claim 1, or a pharmaceutically acceptable salt or solvate thereof, wherein at least one R² group is at the 6 position of the morpholinyl ring, optionally wherein each R² substituent is at the 6 position of the morpholinyl ring.

20. A compound according to claim 1, or a pharmaceutically acceptable salt or solvate thereof, wherein at least one R² group is at the 2 position of the morpholinyl ring, optionally wherein each R² substituent is at the 2 position of the morpholinyl ring.

21. A compound according to claim 1, or a pharmaceutically acceptable salt or solvate thereof, wherein at least one R² group is at the 5 position of the morpholinyl ring, optionally wherein each R² substituent is at the 5 position of the morpholinyl ring.

22. A compound according to claim 1, or a pharmaceutically acceptable salt or solvate thereof, wherein R⁴ is chlorine; or wherein R⁴ is H or C₁-C₆ alkyl; or wherein R⁴ is H or methyl.

23. (canceled)

24. (canceled)

25. A compound according to claim 1, or a pharmaceutically acceptable salt or solvate thereof, wherein R³ is optionally substituted C₃-C₆ cycloalkyl, wherein each of the one or more optional substituents is independently selected from halo; C₁-C₂ alkyl optionally substituted with F; and C₃-C₆ cycloalkyl.

26. A compound according to claim 1, or a pharmaceutically acceptable salt or solvate thereof, wherein R³ is optionally substituted cyclopropyl, wherein each of the one or more optional substituents is independently selected from methyl, CHF₂, CF₃, and cyclopropyl;
or wherein R³ is an optionally substituted cyclopropyl group of general formula: 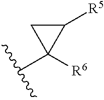
wherein R⁵ and R⁶ are each independently selected from hydrogen, halo; C₁-C₂ alkyl optionally substituted with F; and C₃-C₆ cycloalkyl.

27. (canceled)

28. A compound according to claim 26, wherein R⁵ and R⁶ are each independently selected from hydrogen, methyl, CHF₂, CF₃ and cyclopropyl, preferably wherein R⁵ is independently selected from hydrogen and CF₃ and R⁶ is independently selected from methyl and cyclopropyl.

29. A compound according to claim 1, or a pharmaceutically acceptable salt or solvate thereof, wherein R³ is optionally substituted phenyl, wherein each of the one or more optional substituents is independently selected from halo, C₁-C₂ alkyl optionally substituted with F, and C₃-C₆ cycloalkyl optionally substituted with methyl or CF₃;
or wherein R³ is optionally substituted C₇-C₁₀ arylalkyl, wherein each of the one or more optional substituents is independently selected from halo; C₁-C₆ alkyl optionally substituted with halo; and C₅-C₆ cycloalkyl optionally substituted with methyl or CF₃;
or wherein R³ is optionally substituted (CH₂)₂-phenyl, wherein each of the one or more optional substituents is independently selected from: halo; C₁-C₆ alkyl optionally substituted with halo; and C₅-C₆ cycloalkyl optionally substituted with methyl or CF₃;
or wherein R³ is an arylalkyl group, each of the one or more optional substituents of the arylalkyl group is independently selected from halo, methyl, ethyl, CHF₂, and CF₃.

30. A compound according to claim 1, or a pharmaceutically acceptable salt or solvate thereof, wherein R³ is optionally substituted phenyl, wherein each of the one or more optional substituents is independently selected from Cl, F, methyl, CF₃, CHF₂, and cyclopropyl optionally substituted with methyl or CF₃.

31. (canceled)

32. (canceled)

33. (canceled)

34. A compound according to claim 1, or a pharmaceutically acceptable salt or solvate thereof, selected from:
rac-6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
rac-6-Chloro-3-((4-hydroxy-1-(1-methylcyclopropane-1-carbonyl)piperidin-4-yl)methyl)-7-(4-((3R,5S)-5-methylmorpholin-3-yl)phenyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-(3-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(3-methyl-4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((2S,3S)-2-methylmorpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((2R,3R)-2-methylmorpholin-3-yl)phenyl)-3,7-dihydro-4H-pyrrolo[2,3-d]pyrimidin-4-one
rac-6-Chloro-3-((1-(4-chlorobenzoyl)-4-hydroxypiperidin-4-yl)methyl)-7-(4-((3R,6R)-6-(trifluoromethyl)morpholin-3-yl)phenyl)-3,7-dihydro-4H-pyrrolo[2,3-d]pyrimidin-4-one
6-Chloro-7-(4-((S)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-7-(4-((R)-6,6-dimethylmorpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-7-(4-(5,5-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-(5,5-dimethylmorpholin-3-yl)phenyl)-3,7-dihydro-4H-pyrrolo[2,3-d]pyrimidin-4-one
rac-3-((1-(4-Chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
7-(4-(7-Oxa-4-azaspiro[2.5]octan-5-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
7-(4-(7-Oxa-4-azaspiro[2.5]octan-5-yl)phenyl)-6-chloro-3-((4-hydroxy-1-(1-methylcyclopropane-1-carbonyl)piperidin-4-yl)methyl)-3,7-dihydro-4H-pyrrolo[2,3-d]pyrimidin-4-one
3-((1-Benzoyl-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one
6-Chloro-3-((1-(4-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
(S)-6-Chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one
3-((1-Benzoyl-4-hydroxypiperidin-4-yl)methyl)-6-chloro-7-(4-((3S,6R)-6-methylmorpholin-3-yl)phenyl)-3,7-dihydro-4H-pyrrolo[2,3-d]pyrimidin-4-one
6-Chloro-3-((1-(4-fluorobenzoyl)-4-hydroxypiperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,5S)-5-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,5R)-5-methylmorpholin-3-yl)phenyl)-3,7-dihydro-4H-pyrrolo[2,3-d]pyrimidin-4-one
(S)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one
(R)-6-Chloro-7-(4-(6,6-dimethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one
6-Chloro-3-((1-(4-chloro-2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((4-hydroxy-1-((1 S,2S)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
(R)-6-Chloro-3-((1-(4-chlorobenzoyl)-4-hydroxypiperidin-4-yl)methyl)-7-(4-(6,6-dimethylmorpholin-3-yl)phenyl)-3,7-dihydro-4H-pyrrolo[2,3-d]pyrimidin-4-one
6-Chloro-3-((1-(3-chlorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(3,4-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(3-fluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(2,3-difluorobenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
(S)-3-((1-([1,1′-Bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one
(R)-3-((1-([1,1′-Bi(cyclopropane)]-1-carbonyl)-4-hydroxypiperidin-4-yl)methyl)-6-chloro-7-(4-(6,6-dimethylmorpholin-3-yl)phenyl)-3,7-dihydro-4H-pyrrolo[2,3-d]pyrimidin-4-one
3-((1-([1,1′-Bi(cyclopropane)]-1-carbonyl)-4-hydroxypiperidin-4-yl)methyl)-6-chloro-7-(4-((3R,6S)-6-methylmorpholin-3-yl)phenyl)-3,7-dihydro-4H-pyrrolo[2,3-d]pyrimidin-4-one
7-(4-(5-Oxa-8-azaspiro[3.5]nonan-7-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one
7-(4-(5-Oxa-8-azaspiro[3.5]nonan-7-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
6-Chloro-7-(4-((3R,6S)-6-ethylmorpholin-3-yl)phenyl)-3-((1-(4-fluorobenzoyl)-4-hydroxypiperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one
6-Chloro-3-((1-(4-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
7-(4-((1R,2S,5S)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
7-(4-((1R,2S,5S)-8-Oxa-3-azabicyclo[3.2.1]octan-2-yl)phenyl)-3-((1-([1,1′-bi(cyclopropane)]-1-carbonyl)-4-hydroxypiperidin-4-yl)methyl)-6-chloro-3,7-dihydro-4H-pyrrolo[2,3-d]pyrimidin-4-one
6-Chloro-3-((1-(4-chloro-2-fluorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(3-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2-chlorobenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-(octahydro-2H-benzo[b][1,4]oxazin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-(octahydrocyclopenta[b][1,4]oxazin-3-yl)phenyl)-3,7-dihydro-4H-pyrrolo[2,3-d]pyrimidin-4-one
6-Chloro-3-((4-hydroxy-1-(4-methylbenzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-(4-(trifluoromethyl)benzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2-chloro-4-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2,4-dimethylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(2,4-dimethylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
7-(4-(2-Oxa-5-azabicyclo[4.1.0]heptan-4-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,6R)-6-cyclopropylmorpholin-3-yl)phenyl)-3,7-dihydro-4H-pyrrolo[2,3-d]pyrimidin-4-one
6-Chloro-3-((4-hydroxy-1-((1 S,2S)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-cinnamoyl-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6S)-6-cyclopropylmorpholin-3-yl)phenyl)-3,7-dihydro-4H-pyrrolo[2,3-d]pyrimidin-4-one
6-Chloro-3-((4-hydroxy-1-(4-(trifluoromethyl)benzoyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-7-(4-((3R,6S)-6-ethylmorpholin-3-yl)phenyl)-3-((4-hydroxy-1-((1 S,2S)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((3R,6S)-6-ethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((R)-6,6-dimethylmorpholin-3-yl)phenyl)-3-((4-hydroxy-1-((1 S,2S)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((S)-6,6-dimethylmorpholin-3-yl)phenyl)-3-((4-hydroxy-1-((1 S,2S)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((R)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-7-(4-((S)-6,6-dimethylmorpholin-3-yl)phenyl)-3-((4-hydroxy-1-((1R,2R)-1-methyl-2-(trifluoromethyl)cyclopropane-1-carbonyl)piperidin-4-yl)methyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3S,6S)-6-methylmorpholin-3-yl)phenyl)-3,7-dihydro-4H-pyrrolo[2,3-d]pyrimidin-4-one
6-Chloro-3-((1-(4-chlorobenzoyl)-4-hydroxypiperidin-4-yl)methyl)-7-(4-((3R,6R)-6-methylmorpholin-3-yl)phenyl)-3,7-dihydro-4H-pyrrolo[2,3-d]pyrimidin-4-one
(S)-6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one
(R)-6-Chloro-3-((4-hydroxy-1-(1-methylcyclopropane-1-carbonyl)piperidin-4-yl)methyl)-7-(4-(morpholin-3-yl)phenyl)-3,7-dihydro-4H-pyrrolo[2,3-d]pyrimidin-4-one
6-Chloro-7-(3-fluoro-4-(morpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
(S)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one
(R)-6-Chloro-3-((4-hydroxy-1-(1-methylcyclopropane-1-carbonyl)piperidin-4-yl)methyl)-7-(3-methyl-4-(morpholin-3-yl)phenyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-(4-methylmorpholin-3-yl)phenyl)-3,7-dihydro-4H-pyrrolo[2,3-d]pyrimidin-4-one
(S)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one
(R)-6-Chloro-3-((1-(4-chlorobenzoyl)-4-hydroxypiperidin-4-yl)methyl)-7-(4-(morpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((S)-morpholin-3-yl)phenyl)-3,7-dihydro-4H-pyrrolo[2,3-d]pyrimidin-4-one
(S)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one
(R)-3-((1-([1,1′-Bi(cyclopropane)]-1-carbonyl)-4-hydroxypiperidin-4-yl)methyl)-6-chloro-7-(4-(morpholin-3-yl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((R)-morpholin-3-yl)phenyl)-3,7-dihydro-4H-pyrrolo[2,3-d]pyrimidin-4-one
6-Chloro-3-((1-((R)-4,4-difluoro-3-phenylbutanoyl)-4-hydroxypiperidin-4-yl)methyl)-7-(4-((S)-morpholin-3-yl)phenyl)-3,7-dihydro-4H-pyrrolo[2,3-d]pyrimidin-4-one
6-Chloro-3-((4-hydroxy-1-(1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one
6-Chloro-3-((4-hydroxy-1-(1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one
6-Chloro-3-((1-((R)-4,4-difluoro-3-phenylbutanoyl)-4-hydroxypiperidin-4-yl)methyl)-7-(4-((R)-morpholin-3-yl)phenyl)-3,7-dihydro-4H-pyrrolo[2,3-d]pyrimidin-4-one
6-Chloro-7-(3-chloro-4-((S)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-7-(3-chloro-4-((R)-morpholin-3-yl)phenyl)-3-((4-hydroxy-1-((R)-4,4,4-trifluoro-3-phenylbutanoyl)piperidin-4-yl)methyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((S)-morpholin-3-yl)-3-(trifluoromethyl)phenyl)-3,7-dihydro-4H-pyrrolo[2,3-d]pyrimidin-4-one
6-Chloro-3-((4-hydroxy-1-((R)-4,4,4-trifluoro-3-phenylbutanoyl)piperidin-4-yl)methyl)-7-(4-((R)-morpholin-3-yl)-3-(trifluoromethyl)phenyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-(morpholin-2-yl)phenyl)-3,7-dihydro-4H-pyrrolo[2,3-d]pyrimidin-4-one.
6-Chloro-3-((1-(4-chloro-2-methylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(4-chloro-2-methylbenzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-(difluoromethyl)benzoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(4-(difluoromethyl)benzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-(4-cyclopropylbenzoyl)-4-hydroxypiperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((4-hydroxy-1-(4-(1-methylcyclopropyl)benzoyl)piperidin-4-yl)methyl)-7-(3-methyl-4-((3S,6R)-6-methylmorpholin-3-yl)phenyl)-3,7-dihydro-4H-pyrrolo[2,3-d]pyrimidin-4-one
6-Chloro-3-((1-((1 S,2S)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one
6-Chloro-3-((1-((1R,2R)-1,2-dimethylcyclopropane-1-carbonyl)-4-hydroxypiperidin-4-yl)methyl)-7-(4-((3R,6S)-6-ethylmorpholin-3-yl)phenyl)-3,7-dihydro-4H-pyrrolo[2,3-d]pyrimidin-4-one
7-(4-((1 S,2R,5R)-8-Oxa-3-azabicyclo[3.2.1]octan-2-yl)phenyl)-6-chloro-3-((1-(4-chlorobenzoyl)-4-hydroxypiperidin-4-yl)methyl)-3,7-dihydro-4H-pyrrolo[2,3-d]pyrimidin-4-one
(R)-6-Chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
(S)-6-Chloro-7-(4-(6,6-dimethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((1-(2,2-dicyclopropylacetyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-7-(4-((3R,6S)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((4-hydroxy-1-(1-methylcyclobutane-1-carbonyl)piperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-3-((1-(3-cyclopropylpropanoyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one
6-Chloro-7-(4-((3S,6R)-6-cyclopropylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-7-(4-((3S,6R)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-7-(4-((3R,6S)-6-ethylmorpholin-3-yl)phenyl)-3-((4-hydroxy-1-(1-methylcyclopropane-1-carbonyl)piperidin-4-yl)methyl)-3,7-dihydro-4H-pyrrolo[2,3-d]pyrimidin-4-one
6-Chloro-3-((4-hydroxy-1-(1-methylcyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((1 S,2S)-1-methyl-2-(trifluoromethyl)cyclopropane-1-carbonyl)piperidin-4-yl)methyl)-7-(4-((3S,6R)-6-methylmorpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((S)-morpholin-3-yl)phenyl)-3,7-dihydro-4H-pyrrolo[2,3-d]pyrimidin-4-one
6-Chloro-3-((4-hydroxy-1-((1R,2R)-1-methyl-2-(trifluoromethyl)cyclopropane-1-carbonyl)piperidin-4-yl)methyl)-7-(4-((R)-morpholin-3-yl)phenyl)-3,7-dihydro-4H-pyrrolo[2,3-d]pyrimidin-4-one
6-Chloro-3-((1-((1 S,2S)-2-(difluoromethyl)-1-methylcyclopropane-1-carbonyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one, and
6-Chloro-3-((1-((1R,2R)-2-(difluoromethyl)-1-methylcyclopropane-1-carbonyl)-4-hydroxypiperidin-4-yl)methyl)-7-(4-((3R,6S)-6-methylmorpholin-3-yl)phenyl)-3,7-dihydro-4H-pyrrolo[2,3-d]pyrimidin-4-one.

35. A pharmaceutical composition comprising a compound according to claim 1, or a pharmaceutically acceptable salt or solvate thereof.

36. (canceled)

37. (canceled)

38. (canceled)

39. A method of treating cancer in a subject comprising administering to the subject an effective amount of a compound according to claim 1, or a pharmaceutically acceptable salt or solvate thereof, or a pharmaceutical composition according to claim 35.

40. The method according to claim 39, wherein the method comprises administering a further therapeutic agent.

41. The method according to claim 39, wherein the method further comprises administering an immune checkpoint inhibitor.
